SEQUENCE LISTING (1289 sequences)

SEQ ID NO: 1 - DNA, 72 bp, synthetic construct
  source 1..72: mol_type = other DNA
gggggtatagctcagtggtagagcgcgtgcttagcatgcacgaggtcctgggttcgatccccagtacctcca

SEQ ID NO: 2 - DNA, 73 bp, synthetic construct
  source 1..73: mol_type = other DNA
ggggaattagctcaagtggtagagcgcttgcttagcacgcaagaggtagtgggatcgatgcccacattctcca

SEQ ID NO: 3 - DNA, 73 bp, synthetic construct
  source 1..73: mol_type = other DNA
ggggaattagctcaaatggtagagcgctcgcttagcatgcgagaggtagcgggatcgatgcccgcattctcca

SEQ ID NO: 4 - DNA, 73 bp, synthetic construct
  source 1..73: mol_type = other DNA
ggggaattagctcaagtggtagagcgcttgcttagcatgcaagaggtagtgggatcgatgcccacattctcca

SEQ ID NO: 5 - DNA, 73 bp, synthetic construct
  source 1..73: mol_type = other DNA
ggggaattagctcaagcggtagagcgcttgcttagcatgcaagaggtagtgggatcgatgcccacattctcca

SEQ ID NO: 6 - DNA, 73 bp, synthetic construct
  source 1..73: mol_type = other DNA
ggggaattagctcaagtggtagagcgcttgcttagcatgcaagaggtagtgggatcaatgcccacattctcca

SEQ ID NO: 7 - DNA, 73 bp, synthetic construct
  source 1..73: mol_type = other DNA
ggggaattagctcaagtggtagagcgctcgcttagcatgcgagaggtagtgggatcgatgcccgcattctcca

SEQ ID NO: 8 - DNA, 73 bp, synthetic construct
  source 1..73: mol_type = other DNA
ggggaattagcccaagtggtagagcgcttgcttagcatgcaagaggtagtgggatcgatgcccacattctcca

SEQ ID NO: 9 - DNA, 72 bp, synthetic construct
  source 1..72: mol_type = other DNA
gggggtgtagctcagtggtagagcgcgtgcttagcatgcacgaggccccgggttcaatccccggcacctcca

SEQ ID NO: 10 - DNA, 72 bp, synthetic construct
  source 1..72: mol_type = other DNA
gggggtgtagctcagtggtagagcgcgtgcttagcatgtacgaggtcccgggttcaatccccggcacctcca

SEQ ID NO: 11 - DNA, 72 bp, synthetic construct
  source 1..72: mol_type = other DNA
ggggatgtagctcagtggtagagcgcatgcttagcatgcatgaggtcccgggttcgatccccagcatctcca

SEQ ID NO: 12 - DNA, 72 bp, synthetic construct
  source 1..72: mol_type = other DNA
gggggtgtagctcagtggtagagcgcgtgcttagcatgcacgaggccctgggttcaatccccagcacctcca

SEQ ID NO: 13 - DNA, 72 bp, synthetic construct
  source 1..72: mol_type = other DNA
gggggtatagctcagcggtagagcgcgtgcttagcatgcacgaggtcctgggttcaatccccaatacctcca

SEQ ID NO: 14 - DNA, 72 bp, synthetic construct
  source 1..72: mol_type = other DNA
gggggtgtagctcagtggtagagcgcgtgcttagcatgcacgaggccccgggttcaatccctggcacctcca

SEQ ID NO: 15 - DNA, 73 bp, synthetic construct
  source 1..73: mol_type = other DNA
gggggattagctcaaatggtagagcgctcgcttagcatgcgagaggtagcgggatcgatgcccgcatcctcca

SEQ ID NO: 16 - DNA, 73 bp, synthetic construct
  source 1..73: mol_type = other DNA
ggggaattagctcaggcggtagagcgctcgcttagcatgcgagaggtagcgggatcgacgcccgcattctcca

SEQ ID NO: 17 - DNA, 72 bp, synthetic construct
  source 1..72: mol_type = other DNA
ggggatgtagctcagtggtagagcgcatgcttcgcatgtatgaggtcccgggttcgatccccggcatctcca

SEQ ID NO: 18 - DNA, 72 bp, synthetic construct
  source 1..72: mol_type = other DNA
ggggatgtagctcagtggtagagcgcatgcttcgcatgtatgaggccccgggttcgatccccggcatctcca

SEQ ID NO: 19 - DNA, 72 bp, synthetic construct
  source 1..72: mol_type = other DNA
ggggatgtagctcagtggtagagcgcgcgcttcgcatgtgtgaggtcccgggttcaatccccggcatctcca

SEQ ID NO: 20 - DNA, 72 bp, synthetic construct
  source 1..72: mol_type = other DNA
gggggtgtagctcagtggtagagcgcgtgcttcgcatgtacgaggccccgggttcgacccccggctcctcca

SEQ ID NO: 21 - DNA, 72 bp, synthetic construct
  source 1..72: mol_type = other DNA
gggggtgtagctcagtggtagagcgcatgctttgcatgtatgaggtcccgggttcgatccccggcacctcca

SEQ ID NO: 22 - DNA, 72 bp, synthetic construct
  source 1..72: mol_type = other DNA
ggggatgtagctcagtggtagagcgcatgctttgcatgtatgaggtcccgggttcgatccccggcatctcca

SEQ ID NO: 23 - DNA, 72 bp, synthetic construct
  source 1..72: mol_type = other DNA
ggggatgtagctcagtggtagagcgcatgctttgcatgtatgaggccccgggttcgatccccggcatctcca

SEQ ID NO: 24 - DNA, 72 bp, synthetic construct
  source 1..72: mol_type = other DNA
ggggatgtagctcagtggtagagcgcatgctttgcacgtatgaggccccgggttcaatccccggcatctcca

SEQ ID NO: 25 - DNA, 72 bp, synthetic construct
  source 1..72: mol_type = other DNA
gggggtgtagctcagtggtagagcgcatgctttgcatgtatgaggcctcgggttcgatccccgacacctcca

SEQ ID NO: 26 - DNA, 72 bp, synthetic construct
  source 1..72: mol_type = other DNA
gggggtgtagctcagtggtagagcacatgctttgcatgtgtgaggccccgggttcgatccccggcacctcca

SEQ ID NO: 27 - DNA, 71 bp, synthetic construct
  source 1..71: mol_type = other DNA
gggggtgtagctcagtggtagagcgcatgctttgcatgtatgaggcctcggttcgatccccgacacctcca

SEQ ID NO: 28 - DNA, 73 bp, synthetic construct
  source 1..73: mol_type = other DNA
gggccagtggcgcaatggataacgcgtctgactacggatcagaagattccaggttcgactcctggctggctcg

SEQ ID NO: 29 - DNA, 73 bp, synthetic construct
  source 1..73: mol_type = other DNA
gggccagtggcgcaatggataacgcgtctgactacggatcagaagattctaggttcgactcctggctggctcg

SEQ ID NO: 30 - DNA, 73 bp, synthetic construct
  source 1..73: mol_type = other DNA
ggccgcgtggcctaatggataaggcgtctgattccggatcagaagattgagggttcgagtcccttcgtggtcg

SEQ ID NO: 31 - DNA, 73 bp, synthetic construct
  source 1..73: mol_type = other DNA
gacccagtggcctaatggataaggcatcagcctccggagctggggattgtgggttcgagtcccatctgggtcg

SEQ ID NO: 32 - DNA, 73 bp, synthetic construct
  source 1..73: mol_type = other DNA
gccccagtggcctaatggataaggcactggcctcctaagccagggattgtgggttcgagtcccacctggggta

SEQ ID NO: 33 - DNA, 73 bp, synthetic construct
  source 1..73: mol_type = other DNA
gccccagtggcctaatggataaggcactggcctcctaagccagggattgtgggttcgagtcccacctggggtg

SEQ ID NO: 34 - DNA, 73 bp, synthetic construct
  source 1..73: mol_type = other DNA
gccccggtggcctaatggataaggcattggcctcctaagccagggattgtgggttcgagtcccacccggggta

SEQ ID NO: 35 - DNA, 73 bp, synthetic construct
  source 1..73: mol_type = other DNA
gccccagtggcctaatggataaggcattggcctcctaagccagggattgtgggttcgagtcccatctggggtg

SEQ ID NO: 36 - DNA, 73 bp, synthetic construct
  source 1..73: mol_type = other DNA
gccccagtggcctgatggataaggtactggcctcctaagccagggattgtgggttcgagttccacctggggta

SEQ ID NO: 37 - DNA, 73 bp, synthetic construct
  source 1..73: mol_type = other DNA
ggccgcgtggcctaatggataaggcgtctgacttcggatcagaagattgcaggttcgagtcctgccgcggtcg

SEQ ID NO: 38 - DNA, 73 bp, synthetic construct
  source 1..73: mol_type = other DNA
gaccacgtggcctaatggataaggcgtctgacttcggatcagaagattgagggttcgaatccctccgtggtta

SEQ ID NO: 39 - DNA, 73 bp, synthetic construct
  source 1..73: mol_type = other DNA
gaccgcgtggcctaatggataaggcgtctgacttcggatcagaagattgagggttcgagtcccttcgtggtcg

SEQ ID NO: 40 - DNA, 73 bp, synthetic construct
  source 1..73: mol_type = other DNA
gaccacgtggcctaatggataaggcgtctgacttcggatcagaagattgagggttcgaatcccttcgtggtta

SEQ ID NO: 41 - DNA, 73 bp, synthetic construct
  source 1..73: mol_type = other DNA
gaccacgtggcctaatggataaggcgtctgacttcggatcagaagattgagggttcgaatcccttcgtggttg

SEQ ID NO: 42 - DNA, 73 bp, synthetic construct
  source 1..73: mol_type = other DNA
ggccgtgtggcctaatggataaggcgtctgacttcggatcaaaagattgcaggtttgagttctgccacggtcg

SEQ ID NO: 43 - DNA, 85 bp, synthetic construct
  source 1..85: mol_type = other DNA
ggctccgtggcgcaatggatagcgcattggacttctagaggctgaaggcattcaaaggttccgggttcgagtcccggcggagtcg

SEQ ID NO: 44 - DNA, 88 bp, synthetic construct
  source 1..88: mol_type = other DNA
ggctctgtggcgcaatggatagcgcattggacttctagtgacgaatagagcaattcaaaggttgtgggttcgaatcccaccagagtcg

SEQ ID NO: 45 - DNA, 91 bp, synthetic construct
  source 1..91: mol_type = other DNA
ggctctgtggcgcaatggatagcgcattggacttctagctgagcctagtgtggtcattcaaaggttgtgggttcgagtcccaccagagtcg

SEQ ID NO: 46 - DNA, 86 bp, synthetic construct
  source 1..86: mol_type = other DNA
ggctctgtggcgcaatggatagcgcattggacttctagatagttagagaaattcaaaggttgtgggttcgagtcccaccagagtcg

SEQ ID NO: 47 - DNA, 74 bp, synthetic construct
  source 1..74: mol_type = other DNA
gtctctgtggcgcaatggacgagcgcgctggacttctaatccagaggttccgggttcgagtcccggcagagatg

SEQ ID NO: 48 - DNA, 87 bp, synthetic construct
  source 1..87: mol_type = other DNA
ggctctgtggcgcaatggatagcgcattggacttctagcctaaatcaagagattcaaaggttgcgggttcgagtccctccagagtcg

SEQ ID NO: 49 - DNA, 74 bp, synthetic construct
  source 1..74: mol_type = other DNA
gtctctgtggcgcaatcggttagcgcgttcggctgttaaccgaaaggttggtggttcgatcccacccagggacg

SEQ ID NO: 50 - DNA, 74 bp, synthetic construct
  source 1..74: mol_type = other DNA
gtctctgtggcgcaatcggctagcgcgtttggctgttaactaaaaggttggcggttcgaacccacccagaggcg

SEQ ID NO: 51 - DNA, 74 bp, synthetic construct
  source 1..74: mol_type = other DNA
gtctctgtggtgcaatcggttagcgcgttccgctgttaaccgaaagcttggtggttcgagcccacccagggatg

SEQ ID NO: 52 - DNA, 74 bp, synthetic construct
  source 1..74: mol_type = other DNA
gtctctgtggcgcaatcggctagcgcgtttggctgttaactaaaaagttggtggttcgaacacacccagaggcg

SEQ ID NO: 53 - DNA, 74 bp, synthetic construct
  source 1..74: mol_type = other DNA
gtctctgtggcgcaatcggttagcgcgttcggctgttaaccgaaaggttggtggttcgagcccacccagggacg

SEQ ID NO: 54 - DNA, 74 bp, synthetic construct
  source 1..74: mol_type = other DNA
gtctctgtggcgcaatcggttagcgcattcggctgttaaccgaaaggttggtggttcgagcccacccagggacg

SEQ ID NO: 55 - DNA, 74 bp, synthetic construct
  source 1..74: mol_type = other DNA
gtctctgtggcgcaatcggttagcgcgttcggctgttaaccgaaagattggtggttcgagcccacccagggacg

SEQ ID NO: 56 - DNA, 74 bp, synthetic construct
  source 1..74: mol_type = other DNA
gtctctgtggcgcaatcggttagcgcgttcggctgttaactgaaaggttggtggttcgagcccacccagggacg

SEQ ID NO: 57 - DNA, 74 bp, synthetic construct
  source 1..74: mol_type = other DNA
gtctctgtggcgcaatgggttagcgcgttcggctgttaaccgaaaggttggtggttcgagcccatccagggacg

SEQ ID NO: 58 - DNA, 74 bp, synthetic construct
  source 1..74: mol_type = other DNA
gtctctgtggcgtagtcggttagcgcgttcggctgttaaccgaaaagttggtggttcgagcccacccaggaacg

SEQ ID NO: 59 - DNA, 74 bp, synthetic construct
  source 1..74: mol_type = other DNA
gtctctgtggcgcaatcggctagcgcgtttggctgttaactaaaaggttggtggttcgaacccacccagaggcg

SEQ ID NO: 60 - DNA, 74 bp, synthetic construct
  source 1..74: mol_type = other DNA
gtctctgtggcgcaatcggttagcgcgttcggctgttaactgaaaggttagtggttcgagcccacccggggacg

SEQ ID NO: 61 - DNA, 72 bp, synthetic construct
  source 1..72: mol_type = other DNA
tcctcgttagtatagtggttagtatccccgcctgtcacgcgggagaccggggttcaattccccgacggggag

SEQ ID NO: 62 - DNA, 72 bp, synthetic construct
  source 1..72: mol_type = other DNA
tcctcgttagtatagtggtgagtatccccgcctgtcacgcgggagaccggggttcgattccccgacggggag

SEQ ID NO: 63 - DNA, 72 bp, synthetic construct
  source 1..72: mol_type = other DNA
tcctcgttagtatagtggtgagtgtccccgtctgtcacgcgggagaccggggttcgattccccgacggggag

SEQ ID NO: 64 - DNA, 72 bp, synthetic construct
  source 1..72: mol_type = other DNA
gggggcatagctcagtggtagagcatttgactgcagatcaagaggtccctggttcaaatccaggtgccccct

SEQ ID NO: 65 - DNA, 72 bp, synthetic construct
  source 1..72: mol_type = other DNA
gggggtatagctcaggggtagagcatttgactgcagatcaagaggtccctggttcaaatccaggtgcccccc

SEQ ID NO: 66 - DNA, 72 bp, synthetic construct
  source 1..72: mol_type = other DNA
gggggtatagcttagcggtagagcatttgactgcagatcaagaggtccccggttcaaatccgggtgccccct

SEQ ID NO: 67 - DNA, 72 bp, synthetic construct
  source 1..72: mol_type = other DNA
gggggtatagcttaggggtagagcatttgactgcagatcaaaaggtccctggttcaaatccaggtgcccctt

SEQ ID NO: 68 - DNA, 72 bp, synthetic construct
  source 1..72: mol_type = other DNA
gggggtatagctcaggggtagagcatttgactgcagatcaagaggtccccagttcaaatctgggtgccccct

SEQ ID NO: 69 - DNA, 72 bp, synthetic construct
  source 1..72: mol_type = other DNA
gggggtatagctcaggggtagagcatttgactgcagatcaagaagtccccggttcaaatccgggtgccccct

SEQ ID NO: 70 - DNA, 72 bp, synthetic construct
  source 1..72: mol_type = other DNA
gggggtatagctcaggggtagagcatttgactgcagatcaagaggtctctggttcaaatccaggtgccccct

SEQ ID NO: 71 - DNA, 72 bp, synthetic construct
  source 1..72: mol_type = other DNA
gggggtatagctcaggggtagagcacttgactgcagatcaagaagtccttggttcaaatccaggtgccccct

SEQ ID NO: 72 - DNA, 72 bp, synthetic construct
  source 1..72: mol_type = other DNA
ggggatatagctcaggggtagagcatttgactgcagatcaagaggtccccggttcaaatccgggtgcccccc

SEQ ID NO: 73 - DNA, 72 bp, synthetic construct
  source 1..72: mol_type = other DNA
gggggtatagttcaggggtagagcatttgactgcagatcaagaggtccctggttcaaatccaggtgccccct

SEQ ID NO: 74 - DNA, 72 bp, synthetic construct
  source 1..72: mol_type = other DNA
gggggtatagctcaggggtagagcatttgactgcaaatcaagaggtccctgattcaaatccaggtgccccct

SEQ ID NO: 75 - DNA, 72 bp, synthetic construct
  source 1..72: mol_type = other DNA
gggggtatagctcagtggtagagcatttgactgcagatcaagaggtccccggttcaaatccgggtgccccct

SEQ ID NO: 76 - DNA, 72 bp, synthetic construct
  source 1..72: mol_type = other DNA
gggcgtatagctcaggggtagagcatttgactgcagatcaagaggtccccagttcaaatctgggtgccccct

SEQ ID NO: 77 - DNA, 72 bp, synthetic construct
  source 1..72: mol_type = other DNA
gggggtatagctcacaggtagagcatttgactgcagatcaagaggtccccggttcaaatctgggtgccccct

SEQ ID NO: 78 - DNA, 70 bp, synthetic construct
  source 1..70: mol_type = other DNA
gggcgtatagctcaggggtagagcatttgactgcagatcaagaggtccccagttcaaatctgggtgccca

SEQ ID NO: 79 - DNA, 72 bp, synthetic construct
  source 1..72: mol_type = other DNA
gggggtatagctcacaggtagagcatttgactgcagatcaagaggtccccggttcaaatccggttactccct

SEQ ID NO: 80 - DNA, 72 bp, synthetic construct
  source 1..72: mol_type = other DNA
gggggtatagctcaggggtagagcacttgactgcagatcaagaggtccctggttcaaatccaggtgccccct

SEQ ID NO: 81 - DNA, 72 bp, synthetic construct
  source 1..72: mol_type = other DNA
gggggtatagctcagtggtagagcatttgactgcagatcaagaggtccctggttcaaatccgggtgccccct

SEQ ID NO: 82 - DNA, 73 bp, synthetic construct
  source 1..73: mol_type = other DNA
gggggtatagctcagtgggtagagcatttgactgcagatcaagaggtccccggttcaaatccgggtgccccct

SEQ ID NO: 83 - DNA, 72 bp, synthetic construct
  source 1..72: mol_type = other DNA
gggggtgtagctcagtggtagagcatttgactgcagatcaagaggtccctggttcaaatccaggtgccccct

SEQ ID NO: 84 - DNA, 73 bp, synthetic construct
  source 1..73: mol_type = other DNA
gggggtatagctcaggtggtagagcatttgactgcagatcaagaggtccccggttcaaatccgggtgccccct

SEQ ID NO: 85 - DNA, 72 bp, synthetic construct
  source 1..72: mol_type = other DNA
gggggtatagctcaggggtagagcatttgactgcagatcaagaggtccccggttcaaatccgggtgccccct

SEQ ID NO: 86 - DNA, 72 bp, synthetic construct
  source 1..72: mol_type = other DNA
gggggtatagctcaggggtagagcatttgactgcagatcaagaggtccctggttcaaatccaggtgccccct

SEQ ID NO: 87 - DNA, 72 bp, synthetic construct
  source 1..72: mol_type = other DNA
ggttccatggtgtaatggttagcactctggactctgaatccagcgatccgagttcaaatctcggtggaacct

SEQ ID NO: 88 - DNA, 72 bp, synthetic construct
  source 1..72: mol_type = other DNA
ggttccatggtgtaatggttagcactctggactctgaatccagcgatccgagttcaagtctcggtggaacct

SEQ ID NO: 89 - DNA, 72 bp, synthetic construct
  source 1..72: mol_type = other DNA
ggttccatggtgtaatggtgagcactctggactctgaatccagcgatccgagttcgagtctcggtggaacct

SEQ ID NO: 90 - DNA, 72 bp, synthetic construct
  source 1..72: mol_type = other DNA
ggttccatggtgtaatggtaagcactctggactctgaatccagcgatccgagttcgagtctcggtggaacct

SEQ ID NO: 91 - DNA, 72 bp, synthetic construct
  source 1..72: mol_type = other DNA
ggttccatggtgtaatggttagcactctggactctgaatccggtaatccgagttcaaatctcggtggaacct

SEQ ID NO: 92 - DNA, 72 bp, synthetic construct
  source 1..72: mol_type = other DNA
ggccccatggtgtaatggtcagcactctggactctgaatccagcgatccgagttcaaatctcggtgggaccc

SEQ ID NO: 93 - DNA, 72 bp, synthetic construct
  source 1..72: mol_type = other DNA
ggttccatggtgtaatggtaagcactctggactctgaatccagccatctgagttcgagtctctgtggaacct

SEQ ID NO: 94 - DNA, 72 bp, synthetic construct
  source 1..72: mol_type = other DNA
ggtcccatggtgtaatggttagcactctggactttgaatccagcgatccgagttcaaatctcggtgggacct

SEQ ID NO: 95 - DNA, 72 bp, synthetic construct
  source 1..72: mol_type = other DNA
ggtcccatggtgtaatggttagcactctggactttgaatccagcaatccgagttcgaatctcggtgggacct

SEQ ID NO: 96 - DNA, 72 bp, synthetic construct
  source 1..72: mol_type = other DNA
ggccccatggtgtaatggttagcactctggactttgaatccagcgatccgagttcaaatctcggtgggacct

SEQ ID NO: 97 - DNA, 72 bp, synthetic construct
  source 1..72: mol_type = other DNA
ggtcccatggtgtaatggttagcactctgggctttgaatccagcaatccgagttcgaatcttggtgggacct

SEQ ID NO: 98 - DNA, 72 bp, synthetic construct
  source 1..72: mol_type = other DNA
tccctggtggtctagtggttaggattcggcgctctcaccgccgcggcccgggttcgattcccggtcagggaa

SEQ ID NO: 99 - DNA, 72 bp, synthetic construct
  source 1..72: mol_type = other DNA
tccctggtggtctagtggttaggattcggcgctctcaccgccgcggcccgggttcgattcccggtcaggaaa

SEQ ID NO: 100 - DNA, 72 bp, synthetic construct
  source 1..72: mol_type = other DNA
tcccatatggtctagcggttaggattcctggttttcacccaggtggcccgggttcgactcccggtatgggaa

SEQ ID NO: 101 - DNA, 72 bp, synthetic construct
  source 1..72: mol_type = other DNA
tcccacatggtctagcggttaggattcctggttttcacccaggcggcccgggttcgactcccggtgtgggaa

SEQ ID NO: 102 - DNA, 72 bp, synthetic construct
  source 1..72: mol_type = other DNA
tccctggtggtctagtggctaggattcggcgctttcaccgccgcggcccgggttcgattcccggccagggaa

SEQ ID NO: 103 - DNA, 72 bp, synthetic construct
  source 1..72: mol_type = other DNA
tccctggtggtctagtggctaggattcggcgctttcaccgccgcggcccgggttcgattcccggtcagggaa

SEQ ID NO: 104 - DNA, 71 bp, synthetic construct
  source 1..71: mol_type = other DNA
gcattggtggttcagtggtagaattctcgcctcccacgcgggagacccgggttcaattcccggccaatgca

SEQ ID NO: 105 - DNA, 71 bp, synthetic construct
  source 1..71: mol_type = other DNA
gcgccgctggtgtagtggtatcatgcaagattcccattcttgcgacccgggttcgattcccgggcggcgca

SEQ ID NO: 106 - DNA, 71 bp, synthetic construct
  source 1..71: mol_type = other DNA
gcattggtggttcaatggtagaattctcgcctcccacgcaggagacccaggttcgattcctggccaatgca

SEQ ID NO: 107 - DNA, 71 bp, synthetic construct
  source 1..71: mol_type = other DNA
gcatgggtggttcagtggtagaattctcgcctgccacgcgggaggcccgggttcgattcccggcccatgca

SEQ ID NO: 108 - DNA, 71 bp, synthetic construct
  source 1..71: mol_type = other DNA
gcattggtggttcagtggtagaattctcgcctgccacgcgggaggcccgggttcgattcccggccaatgca

SEQ ID NO: 109 - DNA, 71 bp, synthetic construct
  source 1..71: mol_type = other DNA
gcattggtggttcagtggtagaattctcgcctgccacgcgggaggcccgggtttgattcccggccagtgca

SEQ ID NO: 110 - DNA, 71 bp, synthetic construct
  source 1..71: mol_type = other DNA
gcataggtggttcagtggtagaattcttgcctgccacgcaggaggcccaggtttgattcctggcccatgca

SEQ ID NO: 111 - DNA, 71 bp, synthetic construct
  source 1..71: mol_type = other DNA
gcattggtggttcagtggtagaattctcgcctgccatgcgggcggccgggcttcgattcctggccaatgca

SEQ ID NO: 112 - DNA, 72 bp, synthetic construct
  source 1..72: mol_type = other DNA
gcgttggtggtatagtggttagcatagctgccttccaagcagttgacccgggttcgattcccggccaacgca

SEQ ID NO: 113 - DNA, 72 bp, synthetic construct
  source 1..72: mol_type = other DNA
gcgttggtggtatagtggtgagcatagctgccttccaagcagttgacccgggttcgattcccggccaacgca

SEQ ID NO: 114 - DNA, 72 bp, synthetic construct
  source 1..72: mol_type = other DNA
gcgttggtggtatagtggtaagcatagctgccttccaagcagttgacccgggttcgattcccggccaacgca

SEQ ID NO: 115 - DNA, 72 bp, synthetic construct
  source 1..72: mol_type = other DNA
gcgttggtggtatagtggtgagcatagttgccttccaagcagttgacccgggctcgattcccgcccaacgca

SEQ ID NO: 116 - DNA, 72 bp, synthetic construct
  source 1..72: mol_type = other DNA
gccgtgatcgtatagtggttagtactctgcgttgtggccgcagcaacctcggttcgaatccgagtcacggca

SEQ ID NO: 117 - DNA, 72 bp, synthetic construct
  source 1..72: mol_type = other DNA
gccatgatcgtatagtggttagtactctgcgctgtggccgcagcaacctcggttcgaatccgagtcacggca

SEQ ID NO: 118 - DNA, 74 bp, synthetic construct
  source 1..74: mol_type = other DNA
ggccggttagctcagttggttagagcgtggcgctaataacgccaaggtcgcgggttcgatccccgtacgggcca

SEQ ID NO: 119 - DNA, 74 bp, synthetic construct
  source 1..74: mol_type = other DNA
ggccggttagctcagttggttagagcgtggtgctaataacgccaaggtcgcgggttcgatccccgtactggcca

SEQ ID NO: 120 - DNA, 74 bp, synthetic construct
  source 1..74: mol_type = other DNA
ggctggttagctcagttggttagagcgtggtgctaataacgccaaggtcgcgggttcgatccccgtactggcca

SEQ ID NO: 121 - DNA, 74 bp, synthetic construct
  source 1..74: mol_type = other DNA
ggccggttagctcagttggttagagcgtggtgctaataacgccaaggtcgcgggttcgaaccccgtacgggcca

SEQ ID NO: 122 - DNA, 74 bp, synthetic construct
  source 1..74: mol_type = other DNA
ggccggttagctcagttggttagagcgtggtgctaataacgccaaggtcgcgggttcgatccccgtacgggcca

SEQ ID NO: 123 - DNA, 74 bp, synthetic construct
  source 1..74: mol_type = other DNA
ggccggttagctcagttggttagagcgtggtgctaataacgctaaggtcgcgggttcgatccccgtactggcca

SEQ ID NO: 124 - DNA, 74 bp, synthetic construct
  source 1..74: mol_type = other DNA
ggccggttagctcagttggtcagagcgtggtgctaataacgccaaggtcgcgggttcgatccccgtacgggcca

SEQ ID NO: 125 - DNA, 74 bp, synthetic construct
  source 1..74: mol_type = other DNA
ggccggttagctcagtcggctagagcgtggtgctaataacgccaaggtcgcgggttcgatccccgtacgggcca

SEQ ID NO: 126 - DNA, 74 bp, synthetic construct
  source 1..74: mol_type = other DNA
ggctggttagttcagttggttagagcgtggtgctaataacgccaaggtcgtgggttcgatccccatatcggcca

SEQ ID NO: 127 - DNA, 74 bp, synthetic construct
  source 1..74: mol_type = other DNA
ggccggttagctcagttggtaagagcgtggtgctgataacaccaaggtcgcgggctcgactcccgcaccggcca

SEQ ID NO: 128 - DNA, 93 bp, synthetic construct
  source 1..93: mol_type = other DNA
gctccagtggcgcaatcggttagcgcgcggtacttatatgacagtgcgagcggagcaatgccgaggttgtgagttcgatcctcacctggagca

SEQ ID NO: 129 - DNA, 93 bp, synthetic construct
  source 1..93: mol_type = other DNA
gctccagtggcgcaatcggttagcgcgcggtacttatacagcagtacatgcagagcaatgccgaggttgtgagttcgagcctcacctggagca

SEQ ID NO: 130 - DNA, 94 bp, synthetic construct
  source 1..94: mol_type = other DNA
gctccagtggcgcaatcggttagcgcgcggtacttatatggcagtatgtgtgcgagtgatgccgaggttgtgagttcgagcctcacctggagca

SEQ ID NO: 131 - DNA, 94 bp, synthetic construct
  source 1..94: mol_type = other DNA
gctccagtggcgcaatcggttagcgcgcggtacttatacaacagtatatgtgcgggtgatgccgaggttgtgagttcgagcctcacctggagca

SEQ ID NO: 132 - DNA, 94 bp, synthetic construct
  source 1..94: mol_type = other DNA
gctccagtggcgcaatcggttagcgcgcggtacttataagacagtgcacctgtgagcaatgccgaggttgtgagttcaagcctcacctggagca

SEQ ID NO: 133 - DNA, 82 bp, synthetic construct
  source 1..82: mol_type = other DNA
ggtagcgtggccgagcggtctaaggcgctggattaaggctccagtctcttcggaggcgtgggttcgaatcccaccgctgcca

SEQ ID NO: 134 - DNA, 82 bp, synthetic construct
  source 1..82: mol_type = other DNA
ggtagcgtggccgagcggtctaaggcgctggattaaggctccagtctcttcgggggcgtgggttcgaatcccaccgctgcca

SEQ ID NO: 135 - DNA, 82 bp, synthetic construct
  source 1..82: mol_type = other DNA
ggtagcgtggccgagcggtctaaggcgctggattaaggctccagtctcttcgggggcgtgggttcaaatcccaccgctgcca

SEQ ID NO: 136 - DNA, 82 bp, synthetic construct
  source 1..82: mol_type = other DNA
ggtagcgtggccgagtggtctaagacgctggattaaggctccagtctcttcgggggcgtgggtttgaatcccaccgctgcca

SEQ ID NO: 137 - DNA, 106 bp, synthetic construct
  source 1..106: mol_type = other DNA
gtcaggatggccgagtggtctaaggcgccagactcaagctaagcttcctccgcggtggggattctggtctccaatggaggcgtgggttcgaatcccacttctgaca

SEQ ID NO: 138 - DNA, 105 bp, synthetic construct
  source 1..105: mol_type = other DNA
gtcaggatggccgagtggtctaaggcgccagactcaagcttggcttcctcgtgttgaggattctggtctccaatggaggcgtgggttcgaatcccacttctgaca

SEQ ID NO: 139 - DNA, 108 bp, synthetic construct
  source 1..108: mol_type = other DNA
gtcaggatggccgagtggtctaaggcgccagactcaagcttactgcttcctgtgttcgggtcttctggtctccgtatggaggcgtgggttcgaatcccacttctgaca

SEQ ID NO: 140 - DNA, 107 bp, synthetic construct
  source 1..107: mol_type = other DNA
gtcaggatggccgagtggtctaaggcgccagactcaagttgctacttcccaggtttggggcttctggtctccgcatggaggcgtgggttcgaatcccacttctgaca

SEQ ID NO: 141 - DNA, 106 bp, synthetic construct
  source 1..106: mol_type = other DNA
gtcaggatggccgagtggtctaaggcgccagactcaaggtaagcaccttgcctgcgggctttctggtctccggatggaggcgtgggttcgaatcccacttctgaca

SEQ ID NO: 142 - DNA, 74 bp, synthetic construct
  source 1..74: mol_type = other DNA
gcctccttagtgcagtaggtagcgcatcagtctcaaaatctgaatggtcctgagttcaagcctcagagggggca

SEQ ID NO: 143 - DNA, 84 bp, synthetic construct
  source 1..84: mol_type = other DNA
gtcaggatggccgagcagtcttaaggcgctgcgttcaaatcgcaccctccgctggaggcgtgggttcgaatcccacttttgaca

SEQ ID NO: 144 - DNA, 83 bp, synthetic construct
  source 1..83: mol_type = other DNA
gtcaggatggccgagcggtctaaggcgctgcgttcaggtcgcagtctcccctggaggcgtgggttcgaatcccactcctgaca

SEQ ID NO: 145 - DNA, 83 bp, synthetic construct
  source 1..83: mol_type = other DNA
gtcaggatggccgagcggtctaaggcgctgcgttcaggtcgcagtctcccctggaggcgtgggttcgaatcccacttctgaca

SEQ ID NO: 146 - DNA, 83 bp, synthetic construct
  source 1..83: mol_type = other DNA
accaggatggccgagtggttaaggcgttggacttaagatccaatggacatatgtccgcgtgggttcgaaccccactcctggta

SEQ ID NO: 147 - DNA, 83 bp, synthetic construct
  source 1..83: mol_type = other DNA
accgggatggccgagtggttaaggcgttggacttaagatccaatgggctggtgcccgcgtgggttcgaaccccactctcggta

SEQ ID NO: 148 - DNA, 83 bp, synthetic construct
  source 1..83: mol_type = other DNA
accagaatggccgagtggttaaggcgttggacttaagatccaatggattcatatccgcgtgggttcgaaccccacttctggta

SEQ ID NO: 149 - DNA, 83 bp, synthetic construct
  source 1..83: mol_type = other DNA
accgggatggctgagtggttaaggcgttggacttaagatccaatggacaggtgtccgcgtgggttcgagccccactcccggta

SEQ ID NO: 150 - DNA, 82 bp, synthetic construct
  source 1..82: mol_type = other DNA
ggtagcgtggccgagcggtctaaggcgctggatttaggctccagtctcttcggaggcgtgggttcgaatcccaccgctgcca

SEQ ID NO: 151 - DNA, 82 bp, synthetic construct
  source 1..82: mol_type = other DNA
ggtagtgtggccgagcggtctaaggcgctggatttaggctccagtctcttcgggggcgtgggttcgaatcccaccactgcca

SEQ ID NO: 152 - DNA, 82 bp, synthetic construct
  source 1..82: mol_type = other DNA
ggtagcgtggccgagtggtctaaggcgctggatttaggctccagtcatttcgatggcgtgggttcgaatcccaccgctgcca

SEQ ID NO: 153 - DNA, 73 bp, synthetic construct
  source 1..73: mol_type = other DNA
gcccggctagctcagtcggtagagcatgggactcttaatcccagggtcgtgggttcgagccccacgttgggcg

SEQ ID NO: 154 - DNA, 73 bp, synthetic construct
  source 1..73: mol_type = other DNA
gcccagctagctcagtcggtagagcataagactcttaatctcagggttgtggattcgtgccccatgctgggtg

SEQ ID NO: 155 - DNA, 74 bp, synthetic construct
  source 1..74: mol_type = other DNA
gcagctagctcagtcggtagagcatgagactcttaatctcagggtcatgggttcgtgccccatgttgggtgcca

SEQ ID NO: 156 - DNA, 73 bp, synthetic construct
  source 1..73: mol_type = other DNA
gcccggctagctcagtcggtagagcatgagactcttaatctcagggtcgtgggttcgagccccacgttgggcg

SEQ ID NO: 157 - DNA, 73 bp, synthetic construct
  source 1..73: mol_type = other DNA
gcccggctagctcagtcggtagagcatgagacccttaatctcagggtcgtgggttcgagccccacgttgggcg

SEQ ID NO: 158 - DNA, 73 bp, synthetic construct
  source 1..73: mol_type = other DNA
gcccggctagctcagtcggtagagcatgggactcttaatctcagggtcgtgggttcgagccccacgttgggcg

SEQ ID NO: 159 - DNA, 73 bp, synthetic construct
  source 1..73: mol_type = other DNA
gcccggctagctcagtcgatagagcatgagactcttaatctcagggtcgtgggttcgagccgcacgttgggcg

SEQ ID NO: 160 - DNA, 73 bp, synthetic construct
  source 1..73: mol_type = other DNA
gcccagctagctcagtcggtagagcatgagactcttaatctcagggtcatgggtttgagccccacgtttggtg

SEQ ID NO: 161 - DNA, 73 bp, synthetic construct
  source 1..73: mol_type = other DNA
gcctggctagctcagtcggcaaagcatgagactcttaatctcagggtcgtgggctcgagctccatgttgggcg

SEQ ID NO: 162 - DNA, 73 bp, synthetic construct
  source 1..73: mol_type = other DNA
gcccgactacctcagtcggtggagcatgggactcttcatcccagggttgtgggttcgagccccacattgggca

SEQ ID NO: 163 - DNA, 73 bp, synthetic construct
  source 1..73: mol_type = other DNA
gcctggatagctcagttggtagagcatcagacttttaatctgagggtccagggttcaagtccctgttcaggca

SEQ ID NO: 164 - DNA, 73 bp, synthetic construct
  source 1..73: mol_type = other DNA
acccagatagctcagtcagtagagcatcagacttttaatctgagggtccaaggttcatgtccctttttgggtg

SEQ ID NO: 165 - DNA, 73 bp, synthetic construct
  source 1..73: mol_type = other DNA
gcctggatagctcagttggtagagcatcagacttttaatctgagggtccagggttcaagtccctgttcaggcg

SEQ ID NO: 166 - DNA, 73 bp, synthetic construct
  source 1..73: mol_type = other DNA
gcccggatagctcagtcggtagagcatcagacttttaatctgagggtccagggttcaagtccctgttcgggcg

SEQ ID NO: 167 - DNA, 73 bp, synthetic construct
  source 1..73: mol_type = other DNA
gcctggatagctcagtcggtagagcatcagacttttaatctgagggtccagggttcaagtccctgttcaggcg

SEQ ID NO: 168 - DNA, 73 bp, synthetic construct
  source 1..73: mol_type = other DNA
gcccggatagctcagtcggtagagcatcagacttttaatctgagggtccggggttcaagtccctgttcgggcg

SEQ ID NO: 169 - DNA, 73 bp, synthetic construct
  source 1..73: mol_type = other DNA
gcctgggtagctcagtcggtagagcatcagacttttaatctgagggtccagggttcaagtccctgtccaggcg

SEQ ID NO: 170 - DNA, 73 bp, synthetic construct
  source 1..73: mol_type = other DNA
gcctggatagctcagttggtagaacatcagacttttaatctgacggtgcagggttcaagtccctgttcaggcg

SEQ ID NO: 171 - DNA, 73 bp, synthetic construct
  source 1..73: mol_type = other DNA
gcctcgttagcgcagtaggtagcgcgtcagtctcataatctgaaggtcgtgagttcgatcctcacacggggca

SEQ ID NO: 172 - DNA, 73 bp, synthetic construct
  source 1..73: mol_type = other DNA
gccctcttagcgcagtgggcagcgcgtcagtctcataatctgaaggtcctgagttcgagcctcagagagggca

SEQ ID NO: 173 - DNA, 73 bp, synthetic construct
  source 1..73: mol_type = other DNA
gcctccttagcgcagtaggcagcgcgtcagtctcataatctgaaggtcctgagttcgaacctcagagggggca

SEQ ID NO: 174 - DNA, 73 bp, synthetic construct
  source 1..73: mol_type = other DNA
gccctcttagcgcagcgggcagcgcgtcagtctcataatctgaaggtcctgagttcgagcctcagagagggca

SEQ ID NO: 175 - DNA, 73 bp, synthetic construct
  source 1..73: mol_type = other DNA
gccctcttagcgcagctggcagcgcgtcagtctcataatctgaaggtcctgagttcaagcctcagagagggca

SEQ ID NO: 176 - DNA, 73 bp, synthetic construct
  source 1..73: mol_type = other DNA
gcctcgttagcgcagtaggcagcgcgtcagtctcataatctgaaggtcgtgagttcgagcctcacacggggca

SEQ ID NO: 177 - DNA, 73 bp, synthetic construct
  source 1..73: mol_type = other DNA
gccctcttagtgcagctggcagcgcgtcagtttcataatctgaaagtcctgagttcaagcctcagagagggca

SEQ ID NO: 178 - DNA, 73 bp, synthetic construct
  source 1..73: mol_type = other DNA
gccgaaatagctcagttgggagagcgttagactgaagatctaaaggtccctggttcgatcccgggtttcggca

SEQ ID NO: 179 - DNA, 73 bp, synthetic construct
  source 1..73: mol_type = other DNA
gccgaaatagctcagttgggagagcgttagactgaagatctaaaggtccctggttcaatcccgggtttcggca

SEQ ID NO: 180 - DNA, 73 bp, synthetic construct
  source 1..73: mol_type = other DNA
gccgagatagctcagttgggagagcgttagactgaagatctaaaggtccctggttcaatcccgggtttcggca

SEQ ID NO: 181 - DNA, 74 bp, synthetic construct
  source 1..74: mol_type = other DNA
gccgaaatagctcagttgggagagcgttagaccgaagatcttaaaggtccctggttcaatcccgggtttcggca

SEQ ID NO: 182 - DNA, 74 bp, synthetic construct
  source 1..74: mol_type = other DNA
gctgaaatagctcagttgggagagcgttagactgaagatcttaaagttccctggttcaaccctgggtttcagcc

SEQ ID NO: 183 - DNA, 72 bp, synthetic construct
  source 1..72: mol_type = other DNA
ggctcgttggtctaggggtatgattctcgcttaggatgcgagaggtcccgggttcaaatcccggacgagccc

SEQ ID NO: 184 - DNA, 72 bp, synthetic construct
  source 1..72: mol_type = other DNA
ggctcgttggtctaggggtatgattctcgcttagggtgcgagaggtcccgggttcaaatcccggacgagccc

SEQ ID NO: 185 - DNA, 72 bp, synthetic construct
  source 1..72: mol_type = other DNA
ggctcgttggtctaggggtatgattctcgcttcgggtgcgagaggtcccgggttcaaatcccggacgagccc

SEQ ID NO: 186 - DNA, 72 bp, synthetic construct
  source 1..72: mol_type = other DNA
ggctcgttggtctaggggtatgattctcgcttcgggtgtgagaggtcccgggttcaaatcccggacgagccc

SEQ ID NO: 187 - DNA, 72 bp, synthetic construct
  source 1..72: mol_type = other DNA
ggctcgttggtctagtggtatgattctcgctttgggtgcgagaggtcccgggttcaaatcccggacgagccc

SEQ ID NO: 188 - DNA, 72 bp, synthetic construct
  source 1..72: mol_type = other DNA
ggctcgttggtctaggggtatgattctcggtttgggtccgagaggtcccgggttcaaatcccggacgagccc

SEQ ID NO: 189 - DNA, 72 bp, synthetic construct
  source 1..72: mol_type = other DNA
ggctcgttggtctaggggtatgattctcgctttgggtgcgagaggtcccgggttcaaatcccggacgagccc

SEQ ID NO: 190 - DNA, 87 bp, synthetic construct
  source 1..87: mol_type = other DNA
gcccggatgatcctcagtggtctggggtgcaggcttcaaacctgtagctgtctagcgacagagtggttcaattccacctttcgggcg

SEQ ID NO: 191 - DNA, 84 bp, synthetic construct
  source 1..84: mol_type = other DNA
gctcggatgatcctcagtggtctggggtgcaggcttcaaacctgtagctgtctagtgacagagtggttcaattccacctttgta

SEQ ID NO: 192 - DNA, 82 bp, synthetic construct
  source 1..82: mol_type = other DNA
gtagtcgtggccgagtggttaaggcgatggactagaaatccattggggtttccccgcgcaggttcgaatcctgccgactacg

SEQ ID NO: 193 - DNA, 82 bp, synthetic construct
  source 1..82: mol_type = other DNA
gtagtcgtggccgagtggttaaggcgatggactagaaatccattggggtctccccgcgcaggttcgaatcctgccgactacg

SEQ ID NO: 194 - DNA, 82 bp, synthetic construct
  source 1..82: mol_type = other DNA
gtagtcgtggccgagtggttaaggcgatggactagaaatccattggggtttccccacgcaggttcgaatcctgccgactacg

SEQ ID NO: 195 - DNA, 82 bp, synthetic construct
  source 1..82: mol_type = other DNA
gtagtcgtggccgagtggttaaggtgatggactagaaacccattggggtctccccgcgcaggttcgaatcctgccgactacg

SEQ ID NO: 196 - DNA, 82 bp, synthetic construct
  source 1..82: mol_type = other DNA
gctgtgatggccgagtggttaaggcgttggactcgaaatccaatggggtctccccgcgcaggttcgaatcctgctcacagcg

SEQ ID NO: 197 - DNA, 82 bp, synthetic construct
  source 1..82: mol_type = other DNA
gctgtgatggccgagtggttaaggcgttggactcgaaatccaatggggtctccccgcgcaggttcaaatcctgctcacagcg

SEQ ID NO: 198 - DNA, 82 bp, synthetic construct
  source 1..82: mol_type = other DNA
gctgtgatggccgagtggttaaggtgttggactcgaaatccaatgggggttccccgcgcaggttcaaatcctgctcacagcg

SEQ ID NO: 199 - DNA, 82 bp, synthetic construct
  source 1..82: mol_type = other DNA
gtcacggtggccgagtggttaaggcgttggactcgaaatccaatggggtttccccgcacaggttcgaatcctgttcgtgacg

SEQ ID NO: 200 - DNA, 82 bp, synthetic construct
  source 1..82: mol_type = other DNA
gacgaggtggccgagtggttaaggcgatggactgctaatccattgtgctctgcacgcgtgggttcgaatcccaccctcgtcg

SEQ ID NO: 201 - DNA, 82 bp, synthetic construct
  source 1..82: mol_type = other DNA
gacgaggtggccgagtggttaaggcgatggactgctaatccattgtgctctgcacgcgtgggttcgaatcccaccttcgtcg

SEQ ID NO: 202 - DNA, 82 bp, synthetic construct
  source 1..82: mol_type = other DNA
gacgaggtggccgagtggttaaggcgatggactgctaatccattgtgctttgcacgcgtgggttcgaatcccatcctcgtcg

SEQ ID NO: 203 - DNA, 82 bp, synthetic construct
  source 1..82: mol_type = other DNA
gacgaggtggccgagtggttaaggcgatggactgctaatccattgtgctctgcacgcgtgggttcgaatcccatcctcgtcg

SEQ ID NO: 204 - DNA, 82 bp, synthetic construct
  source 1..82: mol_type = other DNA
gacgaggtggccgagtggttaaggcgatggactgctaatccattgtgctctgcacacgtgggttcgaatcccatcctcgtcg

SEQ ID NO: 205 - DNA, 84 bp, synthetic construct
  source 1..84: mol_type = other DNA
ggagaggcctggccgagtggttaaggcgatggactgctaatccattgtgctctgcacgcgtgggttcgaatcccatcctcgtcg

SEQ ID NO: 206 - DNA, 82 bp, synthetic construct
  source 1..82: mol_type = other DNA
gcagcgatggccgagtggttaaggcgttggacttgaaatccaatggggtctccccgcgcaggttcgaaccctgctcgctgcg

SEQ ID NO: 207 - DNA, 82 bp, synthetic construct
  source 1..82: mol_type = other DNA
gtagtcgtggccgagtggttaaggcgatggacttgaaatccattggggtttccccgcgcaggttcgaatcctgccgactacg

SEQ ID NO: 208 - DNA, 82 bp, synthetic construct
  source 1..82: mol_type = other DNA
gtagtcgtggccgagtggttaaggcgatggacttgaaatccattggggtctccccgcgcaggttcgaatcctgccgactacg

SEQ ID NO: 209 - DNA, 82 bp, synthetic construct
  source 1..82: mol_type = other DNA
gtagtcgtggccgagtggttaaggcgatggacttgaaatccattggggtttccccgcgcaggttcgaatcctgtcggctacg

SEQ ID NO: 210 - DNA, 74 bp, synthetic construct
  source 1..74: mol_type = other DNA
ggcgccgtggcttagttggttaaagcgcctgtctagtaaacaggagatcctgggttcgaatcccagcggtgcct

SEQ ID NO: 211 - DNA, 74 bp, synthetic construct
  source 1..74: mol_type = other DNA
ggctccgtggcttagctggttaaagcgcctgtctagtaaacaggagatcctgggttcgaatcccagcggggcct

SEQ ID NO: 212 - DNA, 74 bp, synthetic construct
  source 1..74: mol_type = other DNA
ggctccgtagcttagttggttaaagcgcctgtctagtaaacaggagatcctgggttcgactcccagcggggcct

SEQ ID NO: 213 - DNA, 74 bp, synthetic construct
  source 1..74: mol_type = other DNA
ggcttcgtggcttagctggttaaagcgcctgtctagtaaacaggagatcctgggttcgaatcccagcgaggcct

SEQ ID NO: 214 - DNA, 74 bp, synthetic construct
  source 1..74: mol_type = other DNA
ggcgccgtggcttagctggttaaagcgcctgtctagtaaacaggagatcctgggttcgaatcccagcggtgcct

SEQ ID NO: 215 - DNA, 74 bp, synthetic construct
  source 1..74: mol_type = other DNA
ggccctgtggcttagctggtcaaagcgcctgtctagtaaacaggagatcctgggttcgaatcccagcggggcct

SEQ ID NO: 216 - DNA, 74 bp, synthetic construct
  source 1..74: mol_type = other DNA
ggctctatggcttagttggttaaagcgcctgtctcgtaaacaggagatcctgggttcgactcccagtggggcct

SEQ ID NO: 217 - DNA, 72 bp, synthetic construct
  source 1..72: mol_type = other DNA
ggcgcggtggccaagtggtaaggcgtcggtctcgtaaaccgaagatcacgggttcgaaccccgtccgtgcct

SEQ ID NO: 218 - DNA, 74 bp, synthetic construct
  source 1..74: mol_type = other DNA
ggctctgtggcttagttggctaaagcgcctgtctcgtaaacaggagatcctgggttcgaatcccagcggggcct

SEQ ID NO: 219 - DNA, 72 bp, synthetic construct
  source 1..72: mol_type = other DNA
ggcgcggtggccaagtggtaaggcgtcggtctcgtaaaccgaagatcgcgggttcgaaccccgtccgtgcct

SEQ ID NO: 220 - DNA, 74 bp, synthetic construct
  source 1..74: mol_type = other DNA
ggccctgtagctcagcggttggagcgctggtctcgtaaacctaggggtcgtgagttcaaatctcaccagggcct

SEQ ID NO: 221 - DNA, 74 bp, synthetic construct
  source 1..74: mol_type = other DNA
ggctctatggcttagttggttaaagcgcctgtcttgtaaacaggagatcctgggttcgaatcccagtagagcct

SEQ ID NO: 222 - DNA, 73 bp, synthetic construct
  source 1..73: mol_type = other DNA
ggctccatagctcagtggttagagcactggtcttgtaaaccaggggtcgcgagttcgatcctcgctggggcct

SEQ ID NO: 223 - DNA, 73 bp, synthetic construct
  source 1..73: mol_type = other DNA
ggctccatagctcaggggttagagcgctggtcttgtaaaccaggggtcgcgagttcaattctcgctggggcct

SEQ ID NO: 224 - DNA, 73 bp, synthetic construct
  source 1..73: mol_type = other DNA
ggctccatagctcaggggttagagcactggtcttgtaaaccaggggtcgcgagttcaaatctcgctggggcct

SEQ ID NO: 225 - DNA, 73 bp, synthetic construct
  source 1..73: mol_type = other DNA
ggccctatagctcaggggttagagcactggtcttgtaaaccaggggtcgcgagttcaaatctcgctggggcct

SEQ ID NO: 226 - DNA, 72 bp, synthetic construct
  source 1..72: mol_type = other DNA
ggctccatagctcaggggttagagcactggtcttgtaaaccagggtcgcgagttcaaatctcgctggggcct

SEQ ID NO: 227 - DNA, 72 bp, synthetic construct
  source 1..72: mol_type = other DNA
ggcctcgtggcgcaacggtagcgcgtctgactccagatcagaaggttgcgtgttcaaatcacgtcggggtca

SEQ ID NO: 228 - DNA, 72 bp, synthetic construct
  source 1..72: mol_type = other DNA
gacctcgtggcgcaatggtagcgcgtctgactccagatcagaaggttgcgtgttcaagtcacgtcggggtca

SEQ ID NO: 229 - DNA, 72 bp, synthetic construct
  source 1..72: mol_type = other DNA
gacctcgtggcgcaacggtagcgcgtctgactccagatcagaaggttgcgtgttcaaatcacgtcggggtca

SEQ ID NO: 230 - DNA, 72 bp, synthetic construct
  source 1..72: mol_type = other DNA
gacctcgtggcgcaacggtagcgcgtctgactccagatcagaaggctgcgtgttcgaatcacgtcggggtca

SEQ ID NO: 231 - DNA, 72 bp, synthetic construct
  source 1..72: mol_type = other DNA
gacctcgtggcgcaacggcagcgcgtctgactccagatcagaaggttgcgtgttcaaatcacgtcggggtca

SEQ ID NO: 232 - DNA, 93 bp, synthetic construct
  source 1..93: mol_type = other DNA
ccttcaatagttcagctggtagagcagaggactatagctacttcctcagtaggagacgtccttaggttgctggttcgattccagcttgaagga

SEQ ID NO: 233 - DNA, 91 bp, synthetic construct
  source 1..91: mol_type = other DNA
ccttcgatagctcagttggtagagcggaggactgtagttggctgtgtccttagacatccttaggtcgctggttcgaatccggctcgaagga

SEQ ID NO: 234 - DNA, 89 bp, synthetic construct
  source 1..89: mol_type = other DNA
ccttcgatagctcagttggtagagcggaggactgtagtggatagggcgtggcaatccttaggtcgctggttcgattccggctcgaagga

SEQ ID NO: 235 - DNA, 89 bp, synthetic construct
  source 1..89: mol_type = other DNA
ccttcgatagctcagttggtagagcggaggactgtaggctcattaagcaaggtatccttaggtcgctggttcgaatccggctcggagga

SEQ ID NO: 236 - DNA, 94 bp, synthetic construct
  source 1..94: mol_type = other DNA
ccttcgatagctcagctggtagagcggaggactgtagattgtatagacatttgcggacatccttaggtcgctggttcgattccagctcgaagga

SEQ ID NO: 237 - DNA, 93 bp, synthetic construct
  source 1..93: mol_type = other DNA
ccttcgatagctcagctggtagagcggaggactgtagctacttcctcagcaggagacatccttaggtcgctggttcgattccggctcgaagga

SEQ ID NO: 238 - DNA, 89 bp, synthetic construct
  source 1..89: mol_type = other DNA
ccttcgatagctcagctggtagagcggaggactgtaggcgcgcgcccgtggccatccttaggtcgctggttcgattccggctcgaagga

SEQ ID NO: 239 - DNA, 94 bp, synthetic construct
  source 1..94: mol_type = other DNA
ccttcgatagctcagctggtagagcggaggactgtagcctgtagaaacatttgtggacatccttaggtcgctggttcgattccggctcgaagga

SEQ ID NO: 240 - DNA, 94 bp, synthetic construct
  source 1..94: mol_type = other DNA
ccttcgatagctcagctggtagagcggaggactgtagattgtacagacatttgcggacatccttaggtcgctggttcgattccggctcgaagga

SEQ ID NO: 241 - DNA, 89 bp, synthetic construct
  source 1..89: mol_type = other DNA
ccttcgatagctcagctggtagagcggaggactgtagtacttaatgtgtggtcatccttaggtcgctggttcgattccggctcgaagga

SEQ ID NO: 242 - DNA, 89 bp, synthetic construct
  source 1..89: mol_type = other DNA
ccttcgatagctcagctggtagagcggaggactgtaggggtttgaatgtggtcatccttaggtcgctggttcgaatccggctcggagga

SEQ ID NO: 243 - DNA, 94 bp, synthetic construct
  source 1..94: mol_type = other DNA
ccttcgatagctcagctggtagagcggaggactgtagactgcggaaacgtttgtggacatccttaggtcgctggttcaattccggctcgaagga

SEQ ID NO: 244 - DNA, 90 bp, synthetic construct
  source 1..90: mol_type = other DNA
ctttcgatagctcagttggtagagcggaggactgtaggttcattaaactaaggcatccttaggtcgctggttcgaatccggctcgaagga

SEQ ID NO: 245 - DNA, 88 bp, synthetic construct
  source 1..88: mol_type = other DNA
tcttcaatagctcagctggtagagcggaggactgtaggtgcacgcccgtggccattcttaggtgctggtttgattccgacttggagag

SEQ ID NO: 246 - DNA, 73 bp, synthetic construct
  source 1..73: mol_type = other DNA
gtttccgtagtgtagtggttatcacgttcgcctaacacgcgaaaggtccccggttcgaaaccgggcggaaaca

SEQ ID NO: 247 - DNA, 73 bp, synthetic construct
  source 1..73: mol_type = other DNA
gtttccgtagtgtagtggtcatcacgttcgcctaacacgcgaaaggtccccggttcgaaaccgggcggaaaca

SEQ ID NO: 248 - DNA, 73 bp, synthetic construct
  source 1..73: mol_type = other DNA
gtttccgtagtgtagtggttatcacgttcgcctaacacgcgaaaggtccctggatcaaaaccaggcggaaaca

SEQ ID NO: 249 - DNA, 73 bp, synthetic construct
  source 1..73: mol_type = other DNA
gtttccgtagtgtagtggttatcacgttcgcctaacacgcgaaaggtccgcggttcgaaaccgggcggaaaca

SEQ ID NO: 250 - DNA, 73 bp, synthetic construct
  source 1..73: mol_type = other DNA
gtttccgtagtgtagtggttatcacgtttgcctaacacgcgaaaggtccccggttcgaaaccgggcagaaaca

SEQ ID NO: 251 - DNA, 72 bp, synthetic construct
  source 1..72: mol_type = other DNA
gggggtgtagctcagtggtagagcgtatgcttaacattcatgaggctctgggttcgatccccagcacttcca

SEQ ID NO: 252 - DNA, 73 bp, synthetic construct
  source 1..73: mol_type = other DNA
gtttccgtagtgtagtggttatcacgttcgcctcacacgcgaaaggtccccggttcgaaaccgggcggaaaca

SEQ ID NO: 253 - DNA, 73 bp, synthetic construct
  source 1..73: mol_type = other DNA
gcttctgtagtgtagtggttatcacgttcgcctcacacgcgaaaggtccccggttcgaaaccgggcagaagca

SEQ ID NO: 254 - DNA, 73 bp, synthetic construct
  source 1..73: mol_type = other DNA
gtttccgtagtgtagcggttatcacattcgcctcacacgcgaaaggtccccggttcgatcccgggcggaaaca

SEQ ID NO: 255 - DNA, 73 bp, synthetic construct
  source 1..73: mol_type = other DNA
gtttccgtagtgtagtggttatcacgttcgcctcacacgcgaaaggtccccggttcgaaactgggcggaaaca

SEQ ID NO: 256 - DNA, 74 bp, synthetic construct
  source 1..74: mol_type = other DNA
gtttccgtagtgtagtggttatcacgttcgcctcacacgcgtaaaggtccccggttcgaaaccgggcggaaaca

SEQ ID NO: 257 - DNA, 73 bp, synthetic construct
  source 1..73: mol_type = other DNA
gtttccgtagtggagtggttatcacgttcgcctcacacgcgaaaggtccccggtttgaaaccaggcggaaaca

SEQ ID NO: 258 - DNA, 73 bp, synthetic construct
  source 1..73: mol_type = other DNA
ggttccatagtgtagtggttatcacgtctgctttacacgcagaaggtcctgggttcgagccccagtggaacca

SEQ ID NO: 259 - DNA, 73 bp, synthetic construct
  source 1..73: mol_type = other DNA
ggttccatagtgtagcggttatcacgtctgctttacacgcagaaggtcctgggttcgagccccagtggaacca

SEQ ID NO: 260 - DNA, 73 bp, synthetic construct
  source 1..73: mol_type = other DNA
ggttccatagtgtagtggttatcacatctgctttacacgcagaaggtcctgggttcaagccccagtggaacca

SEQ ID NO: 261 - DNA, 73 bp, synthetic construct
  source 1..73: mol_type = other DNA
gtttccgtggtgtagtggttatcacattcgccttacacgcgaaaggtcctcgggtcgaaaccgagcggaaaca

SEQ ID NO: 262 - DNA, 72 bp, synthetic construct
  source 1..72: mol_type = other DNA
agcagagtggcgcagcggaagcgtgctgggcccataacccagaggtcgatggatcgaaaccatcctctgcta

SEQ ID NO: 263 - DNA, 72 bp, synthetic construct
  source 1..72: mol_type = other DNA
agcagagtggcgcagcggaagcgtgctgggcccataacccagaggtcgatggatctaaaccatcctctgcta

SEQ ID NO: 264 - DNA, 73 bp, synthetic construct
  source 1..73: mol_type = other DNA
tccctggtggtctagtggctaggattcggcgctttcaccgccgcggcccgggttcgattcccggtcagggaat

SEQ ID NO: 265 - DNA, 72 bp, synthetic construct
  source 1..72: mol_type = other DNA
gcgttggtggtttagtggtagaattctcgcctcccatgcgggagacccgggttcaattcccggccactgcac

SEQ ID NO: 266 - DNA, 72 bp, synthetic construct
  source 1..72: mol_type = other DNA
ggccttggtggtgcagtggtagaattctcgcctcccacgtgggagacccgggttcaattcccggccaatgca

SEQ ID NO: 267 - DNA, 73 bp, synthetic construct
  source 1..73: mol_type = other DNA
gtccctggtggtctagtggctaggattcggcgctttcaccgccgcggcccgggttcgattcccggccagggaa

SEQ ID NO: 268 - DNA, 75 bp, synthetic construct
  source 1..75: mol_type = other DNA
tgtctctgtggcgcaatcggttagcgcgttcggctgttaaccgaaagattggtggttcgagcccacccagggacg

SEQ ID NO: 269 - DNA, 86 bp, synthetic construct
  source 1..86: mol_type = other DNA
tggctccgtggcgcaatggatagcgcattggacttctagaggctgaaggcattcaaaggttccgggttcgagtcccggcggagtcg

SEQ ID NO: 270 - DNA, 74 bp, synthetic construct
  source 1..74: mol_type = other DNA
gcccggctagctcagtcggtagagcatgagactcttaatctcagggtcgtgggttcgagccccacgttgggcgc

SEQ ID NO: 271 - DNA, 73 bp, synthetic construct
  source 1..73: mol_type = other DNA
gccgtgatcgtatagtggttagtactctgcgttgtggccgcagcaacctcggttcgaatccgagtcacggcag

SEQ ID NO: 272 - DNA, 73 bp, synthetic construct
  source 1..73: mol_type = other DNA
gcgttggtggtatagtggtgagcatagctgccttccaagcagttgacccgggttcgattcccggccaacgcag

SEQ ID NO: 273 - DNA, 73 bp, synthetic construct
  source 1..73: mol_type = other DNA
tccctggtggtctagtggttaggattcggcgctctcaccgccgcggcccgggttcgattcccggtcagggaaa

SEQ ID NO: 274 - DNA, 73 bp, synthetic construct
  source 1..73: mol_type = other DNA
aggttccatggtgtaatggtgagcactctggactctgaatccagcgatccgagttcgagtctcggtggaacct

SEQ ID NO: 275 - DNA, 75 bp, synthetic construct
  source 1..75: mol_type = other DNA
tgtctctgtggcgtagtcggttagcgcgttcggctgttaaccgaaaagttggtggttcgagcccacccaggaacg

SEQ ID NO: 276 - DNA, 75 bp, synthetic construct
  source 1..75: mol_type = other DNA
tgtctctgtggcgcaatcggttagcgcgttcggctgttaaccgaaaggttggtggttcgagcccacccagggacg

SEQ ID NO: 277 - DNA, 75 bp, synthetic construct
  source 1..75: mol_type = other DNA
gtctctgtggcgcaatcggttagcgcattcggctgttaaccgaaaggttggtggttcgagcccacccagggacgc

SEQ ID NO: 278 - DNA, 75 bp, synthetic construct
  source 1..75: mol_type = other DNA
gtctctgtggcgcaatgggttagcgcgttcggctgttaaccgaaaggttggtggttcgagcccatccagggacgc

SEQ ID NO: 279 - DNA, 72 bp, synthetic construct
  source 1..72: mol_type = other DNA
gcactggtggttcagtggtagaattctcgcctcacacgcgggacacccgggttcaattcccggtcaaggcaa

SEQ ID NO: 280 - DNA, 74 bp, synthetic construct
  source 1..74: mol_type = other DNA
gtttccgtagtgtagtggttatcacgttcgcctcacacgcgaaaggtccccggttcgaaactgggcggaaacag

SEQ ID NO: 281 - DNA, 72 bp, synthetic construct
  source 1..72: mol_type = other DNA
gcactggtggttcagtggtagaattctcgcctcccacgcgggagacccgggtttaattcccggtcaagataa

SEQ ID NO: 282 - DNA, 75 bp, synthetic construct
  source 1..75: mol_type = other DNA
gtttccgtagtgtagtggttatcacgttcgcctcacacgcgtaaaggtccccggttcgaaaccgggcggaaacat

SEQ ID NO: 283 - DNA, 73 bp, synthetic construct
  source 1..73: mol_type = other DNA
tagcagagtggcgcagcggaagcgtgctgggcccataacccagaggtcgatggatcgaaaccatcctctgcta

SEQ ID NO: 284 - DNA, 74 bp, synthetic construct
  source 1..74: mol_type = other DNA
gtttccgtagtgtagtggttatcacgttcgcctcacacgcgaaaggtccccggttcgaaaccgggcggaaacaa

SEQ ID NO: 285 - DNA, 73 bp, synthetic construct
  source 1..73: mol_type = other DNA
tcctcgttagtatagtggtgagtatccccgcctgtcacgcgggagaccggggttcgattccccgacggggagg

SEQ ID NO: 286 - DNA, 72 bp, synthetic construct
  source 1..72: mol_type = other DNA
tgcatgggtggttcagtggtagaattctcgcctgccacgcgggaggcccgggttcgattcccggcccatgca

SEQ ID NO: 287 - DNA, 73 bp, synthetic construct
  source 1..73: mol_type = other DNA
tccctggtggtctagtggttaggattcggcgctctcaccgccgcggcccgggttcgattcccggtcagggaag

SEQ ID NO: 288 - DNA, 73 bp, synthetic construct
  source 1..73: mol_type = other DNA
atccttgttactatagtggtgagtatctctgcctgtcatgcgtgagagagggggtcgattccccgacggggag

SEQ ID NO: 289 - DNA, 72 bp, synthetic construct
  source 1..72: mol_type = other DNA
gcattggtggttcagtggtagaattctcgcctgccacgcgggaggcccgggttcgattcccggccaatgcac

SEQ ID NO: 290 - DNA, 84 bp, synthetic construct
  source 1..84: mol_type = other DNA
gtcaggatggccgagcggtctaaggcgctgcgttcaggtcgcagtctcccctggaggcgtgggttcgaatcccactcctgacaa

SEQ ID NO: 291 - DNA, 73 bp, synthetic construct
  source 1..73: mol_type = other DNA
cgcgttggtggtatagtggtgagcatagctgccttccaagcagttgacccgggttcgattcccggccaacgca

SEQ ID NO: 292 - DNA, 75 bp, synthetic construct
  source 1..75: mol_type = other DNA
cgtctctgtggcgcaatcggttagcgcgttcggctgttaaccgaaaggttggtggttcgatcccacccagggacg

SEQ ID NO: 293 - DNA, 73 bp, synthetic construct
  source 1..73: mol_type = other DNA
cgcgttggtggtgtagtggtgagcacagctgcctttcaagcagttaacgcgggttcgattcccgggtaacgaa

SEQ ID NO: 294 - DNA, 73 bp, synthetic construct
  source 1..73: mol_type = other DNA
cggctcgttggtctaggggtatgattctcgcttcgggtgcgagaggtcccgggttcaaatcccggacgagccc

SEQ ID NO: 295 - DNA, 73 bp, synthetic construct
  source 1..73: mol_type = other DNA
ggctcgttggtctaggggtatgattctcgcttagggtgcgagaggtcccgggttcaaatcccggacgagccct

SEQ ID NO: 296 - DNA, 74 bp, synthetic construct
  source 1..74: mol_type = other DNA
cgcccggatagctcagtcggtagagcatcagacttttaatctgagggtccagggttcaagtccctgttcgggcg

SEQ ID NO: 297 - DNA, 74 bp, synthetic construct
  source 1..74: mol_type = other DNA
gcccggatagctcagtcggtagagcatcagacttttaatctgagggtccagggttcaagtccctgttcgggcgt

SEQ ID NO: 298 - DNA, 107 bp, synthetic construct
  source 1..107: mol_type = other DNA
tgtcaggatggccgagtggtctaaggcgccagactcaaggtaagcaccttgcctgcgggctttctggtctccggatggaggcgtgggttcgaatcccacttctgaca

SEQ ID NO: 299 - DNA, 73 bp, synthetic construct
  source 1..73: mol_type = other DNA
ttccctggtggtctagtggttaggattcggcgctctcaccgccgcggcccgggttcgattcccggtcaggaaa

SEQ ID NO: 300 - DNA, 90 bp, synthetic construct
  source 1..90: mol_type = other DNA
gccttcgatagctcagttggtagagcggaggactgtagtggatagggcgtggcaatccttaggtcgctggttcgattccggctcgaagga

SEQ ID NO: 301 - DNA, 74 bp, synthetic construct
  source 1..74: mol_type = other DNA
cgggggattagctcaaatggtagagcgctcgcttagcatgcgagaggtagcgggatcgatgcccgcatcctcca

SEQ ID NO: 302 - DNA, 94 bp, synthetic construct
  source 1..94: mol_type = other DNA
agctccagtggcgcaatcggttagcgcgcggtacttatacagcagtacatgcagagcaatgccgaggttgtgagttcgagcctcacctggagca

SEQ ID NO: 303 - DNA, 72 bp, synthetic construct
  source 1..72: mol_type = other DNA
gcgccgctggtgtagtggtatcatgcaagattcccattcttgcgacccgggttcgattcccgggcggcgcat

SEQ ID NO: 304 - DNA, 73 bp, synthetic construct
  source 1..73: mol_type = other DNA
tcccatatggtctagcggttaggattcctggttttcacccaggtggcccgggttcgactcccggtatgggaac

SEQ ID NO: 305 - DNA, 73 bp, synthetic construct
  source 1..73: mol_type = other DNA
gggggatgtagctcagtggtagagcgcgcgcttcgcatgtgtgaggtcccgggttcaatccccggcatctcca

SEQ ID NO: 306 - DNA, 72 bp, synthetic construct
  source 1..72: mol_type = other DNA
gcattggtggttcagtggtagaattctcgcctgccacgcgggaggcccgggttcgattcccggccaatgcaa

SEQ ID NO: 307 - DNA, 74 bp, synthetic construct
  source 1..74: mol_type = other DNA
gggccagtggcgcaatggataacgcgtctgactacggatcagaagattctaggttcgactcctggctggctcgc

SEQ ID NO: 308 - DNA, 74 bp, synthetic construct
  source 1..74: mol_type = other DNA
ggtttccgtagtgtagtggttatcacgttcgcctaacacgcgaaaggtccccggttcgaaaccgggcggaaaca

SEQ ID NO: 309 - DNA, 74 bp, synthetic construct
  source 1..74: mol_type = other DNA
agtttccgtagtgtagtggttatcacgttcgcctaacacgcgaaaggtccccggttcgaaaccgggcggaaaca

SEQ ID NO: 310 - DNA, 83 bp, synthetic construct
  source 1..83: mol_type = other DNA
aggtagcgtggccgagcggtctaaggcgctggattaaggctccagtctcttcgggggcgtgggttcgaatcccaccgctgcca

SEQ ID NO: 311 - DNA, 74 bp, synthetic construct
  source 1..74: mol_type = other DNA
gtttccgtagtgtagtggtcatcacgttcgcctaacacgcgaaaggtccccggttcgaaaccgggcggaaacat

SEQ ID NO: 312 - DNA, 73 bp, synthetic construct
  source 1..73: mol_type = other DNA
ggctcgttggtctaggggtatgattctcgctttgggtgcgagaggtcccgggttcaaatcccggacgagccca

SEQ ID NO: 313 - DNA, 73 bp, synthetic construct
  source 1..73: mol_type = other DNA
ggctccatagctcaggggttagagcactggtcttgtaaaccagggtcgcgagttcaaatctcgctggggcctg

SEQ ID NO: 314 - DNA, 73 bp, synthetic construct
  source 1..73: mol_type = other DNA
tggggatgtagctcagtggtagagcgcatgctttgcatgtatgaggccccgggttcgatccccggcatctcca

SEQ ID NO: 315 - DNA, 74 bp, synthetic construct
  source 1..74: mol_type = other DNA
cgcccggctagctcagtcggtagagcatgagactcttaatctcagggtcgtgggttcgagccccacgttgggcg

SEQ ID NO: 316 - DNA, 74 bp, synthetic construct
  source 1..74: mol_type = other DNA
gtttccgtagtgtagtggttatcacgttcgcctaacacgcgaaaggtccccggttcgaaaccgggcggaaacaa

SEQ ID NO: 317 - DNA, 74 bp, synthetic construct
  source 1..74: mol_type = other DNA
gcccggctagctcagtcggtagagcatgagactcttaatctcagggtcgtgggttcgagccccacgttgggcgt

SEQ ID NO: 318 - DNA, 74 bp, synthetic construct
  source 1..74: mol_type = other DNA
gtttccgtagtgtagtggttatcacgttcgcctcacacgcgaaaggtccccggttcgaaaccgggcggaaacac

SEQ ID NO: 319 - DNA, 73 bp, synthetic construct
  source 1..73: mol_type = other DNA
cagcagagtggcgcagcggaagcgtgctgggcccataacccagaggtcgatggatcgaaaccatcctctgcta

SEQ ID NO: 320 - DNA, 85 bp, synthetic construct
  source 1..85: mol_type = other DNA
ggagaggcctggccgagtggttaaggcgatggactgctaatccattgtgctctgcacgcgtgggttcgaatcccatcctcgtcgc

SEQ ID NO: 321 - DNA, 73 bp, synthetic construct
  source 1..73: mol_type = other DNA
ggccccatggtgtaatggttagcactctggactttgaatccagcgatccgagttcaaatctcggtgggacctg

SEQ ID NO: 322 - DNA, 73 bp, synthetic construct
  source 1..73: mol_type = other DNA
ggccccatggtgtaatggttagcactctggactttgaatccagcgatccgagttcaaatctcggtgggaccta

SEQ ID NO: 323 - DNA, 83 bp, synthetic construct
  source 1..83: mol_type = other DNA
gtagtcgtggccgagtggttaaggcgatggacttgaaatccattggggtctccccgcgcaggttcgaatcctgccgactacgg

SEQ ID NO: 324 - DNA, 73 bp, synthetic construct
  source 1..73: mol_type = other DNA
agcagagtggcgcagcggaagcgtgctgggcccataacccagaggtcgatggatcgaaaccatcctctgctat

SEQ ID NO: 325 - DNA, 74 bp, synthetic construct
  source 1..74: mol_type = other DNA
ggaccacgtggcctaatggataaggcgtctgacttcggatcagaagattgagggttcgaatccctccgtggtta

SEQ ID NO: 326 - DNA, 83 bp, synthetic construct
  source 1..83: mol_type = other DNA
tgtagtcgtggccgagtggttaaggcgatggactagaaatccattggggtctccccgcgcaggttcgaatcctgccgactacg

SEQ ID NO: 327 - DNA, 73 bp, synthetic construct
  source 1..73: mol_type = other DNA
agcagagtggcgcagcggaagcgtgctgggcccataacccagaggtcgatggatcgaaaccatcctctgctag

SEQ ID NO: 328 - DNA, 84 bp, synthetic construct
  source 1..84: mol_type = other DNA
cgtcaggatggccgagcggtctaaggcgctgcgttcaggtcgcagtctcccctggaggcgtgggttcgaatcccactcctgaca

SEQ ID NO: 329 - DNA, 75 bp, synthetic construct
  source 1..75: mol_type = other DNA
ggctccgtggcttagctggttaaagcgcctgtctagtaaacaggagatcctgggttcgaatcccagcggggcctg

SEQ ID NO: 330 - DNA, 74 bp, synthetic construct
  source 1..74: mol_type = other DNA
agggccagtggcgcaatggataacgcgtctgactacggatcagaagattccaggttcgactcctggctggctcg

SEQ ID NO: 331 - DNA, 74 bp, synthetic construct
  source 1..74: mol_type = other DNA
ggtttccgtagtgtagtggttatcacgttcgcctcacacgcgaaaggtccccggttcgaaaccgggcggaaaca

SEQ ID NO: 332 - DNA, 73 bp, synthetic construct
  source 1..73: mol_type = other DNA
aggggatgtagctcagtggtagagcgcatgcttcgcatgtatgaggtcccgggttcgatccccggcatctcca

SEQ ID NO: 333 - DNA, 75 bp, synthetic construct
  source 1..75: mol_type = other DNA
tggccggttagctcagttggttagagcgtggtgctaataacgccaaggtcgcgggttcgatccccgtacgggcca

SEQ ID NO: 334 - DNA, 73 bp, synthetic construct
  source 1..73: mol_type = other DNA
cggctcgttggtctaggggtatgattctcgcttagggtgcgagaggtcccgggttcaaatcccggacgagccc

SEQ ID NO: 335 - DNA, 74 bp, synthetic construct
  source 1..74: mol_type = other DNA
agcccggctagctcagtcggtagagcatgagactcttaatctcagggtcgtgggttcgagccccacgttgggcg

SEQ ID NO: 336 - DNA, 92 bp, synthetic construct
  source 1..92: mol_type = other DNA
tccttcgatagctcagttggtagagcggaggactgtagttggctgtgtccttagacatccttaggtcgctggttcgaatccggctcgaagga

SEQ ID NO: 337 - DNA, 74 bp, synthetic construct
  source 1..74: mol_type = other DNA
ggggaattagctcaaatggtagagcgctcgcttagcatgcgagaggtagcgggatcgatgcccgcattctccag

SEQ ID NO: 338 - DNA, 74 bp, synthetic construct
  source 1..74: mol_type = other DNA
cgccctcttagcgcagcgggcagcgcgtcagtctcataatctgaaggtcctgagttcgagcctcagagagggca

SEQ ID NO: 339 - DNA, 95 bp, synthetic construct
  source 1..95: mol_type = other DNA
tgctccagtggcgcaatcggttagcgcgcggtacttatatggcagtatgtgtgcgagtgatgccgaggttgtgagttcgagcctcacctggagca

SEQ ID NO: 340 - DNA, 73 bp, synthetic construct
  source 1..73: mol_type = other DNA
tgccgtgatcgtatagtggttagtactctgcgttgtggccgcagcaacctcggttcgaatccgagtcacggca

SEQ ID NO: 341 - DNA, 75 bp, synthetic construct
  source 1..75: mol_type = other DNA
ggccggttagctcagttggttagagcgtggtgctaataacgccaaggtcgcgggttcgatccccgtacgggccac

SEQ ID NO: 342 - DNA, 74 bp, synthetic construct
  source 1..74: mol_type = other DNA
agtttccgtagtgtagtggttatcacgtttgcctaacacgcgaaaggtccccggttcgaaaccgggcagaaaca

SEQ ID NO: 343 - DNA, 74 bp, synthetic construct
  source 1..74: mol_type = other DNA
gcttctgtagtgtagtggttatcacgttcgcctcacacgcgaaaggtccccggttcgaaaccgggcagaagcaa

SEQ ID NO: 344 - DNA, 73 bp, synthetic construct
  source 1..73: mol_type = other DNA
ttcctcgttagtatagtggtgagtatccccgcctgtcacgcgggagaccggggttcgattccccgacggggag

SEQ ID NO: 345 - DNA, 83 bp, synthetic construct
  source 1..83: mol_type = other DNA
gtagtcgtggccgagtggttaaggcgatggacttgaaatccattggggtttccccgcgcaggttcgaatcctgtcggctacgg

SEQ ID NO: 346 - DNA, 73 bp, synthetic construct
  source 1..73: mol_type = other DNA
aggttccatggtgtaatggttagcactctggactctgaatccagcgatccgagttcaaatctcggtggaacct

SEQ ID NO: 347 - DNA, 73 bp, synthetic construct
  source 1..73: mol_type = other DNA
tcctcgttagtatagtggtgagtgtccccgtctgtcacgcgggagaccggggttcgattccccgacggggaga

SEQ ID NO: 348 - DNA, 74 bp, synthetic construct
  source 1..74: mol_type = other DNA
gtttccgtagtgtagtggttatcacgttcgcctaacacgcgaaaggtccctggatcaaaaccaggcggaaacaa

SEQ ID NO: 349 - DNA, 75 bp, synthetic construct
  source 1..75: mol_type = other DNA
cggccggttagctcagttggttagagcgtggtgctaataacgccaaggtcgcgggttcgatccccgtactggcca

SEQ ID NO: 350 - DNA, 73 bp, synthetic construct
  source 1..73: mol_type = other DNA
ggccccatggtgtaatggtcagcactctggactctgaatccagcgatccgagttcaaatctcggtgggaccca

SEQ ID NO: 351 - DNA, 73 bp, synthetic construct
  source 1..73: mol_type = other DNA
ggccccatggtgtaatggttagcactctggactttgaatccagcgatccgagttcaaatctcggtgggacctt

SEQ ID NO: 352 - DNA, 73 bp, synthetic construct
  source 1..73: mol_type = other DNA
tgggggtgtagctcagtggtagagcgcgtgcttagcatgtacgaggtcccgggttcaatccccggcacctcca

SEQ ID NO: 353 - DNA, 73 bp, synthetic construct
  source 1..73: mol_type = other DNA
ggggatgtagctcagtggtagagcgcatgcttagcatgcatgaggtcccgggttcgatccccagcatctccag

SEQ ID NO: 354 - DNA, 73 bp, synthetic construct
  source 1..73: mol_type = other DNA
agggggtgtagctcagtggtagagcgcgtgcttcgcatgtacgaggccccgggttcgacccccggctcctcca

SEQ ID NO: 355 - DNA, 73 bp, synthetic construct
  source 1..73: mol_type = other DNA
gggggtgtagctcagtggtagagcgcgtgcttagcatgcacgaggccccgggttcaatccccggcacctccat

SEQ ID NO: 356 - DNA, 73 bp, synthetic construct
  source 1..73: mol_type = other DNA
gggggtgtagctcagtggtagagcgcgtgcttagcatgcacgaggccccgggttcaatccccggcacctccag

SEQ ID NO: 357 - DNA, 107 bp, synthetic construct
  source 1..107: mol_type = other DNA
gtcaggatggccgagtggtctaaggcgccagactcaagctaagcttcctccgcggtggggattctggtctccaatggaggcgtgggttcgaatcccacttctgacac

SEQ ID NO: 358 - DNA, 106 bp, synthetic construct
  source 1..106: mol_type = other DNA
tgtcaggatggccgagtggtctaaggcgccagactcaagcttggcttcctcgtgttgaggattctggtctccaatggaggcgtgggttcgaatcccacttctgaca

SEQ ID NO: 359 - DNA, 73 bp, synthetic construct
  source 1..73: mol_type = other DNA
ggttccatggtgtaatggttagcactctggactctgaatccagcgatccgagttcaaatctcggtggaacctt

SEQ ID NO: 360 - DNA, 83 bp, synthetic construct
  source 1..83: mol_type = other DNA
ggtagcgtggccgagcggtctaaggcgctggattaaggctccagtctcttcgggggcgtgggttcgaatcccaccgctgccag

SEQ ID NO: 361 - DNA, 74 bp, synthetic construct
  source 1..74: mol_type = other DNA
tgcctccttagcgcagtaggcagcgcgtcagtctcataatctgaaggtcctgagttcgaacctcagagggggca

SEQ ID NO: 362 - DNA, 74 bp, synthetic construct
  source 1..74: mol_type = other DNA
agcccggatagctcagtcggtagagcatcagacttttaatctgagggtccagggttcaagtccctgttcgggcg

SEQ ID NO: 363 - DNA, 74 bp, synthetic construct
  source 1..74: mol_type = other DNA
gcctccttagcgcagtaggcagcgcgtcagtctcataatctgaaggtcctgagttcgaacctcagagggggcag

SEQ ID NO: 364 - DNA, 73 bp, synthetic construct
  source 1..73: mol_type = other DNA
ttccctggtggtctagtggttaggattcggcgctctcaccgccgcggcccgggttcgattcccggtcagggaa

SEQ ID NO: 365 - DNA, 84 bp, synthetic construct
  source 1..84: mol_type = other DNA
caccaggatggccgagtggttaaggcgttggacttaagatccaatggacatatgtccgcgtgggttcgaaccccactcctggta

SEQ ID NO: 366 - DNA, 73 bp, synthetic construct
  source 1..73: mol_type = other DNA
tggctcgttggtctaggggtatgattctcgcttagggtgcgagaggtcccgggttcaaatcccggacgagccc

SEQ ID NO: 367 - DNA, 74 bp, synthetic construct
  source 1..74: mol_type = other DNA
agccccagtggcctaatggataaggcattggcctcctaagccagggattgtgggttcgagtcccatctggggtg

SEQ ID NO: 368 - DNA, 73 bp, synthetic construct
  source 1..73: mol_type = other DNA
ggggatatagctcaggggtagagcatttgactgcagatcaagaggtccccggttcaaatccgggtgccccccc

SEQ ID NO: 369 - DNA, 94 bp, synthetic construct
  source 1..94: mol_type = other DNA
cccttcgatagctcagctggtagagcggaggactgtagctacttcctcagcaggagacatccttaggtcgctggttcgattccggctcgaagga

SEQ ID NO: 370 - DNA, 90 bp, synthetic construct
  source 1..90: mol_type = other DNA
cccttcgatagctcagctggtagagcggaggactgtaggcgcgcgcccgtggccatccttaggtcgctggttcgattccggctcgaagga

SEQ ID NO: 371 - DNA, 74 bp, synthetic construct
  source 1..74: mol_type = other DNA
tgggggattagctcaaatggtagagcgctcgcttagcatgcgagaggtagcgggatcgatgcccgcatcctcca

SEQ ID NO: 372 - DNA, 83 bp, synthetic construct
  source 1..83: mol_type = other DNA
gtagtcgtggccgagtggttaaggcgatggactagaaatccattggggtctccccgcgcaggttcgaatcctgccgactacgg

SEQ ID NO: 373 - DNA, 74 bp, synthetic construct
  source 1..74: mol_type = other DNA
gcctcgttagcgcagtaggtagcgcgtcagtctcataatctgaaggtcgtgagttcgatcctcacacggggcac

SEQ ID NO: 374 - DNA, 92 bp, synthetic construct
  source 1..92: mol_type = other DNA
ggctctgtggcgcaatggatagcgcattggacttctagctgagcctagtgtggtcattcaaaggttgtgggttcgagtcccaccagagtcga

SEQ ID NO: 375 - DNA, 75 bp, synthetic construct
  source 1..75: mol_type = other DNA
gtctctgtggcgcaatcggttagcgcgttcggctgttaaccgaaaggttggtggttcgagcccacccagggacgc

SEQ ID NO: 376 - DNA, 83 bp, synthetic construct
  source 1..83: mol_type = other DNA
ggcagcgatggccgagtggttaaggcgttggacttgaaatccaatggggtctccccgcgcaggttcgaaccctgctcgctgcg

SEQ ID NO: 377 - DNA, 74 bp, synthetic construct
  source 1..74: mol_type = other DNA
ggttccatagtgtagtggttatcacgtctgctttacacgcagaaggtcctgggttcgagccccagtggaaccat

SEQ ID NO: 378 - DNA, 74 bp, synthetic construct
  source 1..74: mol_type = other DNA
ggttccatagtgtagcggttatcacgtctgctttacacgcagaaggtcctgggttcgagccccagtggaaccac

SEQ ID NO: 379 - DNA, 87 bp, synthetic construct
  source 1..87: mol_type = other DNA
tggctctgtggcgcaatggatagcgcattggacttctagatagttagagaaattcaaaggttgtgggttcgagtcccaccagagtcg

SEQ ID NO: 380 - DNA, 84 bp, synthetic construct
  source 1..84: mol_type = other DNA
taccagaatggccgagtggttaaggcgttggacttaagatccaatggattcatatccgcgtgggttcgaaccccacttctggta

SEQ ID NO: 381 - DNA, 74 bp, synthetic construct
  source 1..74: mol_type = other DNA
ggcccggatagctcagtcggtagagcatcagacttttaatctgagggtccggggttcaagtccctgttcgggcg

SEQ ID NO: 382 - DNA, 74 bp, synthetic construct
  source 1..74: mol_type = other DNA
gccgaaatagctcagttgggagagcgttagactgaagatctaaaggtccctggttcgatcccgggtttcggcag

SEQ ID NO: 383 - DNA, 74 bp, synthetic construct
  source 1..74: mol_type = other DNA
gcccggatagctcagtcggtagagcatcagacttttaatctgagggtccagggttcaagtccctgttcgggcgg

SEQ ID NO: 384 - DNA, 74 bp, synthetic construct
  source 1..74: mol_type = other DNA
gccgaaatagctcagttgggagagcgttagactgaagatctaaaggtccctggttcaatcccgggtttcggcag

SEQ ID NO: 385 - DNA, 83 bp, synthetic construct
  source 1..83: mol_type = other DNA
ggacgaggtggccgagtggttaaggcgatggactgctaatccattgtgctttgcacgcgtgggttcgaatcccatcctcgtcg

SEQ ID NO: 386 - DNA, 73 bp, synthetic construct
  source 1..73: mol_type = other DNA
ggctcgttggtctaggggtatgattctcggtttgggtccgagaggtcccgggttcaaatcccggacgagcccc

SEQ ID NO: 387 - DNA, 83 bp, synthetic construct
  source 1..83: mol_type = other DNA
agtcacggtggccgagtggttaaggcgttggactcgaaatccaatggggtttccccgcacaggttcgaatcctgttcgtgacg

SEQ ID NO: 388 - DNA, 73 bp, synthetic construct
  source 1..73: mol_type = other DNA
ctcctcgttagtatagtggttagtatccccgcctgtcacgcgggagaccggggttcaattccccgacggggag

SEQ ID NO: 389 - DNA, 73 bp, synthetic construct
  source 1..73: mol_type = other DNA
ggacctcgtggcgcaacggtagcgcgtctgactccagatcagaaggctgcgtgttcgaatcacgtcggggtca

SEQ ID NO: 390 - DNA, 73 bp, synthetic construct
  source 1..73: mol_type = other DNA
ggggatgtagctcagtggtagagcgcatgctttgcatgtatgaggccccgggttcgatccccggcatctccat

SEQ ID NO: 391 - DNA, 74 bp, synthetic construct
  source 1..74: mol_type = other DNA
gccgaaatagctcagttgggagagcgttagactgaagatctaaaggtccctggttcgatcccgggtttcggcac

SEQ ID NO: 392 - DNA, 73 bp, synthetic construct
  source 1..73: mol_type = other DNA
aggggatgtagctcagtggtagagcgcatgctttgcacgtatgaggccccgggttcaatccccggcatctcca

SEQ ID NO: 393 - DNA, 75 bp, synthetic construct
  source 1..75: mol_type = other DNA
gtctctgtggcgcaatcggttagcgcgttcggctgttaaccgaaaggttggtggttcgagcccacccagggacgg

SEQ ID NO: 394 - DNA, 73 bp, synthetic construct
  source 1..73: mol_type = other DNA
tcccacatggtctagcggttaggattcctggttttcacccaggcggcccgggttcgactcccggtgtgggaac

SEQ ID NO: 395 - DNA, 74 bp, synthetic construct
  source 1..74: mol_type = other DNA
ggctccatagctcaggggttagagcgctggtcttgtaaaccaggggtcgcgagttcaattctcgctggggcctg

SEQ ID NO: 396 - DNA, 83 bp, synthetic construct
  source 1..83: mol_type = other DNA
tggtagtgtggccgagcggtctaaggcgctggatttaggctccagtctcttcgggggcgtgggttcgaatcccaccactgcca

SEQ ID NO: 397 - DNA, 74 bp, synthetic construct
  source 1..74: mol_type = other DNA
ggctccatagctcaggggttagagcactggtcttgtaaaccaggggtcgcgagttcaaatctcgctggggcctc

SEQ ID NO: 398 - DNA, 73 bp, synthetic construct
  source 1..73: mol_type = other DNA
tggctcgttggtctagtggtatgattctcgctttgggtgcgagaggtcccgggttcaaatcccggacgagccc

SEQ ID NO: 399 - DNA, 95 bp, synthetic construct
  source 1..95: mol_type = other DNA
ccttcgatagctcagctggtagagcggaggactgtagattgtacagacatttgcggacatccttaggtcgctggttcgattccggctcgaaggaa

SEQ ID NO: 400 - DNA, 74 bp, synthetic construct
  source 1..74: mol_type = other DNA
aggccctatagctcaggggttagagcactggtcttgtaaaccaggggtcgcgagttcaaatctcgctggggcct

SEQ ID NO: 401 - DNA, 90 bp, synthetic construct
  source 1..90: mol_type = other DNA
tccttcgatagctcagctggtagagcggaggactgtagtacttaatgtgtggtcatccttaggtcgctggttcgattccggctcgaagga

SEQ ID NO: 402 - DNA, 73 bp, synthetic construct
  source 1..73: mol_type = other DNA
tggctcgttggtctaggggtatgattctcgctttgggtgcgagaggtcccgggttcaaatcccggacgagccc

SEQ ID NO: 403 - DNA, 74 bp, synthetic construct
  source 1..74: mol_type = other DNA
gcccggctagctcagtcggtagagcatgggactcttaatcccagggtcgtgggttcgagccccacgttgggcgc

SEQ ID NO: 404 - DNA, 75 bp, synthetic construct
  source 1..75: mol_type = other DNA
cggccggttagctcagttggttagagcgtggtgctaataacgccaaggtcgcgggttcgatccccgtacgggcca

SEQ ID NO: 405 - DNA, 73 bp, synthetic construct
  source 1..73: mol_type = other DNA
tcccacatggtctagcggttaggattcctggttttcacccaggcggcccgggttcgactcccggtgtgggaat

SEQ ID NO: 406 - DNA, 83 bp, synthetic construct
  source 1..83: mol_type = other DNA
gacgaggtggccgagtggttaaggcgatggactgctaatccattgtgctctgcacgcgtgggttcgaatcccatcctcgtcga

SEQ ID NO: 407 - DNA, 73 bp, synthetic construct
  source 1..73: mol_type = other DNA
gccgtgatcgtatagtggttagtactctgcgttgtggccgcagcaacctcggttcgaatccgagtcacggcat

SEQ ID NO: 408 - DNA, 73 bp, synthetic construct
  source 1..73: mol_type = other DNA
cgccgtgatcgtatagtggttagtactctgcgttgtggccgcagcaacctcggttcgaatccgagtcacggca

SEQ ID NO: 409 - DNA, 73 bp, synthetic construct
  source 1..73: mol_type = other DNA
ggttccatggtgtaatggttagcactctggactctgaatccagcgatccgagttcaaatctcggtggaacctg

SEQ ID NO: 410 - DNA, 74 bp, synthetic construct
  source 1..74: mol_type = other DNA
tgcccggctagctcagtcggtagagcatgggactcttaatcccagggtcgtgggttcgagccccacgttgggcg

SEQ ID NO: 411 - DNA, 74 bp, synthetic construct
  source 1..74: mol_type = other DNA
gggccgcgtggcctaatggataaggcgtctgacttcggatcagaagattgcaggttcgagtcctgccgcggtcg

SEQ ID NO: 412 - DNA, 72 bp, synthetic construct
  source 1..72: mol_type = other DNA
gcgccgctggtgtagtggtatcatgcaagattcccattcttgcgacccgggttcgattcccgggcggcgcac

SEQ ID NO: 413 - DNA, 74 bp, synthetic construct
  source 1..74: mol_type = other DNA
gggccgcgtggcctaatggataaggcgtctgattccggatcagaagattgagggttcgagtcccttcgtggtcg

SEQ ID NO: 414 - DNA, 74 bp, synthetic construct
  source 1..74: mol_type = other DNA
cgccccggtggcctaatggataaggcattggcctcctaagccagggattgtgggttcgagtcccacccggggta

SEQ ID NO: 415 - DNA, 74 bp, synthetic construct
  source 1..74: mol_type = other DNA
gcccggctagctcagtcggtagagcatgagacccttaatctcagggtcgtgggttcgagccccacgttgggcgt

SEQ ID NO: 416 - DNA, 73 bp, synthetic construct
  source 1..73: mol_type = other DNA
aggcgcggtggccaagtggtaaggcgtcggtctcgtaaaccgaagatcacgggttcgaaccccgtccgtgcct

SEQ ID NO: 417 - DNA, 83 bp, synthetic construct
  source 1..83: mol_type = other DNA
ggtagcgtggccgagtggtctaaggcgctggatttaggctccagtcatttcgatggcgtgggttcgaatcccaccgctgccac

SEQ ID NO: 418 - DNA, 83 bp, synthetic construct
  source 1..83: mol_type = other DNA
gggtagcgtggccgagcggtctaaggcgctggattaaggctccagtctcttcgggggcgtgggttcgaatcccaccgctgcca

SEQ ID NO: 419 - DNA, 84 bp, synthetic construct
  source 1..84: mol_type = other DNA
agtcaggatggccgagcggtctaaggcgctgcgttcaggtcgcagtctcccctggaggcgtgggttcgaatcccacttctgaca

SEQ ID NO: 420 - DNA, 84 bp, synthetic construct
  source 1..84: mol_type = other DNA
gtcaggatggccgagcggtctaaggcgctgcgttcaggtcgcagtctcccctggaggcgtgggttcgaatcccacttctgacag

SEQ ID NO: 421 - DNA, 74 bp, synthetic construct
  source 1..74: mol_type = other DNA
gcctcgttagcgcagtaggcagcgcgtcagtctcataatctgaaggtcgtgagttcgagcctcacacggggcag

SEQ ID NO: 422 - DNA, 83 bp, synthetic construct
  source 1..83: mol_type = other DNA
ggtagcgtggccgagcggtctaaggcgctggatttaggctccagtctcttcggaggcgtgggttcgaatcccaccgctgccag

SEQ ID NO: 423 - DNA, 89 bp, synthetic construct
  source 1..89: mol_type = other DNA
tggctctgtggcgcaatggatagcgcattggacttctagtgacgaatagagcaattcaaaggttgtgggttcgaatcccaccagagtcg

SEQ ID NO: 424 - DNA, 72 bp, synthetic construct
  source 1..72: mol_type = other DNA
cgcattggtggttcagtggtagaattctcgcctgccacgcgggaggcccgggttcgattcccggccaatgca

SEQ ID NO: 425 - DNA, 83 bp, synthetic construct
  source 1..83: mol_type = other DNA
gctgtgatggccgagtggttaaggcgttggactcgaaatccaatggggtctccccgcgcaggttcgaatcctgctcacagcgt

SEQ ID NO: 426 - DNA, 75 bp, synthetic construct
  source 1..75: mol_type = other DNA
ggcgccgtggcttagctggttaaagcgcctgtctagtaaacaggagatcctgggttcgaatcccagcggtgcctg

SEQ ID NO: 427 - DNA, 73 bp, synthetic construct
  source 1..73: mol_type = other DNA
cgacctcgtggcgcaacggtagcgcgtctgactccagatcagaaggttgcgtgttcaaatcacgtcggggtca

SEQ ID NO: 428 - DNA, 83 bp, synthetic construct
  source 1..83: mol_type = other DNA
agacgaggtggccgagtggttaaggcgatggactgctaatccattgtgctctgcacgcgtgggttcgaatcccatcctcgtcg

SEQ ID NO: 429 - DNA, 75 bp, synthetic construct
  source 1..75: mol_type = other DNA
cggcgccgtggcttagttggttaaagcgcctgtctagtaaacaggagatcctgggttcgaatcccagcggtgcct

SEQ ID NO: 430 - DNA, 73 bp, synthetic construct
  source 1..73: mol_type = other DNA
ggcctcgtggcgcaacggtagcgcgtctgactccagatcagaaggttgcgtgttcaaatcacgtcggggtcaa

SEQ ID NO: 431 - DNA, 73 bp, synthetic construct
  source 1..73: mol_type = other DNA
agcgttggtggtatagtggtaagcatagctgccttccaagcagttgacccgggttcgattcccggccaacgca

SEQ ID NO: 432 - DNA, 73 bp, synthetic construct
  source 1..73: mol_type = other DNA
tcctcgttagtatagtggtgagtatccccgcctgtcacgcgggagaccggggttcgattccccgacggggaga

SEQ ID NO: 433 - DNA, 73 bp, synthetic construct
  source 1..73: mol_type = other DNA
ggctcgttggtctaggggtatgattctcgcttcgggtgcgagaggtcccgggttcaaatcccggacgagccct

SEQ ID NO: 434 - DNA, 75 bp, synthetic construct
  source 1..75: mol_type = other DNA
ggcgccgtggcttagttggttaaagcgcctgtctagtaaacaggagatcctgggttcgaatcccagcggtgcctt

SEQ ID NO: 435 - DNA, 83 bp, synthetic construct
  source 1..83: mol_type = other DNA
gtagtcgtggccgagtggttaaggcgatggactagaaatccattggggtctccccgcgcaggttcgaatcctgccgactacgt

SEQ ID NO: 436 - DNA, 73 bp, synthetic construct
  source 1..73: mol_type = other DNA
tgacctcgtggcgcaatggtagcgcgtctgactccagatcagaaggttgcgtgttcaagtcacgtcggggtca

SEQ ID NO: 437 - DNA, 73 bp, synthetic construct
  source 1..73: mol_type = other DNA
aggcgcggtggccaagtggtaaggcgtcggtctcgtaaaccgaagatcgcgggttcgaaccccgtccgtgcct

SEQ ID NO: 438 - DNA, 73 bp, synthetic construct
  source 1..73: mol_type = other DNA
agggggtatagctcagtggtagagcatttgactgcagatcaagaggtccccggttcaaatccgggtgccccct

SEQ ID NO: 439 - DNA, 73 bp, synthetic construct
  source 1..73: mol_type = other DNA
gggggtatagctcagtggtagagcatttgactgcagatcaagaggtccctggttcaaatccgggtgccccctc

SEQ ID NO: 440 - DNA, 73 bp, synthetic construct
  source 1..73: mol_type = other DNA
gggggtatagctcagtggtagagcatttgactgcagatcaagaggtccccggttcaaatccgggtgccccctc

SEQ ID NO: 441 - DNA, 73 bp, synthetic construct
  source 1..73: mol_type = other DNA
aggtcccatggtgtaatggttagcactctggactttgaatccagcgatccgagttcaaatctcggtgggacct

SEQ ID NO: 442 - DNA, 74 bp, synthetic construct
  source 1..74: mol_type = other DNA
gacccagtggcctaatggataaggcatcagcctccggagctggggattgtgggttcgagtcccatctgggtcgc

SEQ ID NO: 443 - DNA, 74 bp, synthetic construct
  source 1..74: mol_type = other DNA
agccccagtggcctaatggataaggcactggcctcctaagccagggattgtgggttcgagtcccacctggggta

SEQ ID NO: 444 - DNA, 74 bp, synthetic construct
  source 1..74: mol_type = other DNA
gccccagtggcctaatggataaggcactggcctcctaagccagggattgtgggttcgagtcccacctggggtgt

SEQ ID NO: 445 - DNA, 74 bp, synthetic construct
  source 1..74: mol_type = other DNA
agaccgcgtggcctaatggataaggcgtctgacttcggatcagaagattgagggttcgagtcccttcgtggtcg

SEQ ID NO: 446 - DNA, 75 bp, synthetic construct
  source 1..75: mol_type = other DNA
cgtctctgtggcgcaatcggttagcgcgttcggctgttaaccgaaaggttggtggttcgagcccacccagggacg

SEQ ID NO: 447 - DNA, 73 bp, synthetic construct
  source 1..73: mol_type = other DNA
ggcgttggtggtatagtggttagcatagctgccttccaagcagttgacccgggttcgattcccggccaacgca

SEQ ID NO: 448 - DNA, 74 bp, synthetic construct
  source 1..74: mol_type = other DNA
gtttccgtagtgtagcggttatcacattcgcctcacacgcgaaaggtccccggttcgatcccgggcggaaacag

SEQ ID NO: 449 - DNA, 75 bp, synthetic construct
  source 1..75: mol_type = other DNA
tggcgccgtggcttagttggttaaagcgcctgtctagtaaacaggagatcctgggttcgaatcccagcggtgcct

SEQ ID NO: 450 - DNA, 94 bp, synthetic construct
  source 1..94: mol_type = other DNA
gctccagtggcgcaatcggttagcgcgcggtacttatatgacagtgcgagcggagcaatgccgaggttgtgagttcgatcctcacctggagcac

SEQ ID NO: 451 - DNA, 72 bp, synthetic construct
  source 1..72: mol_type = other DNA
gcatgggtggttcagtggtagaattctcgcctgccacgcgggaggcccgggttcgattcccggcccatgcag

SEQ ID NO: 452 - DNA, 18 bp, synthetic construct
  source 1..18: mol_type = other DNA
aaaatataaatatatttc

SEQ ID NO: 453-454 - skipped (2 entries)

SEQ ID NO: 455 - DNA, 15 bp, synthetic construct
  source 1..15: mol_type = other DNA
aattcttcggaatgt

SEQ ID NO: 456-460 - skipped (5 entries)

SEQ ID NO: 461 - DNA, 20 bp, synthetic construct
  source 1..20: mol_type = other DNA
caggcgggttctgcccgcgc

SEQ ID NO: 462 - DNA, 18 bp, synthetic construct
  source 1..18: mol_type = other DNA
catacctgcaagggtatc

SEQ ID NO: 463 - DNA, 15 bp, synthetic construct
  source 1..15: mol_type = other DNA
cgaccgcaaggttgt

SEQ ID NO: 464 - DNA, 15 bp, synthetic construct
  source 1..15: mol_type = other DNA
cgaccttgcggtcat

SEQ ID NO: 465 - DNA, 18 bp, synthetic construct
  source 1..18: mol_type = other DNA
cgatgctaatcacatcgt

SEQ ID NO: 466 - DNA, 15 bp, synthetic construct
  source 1..15: mol_type = other DNA
cgatggtgacatcat

SEQ ID NO: 467 - DNA, 16 bp, synthetic construct
  source 1..16: mol_type = other DNA
cgatggtttacatcgt

SEQ ID NO: 468 - DNA, 13 bp, synthetic construct
  source 1..13: mol_type = other DNA
cgccgtaaggtgt

SEQ ID NO: 469 - DNA, 12 bp, synthetic construct
  source 1..12: mol_type = other DNA
cgccttaggtgt

SEQ ID NO: 470 - DNA, 15 bp, synthetic construct
  source 1..15: mol_type = other DNA
cgcctttcgacgcgt

SEQ ID NO: 471 - DNA, 13 bp, synthetic construct
  source 1..13: mol_type = other DNA
cgcttcacggcgt

SEQ ID NO: 472 - DNA, 15 bp, synthetic construct
  source 1..15: mol_type = other DNA
cggcagcaatgctgt

SEQ ID NO: 473 - DNA, 13 bp, synthetic construct
  source 1..13: mol_type = other DNA
cggctccgccttc

SEQ ID NO: 474 - DNA, 16 bp, synthetic construct
  source 1..16: mol_type = other DNA
cgggtatcacagggtc

SEQ ID NO: 475 - DNA, 17 bp, synthetic construct
  source 1..17: mol_type = other DNA
cggtgcgcaagcgctgt

SEQ ID NO: 476 - DNA, 18 bp, synthetic construct
  source 1..18: mol_type = other DNA
cgtacgggtgaccgtacc

SEQ ID NO: 477 - DNA, 13 bp, synthetic construct
  source 1..13: mol_type = other DNA
cgtcaaagacttc

SEQ ID NO: 478 - DNA, 13 bp, synthetic construct
  source 1..13: mol_type = other DNA
cgtcgtaagactt

SEQ ID NO: 479 - DNA, 14 bp, synthetic construct
  source 1..14: mol_type = other DNA
cgttgaataaacgt

SEQ ID NO: 480-485 - skipped (6 entries)

SEQ ID NO: 486 - DNA, 19 bp, synthetic construct
  source 1..19: mol_type = other DNA
taactagatactttcagat

SEQ ID NO: 487 - DNA, 15 bp, synthetic construct
  source 1..15: mol_type = other DNA
tactcgtatgggtgc

SEQ ID NO: 488 - DNA, 13 bp, synthetic construct
  source 1..13: mol_type = other DNA
tactttgcggtgt

SEQ ID NO: 489 - DNA, 19 bp, synthetic construct
  source 1..19: mol_type = other DNA
taggcgagtaacatcgtgc

SEQ ID NO: 490 - DNA, 19 bp, synthetic construct
  source 1..19: mol_type = other DNA
taggcgtgaatagcgcctc

SEQ ID NO: 491 - DNA, 19 bp, synthetic construct
  source 1..19: mol_type = other DNA
taggtcgcgagagcggcgc

SEQ ID NO: 492 - DNA, 19 bp, synthetic construct
  source 1..19: mol_type = other DNA
taggtcgcgtaagcggcgc

SEQ ID NO: 493 - DNA, 17 bp, synthetic construct
  source 1..17: mol_type = other DNA
taggtggttatccacgc

SEQ ID NO: 494-495 - skipped (2 entries)

SEQ ID NO: 496 - DNA, 18 bp, synthetic construct
  source 1..18: mol_type = other DNA
tatacgtgaaagcgtatc

SEQ ID NO: 497 - DNA, 21 bp, synthetic construct
  source 1..21: mol_type = other DNA
tatagggtcaaaaactctatc

SEQ ID NO: 498 - DNA, 20 bp, synthetic construct
  source 1..20: mol_type = other DNA
tatgcagaaatacctgcatc

SEQ ID NO: 499 - DNA, 15 bp, synthetic construct
  source 1..15: mol_type = other DNA
tccccatacgggggc

SEQ ID NO: 500 - DNA, 14 bp, synthetic construct
  source 1..14: mol_type = other DNA
tcccgaaggggttc

SEQ ID NO: 501 - DNA, 15 bp, synthetic construct
  source 1..15: mol_type = other DNA
tctacgtatgtgggc

SEQ ID NO: 502 - DNA, 14 bp, synthetic construct
  source 1..14: mol_type = other DNA
tctcataggagttc

SEQ ID NO: 503 - DNA, 14 bp, synthetic construct
  source 1..14: mol_type = other DNA
tctcctctggaggc

SEQ ID NO: 504 - DNA, 15 bp, synthetic construct
  source 1..15: mol_type = other DNA
tcttagcaataaggt

SEQ ID NO: 505 - DNA, 14 bp, synthetic construct
  source 1..14: mol_type = other DNA
tcttgtaggagttc

SEQ ID NO: 506 - DNA, 15 bp, synthetic construct
  source 1..15: mol_type = other DNA
tgaacgtaagttcgc

SEQ ID NO: 507 - DNA, 16 bp, synthetic construct
  source 1..16: mol_type = other DNA
tgaactgcgaggttcc

SEQ ID NO: 508 - skipped

SEQ ID NO: 509 - DNA, 15 bp, synthetic construct
  source 1..15: mol_type = other DNA
tgaccgaaaggtcgt

SEQ ID NO: 510 - DNA, 15 bp, synthetic construct
  source 1..15: mol_type = other DNA
tgaccgcaaggtcgt

SEQ ID NO: 511 - DNA, 14 bp, synthetic construct
  source 1..14: mol_type = other DNA
tgagctctgctctc

SEQ ID NO: 512 - DNA, 18 bp, synthetic construct
  source 1..18: mol_type = other DNA
tgaggcctcacggcctac

SEQ ID NO: 513 - DNA, 15 bp, synthetic construct
  source 1..15: mol_type = other DNA
tgagggcaacttcgt

SEQ ID NO: 514 - DNA, 16 bp, synthetic construct
  source 1..16: mol_type = other DNA
tgagggtcatacctcc

SEQ ID NO: 515 - DNA, 18 bp, synthetic construct
  source 1..18: mol_type = other DNA
tgagggtgcaaatcctcc

SEQ ID NO: 516 - DNA, 13 bp, synthetic construct
  source 1..13: mol_type = other DNA
tgccgaaaggcgt

SEQ ID NO: 517 - DNA, 13 bp, synthetic construct
  source 1..13: mol_type = other DNA
tgccgtaaggcgt

SEQ ID NO: 518 - DNA, 14 bp, synthetic construct
  source 1..14: mol_type = other DNA
tgcggtctccgcgc

SEQ ID NO: 519 - DNA, 11 bp, synthetic construct
  source 1..11: mol_type = other DNA
tgctagagcat

SEQ ID NO: 520 - DNA, 16 bp, synthetic construct
  source 1..16: mol_type = other DNA
tgctcgtatagagctc

SEQ ID NO: 521 - DNA, 15 bp, synthetic construct
  source 1..15: mol_type = other DNA
tggacaattgtctgc

SEQ ID NO: 522 - DNA, 15 bp, synthetic construct
  source 1..15: mol_type = other DNA
tggacagatgtccgt

SEQ ID NO: 523 - DNA, 15 bp, synthetic construct
  source 1..15: mol_type = other DNA
tggacaggtgtccgc

SEQ ID NO: 524 - DNA, 15 bp, synthetic construct
  source 1..15: mol_type = other DNA
tggacggttgtccgc

SEQ ID NO: 525 - DNA, 13 bp, synthetic construct
  source 1..13: mol_type = other DNA
tggacttgtggtc

SEQ ID NO: 526 - DNA, 16 bp, synthetic construct
  source 1..16: mol_type = other DNA
tggagattctctccgc

SEQ ID NO: 527 - DNA, 14 bp, synthetic construct
  source 1..14: mol_type = other DNA
tggcataggcctgc

SEQ ID NO: 528 - DNA, 14 bp, synthetic construct
  source 1..14: mol_type = other DNA
tggcttatgtctac

SEQ ID NO: 529 - DNA, 16 bp, synthetic construct
  source 1..16: mol_type = other DNA
tgggagttaatcccgt

SEQ ID NO: 530 - DNA, 14 bp, synthetic construct
  source 1..14: mol_type = other DNA
tgggatcttcccgc

SEQ ID NO: 531 - DNA, 16 bp, synthetic construct
  source 1..16: mol_type = other DNA
tgggcagaaatgtctc

SEQ ID NO: 532 - DNA, 15 bp, synthetic construct
  source 1..15: mol_type = other DNA
tgggcgttcgcccgc

SEQ ID NO: 533 - DNA, 14 bp, synthetic construct
  source 1..14: mol_type = other DNA
tgggcttcgcccgc

SEQ ID NO: 534 - DNA, 16 bp, synthetic construct
  source 1..16: mol_type = other DNA
tgggggataaccccgt

SEQ ID NO: 535 - DNA, 15 bp, synthetic construct
  source 1..15: mol_type = other DNA
tgggggtttccccgt

SEQ ID NO: 536 - skipped

SEQ ID NO: 537 - DNA, 15 bp, synthetic construct
  source 1..15: mol_type = other DNA
tggtggcaacaccgt

SEQ ID NO: 538 - DNA, 14 bp, synthetic construct
  source 1..14: mol_type = other DNA
tggtttatagccgt

SEQ ID NO: 539 - DNA, 19 bp, synthetic construct
  source 1..19: mol_type = other DNA
tgtacggtaataccgtacc

SEQ ID NO: 540 - DNA, 15 bp, synthetic construct
  source 1..15: mol_type = other DNA
tgtccgcaaggacgt

SEQ ID NO: 541 - DNA, 15 bp, synthetic construct
  source 1..15: mol_type = other DNA
tgtcctaacggacgt

SEQ ID NO: 542 - DNA, 18 bp, synthetic construct
  source 1..18: mol_type = other DNA
tgtcctattaacggacgt

SEQ ID NO: 543 - DNA, 16 bp, synthetic construct
  source 1..16: mol_type = other DNA
tgtccttcacgggcgt

SEQ ID NO: 544 - DNA, 13 bp, synthetic construct
  source 1..13: mol_type = other DNA
tgtcttaggacgt

SEQ ID NO: 545 - DNA, 18 bp, synthetic construct
  source 1..18: mol_type = other DNA
tgtgcgttaacgcgtacc

SEQ ID NO: 546 - DNA, 16 bp, synthetic construct
  source 1..16: mol_type = other DNA
tgtgtcgcaaggcacc

SEQ ID NO: 547 - DNA, 15 bp, synthetic construct
  source 1..15: mol_type = other DNA
tgttcgtaaggactt

SEQ ID NO: 548 - DNA, 16 bp, synthetic construct
  source 1..16: mol_type = other DNA
ttcacagaaatgtgtc

SEQ ID NO: 549 - DNA, 14 bp, synthetic construct
  source 1..14: mol_type = other DNA
ttccctcgtggagt

SEQ ID NO: 550 - DNA, 14 bp, synthetic construct
  source 1..14: mol_type = other DNA
ttccctctgggagc

SEQ ID NO: 551 - DNA, 14 bp, synthetic construct
  source 1..14: mol_type = other DNA
ttcccttgtggatc

SEQ ID NO: 552 - DNA, 13 bp, synthetic construct
  source 1..13: mol_type = other DNA
ttccttcgggagc

SEQ ID NO: 553 - DNA, 15 bp, synthetic construct
  source 1..15: mol_type = other DNA
ttctagcaatagagt

SEQ ID NO: 554 - DNA, 16 bp, synthetic construct
  source 1..16: mol_type = other DNA
ttctccactggggagc

SEQ ID NO: 555 - DNA, 15 bp, synthetic construct
  source 1..15: mol_type = other DNA
ttctcgagagggagc

SEQ ID NO: 556 - DNA, 15 bp, synthetic construct
  source 1..15: mol_type = other DNA
ttctcgtatgagagc

SEQ ID NO: 557 - DNA, 19 bp, synthetic construct
  source 1..19: mol_type = other DNA
tttaaggttttcccttaac

SEQ ID NO: 558 - DNA, 14 bp, synthetic construct
  source 1..14: mol_type = other DNA
tttcattgtggagt

SEQ ID NO: 559 - DNA, 14 bp, synthetic construct
  source 1..14: mol_type = other DNA
tttcgaaggaatcc

SEQ ID NO: 560 - DNA, 13 bp, synthetic construct
  source 1..13: mol_type = other DNA
tttcttcggaagc

SEQ ID NO: 561 - DNA, 16 bp, synthetic construct
  source 1..16: mol_type = other DNA
tttggggcaactcaac

SEQ ID NO: 562-621 - skipped (60 entries)

SEQ ID NO: 622 - RNA, 76 nt, synthetic construct
  source 1..76: mol_type = other RNA
ggctccgtggcgcaatggatagcgcattggacttcaaattcaaaggttccgggttcgagtcccggcggagtcgcca

SEQ ID NO: 623 - RNA, 85 nt, synthetic construct
  source 1..85: mol_type = other RNA
gacgaggtggccgagtggttaaggcgatggactctaaatccattgtgctctgcacgcgtgggttcgaatcccatcctcgtcgcca

SEQ ID NO: 624 - RNA, 75 nt, synthetic construct
  source 1..75: mol_type = other RNA
ggttccatggtgtaatggtaagcactctggactttaaatccagcgatccgagttcgagtctcggtggaacctcca

SEQ ID NO: 625 - RNA, 85 nt, synthetic construct
  source 1..85: mol_type = other RNA
gacgaggtggccgagtggttaaggcgatggacttcaaatccattgtgctctgcacgcgtgggttcgaatcccatcctcgtcgcca

SEQ ID NO: 626 - RNA, 76 nt, synthetic construct; identical to SEQ ID NO: 622
  source 1..76: mol_type = other RNA
  modified_base 1: mod_base = OTHER; note = 2'-O-methyl modified nucleotide
  modified_base 50: mod_base = OTHER; note = 2'-O-methyl modified nucleotide
  modified_base 52: mod_base = OTHER; note = 2'-O-methyl modified nucleotide
  modified_base 73: mod_base = OTHER; note = 2'-O-methyl modified nucleotide

SEQ ID NO: 627 - RNA, 76 nt, synthetic construct; identical to SEQ ID NO: 622
  source 1..76: mol_type = other RNA
  modified_base 1: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 50: mod_base = OTHER; note = 2'-O-methyl modified nucleotide
  modified_base 52: mod_base = OTHER; note = 2'-O-methyl modified nucleotide
  modified_base 73: mod_base = OTHER; note = 2'-O-methyl modified nucleotide

SEQ ID NO: 628 - RNA, 76 nt, synthetic construct; identical to SEQ ID NO: 622
  source 1..76: mol_type = other RNA
  modified_base 1: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-fluoro modified nucleotide
  modified_base 20: mod_base = OTHER; note = 2'-O-methyl modified nucleotide
  modified_base 73: mod_base = OTHER; note = 2'-O-methyl modified nucleotide

SEQ ID NO: 629 - DNA, 76 bp, synthetic construct
  source 1..76: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 30: mod_base = OTHER; note = nucleotide with phosphorothioate linkage
  modified_base 44: mod_base = OTHER; note = nucleotide with phosphorothioate linkage
  modified_base 61: mod_base = OTHER; note = 2'-O-methyl modified nucleotide
  modified_base 74: mod_base = OTHER; note = nucleotide with phosphorothioate linkage
  modified_base 75: mod_base = OTHER; note = Nucleotide with phosphorothioate linkage
  modified_base 76: mod_base = OTHER; note = 2'-O-methyl modified nucleotide
  misc_feature 1..44: note = RNA
  misc_feature 45: note = DNA
  misc_feature 46..74: note = RNA
  misc_feature 75: note = DNA
  misc_feature 76: note = RNA
ggctccgtggcgcaatggatagcgcattggacttcaaattcaaaggttccgggttcgagtcccggcggagtcgcca

SEQ ID NO: 630 - DNA, 76 bp, synthetic construct; identical to SEQ ID NO: 629
  source 1..76: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 19: mod_base = OTHER; note = nucleotide with phosphorothioate linkage
  modified_base 29: mod_base = OTHER; note = 2'-O-(2-methoxyethyl) modified nucleotide
  modified_base 68: mod_base = OTHER; note = 2'-fluoro modified nucleotide
  modified_base 72: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide
  misc_feature 1..19: note = RNA
  misc_feature 20: note = DNA
  misc_feature 21..43: note = RNA
  misc_feature 44: note = DNA
  misc_feature 45..76: note = RNA

SEQ ID NO: 631 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 74: mod_base = OTHER; note = nucleotide with phosphorothioate linkage
  modified_base 75: mod_base = OTHER; note = 2'-O-methyl modified nucleotide

SEQ ID NO: 632 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 74: mod_base = OTHER; note = nucleotide with phosphorothioate linkage
  modified_base 75: mod_base = OTHER; note = 2'-O-methyl modified nucleotide

SEQ ID NO: 633 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 45: mod_base = OTHER; note = 2'-fluoro modified nucleotide
  modified_base 57: mod_base = OTHER; note = 2'-fluoro modified nucleotide
  modified_base 68: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide

SEQ ID NO: 634 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 33: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide

SEQ ID NO: 635 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 18: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide

SEQ ID NO: 636 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 25: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide

SEQ ID NO: 637 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 22: mod_base = OTHER; note = 2'-O-methyl modified nucleotide
  modified_base 52: mod_base = OTHER; note = 2'-O-methyl modified nucleotide
  modified_base 74: mod_base = OTHER; note = nucleotide with phosphorothioate linkage
  modified_base 75: mod_base = OTHER; note = 2'-O-methyl modified nucleotide

SEQ ID NO: 638 - DNA, 76 bp, synthetic construct; identical to SEQ ID NO: 629
  source 1..76: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 52: mod_base = OTHER; note = 2'-O-methyl modified nucleotide
  modified_base 63: mod_base = OTHER; note = nucleotide with phosphorothioate linkage
  modified_base 74: mod_base = OTHER; note = nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide
  misc_feature 1..63: note = RNA
  misc_feature 64: note = DNA
  misc_feature 65..76: note = RNA

SEQ ID NO: 639 - DNA, 76 bp, synthetic construct; identical to SEQ ID NO: 629
  source 1..76: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 9: mod_base = OTHER; note = nucleotide with phosphorothioate linkage
  modified_base 52: mod_base = OTHER; note = 2'-O-methyl modified nucleotide
  modified_base 59: mod_base = OTHER; note = 2'-O-(2-methoxyethyl) modified nucleotide
  modified_base 74: mod_base = OTHER; note = nucleotide with phosphorothioate linkage
  modified_base 75: mod_base = OTHER; note = 2'-O-methyl modified nucleotide
  misc_feature 1..9: note = RNA
  misc_feature 10: note = DNA
  misc_feature 11..76: note = RNA

SEQ ID NO: 640 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 13: mod_base = OTHER; note = 2'-fluoro modified nucleotide
  modified_base 14: mod_base = OTHER; note = 2'-fluoro modified nucleotide
  modified_base 54: mod_base = OTHER; note = 2'-fluoro modified nucleotide
  modified_base 59: mod_base = OTHER; note = 2'-O-methyl modified nucleotide
  modified_base 75: mod_base = OTHER; note = nucleotide with phosphorothioate linkage
  modified_base 76: mod_base = OTHER; note = 2'-O-methyl modified nucleotide

SEQ ID NO: 641 - DNA, 76 bp, synthetic construct; identical to SEQ ID NO: 629
  source 1..76: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 27: mod_base = OTHER; note = nucleotide with phosphorothioate linkage
  modified_base 51: mod_base = OTHER; note = nucleotide with phosphorothioate linkage
  modified_base 59: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide
  misc_feature 1..51: note = RNA
  misc_feature 52: note = DNA
  misc_feature 53..76: note = RNA

SEQ ID NO: 642 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 4: mod_base = OTHER; note = 2'-O-methyl modified nucleotide
  modified_base 74: mod_base = OTHER; note = nucleotide with phosphorothioate linkage
  modified_base 75: mod_base = OTHER; note = 2'-O-methyl modified nucleotide

SEQ ID NO: 643 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 5: mod_base = OTHER; note = 2'-O-methyl modified nucleotide
  modified_base 61: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide

SEQ ID NO: 644 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 13: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide

SEQ ID NO: 645 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 33: mod_base = OTHER; note = 2'-fluoro modified nucleotide
  modified_base 53: mod_base = OTHER; note = 2'-O-methyl modified nucleotide
  modified_base 74: mod_base = OTHER; note = nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide

SEQ ID NO: 646 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 64: mod_base = OTHER; note = nucleotide with phosphorothioate linkage
  modified_base 65: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide

SEQ ID NO: 647 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 43: mod_base = OTHER; note = nucleotide with phosphorothioate linkage
  modified_base 55: mod_base = OTHER; note = nucleotide with phosphorothioate linkage
  modified_base 74: mod_base = OTHER; note = nucleotide with phosphorothioate linkage
  modified_base 75: mod_base = OTHER; note = 2'-O-methyl modified nucleotide

SEQ ID NO: 648 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 16: mod_base = OTHER; note = nucleotide with phosphorothioate linkage
  modified_base 17: mod_base = OTHER; note = 2'-O-(2-methoxyethyl) modified nucleotide
  modified_base 25: mod_base = OTHER; note = 2'-fluoro modified nucleotide
  modified_base 30: mod_base = OTHER; note = nucleotide with phosphorothioate linkage
  modified_base 44: mod_base = OTHER; note = 2'-fluoro modified nucleotide
  modified_base 52: mod_base = OTHER; note = nucleotide with phosphorothioate linkage
  modified_base 74: mod_base = OTHER; note = nucleotide with phosphorothioate linkage
  modified_base 75: mod_base = OTHER; note = 2'-O-methyl modified nucleotide

SEQ ID NO: 649 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 66: mod_base = OTHER; note = nucleotide with phosphorothioate linkage
  modified_base 67: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide

SEQ ID NO: 650 - RNA, 76 nt, synthetic construct; identical to SEQ ID NO: 622
  source 1..76: mol_type = other RNA
  modified_base 1: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-fluoro modified nucleotide
  modified_base 20: mod_base = OTHER; note = 2'-O-methyl modified nucleotide
  modified_base 50: mod_base = OTHER; note = 2'-O-methyl modified nucleotide
  modified_base 52: mod_base = OTHER; note = 2'-O-methyl modified nucleotide
  modified_base 73: mod_base = OTHER; note = 2'-O-methyl modified nucleotide

SEQ ID NO: 651 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 49: mod_base = OTHER; note = 2'-O-methyl modified nucleotide
  modified_base 50: mod_base = OTHER; note = 2'-O-methyl modified nucleotide
  modified_base 51: mod_base = OTHER; note = 2'-O-methyl modified nucleotide
  modified_base 52: mod_base = OTHER; note = 2'-O-methyl modified nucleotide
  modified_base 53: mod_base = OTHER; note = 2'-O-methyl modified nucleotide
  modified_base 61: mod_base = OTHER; note = 2'-O-methyl modified nucleotide
  modified_base 62: mod_base = OTHER; note = 2'-O-methyl modified nucleotide
  modified_base 63: mod_base = OTHER; note = 2'-fluoro modified nucleotide
  modified_base 64: mod_base = OTHER; note = 2'-O-methyl modified nucleotide
  modified_base 65: mod_base = OTHER; note = 2'-O-methyl modified nucleotide
  modified_base 66: mod_base = OTHER; note = 2'-O-methyl modified nucleotide
  modified_base 67: mod_base = OTHER; note = 2'-O-methyl modified nucleotide
  modified_base 68: mod_base = OTHER; note = 2'-O-methyl modified nucleotide
  modified_base 69: mod_base = OTHER; note = 2'-fluoro modified nucleotide
  modified_base 71: mod_base = OTHER; note = 2'-O-methyl modified nucleotide

SEQ ID NO: 652 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 49: mod_base = OTHER; note = 2'-O-methyl modified nucleotide
  modified_base 50: mod_base = OTHER; note = 2'-O-methyl modified nucleotide
  modified_base 51: mod_base = OTHER; note = 2'-O-methyl modified nucleotide
  modified_base 52: mod_base = OTHER; note = 2'-O-methyl modified nucleotide
  modified_base 53: mod_base = OTHER; note = 2'-O-methyl modified nucleotide
  modified_base 61: mod_base = OTHER; note = 2'-O-methyl modified nucleotide
  modified_base 62: mod_base = OTHER; note = 2'-O-methyl modified nucleotide
  modified_base 63: mod_base = OTHER; note = 2'-fluoro modified nucleotide
  modified_base 64: mod_base = OTHER; note = 2'-O-methyl modified nucleotide
  modified_base 65: mod_base = OTHER; note = 2'-O-methyl modified nucleotide
  modified_base 66: mod_base = OTHER; note = 2'-O-methyl modified nucleotide
  modified_base 67: mod_base = OTHER; note = 2'-O-methyl modified nucleotide
  modified_base 68: mod_base = OTHER; note = 2'-O-methyl modified nucleotide
  modified_base 69: mod_base = OTHER; note = 2'-fluoro modified nucleotide
  modified_base 71: mod_base = OTHER; note = 2'-O-methyl modified nucleotide
  modified_base 72: mod_base = OTHER; note = 2'-O-methyl modified nucleotide

SEQ ID NO: 653 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 54: mod_base = OTHER; note = 2'-O-methyl modified nucleotide
  modified_base 56: mod_base = OTHER; note = 2'-O-methyl modified nucleotide
  modified_base 57: mod_base = OTHER; note = 2'-fluoro modified nucleotide
  modified_base 59: mod_base = OTHER; note = 2'-O-methyl modified nucleotide
  modified_base 73: mod_base = OTHER; note = 2'-O-methyl modified nucleotide

SEQ ID NO: 654 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 54: mod_base = OTHER; note = 2'-O-methyl modified nucleotide with phosphorothioate linkage
  modified_base 56: mod_base = OTHER; note = 2'-O-methyl modified nucleotide
  modified_base 57: mod_base = OTHER; note = 2'-fluoro modified nucleotide
  modified_base 59: mod_base = OTHER; note = 2'-O-methyl modified nucleotide
  modified_base 73: mod_base = OTHER; note = 2'-O-methyl modified nucleotide

SEQ ID NO: 655 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 44: mod_base = OTHER; note = 2'-O-methyl modified nucleotide
  modified_base 45: mod_base = OTHER; note = 2'-O-methyl modified nucleotide
  modified_base 46: mod_base = OTHER; note = 2'-fluoro modified nucleotide
  modified_base 48: mod_base = OTHER; note = 2'-fluoro modified nucleotide
  modified_base 73: mod_base = OTHER; note = 2'-O-methyl modified nucleotide

SEQ ID NO: 656 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 43: mod_base = OTHER; note = 2'-O-methyl modified nucleotide
  modified_base 44: mod_base = OTHER; note = 2'-O-methyl modified nucleotide
  modified_base 45: mod_base = OTHER; note = 2'-O-methyl modified nucleotide
  modified_base 46: mod_base = OTHER; note = 2'-fluoro modified nucleotide
  modified_base 48: mod_base = OTHER; note = 2'-fluoro modified nucleotide
  modified_base 49: mod_base = OTHER; note = 2'-O-methyl modified nucleotide
  modified_base 50: mod_base = OTHER; note = 2'-O-methyl modified nucleotide
  modified_base 73: mod_base = OTHER; note = 2'-O-methyl modified nucleotide

SEQ ID NO: 657 - RNA, 76 nt, synthetic construct; identical to SEQ ID NO: 622
  source 1..76: mol_type = other RNA

SEQ ID NO: 658 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide

SEQ ID NO: 659 - RNA, 76 nt, synthetic construct; identical to SEQ ID NO: 622
  source 1..76: mol_type = other RNA
  modified_base 1: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 33: mod_base = OTHER; note = 2'-fluoro modified nucleotide
  modified_base 50: mod_base = OTHER; note = 2'-O-methyl modified nucleotide
  modified_base 52: mod_base = OTHER; note = 2'-O-methyl modified nucleotide
  modified_base 55: mod_base = OTHER; note = nucleotide with phosphorothioate linkage
  modified_base 73: mod_base = OTHER; note = 2'-O-methyl modified nucleotide

SEQ ID NO: 660 - RNA, 76 nt, synthetic construct; identical to SEQ ID NO: 622
  source 1..76: mol_type = other RNA
  modified_base 1: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 33: mod_base = OTHER; note = 2'-fluoro modified nucleotide
  modified_base 50: mod_base = OTHER; note = 2'-O-methyl modified nucleotide
  modified_base 52: mod_base = OTHER; note = 2'-O-methyl modified nucleotide
  modified_base 73: mod_base = OTHER; note = 2'-O-methyl modified nucleotide

SEQ ID NO: 661 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 17: mod_base = OTHER; note = 2'-O-methyl modified nucleotide
  modified_base 33: mod_base = OTHER; note = 2'-fluoro modified nucleotide
  modified_base 35: mod_base = OTHER; note = nucleotide with phosphorothioate linkage
  modified_base 37: mod_base = OTHER; note = nucleotide with phosphorothioate linkage
  modified_base 38: mod_base = OTHER; note = nucleotide with phosphorothioate linkage
  modified_base 52: mod_base = OTHER; note = 2'-O-methyl modified nucleotide
  modified_base 57: mod_base = OTHER; note = 2'-O-methyl modified nucleotide
  modified_base 62: mod_base = OTHER; note = 2'-O-methyl modified nucleotide
  modified_base 73: mod_base = OTHER; note = 2'-O-methyl modified nucleotide
  modified_base 75: mod_base = OTHER; note = 2'-fluoro modified nucleotide with phosphorothioate linkage

SEQ ID NO: 662 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 33: mod_base = OTHER; note = 2'-fluoro modified nucleotide
  modified_base 35: mod_base = OTHER; note = nucleotide with phosphorothioate linkage
  modified_base 37: mod_base = OTHER; note = nucleotide with phosphorothioate linkage
  modified_base 38: mod_base = OTHER; note = nucleotide with phosphorothioate linkage
  modified_base 52: mod_base = OTHER; note = 2'-O-methyl modified nucleotide
  modified_base 56: mod_base = OTHER; note = 2'-O-methyl modified nucleotide
  modified_base 62: mod_base = OTHER; note = 2'-O-methyl modified nucleotide
  modified_base 65: mod_base = OTHER; note = 2'-O-methyl modified nucleotide
  modified_base 73: mod_base = OTHER; note = 2'-O-methyl modified nucleotide
  modified_base 75: mod_base = OTHER; note = 2'-fluoro modified nucleotide with phosphorothioate linkage

SEQ ID NO: 663 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 17: mod_base = OTHER; note = 2'-O-methyl modified nucleotide
  modified_base 25: mod_base = OTHER; note = 2'-O-methyl modified nucleotide
  modified_base 33: mod_base = OTHER; note = 2'-fluoro modified nucleotide
  modified_base 35: mod_base = OTHER; note = nucleotide with phosphorothioate linkage
  modified_base 37: mod_base = OTHER; note = nucleotide with phosphorothioate linkage
  modified_base 38: mod_base = OTHER; note = nucleotide with phosphorothioate linkage
  modified_base 62: mod_base = OTHER; note = 2'-O-methyl modified nucleotide
  modified_base 73: mod_base = OTHER; note = 2'-O-methyl modified nucleotide
  modified_base 75: mod_base = OTHER; note = 2'-fluoro modified nucleotide with phosphorothioate linkage

SEQ ID NO: 664 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 33: mod_base = OTHER; note = 2'-fluoro modified nucleotide
  modified_base 52: mod_base = OTHER; note = 2'-O-(2-methoxyethyl) modified nucleotide
  modified_base 54: mod_base = OTHER; note = nucleotide with phosphorothioate linkage
  modified_base 55: mod_base = OTHER; note = nucleotide with phosphorothioate linkage

SEQ ID NO: 665 - RNA, 76 nt, synthetic construct; identical to SEQ ID NO: 622
  source 1..76: mol_type = other RNA
  modified_base 1: mod_base = OTHER; note = 2'-O-methyl modified nucleotide
  modified_base 10: mod_base = OTHER; note = 2'-O-methyl modified nucleotide
  modified_base 13: mod_base = OTHER; note = 2'-O-methyl modified nucleotide
  modified_base 17: mod_base = OTHER; note = nucleotide with phosphorothioate linkage
  modified_base 18: mod_base = OTHER; note = 2'-O-methyl modified nucleotide
  modified_base 19: mod_base = OTHER; note = nucleotide with phosphorothioate linkage
  modified_base 20: mod_base = OTHER; note = 2'-O-methyl modified nucleotide
  modified_base 25: mod_base = OTHER; note = 2'-O-methyl modified nucleotide
  modified_base 29: mod_base = OTHER; note = 2'-O-methyl modified nucleotide
  modified_base 33: mod_base = OTHER; note = 2'-fluoro modified nucleotide
  modified_base 35: mod_base = OTHER; note = nucleotide with phosphorothioate linkage
  modified_base 37: mod_base = OTHER; note = nucleotide with phosphorothioate linkage
  modified_base 38: mod_base = OTHER; note = nucleotide with phosphorothioate linkage
  modified_base 41: mod_base = OTHER; note = 2'-fluoro modified nucleotide
  modified_base 43: mod_base = OTHER; note = 2'-O-methyl modified nucleotide
  modified_base 44: mod_base = OTHER; note = 2'-fluoro modified nucleotide
  modified_base 46: mod_base = OTHER; note = nucleotide with phosphorothioate linkage
  modified_base 50: mod_base = OTHER; note = 2'-O-methyl modified nucleotide
  modified_base 52: mod_base = OTHER; note = 2'-O-methyl modified nucleotide
  modified_base 54: mod_base = OTHER; note = nucleotide with phosphorothioate linkage
  modified_base 55: mod_base = OTHER; note = nucleotide with phosphorothioate linkage
  modified_base 56: mod_base = OTHER; note = 2'-O-methyl modified nucleotide
  modified_base 57: mod_base = OTHER; note = nucleotide with phosphorothioate linkage
  modified_base 62: mod_base = OTHER; note = 2'-O-methyl modified nucleotide
  modified_base 65: mod_base = OTHER; note = 2'-O-methyl modified nucleotide
  modified_base 71: mod_base = OTHER; note = 2'-O-methyl modified nucleotide
  modified_base 73: mod_base = OTHER; note = 2'-O-methyl modified nucleotide

SEQ ID NO: 666 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 73: mod_base = OTHER; note = nucleotide with phosphorothioate linkage
  modified_base 74: mod_base = OTHER; note = nucleotide with phosphorothioate linkage
  modified_base 75: mod_base = OTHER; note = 2'-O-methyl modified nucleotide

SEQ ID NO: 667 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 73: mod_base = OTHER; note = nucleotide with phosphorothioate linkage
  modified_base 74: mod_base = OTHER; note = nucleotide with phosphorothioate linkage
  modified_base 75: mod_base = OTHER; note = 2'-O-methyl modified nucleotide

SEQ ID NO: 668 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 49: mod_base = OTHER; note = 2'-O-methyl modified nucleotide
  modified_base 50: mod_base = OTHER; note = 2'-O-methyl modified nucleotide
  modified_base 51: mod_base = OTHER; note = 2'-O-methyl modified nucleotide
  modified_base 52: mod_base = OTHER; note = 2'-O-methyl modified nucleotide
  modified_base 53: mod_base = OTHER; note = 2'-O-methyl modified nucleotide
  modified_base 54: mod_base = OTHER; note = 2'-O-methyl modified nucleotide with phosphorothioate linkage
  modified_base 55: mod_base = OTHER; note = nucleotide with phosphorothioate linkage
  modified_base 56: mod_base = OTHER; note = 2'-O-methyl modified nucleotide
  modified_base 57: mod_base = OTHER; note = 2'-fluoro modified nucleotide
  modified_base 59: mod_base = OTHER; note = 2'-O-methyl modified nucleotide
  modified_base 73: mod_base = OTHER; note = 2'-O-methyl modified nucleotide

SEQ ID NO: 669 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 33: mod_base = OTHER; note = 2'-fluoro modified nucleotide
  modified_base 35: mod_base = OTHER; note = nucleotide with phosphorothioate linkage
  modified_base 37: mod_base = OTHER; note = nucleotide with phosphorothioate linkage
  modified_base 38: mod_base = OTHER; note = nucleotide with phosphorothioate linkage
  modified_base 52: mod_base = OTHER; note = 2'-O-methyl modified nucleotide
  modified_base 56: mod_base = OTHER; note = 2'-O-methyl modified nucleotide
  modified_base 57: mod_base = OTHER; note = 2'-fluoro modified nucleotide
  modified_base 62: mod_base = OTHER; note = 2'-O-methyl modified nucleotide
  modified_base 63: mod_base = OTHER; note = 2'-fluoro modified nucleotide
  modified_base 73: mod_base = OTHER; note = 2'-O-methyl modified nucleotide
  modified_base 75: mod_base = OTHER; note = 2'-fluoro modified nucleotide with phosphorothioate linkage

SEQ ID NO: 670 - RNA, 76 nt, synthetic construct; identical to SEQ ID NO: 622
  source 1..76: mol_type = other RNA
  modified_base 1: mod_base = OTHER; note = 2'-O-methyl modified nucleotide
  modified_base 10: mod_base = OTHER; note = 2'-O-methyl modified nucleotide
  modified_base 13: mod_base = OTHER; note = 2'-O-methyl modified nucleotide
  modified_base 17: mod_base = OTHER; note = nucleotide with phosphorothioate linkage
  modified_base 18: mod_base = OTHER; note = 2'-O-methyl modified nucleotide
  modified_base 19: mod_base = OTHER; note = nucleotide with phosphorothioate linkage
  modified_base 20: mod_base = OTHER; note = 2'-O-methyl modified nucleotide
  modified_base 25: mod_base = OTHER; note = 2'-O-methyl modified nucleotide
  modified_base 29: mod_base = OTHER; note = 2'-O-methyl modified nucleotide
  modified_base 33: mod_base = OTHER; note = 2'-fluoro modified nucleotide
  modified_base 35: mod_base = OTHER; note = nucleotide with phosphorothioate linkage
  modified_base 37: mod_base = OTHER; note = nucleotide with phosphorothioate linkage
  modified_base 38: mod_base = OTHER; note = nucleotide with phosphorothioate linkage
  modified_base 41: mod_base = OTHER; note = 2'-fluoro modified nucleotide
  modified_base 43: mod_base = OTHER; note = 2'-O-methyl modified nucleotide
  modified_base 44: mod_base = OTHER; note = 2'-fluoro modified nucleotide
  modified_base 46: mod_base = OTHER; note = nucleotide with phosphorothioate linkage
  modified_base 50: mod_base = OTHER; note = 2'-O-methyl modified nucleotide
  modified_base 52: mod_base = OTHER; note = 2'-O-methyl modified nucleotide
  modified_base 54: mod_base = OTHER; note = nucleotide with phosphorothioate linkage
  modified_base 55: mod_base = OTHER; note = nucleotide with phosphorothioate linkage
  modified_base 56: mod_base = OTHER; note = 2'-O-methyl modified nucleotide
  modified_base 57: mod_base = OTHER; note = nucleotide with phosphorothioate linkage
  modified_base 62: mod_base = OTHER; note = 2'-O-methyl modified nucleotide
  modified_base 63: mod_base = OTHER; note = 2'-fluoro modified nucleotide
  modified_base 65: mod_base = OTHER; note = 2'-O-methyl modified nucleotide
  modified_base 71: mod_base = OTHER; note = 2'-fluoro modified nucleotide with phosphorothioate linkage
  modified_base 73: mod_base = OTHER; note = 2'-O-methyl modified nucleotide

SEQ ID NO: 671 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 13: mod_base = OTHER; note = 2'-fluoro modified nucleotide
  modified_base 14: mod_base = OTHER; note = 2'-fluoro modified nucleotide
  modified_base 54: mod_base = OTHER; note = 2'-fluoro modified nucleotide
  modified_base 59: mod_base = OTHER; note = 2'-O-methyl modified nucleotide
  modified_base 73: mod_base = OTHER; note = nucleotide with phosphorothioate linkage
  modified_base 75: mod_base = OTHER; note = nucleotide with phosphorothioate linkage
  modified_base 76: mod_base = OTHER; note = 2'-O-methyl modified nucleotide

SEQ ID NO: 672 - DNA, 76 bp, synthetic construct; identical to SEQ ID NO: 629
  source 1..76: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 27: mod_base = OTHER; note = nucleotide with phosphorothioate linkage
  modified_base 51: mod_base = OTHER; note = nucleotide with phosphorothioate linkage
  modified_base 59: mod_base = OTHER; note = 2'-fluoro modified nucleotide
  modified_base 73: mod_base = OTHER; note = nucleotide with phosphorothioate linkage
  modified_base 74: mod_base = OTHER; note = nucleotide with phosphorothioate linkage
  modified_base 75: mod_base = OTHER; note = 2'-O-methyl modified nucleotide
  misc_feature 1..51: note = RNA
  misc_feature 52: note = DNA
  misc_feature 53..76: note = RNA

SEQ ID NO: 673 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
  modified_base 49: mod_base = OTHER; note = 2'-O-methyl modified nucleotide
  modified_base 50: mod_base = OTHER; note = 2'-O-methyl modified nucleotide
  modified_base 51: mod_base = OTHER; note = 2'-O-methyl modified nucleotide
  modified_base 52: mod_base = OTHER; note = 2'-O-methyl modified nucleotide
  modified_base 53: mod_base = OTHER; note = 2'-O-methyl modified nucleotide
  modified_base 61: mod_base = OTHER; note = 2'-O-methyl modified nucleotide
  modified_base 62: mod_base = OTHER; note = 2'-O-methyl modified nucleotide
  modified_base 63: mod_base = OTHER; note = 2'-fluoro modified nucleotide
  modified_base 64: mod_base = OTHER; note = 2'-O-methyl modified nucleotide
  modified_base 65: mod_base = OTHER; note = 2'-O-methyl modified nucleotide
  modified_base 66: mod_base = OTHER; note = 2'-O-methyl modified nucleotide
  modified_base 67: mod_base = OTHER; note = 2'-O-methyl modified nucleotide
  modified_base 68: mod_base = OTHER; note = 2'-O-methyl modified nucleotide
  modified_base 69: mod_base = OTHER; note = 2'-fluoro modified nucleotide
  modified_base 71: mod_base = OTHER; note = 2'-O-methyl modified nucleotide

SEQ ID NO: 674 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 45: mod_base = OTHER; note = 2'-fluoro modified nucleotide
  modified_base 57: mod_base = OTHER; note = 2'-fluoro modified nucleotide
  modified_base 68: mod_base = OTHER; note = 2'-fluoro modified nucleotide
  modified_base 73: mod_base = OTHER; note = nucleotide with phosphorothioate linkage
  modified_base 74: mod_base = OTHER; note = nucleotide with phosphorothioate linkage
  modified_base 75: mod_base = OTHER; note = 2'-O-methyl modified nucleotide with phosphorothioate linkage
  modified_base 76: mod_base = OTHER; note = 2'-O-methyl modified nucleotide

SEQ ID NO: 675 - RNA, 76 nt, synthetic construct; identical to SEQ ID NO: 622
  source 1..76: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 13: mod_base = OTHER; note = 2'-O-methyl modified nucleotide
  modified_base 33: mod_base = OTHER; note = 2'-fluoro modified nucleotide
  modified_base 37: mod_base = OTHER; note = nucleotide with phosphorothioate linkage
  modified_base 52: mod_base = OTHER; note = 2'-O-methyl modified nucleotide
  modified_base 62: mod_base = OTHER; note = 2'-O-methyl modified nucleotide
  modified_base 73: mod_base = OTHER; note = 2'-O-methyl modified nucleotide
  modified_base 75: mod_base = OTHER; note = 2'-fluoro modified nucleotide with phosphorothioate linkage

SEQ ID NO: 676 - RNA, 75 nt, synthetic construct
  source 1..75: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 12: mod_base = OTHER; note = 2'-O-methyl modified nucleotide
  modified_base 13: mod_base = OTHER; note = nucleotide with phosphorothioate linkage
  modified_base 16: mod_base = OTHER; note = 2'-fluoro modified nucleotide
  modified_base 22: mod_base = OTHER; note = 2'-fluoro modified nucleotide
  modified_base 23: mod_base = OTHER; note = 2'-fluoro modified nucleotide
  modified_base 28: mod_base = OTHER; note = 2'-fluoro modified nucleotide
  modified_base 29: mod_base = OTHER; note = 2'-O-methyl modified nucleotide
  modified_base 44: mod_base = OTHER; note = 2'-fluoro modified nucleotide
  modified_base 53: mod_base = OTHER; note = 2'-fluoro modified nucleotide with phosphorothioate linkage
  modified_base 65: mod_base = OTHER; note = nucleotide with phosphorothioate linkage
  modified_base 66: mod_base = OTHER; note = nucleotide with phosphorothioate linkage
  modified_base 67: mod_base = OTHER; note = 2'-fluoro modified nucleotide
  modified_base 73: mod_base = OTHER; note = nucleotide with phosphorothioate linkage
  modified_base 74: mod_base = OTHER; note = 2'-O-methyl modified nucleotide
  modified_base 75: mod_base = OTHER; note = 2'-fluoro modified nucleotide
ggttccatggtgtaatggtaagcactctggactctaaatccagcgatccgagttcgagtctcggtggaacctcca

SEQ ID NO: 677 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 13: mod_base = OTHER; note = 2'-fluoro modified nucleotide with phosphorothioate linkage
  modified_base 19: mod_base = OTHER; note = nucleotide with phosphorothioate linkage
  modified_base 20: mod_base = OTHER; note = 2'-fluoro modified nucleotide
  modified_base 28: mod_base = OTHER; note = 2'-fluoro modified nucleotide
  modified_base 40: mod_base = OTHER; note = 2'-fluoro modified nucleotide
  modified_base 42: mod_base = OTHER; note = 2'-fluoro modified nucleotide
  modified_base 52: mod_base = OTHER; note = 2'-fluoro modified nucleotide with phosphorothioate linkage
  modified_base 67: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-fluoro modified nucleotide

SEQ ID NO: 678 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 11: mod_base = OTHER; note = nucleotide with phosphorothioate linkage
  modified_base 12: mod_base = OTHER; note = 2'-fluoro modified nucleotide
  modified_base 15: mod_base = OTHER; note = 2'-fluoro modified nucleotide
  modified_base 19: mod_base = OTHER; note = 2'-fluoro modified nucleotide
  modified_base 22: mod_base = OTHER; note = nucleotide with phosphorothioate linkage
  modified_base 23: mod_base = OTHER; note = 2'-fluoro modified nucleotide
  modified_base 28: mod_base = OTHER; note = 2'-fluoro modified nucleotide
  modified_base 29: mod_base = OTHER; note = 2'-O-methyl modified nucleotide
  modified_base 40: mod_base = OTHER; note = 2'-O-methyl modified nucleotide
  modified_base 48: mod_base = OTHER; note = 2'-fluoro modified nucleotide
  modified_base 62: mod_base = OTHER; note = nucleotide with phosphorothioate linkage
  modified_base 68: mod_base = OTHER; note = 2'-fluoro modified nucleotide
  modified_base 73: mod_base = OTHER; note = nucleotide with phosphorothioate linkage
  modified_base 74: mod_base = OTHER; note = 2'-fluoro modified nucleotide with phosphorothioate linkage

SEQ ID NO: 679 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 14: mod_base = OTHER; note = 2'-fluoro modified nucleotide
  modified_base 16: mod_base = OTHER; note = 2'-fluoro modified nucleotide
  modified_base 22: mod_base = OTHER; note = 2'-fluoro modified nucleotide
  modified_base 24: mod_base = OTHER; note = 2'-fluoro modified nucleotide
  modified_base 40: mod_base = OTHER; note = nucleotide with phosphorothioate linkage
  modified_base 43: mod_base = OTHER; note = 2'-fluoro modified nucleotide
  modified_base 44: mod_base = OTHER; note = 2'-fluoro modified nucleotide
  modified_base 45: mod_base = OTHER; note = 2'-fluoro modified nucleotide with phosphorothioate linkage
  modified_base 51: mod_base = OTHER; note = 2'-fluoro modified nucleotide
  modified_base 60: mod_base = OTHER; note = 2'-fluoro modified nucleotide
  modified_base 73: mod_base = OTHER; note = nucleotide with phosphorothioate linkage

SEQ ID NO: 680 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide
  modified_base 19: mod_base = OTHER; note = nucleotide with phosphorothioate linkage
  modified_base 20: mod_base = OTHER; note = 2'-fluoro modified nucleotide
  modified_base 23: mod_base = OTHER; note = nucleotide with phosphorothioate linkage
  modified_base 24: mod_base = OTHER; note = 2'-fluoro modified nucleotide
  modified_base 28: mod_base = OTHER; note = 2'-fluoro modified nucleotide
  modified_base 38: mod_base = OTHER; note = nucleotide with phosphorothioate linkage
  modified_base 39: mod_base = OTHER; note = 2'-fluoro modified nucleotide
  modified_base 51: mod_base = OTHER; note = nucleotide with phosphorothioate linkage
  modified_base 60: mod_base = OTHER; note = 2'-fluoro modified nucleotide
  modified_base 65: mod_base = OTHER; note = nucleotide with phosphorothioate linkage
  modified_base 73: mod_base = OTHER; note = nucleotide with phosphorothioate linkage
  modified_base 74: mod_base = OTHER; note = 2'-O-methyl modified nucleotide

SEQ ID NO: 681 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 13: mod_base = OTHER; note = nucleotide with phosphorothioate linkage
  modified_base 14: mod_base = OTHER; note = 2'-fluoro modified nucleotide
  modified_base 22: mod_base = OTHER; note = 2'-fluoro modified nucleotide
  modified_base 23: mod_base = OTHER; note = 2'-fluoro modified nucleotide
  modified_base 27: mod_base = OTHER; note = 2'-fluoro modified nucleotide with phosphorothioate linkage
  modified_base 40: mod_base = OTHER; note = 2'-O-methyl modified nucleotide with phosphorothioate linkage
  modified_base 41: mod_base = OTHER; note = 2'-fluoro modified nucleotide
  modified_base 44: mod_base = OTHER; note = 2'-fluoro modified nucleotide
  modified_base 55: mod_base = OTHER; note = nucleotide with phosphorothioate linkage
  modified_base 73: mod_base = OTHER; note = nucleotide with phosphorothioate linkage
  modified_base 74: mod_base = OTHER; note = nucleotide with phosphorothioate linkage

SEQ ID NO: 682 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 19: mod_base = OTHER; note = 2'-fluoro modified nucleotide
  modified_base 23: mod_base = OTHER; note = 2'-fluoro modified nucleotide
  modified_base 27: mod_base = OTHER; note = nucleotide with phosphorothioate linkage
  modified_base 28: mod_base = OTHER; note = 2'-fluoro modified nucleotide
  modified_base 40: mod_base = OTHER; note = nucleotide with phosphorothioate linkage
  modified_base 41: mod_base = OTHER; note = 2'-fluoro modified nucleotide
  modified_base 43: mod_base = OTHER; note = 2'-fluoro modified nucleotide
  modified_base 44: mod_base = OTHER; note = 2'-fluoro modified nucleotide
  modified_base 58: mod_base = OTHER; note = nucleotide with phosphorothioate linkage
  modified_base 59: mod_base = OTHER; note = 2'-fluoro modified nucleotide
  modified_base 66: mod_base = OTHER; note = 2'-fluoro modified nucleotide
  modified_base 67: mod_base = OTHER; note = 2'-fluoro modified nucleotide
  modified_base 68: mod_base = OTHER; note = 2'-fluoro modified nucleotide
  modified_base 69: mod_base = OTHER; note = nucleotide with phosphorothioate linkage
  modified_base 73: mod_base = OTHER; note = nucleotide with phosphorothioate linkage

SEQ ID NO: 683 - RNA, 75 nt, synthetic construct; identical to SEQ ID NO: 676
  source 1..75: mol_type = other RNA

SEQ ID NO: 684 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 12: mod_base = OTHER; note = nucleotide with phosphorothioate linkage
  modified_base 13: mod_base = OTHER; note = 2'-fluoro modified nucleotide with phosphorothioate linkage
  modified_base 23: mod_base = OTHER; note = nucleotide with phosphorothioate linkage
  modified_base 24: mod_base = OTHER; note = 2'-fluoro modified nucleotide
  modified_base 43: mod_base = OTHER; note = 2'-fluoro modified nucleotide
  modified_base 51: mod_base = OTHER; note = nucleotide with phosphorothioate linkage
  modified_base 58: mod_base = OTHER; note = 2'-O-methyl modified nucleotide with phosphorothioate linkage
  modified_base 60: mod_base = OTHER; note = 2'-fluoro modified nucleotide
  modified_base 67: mod_base = OTHER; note = 2'-fluoro modified nucleotide
  modified_base 69: mod_base = OTHER; note = 2'-fluoro modified nucleotide
  modified_base 73: mod_base = OTHER; note = nucleotide with phosphorothioate linkage
  modified_base 74: mod_base = OTHER; note = 2'-O-methyl modified nucleotide

SEQ ID NO: 685 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 4: mod_base = OTHER; note = 2'-fluoro modified nucleotide with phosphorothioate linkage
  modified_base 28: mod_base = OTHER; note = 2'-O-methyl modified nucleotide
  modified_base 43: mod_base = OTHER; note = 2'-fluoro modified nucleotide
  modified_base 46: mod_base = OTHER; note = 2'-fluoro modified nucleotide
  modified_base 55: mod_base = OTHER; note = 2'-fluoro modified nucleotide
  modified_base 60: mod_base = OTHER; note = 2'-fluoro modified nucleotide
  modified_base 61: mod_base = OTHER; note = 2'-fluoro modified nucleotide
  modified_base 73: mod_base = OTHER; note = 2'-fluoro modified nucleotide with phosphorothioate linkage

SEQ ID NO: 686 - RNA, 75 nt, synthetic construct; identical to SEQ ID NO: 676
  source 1..75: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 11: mod_base = OTHER; note = 2'-fluoro modified nucleotide
  modified_base 13: mod_base = OTHER; note = 2'-fluoro modified nucleotide
  modified_base 19: mod_base = OTHER; note = 2'-fluoro modified nucleotide
  modified_base 23: mod_base = OTHER; note = 2'-fluoro modified nucleotide
  modified_base 51: mod_base = OTHER; note = 2'-fluoro modified nucleotide
  modified_base 63: mod_base = OTHER; note = nucleotide with phosphorothioate linkage
  modified_base 64: mod_base = OTHER; note = 2'-fluoro modified nucleotide
  modified_base 65: mod_base = OTHER; note = 2'-fluoro modified nucleotide
  modified_base 66: mod_base = OTHER; note = nucleotide with phosphorothioate linkage
  modified_base 67: mod_base = OTHER; note = 2'-fluoro modified nucleotide
  modified_base 74: mod_base = OTHER; note = nucleotide with phosphorothioate linkage
  modified_base 75: mod_base = OTHER; note = 2'-fluoro modified nucleotide

SEQ ID NO: 687 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 12: mod_base = OTHER; note = 2'-O-methyl modified nucleotide
  modified_base 13: mod_base = OTHER; note = nucleotide with phosphorothioate linkage
  modified_base 22: mod_base = OTHER; note = 2'-fluoro modified nucleotide
  modified_base 23: mod_base = OTHER; note = 2'-fluoro modified nucleotide
  modified_base 28: mod_base = OTHER; note = 2'-fluoro modified nucleotide
  modified_base 43: mod_base = OTHER; note = 2'-fluoro modified nucleotide
  modified_base 50: mod_base = OTHER; note = 2'-fluoro modified nucleotide with phosphorothioate linkage
  modified_base 59: mod_base = OTHER; note = 2'-fluoro modified nucleotide
  modified_base 65: mod_base = OTHER; note = nucleotide with phosphorothioate linkage
  modified_base 73: mod_base = OTHER; note = nucleotide with phosphorothioate linkage
  modified_base 74: mod_base = OTHER; note = 2'-O-methyl modified nucleotide

SEQ ID NO: 688 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 18: mod_base = OTHER; note = 2'-fluoro modified nucleotide
  modified_base 19: mod_base = OTHER; note = 2'-fluoro modified nucleotide
  modified_base 20: mod_base = OTHER; note = 2'-fluoro modified nucleotide
  modified_base 23: mod_base = OTHER; note = nucleotide with phosphorothioate linkage
  modified_base 24: mod_base = OTHER; note = 2'-fluoro modified nucleotide with phosphorothioate linkage
  modified_base 25: mod_base = OTHER; note = nucleotide with phosphorothioate linkage
  modified_base 27: mod_base = OTHER; note = 2'-fluoro modified nucleotide
  modified_base 31: mod_base = OTHER; note = 2'-fluoro modified nucleotide
  modified_base 34: mod_base = OTHER; note = 2'-fluoro modified nucleotide
  modified_base 44: mod_base = OTHER; note = 2'-fluoro modified nucleotide
  modified_base 48: mod_base = OTHER; note = 2'-fluoro modified nucleotide with phosphorothioate linkage
  modified_base 63: mod_base = OTHER; note = 2'-fluoro modified nucleotide with phosphorothioate linkage
  modified_base 67: mod_base = OTHER; note = 2'-fluoro modified nucleotide
  modified_base 71: mod_base = OTHER; note = 2'-fluoro modified nucleotide
  modified_base 75: mod_base = OTHER; note = 2'-O-methyl modified nucleotide

SEQ ID NO: 689 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 12: mod_base = OTHER; note = 2'-O-methyl modified nucleotide
  modified_base 13: mod_base = OTHER; note = nucleotide with phosphorothioate linkage
  modified_base 22: mod_base = OTHER; note = 2'-fluoro modified nucleotide
  modified_base 23: mod_base = OTHER; note = 2'-fluoro modified nucleotide
  modified_base 27: mod_base = OTHER; note = nucleotide with phosphorothioate linkage
  modified_base 28: mod_base = OTHER; note = 2'-fluoro modified nucleotide
  modified_base 40: mod_base = OTHER; note = nucleotide with phosphorothioate linkage
  modified_base 41: mod_base = OTHER; note = 2'-fluoro modified nucleotide
  modified_base 46: mod_base = OTHER; note = 2'-fluoro modified nucleotide
  modified_base 55: mod_base = OTHER; note = 2'-O-methyl modified nucleotide
  modified_base 63: mod_base = OTHER; note = nucleotide with phosphorothioate linkage
  modified_base 64: mod_base = OTHER; note = nucleotide with phosphorothioate linkage
  modified_base 67: mod_base = OTHER; note = 2'-fluoro modified nucleotide
  modified_base 73: mod_base = OTHER; note = nucleotide with phosphorothioate linkage
  modified_base 74: mod_base = OTHER; note = 2'-O-methyl modified nucleotide

SEQ ID NO: 690 - RNA, 75 nt, synthetic construct; identical to SEQ ID NO: 676
  source 1..75: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 12: mod_base = OTHER; note = nucleotide with phosphorothioate linkage
  modified_base 19: mod_base = OTHER; note = 2'-fluoro modified nucleotide
  modified_base 22: mod_base = OTHER; note = 2'-fluoro modified nucleotide
  modified_base 23: mod_base = OTHER; note = 2'-fluoro modified nucleotide
  modified_base 31: mod_base = OTHER; note = 2'-O-methyl modified nucleotide
  modified_base 50: mod_base = OTHER; note = nucleotide with phosphorothioate linkage
  modified_base 51: mod_base = OTHER; note = 2'-fluoro modified nucleotide
  modified_base 63: mod_base = OTHER; note = nucleotide with phosphorothioate linkage
  modified_base 66: mod_base = OTHER; note = 2'-fluoro modified nucleotide
  modified_base 73: mod_base = OTHER; note = nucleotide with phosphorothioate linkage
  modified_base 74: mod_base = OTHER; note = 2'-O-methyl modified nucleotide

SEQ ID NO: 691-1199 - skipped (509 entries)

SEQ ID NO: 1200 - DNA, 73 bp, synthetic construct; identical to SEQ ID NO: 4
  source 1..73: mol_type = other DNA

SEQ ID NO: 1201 - DNA, 72 bp, synthetic construct; identical to SEQ ID NO: 17
  source 1..72: mol_type = other DNA

SEQ ID NO: 1202 - DNA, 72 bp, synthetic construct
  source 1..72: mol_type = other DNA
gggggtgtagctcagtggtagagcgcatgctttgcatgtatgaggccccgggttcgatccccggcacctcca

SEQ ID NO: 1203 - DNA, 73 bp, synthetic construct; identical to SEQ ID NO: 28
  source 1..73: mol_type = other DNA

SEQ ID NO: 1204 - DNA, 73 bp, synthetic construct; identical to SEQ ID NO: 30
  source 1..73: mol_type = other DNA

SEQ ID NO: 1205 - DNA, 73 bp, synthetic construct; identical to SEQ ID NO: 32
  source 1..73: mol_type = other DNA

SEQ ID NO: 1206 - DNA, 73 bp, synthetic construct
  source 1..73: mol_type = other DNA
gaccgcgtggcctaatggataaggcgtctgacttcggatcagaagattgagggttcgagtccctccgtggtcg

SEQ ID NO: 1207 - DNA, 74 bp, synthetic construct
  source 1..74: mol_type = other DNA
  misc_difference 21: note = a, c, g, t, unknown or other
  misc_difference 65: note = a, c, g, t, unknown or other
ggctctgtggcgcaatggatnagcgcattggacttctaattcaaaggttgcgggttcgagtcccnccagagtcg

SEQ ID NO: 1208 - DNA, 75 bp, synthetic construct
  source 1..75: mol_type = other DNA
  misc_difference 43: note = a, c, g, t, unknown or other
gtctctgtggcgcaatcggttagcgcgttcggctgttaaccgnaaaggttggtggttcgagcccacccagggacg

SEQ ID NO: 1209 - DNA, 72 bp, synthetic construct; identical to SEQ ID NO: 62
  source 1..72: mol_type = other DNA

SEQ ID NO: 1210 - DNA, 73 bp, synthetic construct
  source 1..73: mol_type = other DNA
  misc_difference 16: note = a, c, g, t, unknown or other
gggggtatagctcagngggtagagcatttgactgcagatcaagaggtccccggttcaaatccgggtgccccct

SEQ ID NO: 1211 - DNA, 72 bp, synthetic construct
  source 1..72: mol_type = other DNA
  misc_difference 20: note = a, c, g, t, unknown or other
ggttccatggtgtaatggtnagcactctggactctgaatccagcgatccgagttcaagtctcggtggaacct

SEQ ID NO: 1212 - DNA, 72 bp, synthetic construct; identical to SEQ ID NO: 94
  source 1..72: mol_type = other DNA

SEQ ID NO: 1213 - DNA, 72 bp, synthetic construct; identical to SEQ ID NO: 98
  source 1..72: mol_type = other DNA

SEQ ID NO: 1214 - DNA, 72 bp, synthetic construct
  source 1..72: mol_type = other DNA
  misc_difference 42: note = a, c, g, t, unknown or other
tccctggtggtctagtggctaggattcggcgctttcaccgcngcggcccgggttcgattcccggtcagggaa

SEQ ID NO: 1215 - DNA, 71 bp, synthetic construct
  source 1..71: mol_type = other DNA
  misc_difference 40: note = a, c, g, t, unknown or other
gcattggtggttcagtggtagaattctcgcctcccacgcnggagacccgggttcgattcccggccaatgca

SEQ ID NO: 1216 - DNA, 71 bp, synthetic construct; identical to SEQ ID NO: 108
  source 1..71: mol_type = other DNA

SEQ ID NO: 1217 - DNA, 72 bp, synthetic construct; identical to SEQ ID NO: 113
  source 1..72: mol_type = other DNA

SEQ ID NO: 1218 - DNA, 74 bp, synthetic construct; identical to SEQ ID NO: 122
  source 1..74: mol_type = other DNA

SEQ ID NO: 1219 - DNA, 74 bp, synthetic construct
  source 1..74: mol_type = other DNA
gctccagtggcgcaatcggttagcgcgcggtacttataatgccgaggttgtgagttcgagcctcacctggagca

SEQ ID NO: 1220 - DNA, 82 bp, synthetic construct; identical to SEQ ID NO: 134
  source 1..82: mol_type = other DNA

SEQ ID NO: 1221 - DNA, 85 bp, synthetic construct
  source 1..85: mol_type = other DNA
  misc_difference 21: note = a, c, g, t, unknown or other
  misc_difference 52: note = a, c, g, t, unknown or other
gtcaggatggccgagtggtcntaaggcgccagactcaagttctggtctccgnatggaggcgtgggttcgaatcccacttctgaca

SEQ ID NO: 1222 - DNA, 83 bp, synthetic construct; identical to SEQ ID NO: 144
  source 1..83: mol_type = other DNA

SEQ ID NO: 1223 - DNA, 83 bp, synthetic construct
  source 1..83: mol_type = other DNA
accaggatggccgagtggttaaggcgttggacttaagatccaatggacagatgtccgcgtgggttcgaaccccactcctggta

SEQ ID NO: 1224 - DNA, 82 bp, synthetic construct
  source 1..82: mol_type = other DNA
  misc_difference 54: note = a, c, g, t, unknown or other
ggtagcgtggccgagcggtctaaggcgctggatttaggctccagtctcttcggnggcgtgggttcgaatcccaccgctgcca

SEQ ID NO: 1225 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
gcccggctagctcagtcggtagagcatgagactcttaatctcagggtcgtgggttcgagccccacgttgggcgnnn

SEQ ID NO: 1226 - DNA, 73 bp, synthetic construct; identical to SEQ ID NO: 167
  source 1..73: mol_type = other DNA

SEQ ID NO: 1227 - DNA, 73 bp, synthetic construct
  source 1..73: mol_type = other DNA
  misc_difference 17: note = a, c, g, t, unknown or other
gccctcttagcgcagtnggcagcgcgtcagtctcataatctgaaggtcctgagttcgagcctcagagagggca

SEQ ID NO: 1228 - DNA, 74 bp, synthetic construct
  source 1..74: mol_type = other DNA
  misc_difference 41: note = a, c, g, t, unknown or other
gccgaaatagctcagttgggagagcgttagactgaagatcntaaaggtccctggttcaatcccgggtttcggca

SEQ ID NO: 1229 - DNA, 72 bp, synthetic construct; identical to SEQ ID NO: 183
  source 1..72: mol_type = other DNA

SEQ ID NO: 1230 - DNA, 72 bp, synthetic construct; identical to SEQ ID NO: 185
  source 1..72: mol_type = other DNA

SEQ ID NO: 1231 - DNA, 72 bp, synthetic construct; identical to SEQ ID NO: 189
  source 1..72: mol_type = other DNA

SEQ ID NO: 1232 - DNA, 82 bp, synthetic construct; identical to SEQ ID NO: 192
  source 1..82: mol_type = other DNA

SEQ ID NO: 1233 - DNA, 82 bp, synthetic construct; identical to SEQ ID NO: 196
  source 1..82: mol_type = other DNA

SEQ ID NO: 1234 - DNA, 84 bp, synthetic construct
  source 1..84: mol_type = other DNA
  misc_difference 8: note = a, c, g, t, unknown or other
  misc_difference 9: note = a, c, g, t, unknown or other
gacgaggnntggccgagtggttaaggcgatggactgctaatccattgtgctctgcacgcgtgggttcgaatcccatcctcgtcg

SEQ ID NO: 1235 - DNA, 82 bp, synthetic construct
  source 1..82: mol_type = other DNA
gtagtcgtggccgagtggttaaggcgatggacttgaaatccattggggtctccccgcgcaggttcgaatcctgccggctacg

SEQ ID NO: 1236 - DNA, 74 bp, synthetic construct; identical to SEQ ID NO: 211
  source 1..74: mol_type = other DNA

SEQ ID NO: 1237 - DNA, 75 bp, synthetic construct
  source 1..75: mol_type = other DNA
  misc_difference 4: note = a, c, g, t, unknown or other
  misc_difference 13: note = a, c, g, t, unknown or other
  misc_difference 17: note = a, c, g, t, unknown or other
  misc_difference 20: note = a, c, g, t, unknown or other
  misc_difference 43: note = a, c, g, t, unknown or other
  misc_difference 51: note = a, c, g, t, unknown or other
  misc_difference 67: note = a, c, g, t, unknown or other
  misc_difference 69: note = a, c, g, t, unknown or other
ggcnctgtggctnagtnggntaaagcgccggtctcgtaaaccnggagatcntgggttcgaatcccancngggcct

SEQ ID NO: 1238 - DNA, 74 bp, synthetic construct
  source 1..74: mol_type = other DNA
  misc_difference 16: note = a, c, g, t, unknown or other
ggctccatagctcagngggttagagcactggtcttgtaaaccaggggtcgcgagttcaaatctcgctggggcct

SEQ ID NO: 1239 - DNA, 72 bp, synthetic construct; identical to SEQ ID NO: 229
  source 1..72: mol_type = other DNA

SEQ ID NO: 1240 - DNA, 73 bp, synthetic construct
  source 1..73: mol_type = other DNA
ccttcgatagctcagctggtagagcggaggactgtagatccttaggtcgctggttcgattccggctcgaagga

SEQ ID NO: 1241 - DNA, 73 bp, synthetic construct; identical to SEQ ID NO: 246
  source 1..73: mol_type = other DNA

SEQ ID NO: 1242 - DNA, 73 bp, synthetic construct; identical to SEQ ID NO: 252
  source 1..73: mol_type = other DNA

SEQ ID NO: 1243 - DNA, 73 bp, synthetic construct; identical to SEQ ID NO: 258
  source 1..73: mol_type = other DNA

SEQ ID NO: 1244 - DNA, 72 bp, synthetic construct; identical to SEQ ID NO: 262
  source 1..72: mol_type = other DNA

SEQ ID NO: 1245 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
ggggaattagctcaagtggtagagcgcttgcttagcatgcaagaggtagtgggatcgatgcccacattctccannn

SEQ ID NO: 1246 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
ggggatgtagctcagtggtagagcgcatgcttcgcatgtatgaggtcccgggttcgatccccggcatctccannn

SEQ ID NO: 1247 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
gggggtgtagctcagtggtagagcgcatgctttgcatgtatgaggccccgggttcgatccccggcacctccannn

SEQ ID NO: 1248 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
gggccagtggcgcaatggataacgcgtctgactacggatcagaagattccaggttcgactcctggctggctcgnnn

SEQ ID NO: 1249 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
ggccgcgtggcctaatggataaggcgtctgattccggatcagaagattgagggttcgagtcccttcgtggtcgnnn

SEQ ID NO: 1250 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
gccccagtggcctaatggataaggcactggcctcctaagccagggattgtgggttcgagtcccacctggggtannn

SEQ ID NO: 1251 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
gaccgcgtggcctaatggataaggcgtctgacttcggatcagaagattgagggttcgagtccctccgtggtcgnnn

SEQ ID NO: 1252 - DNA, 77 bp, synthetic construct
  source 1..77: mol_type = other DNA
  misc_difference 21: note = a, c, g, t, unknown or other
  misc_difference 65: note = a, c, g, t, unknown or other
  misc_difference 75: note = a, c, g, t, unknown or other
  misc_difference 76: note = a, c, g, t, unknown or other
  misc_difference 77: note = a, c, g, t, unknown or other
ggctctgtggcgcaatggatnagcgcattggacttctaattcaaaggttgcgggttcgagtcccnccagagtcgnnn

SEQ ID NO: 1253 - DNA, 78 bp, synthetic construct
  source 1..78: mol_type = other DNA
  misc_difference 43: note = a, c, g, t, unknown or other
  misc_difference 76: note = a, c, g, t, unknown or other
  misc_difference 77: note = a, c, g, t, unknown or other
  misc_difference 78: note = a, c, g, t, unknown or other
gtctctgtggcgcaatcggttagcgcgttcggctgttaaccgnaaaggttggtggttcgagcccacccagggacgnnn

SEQ ID NO: 1254 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
tcctcgttagtatagtggtgagtatccccgcctgtcacgcgggagaccggggttcgattccccgacggggagnnn

SEQ ID NO: 1255 - DNA, 76 bp, synthetic construct
  source 1..76: mol_type = other DNA
  misc_difference 16: note = a, c, g, t, unknown or other
  misc_difference 74: note = a, c, g, t, unknown or other
  misc_difference 75: note = a, c, g, t, unknown or other
  misc_difference 76: note = a, c, g, t, unknown or other
gggggtatagctcagngggtagagcatttgactgcagatcaagaggtccccggttcaaatccgggtgccccctnnn

SEQ ID NO: 1256 - DNA, 75 bp, synthetic construct
  source 1..75: mol_type = other DNA
  misc_difference 20: note = a, c, g, t, unknown or other
  misc_difference 73: note = a, c, g, t, unknown or other
  misc_difference 74: note = a, c, g, t, unknown or other
  misc_difference 75: note = a, c, g, t, unknown or other
ggttccatggtgtaatggtnagcactctggactctgaatccagcgatccgagttcaagtctcggtggaacctnnn

SEQ ID NO: 1257 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
ggtcccatggtgtaatggttagcactctggactttgaatccagcgatccgagttcaaatctcggtgggacctnnn

SEQ ID NO: 1258 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
tccctggtggtctagtggttaggattcggcgctctcaccgccgcggcccgggttcgattcccggtcagggaannn

SEQ ID NO: 1259 - DNA, 75 bp, synthetic construct
  source 1..75: mol_type = other DNA
  misc_difference 42: note = a, c, g, t, unknown or other
  misc_difference 73: note = a, c, g, t, unknown or other
  misc_difference 74: note = a, c, g, t, unknown or other
  misc_difference 75: note = a, c, g, t, unknown or other
tccctggtggtctagtggctaggattcggcgctttcaccgcngcggcccgggttcgattcccggtcagggaannn

SEQ ID NO: 1260 - DNA, 74 bp, synthetic construct
  source 1..74: mol_type = other DNA
  misc_difference 40: note = a, c, g, t, unknown or other
  misc_difference 72: note = a, c, g, t, unknown or other
  misc_difference 73: note = a, c, g, t, unknown or other
  misc_difference 74: note = a, c, g, t, unknown or other
gcattggtggttcagtggtagaattctcgcctcccacgcnggagacccgggttcgattcccggccaatgcannn

SEQ ID NO: 1261 - DNA, 74 bp, synthetic construct
  source 1..74: mol_type = other DNA
  misc_difference 72: note = a, c, g, t, unknown or other
  misc_difference 73: note = a, c, g, t, unknown or other
  misc_difference 74: note = a, c, g, t, unknown or other
gcattggtggttcagtggtagaattctcgcctgccacgcgggaggcccgggttcgattcccggccaatgcannn

SEQ ID NO: 1262 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
gcgttggtggtatagtggtgagcatagctgccttccaagcagttgacccgggttcgattcccggccaacgcannn

SEQ ID NO: 1263 - DNA, 77 bp, synthetic construct
  source 1..77: mol_type = other DNA
  misc_difference 75: note = a, c, g, t, unknown or other
  misc_difference 76: note = a, c, g, t, unknown or other
  misc_difference 77: note = a, c, g, t, unknown or other
ggccggttagctcagttggttagagcgtggtgctaataacgccaaggtcgcgggttcgatccccgtacgggccannn

SEQ ID NO: 1264 - DNA, 77 bp, synthetic construct
  source 1..77: mol_type = other DNA
  misc_difference 75: note = a, c, g, t, unknown or other
  misc_difference 76: note = a, c, g, t, unknown or other
  misc_difference 77: note = a, c, g, t, unknown or other
gctccagtggcgcaatcggttagcgcgcggtacttataatgccgaggttgtgagttcgagcctcacctggagcannn

SEQ ID NO: 1265 - DNA, 85 bp, synthetic construct
  source 1..85: mol_type = other DNA
  misc_difference 83: note = a, c, g, t, unknown or other
  misc_difference 84: note = a, c, g, t, unknown or other
  misc_difference 85: note = a, c, g, t, unknown or other
ggtagcgtggccgagcggtctaaggcgctggattaaggctccagtctcttcgggggcgtgggttcgaatcccaccgctgccannn

SEQ ID NO: 1266 - DNA, 88 bp, synthetic construct
  source 1..88: mol_type = other DNA
  misc_difference 21: note = a, c, g, t, unknown or other
  misc_difference 52: note = a, c, g, t, unknown or other
  misc_difference 86: note = a, c, g, t, unknown or other
  misc_difference 87: note = a, c, g, t, unknown or other
  misc_difference 88: note = a, c, g, t, unknown or other
gtcaggatggccgagtggtcntaaggcgccagactcaagttctggtctccgnatggaggcgtgggttcgaatcccacttctgacannn

SEQ ID NO: 1267 - DNA, 86 bp, synthetic construct
  source 1..86: mol_type = other DNA
  misc_difference 84: note = a, c, g, t, unknown or other
  misc_difference 85: note = a, c, g, t, unknown or other
  misc_difference 86: note = a, c, g, t, unknown or other
gtcaggatggccgagcggtctaaggcgctgcgttcaggtcgcagtctcccctggaggcgtgggttcgaatcccactcctgacannn

SEQ ID NO: 1268 - DNA, 86 bp, synthetic construct
  source 1..86: mol_type = other DNA
  misc_difference 84: note = a, c, g, t, unknown or other
  misc_difference 85: note = a, c, g, t, unknown or other
  misc_difference 86: note = a, c, g, t, unknown or other
accaggatggccgagtggttaaggcgttggacttaagatccaatggacagatgtccgcgtgggttcgaaccccactcctggtannn

SEQ ID NO: 1269 - DNA, 85 bp, synthetic construct
  source 1..85: mol_type = other DNA
  misc_difference 54: note = a, c, g, t, unknown or other
  misc_difference 83: note = a, c, g, t, unknown or other
  misc_difference 84: note = a, c, g, t, unknown or other
  misc_difference 85: note = a, c, g, t, unknown or other
ggtagcgtggccgagcggtctaaggcgctggatttaggctccagtctcttcggnggcgtgggttcgaatcccaccgctgccannn

SEQ ID NO: 1270 - DNA, 79 bp, synthetic construct
  source 1..79: mol_type = other DNA
  misc_difference 74: note = a, c, g, t, unknown or other
  misc_difference 75: note = a, c, g, t, unknown or other
  misc_difference 76: note = a, c, g, t, unknown or other
  misc_difference 77: note = a, c, g, t, unknown or other
  misc_difference 78: note = a, c, g, t, unknown or other
  misc_difference 79: note = a, c, g, t, unknown or other
gcccggctagctcagtcggtagagcatgagactcttaatctcagggtcgtgggttcgagccccacgttgggcgnnnnnn

SEQ ID NO: 1271 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
gcctggatagctcagtcggtagagcatcagacttttaatctgagggtccagggttcaagtccctgttcaggcgnnn

SEQ ID NO: 1272 - DNA, 76 bp, synthetic construct
  source 1..76: mol_type = other DNA
  misc_difference 17: note = a, c, g, t, unknown or other
  misc_difference 74: note = a, c, g, t, unknown or other
  misc_difference 75: note = a, c, g, t, unknown or other
  misc_difference 76: note = a, c, g, t, unknown or other
gccctcttagcgcagtnggcagcgcgtcagtctcataatctgaaggtcctgagttcgagcctcagagagggcannn

SEQ ID NO: 1273 - DNA, 77 bp, synthetic construct
  source 1..77: mol_type = other DNA
  misc_difference 41: note = a, c, g, t, unknown or other
  misc_difference 75: note = a, c, g, t, unknown or other
  misc_difference 76: note = a, c, g, t, unknown or other
  misc_difference 77: note = a, c, g, t, unknown or other
gccgaaatagctcagttgggagagcgttagactgaagatcntaaaggtccctggttcaatcccgggtttcggcannn

SEQ ID NO: 1274 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
ggctcgttggtctaggggtatgattctcgcttaggatgcgagaggtcccgggttcaaatcccggacgagcccnnn

SEQ ID NO: 1275 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
ggctcgttggtctaggggtatgattctcgcttcgggtgcgagaggtcccgggttcaaatcccggacgagcccnnn

SEQ ID NO: 1276 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
ggctcgttggtctaggggtatgattctcgctttgggtgcgagaggtcccgggttcaaatcccggacgagcccnnn

SEQ ID NO: 1277 - DNA, 85 bp, synthetic construct
  source 1..85: mol_type = other DNA
  misc_difference 83: note = a, c, g, t, unknown or other
  misc_difference 84: note = a, c, g, t, unknown or other
  misc_difference 85: note = a, c, g, t, unknown or other
gtagtcgtggccgagtggttaaggcgatggactagaaatccattggggtttccccgcgcaggttcgaatcctgccgactacgnnn

SEQ ID NO: 1278 - DNA, 85 bp, synthetic construct
  source 1..85: mol_type = other DNA
  misc_difference 83: note = a, c, g, t, unknown or other
  misc_difference 84: note = a, c, g, t, unknown or other
  misc_difference 85: note = a, c, g, t, unknown or other
gctgtgatggccgagtggttaaggcgttggactcgaaatccaatggggtctccccgcgcaggttcgaatcctgctcacagcgnnn

SEQ ID NO: 1279 - DNA, 87 bp, synthetic construct
  source 1..87: mol_type = other DNA
  misc_difference 8: note = a, c, g, t, unknown or other
  misc_difference 9: note = a, c, g, t, unknown or other
  misc_difference 85: note = a, c, g, t, unknown or other
  misc_difference 86: note = a, c, g, t, unknown or other
  misc_difference 87: note = a, c, g, t, unknown or other
gacgaggnntggccgagtggttaaggcgatggactgctaatccattgtgctctgcacgcgtgggttcgaatcccatcctcgtcgnnn

SEQ ID NO: 1280 - DNA, 85 bp, synthetic construct
  source 1..85: mol_type = other DNA
  misc_difference 83: note = a, c, g, t, unknown or other
  misc_difference 84: note = a, c, g, t, unknown or other
  misc_difference 85: note = a, c, g, t, unknown or other
gtagtcgtggccgagtggttaaggcgatggacttgaaatccattggggtctccccgcgcaggttcgaatcctgccggctacgnnn

SEQ ID NO: 1281 - DNA, 77 bp, synthetic construct
  source 1..77: mol_type = other DNA
  misc_difference 75: note = a, c, g, t, unknown or other
  misc_difference 76: note = a, c, g, t, unknown or other
  misc_difference 77: note = a, c, g, t, unknown or other
ggctccgtggcttagctggttaaagcgcctgtctagtaaacaggagatcctgggttcgaatcccagcggggcctnnn

SEQ ID NO: 1282 - DNA, 78 bp, synthetic construct
  source 1..78: mol_type = other DNA
  misc_difference 4: note = a, c, g, t, unknown or other
  misc_difference 13: note = a, c, g, t, unknown or other
  misc_difference 17: note = a, c, g, t, unknown or other
  misc_difference 20: note = a, c, g, t, unknown or other
  misc_difference 43: note = a, c, g, t, unknown or other
  misc_difference 51: note = a, c, g, t, unknown or other
  misc_difference 67: note = a, c, g, t, unknown or other
  misc_difference 69: note = a, c, g, t, unknown or other
  misc_difference 76: note = a, c, g, t, unknown or other
  misc_difference 77: note = a, c, g, t, unknown or other
  misc_difference 78: note = a, c, g, t, unknown or other
ggcnctgtggctnagtnggntaaagcgccggtctcgtaaaccnggagatcntgggttcgaatcccancngggcctnnn

SEQ ID NO: 1283 - DNA, 77 bp, synthetic construct
  source 1..77: mol_type = other DNA
  misc_difference 16: note = a, c, g, t, unknown or other
  misc_difference 75: note = a, c, g, t, unknown or other
  misc_difference 76: note = a, c, g, t, unknown or other
  misc_difference 77: note = a, c, g, t, unknown or other
ggctccatagctcagngggttagagcactggtcttgtaaaccaggggtcgcgagttcaaatctcgctggggcctnnn

SEQ ID NO: 1284 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
gacctcgtggcgcaacggtagcgcgtctgactccagatcagaaggttgcgtgttcaaatcacgtcggggtcannn

SEQ ID NO: 1285 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
ccttcgatagctcagctggtagagcggaggactgtagatccttaggtcgctggttcgattccggctcgaaggannn

SEQ ID NO: 1286 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
gtttccgtagtgtagtggttatcacgttcgcctaacacgcgaaaggtccccggttcgaaaccgggcggaaacannn

SEQ ID NO: 1287 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
gtttccgtagtgtagtggttatcacgttcgcctcacacgcgaaaggtccccggttcgaaaccgggcggaaacannn

SEQ ID NO: 1288 - DNA, 76 bp, synthetic construct
  source 1..76: mol_type = other DNA
  misc_difference 74: note = a, c, g, t, unknown or other
  misc_difference 75: note = a, c, g, t, unknown or other
  misc_difference 76: note = a, c, g, t, unknown or other
ggttccatagtgtagtggttatcacgtctgctttacacgcagaaggtcctgggttcgagccccagtggaaccannn

SEQ ID NO: 1289 - DNA, 75 bp, synthetic construct
  source 1..75: mol_type = other DNA
  misc_difference 73: note = a, c, g, t, unknown or other
  misc_difference 74: note = a, c, g, t, unknown or other
  misc_difference 75: note = a, c, g, t, unknown or other
agcagagtggcgcagcggaagcgtgctgggcccataacccagaggtcgatggatcgaaaccatcctctgctannn

(19) United States
(12) Patent Application Publication
(10) Pub. No.: US 2025/0295687 A1
(43) Pub. Date: Sep. 25, 2025

(54) TREM COMPOSITIONS AND METHODS OF USE FOR TREATING PROLIFERATIVE DISORDERS

(71) Applicant: Flagship Pioneering Innovations VI, LLC, Cambridge, MA (US)

(72) Inventors: Theonie Anastassiadis, Somerville, MA (US); Neil Kubica, Swampscott, MA (US); Isabelle Myriam Sansal, Somerville, MA (US); Nikolai Naryshkin, East Brunswick, NJ (US); Eva Asp, Cambridge, MA (US); YounKyoung Lee, Cambridge, MA (US); Armand Gatien Ngounou Wetie, Woburn, MA (US)

(21) Appl. No.: 18/864,142

(22) PCT Filed: May 9, 2023
(86) PCT No.: PCT/US2023/021581

§ 371 Date: Nov. 8, 2024

Related U.S. Application Data
(60) Provisional application No. 63/339,880, filed on May 9, 2022.

(51) Int. Cl. A61K 31/7105; A61K 31/7115; A61K 31/712; A61K 31/7125; A61P 35/00; C12Q 1/6886

(52) U.S. Cl. CPC A61K 31/7105; A61K 31/7115; A61K 31/712; A61K 31/7125; A61P 35/00; C12Q 1/6886; C12Q 2600/156

(57) ABSTRACT

The disclosure relates generally to tRNA-based effector molecules (TREMs) and compositions thereof useful for the treatment or prevention of a proliferative disease or disorder (e.g., a cancer) in a subject.

### CLAIM OF PRIORITY

This application claims priority to U.S. Provisional Application No. 63/339,880, filed on May 9, 2022. The entire contents of this application are hereby incorporated by reference.

### BACKGROUND

Transfer RNAs (tRNAs) are complex, naturally occurring RNA molecules that possess a number of functions including initiation and elongation of proteins.

### SUMMARY

The present disclosure features tRNA-based effector molecules (TREMs) and compositions thereof useful for the treatment or prevention of a proliferative disease or disorder (e.g., a cancer) in a subject. As disclosed herein, TREMs are complex molecules which can mediate a variety of cellular processes. A TREM many be formulated in a composition, e.g., a pharmaceutical composition, for local delivery to a cell, a tissue, or to a subject having a proliferative disease or disorder (e.g., a cancer). In an embodiment, the TREMs described herein are administered locally (e.g., intratumorally) to a subject having cancer. In an embodiment, the TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein independently, [L1] and [VL Domain], are optional.

In an embodiment, a TREM has the ability to: (i) support protein synthesis, (ii) be charged by a synthetase, (iii) be bound by an elongation factor, (iv) introduce an amino acid into a peptide chain, (v) support elongation, or (vi) support initiation. In an embodiment, the TREM comprises feature (i). In an embodiment, the TREM comprises feature (ii). In an embodiment, the TREM comprises feature (iii). In an embodiment, the TREM comprises feature (iv). In an embodiment, the TREM comprises feature (v). In an embodiment, the TREM comprises feature (vi). In an embodiment, the TREM comprises all of features (i)-(vi) or a combination thereof.

A TREM may or may not comprise a non-naturally occurring modification. In an embodiment, the TREM comprises a non-naturally occurring modification. In an embodiment, the TREM does not comprise a non-naturally occurring modification. In an embodiment, the TREM induces an immune response in a cell, tissue or subject, e.g., compared to a reference. In an embodiment, the TREM comprises a non-naturally occurring modification. In an embodiment, the TREM comprises at least 1, 2, 3, 4, 5, 6, 7, 8, 9, 10, 11, 12, 13, 14, 15, 20, 25, 30, 35, 40, 45, 50, 55, 60, 65, 70, or more non-naturally occurring modifications. In an embodiment, the non-naturally occurring modification induces an immune response in a cell, tissue, or subject, e.g., compared to a reference.

### BRIEF DESCRIPTION OF THE DRAWINGS

FIG. 1 is Western blotting of protein samples from Calu-6 lung cancer cells treated with controls (ataluren or G418) and exemplary TREMS, specifically Ser-TAG, Ser-TGA, and Arg-TGA. Cells were also left untreated or treated with mock (vehicle) as controls.

FIG. 2 is a graph illustrating quantification of full length p53 protein levels as normalized to 0-tubulin.

FIG. 3 is a graph illustrating quantification of the fraction of full length p53 protein as a measure of PTC suppression.

FIG. 4 is a graph illustrating quantification of p21 protein levels as normalized to 0-tubulin.

FIG. 5 is a set of graphs illustrating in vivo PTC readthrough and target engagement of a TREM. FIG. 5A is a graph depicting dose-dependent expression of luciferase in the liver from a plasmid following hydrodynamic delivery. FIG. 5B is a graph illustrating rescue of a luciferase gene with a PTC mutation with a plasmid expressing the corresponding TREM.

FIG. 6 is a table summarizing exemplary TREMs, TREM core fragments, and TREM fragments described herein. The sequence of each TREM, TREM core fragment, and TREM 25 fragment is provided, and the chemical modification profile is annotated as follows: r: ribonucleotide; m: 2′-OMe; *: PS linkage; f: 2′-fluoro; moe: 2′-moe; d: deoxyribonucleotide; 5MeC: 5-methylcytosine. Thus, for example, mA represents 2′-O-methyl adenosine, moe5MeC represents 2′-MOE nucleotide with 5-methylcytosine nucleobase, and dA represents an adenosine deoxyribonucleotide. The table also provides mass spectrometric characterization of each TREM, TREM core fragment, and TREM fragment, along with results from the activity screens described in Example 5. The results from the activity screens are in the columns titled “A”.

### DETAILED DESCRIPTION OF CERTAIN EMBODIMENTS

The present disclosure features tRNA-based effector molecules (TREMs) and compositions thereof useful for the treatment or prevention of a proliferative disease or disorder (e.g., a cancer) in a subject. As disclosed herein, TREMs are complex molecules which can mediate a variety of cellular processes. In an embodiment, the TREMs described herein are administered locally (e.g., intratumorally) to a subject having cancer. In an embodiment, the TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein independently, [L1] and [VL Domain], are optional.

### Definitions

As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendo-theliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is a solid tumor, such as a sarcoma or a carcinoma (e.g., lung cancer, brain cancer, breast cancer, bladder cancer, prostate cancer, colon cancer, rectal cancer).

As used herein, the terms “increasing” and “decreasing” refer to modulating that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

“Decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

“Increased expression,” as that term is used herein, refers to an increase in comparison to a reference, e.g., in the case where altered control region, or addition of an agent, results in an increased expression of the subject product, it is increased relative to an otherwise similar cell without the alteration or addition.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

A “modification,” as that term is used herein with reference to a nucleotide, refers to a modification of the chemical structure, e.g., a covalent modification, of the subject nucleotide. The modification can be naturally occurring or non-naturally occurring. In an embodiment, the modification is non-naturally occurring. In an embodiment, the modification is naturally occurring. In an embodiment, the modification is a synthetic modification. In an embodiment, the modification is a modification provided in Tables 4, 5, 6, 7, or 8.

A “non-naturally occurring modification,” as that term is used herein with reference to a nucleotide, refers to a modification that: (a) a cell, e.g., a human cell, does not make on an endogenous tRNA; or (b) a cell, e.g., a human cell, can make on an endogenous tRNA but wherein such modification is in a location in which it does not occur on a native tRNA, e.g., the modification is in a domain, linker or arm, or on a nucleotide and/or at a position within a domain, linker or arm, which does not have such modification in nature. In either case, the modification is added synthetically, e.g., in a cell free reaction, e.g., in a solid state or liquid phase synthetic reaction. In an embodiment, the non-naturally occurring modification is a modification that is not present (in identity, location or position) if a sequence of the TREM is expressed in a mammalian cell, e.g., a HEK293 cell line. Exemplary non-naturally occurring modifications are found in Tables 4, 5, 6, 7, or 8.

A “nucleotide,” as that term is used herein, refers to an entity comprising a sugar, typically a pentameric sugar; a nucleobase; and a phosphate linking group. In an embodiment, a nucleotide comprises a naturally occurring, e.g., naturally occurring in a human cell, nucleotide, e.g., an adenine, thymine, guanine, cytosine, or uracil nucleotide.

A “non-naturally modified nucleotide,” as that term is used herein, refers a nucleotide comprising a non-naturally occurring modification on or of a sugar, nucleobase, or phosphate moiety.

A “naturally occurring nucleotide,” as that term is used herein, refers to a nucleotide that does not comprise a non-naturally occurring modification. In an embodiment, it includes a naturally occurring modification.

A “post-transcriptional processing,” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment the modification is made in vivo, e.g., in a cell used to produce a TREM. In an embodiment the modification is made ex vivo, e.g., it is made on a TREM isolated or obtained from the cell which produced the TREM. In an embodiment, the post-transcriptional modification is selected from a modification listed in Tables 4, 5, 6, 7, or 8.

A “premature termination codon” or “PTC” as those terms are used herein, refer to a stop codon that occurs in an open reading frame (ORF) of a DNA or mRNA. In an embodiment, a PTC occurs at a position upstream of a naturally occurring stop codon in an ORF. In an embodiment, a PTC that occurs upstream of a naturally occurring stop codon, e.g., in an ORF, results in modulation of a production parameter of the corresponding mRNA or polypeptide encoded by the ORF. In an embodiment, a PTC can differ (or arise) from a pre-mutation sequence by a point mutation, e.g., a nonsense mutation. In an embodiment, a PTC can differ (or arise) from a pre-mutation sequence by a genetic change, e.g., abnormality, other than a point mutation, e.g., a frameshift, a deletion, an insertion, a rearrangement, an inversion, a translocation, a duplication, or a transversion. In an embodiment, a PTC results in the production of a truncated protein which lacks a native activity or which is associated with a mutant, disease, or other unwanted phenotype. In an embodiment, the ORF comprising the PTC is an ORF from a tumor suppressor gene. In an embodiment, the mutation giving rise to the PTC is a driver mutation, e.g., a mutation that provides a growth advantage to a tumor cell.

A “subject,” as this term is used herein, includes any organism, such as a human or other animal. In embodiments, the subject is a vertebrate animal (e.g., mammal, bird, fish, reptile, or amphibian). In embodiments, the subject is a mammal, e.g., a human. In embodiments, the method subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). The subject may be a male or female of any age group, e.g., a pediatric subject (e.g., infant, child, adolescent) or adult subject (e.g., young adult, middle-aged adult, or senior adult)). A non-human subject may be a transgenic animal.

A “tRNA-based effector molecule” or “TREM,” as that term is used herein, refers to an RNA molecule comprising a structure or property from (a)-(v) below, and which is a recombinant TREM, a synthetic TREM, or a TREM expressed from a heterologous cell. The TREMs described in the present invention are synthetic molecules and are made, e.g., in a cell free reaction, e.g., in a solid state or liquid phase synthetic reaction. TREMs are chemically distinct, e.g., in terms of primary sequence, type or location of modifications from the endogenous tRNA molecules made in cells, e.g., in mammalian cells, e.g., in human cells. A TREM can have a plurality (e.g., 2, 3, 4, 5, 6, 7, 8, 9) of the structures and functions of (a)-(v).

In an embodiment, a TREM is non-native, as evaluated by structure or the way in which it was made.

In an embodiment, a TREM comprises one or more of the following structures or properties:
(a′) an optional linker region of a consensus sequence provided in the “Consensus Sequence” section, e.g., a Linker 1 region;
(a) an amino acid attachment domain that binds an amino acid, e.g., an acceptor stem 25 domain (AStD), wherein an AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment the AStD has at least 75, 80, 85, 85, 90, 95, or 100% identity with a naturally occurring AStD, e.g., an AStD encoded by a nucleic acid in Table 3. In an embodiment, the TREM can comprise a fragment or analog of an AStD, e.g., an AStD encoded by a nucleic acid in Table 3, which fragment in embodiments has AStD activity and in other embodiments does not have AStD activity. (One of ordinary skill can determine the relevant corresponding sequence for any of the domains, stems, loops, or other sequence features mentioned herein from a sequence encoded by a nucleic acid in Table 3 e.g., one of ordinary skill can determine the sequence which corresponds to an AStD from a tRNA sequence encoded by a nucleic acid in Table 3.)

In an embodiment the AStD falls under the corresponding sequence of a consensus sequence provided in the “Consensus Sequence” section, or differs from the consensus sequence by no more than 1, 2, 5, or 10 positions;
In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula I_{ZZZ}, wherein ZZZ indicates any of the twenty amino acids;
In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula II_{ZZZ}, wherein ZZZ indicates any of the twenty amino acids;
In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula III_{ZZZ}, wherein ZZZ indicates any of the twenty amino acids;
(a′-1) a linker comprising residues R₈-R₉ of a consensus sequence provided in the “Consensus Sequence” section, e.g., a Linker 2 region;
(b) a dihydrouridine hairpin domain (DHD), wherein a DHD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment the DHD has at least 75, 80, 85, 85, 90, 95, or 100% identity with a naturally occurring DHD, e.g., a DHD encoded by a nucleic acid in Table 3. In an embodiment, the TREM can comprise a fragment or analog of a DHD, e.g., a DHD encoded by a nucleic acid in Table 3, which fragment in embodiments has DHD activity and in other embodiments does not have DHD activity.

In an embodiment the DHD falls under the corresponding sequence of a consensus sequence provided in the “Consensus Sequence” section, or differs from the consensus sequence by no more than 1, 2, 5, or 10 positions;
In an embodiment, the DHD comprises residues R₁₀-R₁₁-R₁₂-R₁₃-R₁₄, R₁₅-R₁₆-R₁₇-R₁₈-R₁₉-R₂₀-R₂₁-R₂₂-R₂₃-R₂₄-R₂₅-R₂₆-R₂₇-R₂₈ of Formula I_{ZZZ}, wherein ZZZ indicates any of the twenty amino acids;
In an embodiment, the DHD comprises residues R₁₀-R₁₁-R₁₂-R₁₃-R₁₄, R₁₅-R₁₆-R₁₇-R₁₈-R₁₉-R₂₀-R₂₁-R₂₂-R₂₃-R₂₄-R₂₅-R₂₆-R₂₇-R₂₈ of Formula II_{ZZZ}, wherein ZZZ indicates any of the twenty amino acids;
In an embodiment, the DHD comprises residues R₁₀-R₁₁-R₁₂-R₁₃-R₁₄, R₁₅-R₁₆-R₁₇-R₁₈-R₁₉-R₂₀-R₂₁-R₂₂-R₂₃-R₂₄-R₂₅-R₂₆-R₂₇-R₂₈ of Formula III_{ZZZ}, wherein ZZZ indicates any of the twenty amino acids;
(b′-1) a linker comprising residue R₂₉ of a consensus sequence provided in the “Consensus Sequence” section, e.g., a Linker 3 region;
(c) an anticodon that binds a respective codon in an mRNA, e.g., an anticodon hairpin domain (ACHD), wherein an ACHD comprises sufficient sequence, e.g., an anticodon triplet, to mediate, e.g., when present in an otherwise wildtype tRNA, pairing (with or without wobble) with a codon. In an embodiment the ACHD has at least 75, 80, 85, 85, 90, 95, or 100% identity with a naturally occurring ACHD, e.g., an ACHD encoded by a nucleic acid in Table 3. In an embodiment, the TREM can comprise a fragment or analog of an ACHD, e.g., an ACHD encoded by a nucleic acid in Table 3, which fragment in embodiments has ACHD activity and in other embodiments does not have ACHD activity.

In an embodiment the ACHD falls under the corresponding sequence of a consensus sequence provided in the “Consensus Sequence” section, or differs from the consensus sequence by no more than 1, 2, 5, or 10 positions;
In an embodiment, the ACHD comprises residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula I_{ZZZ}, wherein ZZZ indicates any of the twenty amino acids;
In an embodiment, the ACHD comprises residues -R₃₀-R₃₁-R₃₂-R₃₃-R₃₄-R₃₅-R₃₆-R₃-R₃₈-R₃₉-R₄₀-R₄₁-R₄₂-R₄₃-R₄₄-R₄₅-R₄₆ of Formula II_{ZZZ}, wherein ZZZ indicates any of the twenty amino acids;
In an embodiment, the ACHD comprises residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula III_{ZZZ}, wherein ZZZ indicates any of the twenty amino acids;
(d) a variable loop domain (VLD), wherein a VLD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment the VLD has at least 75, 80, 85, 85, 90, 95, or 100% identity with a naturally occurring VLD, e.g., a VLD encoded by a nucleic acid in Table 3. In an embodiment, the TREM can comprise a fragment or analog of a VLD, e.g., a VLD encoded by a nucleic acid in Table 3, which fragment in embodiments has VLD activity and in other embodiments does not have VLD activity.

In an embodiment the VLD falls under the corresponding sequence of a consensus sequence provided in the “Consensus Sequence” section.

In an embodiment, the VLD comprises residue -[R₄₇]ₓ of a consensus sequence provided in the “Consensus Sequence” section, wherein x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271);
(e) a thymine hairpin domain (THD), wherein a THD comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment the THD has at least 75, 80, 85, 85, 90, 95, or 100% identity with a naturally occurring THD, e.g., a THD encoded by a nucleic acid in Table 3. In an embodiment, the TREM can comprise a fragment or analog of a THD, e.g., a THD encoded by a nucleic acid in Table 3, which fragment in embodiments has THD activity and in other embodiments does not have THD activity.

In an embodiment the THD falls under the corresponding sequence of a consensus sequence provided in the “Consensus Sequence” section, or differs from the consensus sequence by no more than 1, 2, 5, or 10 positions;
In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula I_{ZZZ}, wherein ZZZ indicates any of the twenty amino acids;
In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula II_{ZZZ}, wherein ZZZ indicates any of the twenty amino acids;
In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula III_{ZZZ}, wherein ZZZ indicates any of the twenty amino acids;
(e′-1) a linker comprising residue R₇₂ of a consensus sequence provided in the “Consensus Sequence” section, e.g., a Linker 4 region;
(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 3. In an embodiment, the TREM can comprise a fragment or analog of a stem or loop structure, e.g., a stem or loop structure encoded by a nucleic acid in Table 3, which fragment in embodiments has activity of a stem or loop structure, and in other embodiments does not have activity of a stem or loop structure;
(g) a tertiary structure, e.g., an L-shaped tertiary structure;
(h) adaptor function, i.e., the TREM mediates acceptance of an amino acid, e.g., its cognate amino acid and transfer of the AA in the initiation or elongation of a polypeptide chain;
(i) cognate adaptor function wherein the TREM mediates acceptance and incorporation of an amino acid (e.g., cognate amino acid) associated in nature with the anti-codon of the TREM to initiate or elongate a polypeptide chain;
(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;
(k) a regulatory function, e.g., an epigenetic function (e.g., gene silencing function or signaling pathway modulation function), cell fate modulation function, mRNA stability modulation function, protein stability modulation function, protein transduction modulation function, or protein compartmentalization function;
(l) a structure which allows for ribosome binding:
(m) a post-transcriptional modification, e.g., a naturally occurring post-transcriptional modification;
(n) the ability to inhibit a functional property of a tRNA, e.g., any of properties (h)-(k) possessed by a tRNA;
(o) the ability to modulate cell fate;
(p) the ability to modulate ribosome occupancy;
(q) the ability to modulate protein translation;
(r) the ability to modulate mRNA stability;
(s) the ability to modulate protein folding and structure;
(t) the ability to modulate protein transduction or compartmentalization;
(u) the ability to modulate protein stability; or
(v) the ability to modulate a signaling pathway, e.g., a cellular signaling pathway.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g).

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (c).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:
(i) an amino acid attachment domain that binds an amino acid (e.g., an AStD, as described in (a) herein; and
(ii) an anticodon that binds a respective codon in an mRNA (e.g., an ACHD, as described in (c) herein).

In an embodiment the TREM comprises a flexible RNA linker which provides for covalent linkage of (i) to (ii).

In an embodiment, the TREM mediates protein translation.

In an embodiment a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 3, or a fragment or functional fragment thereof. In an embodiment, a TREM comprises an RNA sequence encoded by a DNA sequence listed in Table 3, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 3, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 3, or a fragment or a functional fragment thereof. In an embodiment, a TREM comprises a TREM domain, e.g., a domain described herein, comprising an RNA sequence encoded by DNA sequence listed in Table 3, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 3, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM (uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′ halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

In an embodiment of any of the TREMs, TREM core fragments, or TREM fragments disclosed herein, the TREM sequence comprises a CCA sequence on a terminus, e.g., the 3′ terminus. In an embodiment, the TREM sequence does not comprise a CCA sequence on a terminus, e.g., the 3′ terminus.

A “TREM core fragment,” as that term is used herein, refers to a portion of the sequence of Formula B: [L1]_{y}-[ASt Domain₁]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1.

A “TREM fragment,” as used herein, refers to a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

A “cognate adaptor function TREM,” as that term is used herein, refers to a TREM which mediates initiation or elongation with the AA (the cognate AA) associated in nature with the anti-codon of the TREM.

An “exogenous TREM,” as that term is used herein, refers to a TREM that:
(a) differs by at least one nucleotide or one post transcriptional modification from the closest sequence tRNA in a reference cell, e.g., a cell into which the exogenous nucleic acid is introduced;
(b) has been introduced into a cell other than the cell in which it was transcribed;
(c) is present in a cell other than one in which it naturally occurs; or
(d) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment an exogenous TREM comprises 1, 2, 3 or 4 of properties (a)-(d).

A “non-cognate adaptor function TREM,” as that term is used herein, refers to a TREM which mediates initiation or elongation with an AA (a non-cognate AA) other than the AA associated in nature with the anti-codon of the TREM. In an embodiment, a non-cognate adaptor function TREM is also referred to as a mischarged TREM (mTREM).

A “non-naturally occurring sequence,” as that term is used herein, refers to a sequence wherein an Adenine is replaced by a residue other than an analog of Adenine, a Cytosine is replaced by a residue other than an analog of Cytosine, a Guanine is replaced by a residue other than an analog of Guanine, and a Uracil is replaced by a residue other than an analog of Uracil. An analog refers to any possible derivative of the ribonucleotides, A, G, C or U. In an embodiment, a sequence having a derivative of any one of ribonucleotides A, G, C or U is a non-naturally occurring sequence.

A “pharmaceutical TREM composition,” as that term is used herein, refers to a TREM composition that is suitable for pharmaceutical use. Typically, a pharmaceutical TREM composition comprises a pharmaceutical excipient. In an embodiment the TREM will be the only active ingredient in the pharmaceutical TREM composition. In embodiments the pharmaceutical TREM composition is free, substantially free, or has less than a pharmaceutically acceptable amount, of host cell proteins, DNA, e.g., host cell DNA, endotoxins, and bacteria.

A “synthetic TREM,” as that term is used herein, refers to a TREM which was synthesized other than in or by a cell having an endogenous nucleic acid encoding the TREM, e.g., a synthetic TREM is synthetized by cell-free solid phase synthesis. A synthetic TREM can have the same, or a different, sequence, or tertiary structure, as a native tRNA.

A “recombinant TREM,” as that term is used herein, refers to a TREM that was expressed in a cell modified by human intervention, having a modification that mediates the production of the TREM, e.g., the cell comprises an exogenous sequence encoding the TREM, or a modification that mediates expression, e.g., transcriptional expression or post-transcriptional modification, of the TREM. A recombinant TREM can have the same, or a different, sequence, set of post-transcriptional modifications, or tertiary structure, as a reference tRNA, e.g., a native tRNA.

A “tRNA”, as that term is used herein, refers to a naturally occurring transfer ribonucleic acid in its native state.

A “TREM composition,” as that term is used herein, refers to a composition comprising a plurality of TREMs, a plurality of TREM core fragments and/or a plurality of TREM fragments. A TREM composition can comprise one or more species of TREMs, TREM core fragments or TREM fragments. In an embodiment, the composition comprises only a single species of TREM, TREM core fragment or TREM fragment. In an embodiment, the TREM composition comprises a first TREM, TREM core fragment or TREM fragment species; and a second TREM, TREM core fragment or TREM fragment species. In an embodiment, the TREM composition comprises X TREM, TREM core fragment or TREM fragment species, wherein X=2, 3, 4, 5, 6, 7, 8, 9, or 10. In an embodiment, the TREM, TREM core fragment or TREM fragment has at least 70, 75, 80, 85, 90, or 95, or has 100%, identity with a sequence encoded by a nucleic acid in Table 3. A TREM composition can comprise one or more species of TREMs, TREM core fragments or TREM fragments. In an embodiment, the TREM composition is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid. In an embodiment, the composition is dry, e.g., a lyophilized material. In an embodiment, the composition is a frozen composition. In an embodiment, the composition is sterile. In an embodiment, the composition comprises at least 0.5 g, 1.0 g, 5.0 g, 10 g, 15 g, 25 g, 50 g, 100 g, 200 g, 400 g, or 500 g (e.g., as determined by dry weight) of TREM.

In an embodiment, at least X % of the TREMs in a TREM composition has a non-naturally occurring modification at a selected position, and X is 80, 90, 95, 96, 97, 98, 99, or 99.5.

In an embodiment, at least X % of the TREMs in a TREM composition has a non-naturally occurring modification at a first position and a non-naturally occurring modification at a second position, and X, independently, is 80, 90, 95, 96, 97, 98, 99, or 99.5. In embodiments, the modification at the first and second position is the same. In embodiments, the modification at the first and second position are different. In embodiments, the nucleotide at the first and second position is the same, e.g., both are adenine. In embodiments, the nucleotide at the first and second position are different, e.g., one is adenine and one is thymine.

In an embodiment, at least X % of the TREMs in a TREM composition has a non-naturally occurring modification at a first position and less than Y % have a non-naturally occurring modification at a second position, wherein X is 80, 90, 95, 96, 97, 98, 99, or 99.5 and Y is 20, 20, 5, 2, 1, 0.1, or 0.01. In embodiments, the nucleotide at the first and second position is the same, e.g., both are adenine. In embodiments the nucleotide at the first and second position are different, e.g., one is adenine and one is thymine.

### Trem, Trem Core Fragment and Trem Fragment

A “tRNA-based effector molecule” or “TREM” refers to an RNA molecule comprising one or more of the properties described herein. A TREM can comprise a non-naturally occurring modification, e.g., as provided in Tables 4, 5, 6, 7, or 8.

In an embodiment, a TREM includes a TREM comprising a sequence of Formula A; a TREM core fragment comprising a sequence of Formula B; or a TREM fragment comprising a portion of a TREM which TREM comprises a sequence of Formula A.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2]. In an embodiment, [VL Domain] is optional. In an embodiment, [L1] is optional.
In an embodiment, a TREM core fragment comprises a sequence of Formula B: [L1]_{y}-[ASt Domain₁]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1. In an embodiment, y=0. In an embodiment, y=1;
In an embodiment, a TREM fragment comprises a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2], and wherein the TREM fragment comprises: one, two, three or all or any combination of the following: a TREM half (e.g., from a cleavage in the ACH Domain, e.g., in the anticodon sequence, e.g., a 5′ half or a 3′ half); a 5′ fragment (e.g., a fragment comprising the 5′ end, e.g., from a cleavage in a DH Domain or the ACH Domain); a 3′ fragment (e.g., a fragment comprising the 3′ end, e.g., from a cleavage in the TH Domain); or an internal fragment (e.g., from a cleavage in any one of the ACH Domain, DH Domain or TH Domain). Exemplary TREM fragments include TREM halves (e.g., from a cleavage in the ACHD, e.g., 5′ TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM, a TREM core fragment or a TREM fragment can be charged with an amino acid (e.g., a cognate amino acid); charged with a non-cognate amino acid (e.g., a mischarged TREM (mTREM)); or not charged with an amino acid (e.g., an uncharged TREM (uTREM)). In an embodiment, a TREM, a TREM core fragment or a TREM fragment can be charged with an amino acid selected from alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In some embodiments, a non-extended anticodon is an anticodon of no more than three nucleotides. In an embodiment, a non-extended codon pairs with no more than three codon nucleotides on a nucleic acid being translated.

In an embodiment, the TREM, TREM core fragment or TREM fragment is a cognate TREM. In an embodiment, the TREM, TREM core fragment or TREM fragment is a non-cognate TREM. In an embodiment, the TREM, TREM core fragment or TREM fragment recognizes a codon provided in Table 1 or Table 2.

TABLE 1
List of codons

AAA
AAC
AAG
AAU
ACA
ACC
ACG
ACU
AGA
AGC
AGG
AGU
AUA
AUC
AUG
AUU
CAA
CAC
CAG
CAU
CCA
CCC
CCG
CCU
CGA
CGC
CGG
CGU
CUA
CUC
CUG
CUU
GAA
GAC
GAG
GAU
GCA
GCC
GCG
GCU
GGA
GGC
GGG
GGU
GUA
GUC
GUG
GUU
UAA
UAC
UAG
UAU
UCA
UCC
UCG
UCU
UGA
UGC
UGG
UGU
UUA
UUC
UUG
UUU

TABLE 2
Amino acids and corresponding codons

| Amino Acid | mRNA codons |
|---|---|
| Alanine | GCU, GCC, GCA, GCG |
| Arginine | CGU, CGC, CGA, CGG, AGA, AGG |
| Asparagine | AAU, AAC |
| Aspartate | GAU, GAC |
| Cysteine | UGU, UGC |
| Glutamate | GAA, GAG |
| Glutamine | CAA, CAG |
| Glycine | GGU, GGC, GGA, GGG |
| Histidine | CAU, CAC |
| Isoleucine | AUU, AUC, AUA |
| Leucine | UUA, UUG, CUU, CUC, CUA, CUG |
| Lysine | AAA, AAG |
| Methionine | AUG |
| Phenylalanine | UUU, UUC |
| Proline | CCU, CCC, CCA, CCG |
| Serine | UCU, UCC, UCA, UCG, AGU, AGC |
| Stop | UAA, UAG, UGA |
| Threonine | ACU, ACC, ACA, ACG |
| Tryptophan | UGG |
| Tyrosine | UAU, UAC |
| Valine | GUU, GUC, GUA, GUG |

In an embodiment, a TREM comprises a ribonucleic acid (RNA) sequence encoded by a deoxyribonucleic acid (DNA) sequence disclosed in Table 3, e.g., any one of SEQ ID NOs: 1-451 disclosed in Table 3. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 3, e.g., any one of SEQ ID NOs: 1-451 disclosed in Table 3. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 3.

In an embodiment, a TREM, a TREM core fragment, or TREM fragment comprises at least 5, 10, 15, 20, 25, or 30 consecutive nucleotides of an RNA sequence encoded by a DNA sequence disclosed in Table 3, e.g., at least 5, 10, 15, 20, 25, or 30 consecutive nucleotides of an RNA sequence encoded by any one of SEQ ID NOs: 1-451 disclosed in Table 3. In an embodiment, a TREM, a TREM core fragment, or TREM fragment comprises at least 5, 10, 15, 20, 25, or 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 3, e.g., any one of SEQ ID NOs: 1-451 disclosed in Table 3. In an embodiment, a TREM, a TREM core fragment, or TREM fragment comprises at least 5, 10, 15, 20, 25, or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 3, e.g., any one of SEQ ID NOs: 1-451 disclosed in Table 3.

In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 3, e.g., any one of SEQ ID NOs: 1-451 disclosed in Table 3. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 3, e.g., any one of SEQ ID NOs: 1-451 disclosed in Table 3. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 3, e.g., any one of SEQ ID NOs: 1-451 disclosed in Table 3.

In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 3, e.g., any one of SEQ ID NOs: 1-451 disclosed in Table 3. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 3, e.g., any one of SEQ ID NOs: 1-451 disclosed in Table 3. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 3, e.g., any one of SEQ ID NOs: 1-451 disclosed in Table 3.

In an embodiment, a TREM core fragment or a TREM fragment comprises a sequence of a length of between 10-90 ribonucleotides (rnt), between 10-80 rnt, between 10-70 rnt, between 10-60 rut, between 10-50 rut, between 10-40 rnt, between 10-30 rut, between 10-20 rnt, between 20-90 rut, between 20-80 rut, 20-70 rnt, between 20-60 rnt, between 20-50 rnt, between 20-40 rnt, between 30-90 rnt, between 30-80 rnt, between 30-70 rnt, between 30-60 rnt, or between 30-50 rnt

TABLE 3
List of tRNA Sequences

| SEQ ID | | |
|---|---|---|
| NO | tRNA name | tRNA sequence |
| 1 | Ala_AGC_chr6:28763 | GGGGGTATAGCTCAGTGGTAGAGCGCGTGCT |
| | 741-28763812 (−) | TAGCATGCACGAGGTCCTGGGTTCGATCCCC |
| 2 | Ala_AGC_chr6:26687 | GGGGAATTAGCTCAAGTGGTAGAGCGCTTGC |
| | 485-26687557 (+) | TTAGCACGCAAGAGGTAGTGGGATCGATGCC |
| 3 | Ala_AGC_chr6:26572 | GGGGAATTAGCTCAAATGGTAGAGCGCTCGC |
| | 092-26572164 (−) | TTAGCATGCGAGAGGTAGCGGGATCGATGCC |
| 4 | Ala_AGC_chr6:26682 | GGGGAATTAGCTCAAGTGGTAGAGCGCTTGC |
| | 715-26682787 (+) | TTAGCATGCAAGAGGTAGTGGGATCGATGCC |
| 5 | Ala_AGC_chr6:26705 | GGGGAATTAGCTCAAGCGGTAGAGCGCTTGC |
| | 606-26705678 (+) | TTAGCATGCAAGAGGTAGTGGGATCGATGCC |
| 6 | Ala_AGC_chr6:26673 | GGGGAATTAGCTCAAGTGGTAGAGCGCTTGC |
| | 590-26673662 (+) | TTAGCATGCAAGAGGTAGTGGGATCAATGCC |
| 7 | Ala_AGC_chr14:8944 | GGGGAATTAGCTCAAGTGGTAGAGCGCTCGC |
| | 5442-89445514 (+) | TTAGCATGCGAGAGGTAGTGGGATCGATGCC |
| 8 | Ala_AGC_chr6:58196 | GGGGAATTAGCCCAAGTGGTAGAGCGCTTGC |
| | 623-58196695 (−) | TTAGCATGCAAGAGGTAGTGGGATCGATGCC |
| 9 | Ala_AGC_chr6:28806 | GGGGGTGTAGCTCAGTGGTAGAGCGCGTGCT |
| | 221-28806292 (−) | TAGCATGCACGAGGCCCCGGGTTCAATCCCC |
| 10 | Ala_AGC_chr6:28574 | GGGGGTGTAGCTCAGTGGTAGAGCGCGTGCT |
| | 933-28575004 (+) | TAGCATGTACGAGGTCCCGGGTTCAATCCCC |
| 11 | Ala_AGC_chr6:28626 | GGGGATGTAGCTCAGTGGTAGAGCGCATGCT |
| | 014-28626085 (−) | TAGCATGCATGAGGTCCCGGGTTCGATCCCC |
| 12 | Ala_AGC_chr6:28678 | GGGGGTGTAGCTCAGTGGTAGAGCGCGTGCT |
| | 366-28678437 (+) | TAGCATGCACGAGGCCCTGGGTTCAATCCCC |
| 13 | Ala_AGC_chr6:28779 | GGGGGTATAGCTCAGCGGTAGAGCGCGTGCT |
| | 849-28779920 (−) | TAGCATGCACGAGGTCCTGGGTTCAATCCCC |
| 14 | Ala_AGC_chr6:28687 | GGGGGTGTAGCTCAGTGGTAGAGCGCGTGCT |
| | 481-28687552 (+) | TAGCATGCACGAGGCCCCGGGTTCAATCCCT |
| 15 | Ala_AGC_chr2:27274 | GGGGGATTAGCTCAAATGGTAGAGCGCTCGC |
| | 082-27274154 (+) | TTAGCATGCGAGAGGTAGCGGGATCGATGCC |
| 16 | Ala_AGC_chr6:26730 | GGGGAATTAGCTCAGGCGGTAGAGCGCTCGC |
| | 737-26730809 (+) | TTAGCATGCGAGAGGTAGCGGGATCGACGCC |
| 17 | Ala_CGC_chr6:26553 | GGGGATGTAGCTCAGTGGTAGAGCGCATGCT |
| | 731-26553802 (+) | TCGCATGTATGAGGTCCCGGGTTCGATCCCC |
| 18 | Ala_CGC_chr6:28641 | GGGGATGTAGCTCAGTGGTAGAGCGCATGCT |
| | 613-28641684 (−) | TCGCATGTATGAGGCCCCGGGTTCGATCCCC |
| 19 | Ala_CGC_chr2:15725 | GGGGATGTAGCTCAGTGGTAGAGCGCGCGCT |
| | 7281-157257352 (+) | TCGCATGTGTGAGGTCCCGGGTTCAATCCCC |
| 20 | Ala_CGC_chr6:28697 | GGGGGTGTAGCTCAGTGGTAGAGCGCGTGCT |
| | 092-28697163 (+) | TCGCATGTACGAGGCCCCGGGTTCGACCCCC |
| 21 | Ala_TGC_chr6:28757 | GGGGGTGTAGCTCAGTGGTAGAGCGCATGCT |
| | 547-28757618 (−) | TTGCATGTATGAGGTCCCGGGTTCGATCCCC |
| 22 | Ala_TGC_chr6:28611 | GGGGATGTAGCTCAGTGGTAGAGCGCATGCT |
| | 222-28611293 (+) | TTGCATGTATGAGGTCCCGGGTTCGATCCCC |
| 23 | Ala_TGC_chr5:18063 | GGGGATGTAGCTCAGTGGTAGAGCGCATGCT |
| | 3868-180633939 (+) | TTGCATGTATGAGGCCCCGGGTTCGATCCCC |
| 24 | Ala_TGC_chr12:1254 | GGGGATGTAGCTCAGTGGTAGAGCGCATGCT |
| | 24512-125424583 (+) | TTGCACGTATGAGGCCCCGGGTTCAATCCCC |
| 25 | Ala_TGC_chr6:28785 | GGGGGTGTAGCTCAGTGGTAGAGCGCATGCT |
| | 012-28785083 (−) | TTGCATGTATGAGGCCTCGGGTTCGATCCCC |
| 26 | Ala_TGC_chr6:28726 | GGGGGTGTAGCTCAGTGGTAGAGCACATGCT |
| | 141-28726212 (−) | TTGCATGTGTGAGGCCCCGGGTTCGATCCCC |
| 27 | Ala_TGC_chr6:28770 | GGGGGTGTAGCTCAGTGGTAGAGCGCATGCT |
| | 577-28770647 (−) | TTGCATGTATGAGGCCTCGGTTCGATCCCCG |
| 28 | Arg_ACG_chr6:26328 | GGGCCAGTGGCGCAATGGATAACGCGTCTGA |
| | 368-26328440 (+) | CTACGGATCAGAAGATTCCAGGTTCGACTCC |
| 29 | Arg_ACG_chr3:45730 | GGGCCAGTGGCGCAATGGATAACGCGTCTGA |
| | 491-45730563 (−) | CTACGGATCAGAAGATTCTAGGTTCGACTCC |
| 30 | Arg_CCG_chr6:28710 | GGCCGCGTGGCCTAATGGATAAGGCGTCTGA |
| | 729-28710801 (−) | TTCCGGATCAGAAGATTGAGGGTTCGAGTCC |
| 31 | Arg_CCG_chr17:6601 | GACCCAGTGGCCTAATGGATAAGGCATCAGC |
| | 6013-66016085 (−) | CTCCGGAGCTGGGGATTGTGGGTTCGAGTCC |
| 32 | Arg_CCT_chr17:7303 | GCCCCAGTGGCCTAATGGATAAGGCACTGGC |
| | 0001-73030073 (+) | CTCCTAAGCCAGGGATTGTGGGTTCGAGTCC |
| 33 | Arg_CCT_chr17:7303 | GCCCCAGTGGCCTAATGGATAAGGCACTGGC |
| | 0526-73030598 (−) | CTCCTAAGCCAGGGATTGTGGGTTCGAGTCC |
| 34 | Arg_CCT_chr16:3202 | GCCCCGGTGGCCTAATGGATAAGGCATTGGC |
| | 901-3202973 (+) | CTCCTAAGCCAGGGATTGTGGGTTCGAGTCC |
| 35 | Arg_CCT_chr7:13902 | GCCCCAGTGGCCTAATGGATAAGGCATTGGC |
| | 5446-139025518 (+) | CTCCTAAGCCAGGGATTGTGGGTTCGAGTCC |
| 36 | Arg_CCT_chr16:3243 | GCCCCAGTGGCCTGATGGATAAGGTACTGGC |
| | 918-3243990 (+) | CTCCTAAGCCAGGGATTGTGGGTTCGAGTTC |
| 37 | Arg_TCG_chr15:8987 | GGCCGCGTGGCCTAATGGATAAGGCGTCTGA |
| | 8304-89878376 (+) | CTTCGGATCAGAAGATTGCAGGTTCGAGTCC |
| 38 | Arg_TCG_chr6:26323 | GACCACGTGGCCTAATGGATAAGGCGTCTGA |
| | 046-26323118 (+) | CTTCGGATCAGAAGATTGAGGGTTCGAATCC |
| 39 | Arg_TCG_chr17:7303 | GACCGCGTGGCCTAATGGATAAGGCGTCTGA |
| | 1208-73031280 (+) | CTTCGGATCAGAAGATTGAGGGTTCGAGTCC |
| 40 | Arg_TCG_chr6:26299 | GACCACGTGGCCTAATGGATAAGGCGTCTGA |
| | 905-26299977 (+) | CTTCGGATCAGAAGATTGAGGGTTCGAATCC |
| 41 | Arg_TCG_chr6:28510 | GACCACGTGGCCTAATGGATAAGGCGTCTGA |
| | 891-28510963 (−) | CTTCGGATCAGAAGATTGAGGGTTCGAATCC |
| 42 | Arg_TCG_chr9:11296 | GGCCGTGTGGCCTAATGGATAAGGCGTCTGA |
| | 0803-112960875 (+) | CTTCGGATCAAAAGATTGCAGGTTTGAGTTC |
| 43 | Arg_TCT_chr1:94313 | GGCTCCGTGGCGCAATGGATAGCGCATTGGA |
| | 129-94313213 (+) | CTTCTAGAGGCTGAAGGCATTCAAAGGTTCC |
| 44 | Arg_TCT_chr17:8024 | GGCTCTGTGGCGCAATGGATAGCGCATTGGA |
| | 243-8024330 (+) | CTTCTAGTGACGAATAGAGCAATTCAAAGGT |
| 45 | Arg_TCT_chr9:13110 | GGCTCTGTGGCGCAATGGATAGCGCATTGGA |
| | 2355-131102445 (−) | CTTCTAGCTGAGCCTAGTGTGGTCATTCAAA |
| 46 | Arg_TCT_chr11:5931 | GGCTCTGTGGCGCAATGGATAGCGCATTGGA |
| | 8767-59318852 (+) | CTTCTAGATAGTTAGAGAAATTCAAAGGTTG |
| 47 | Arg_TCT_chr1:15911 | GTCTCTGTGGCGCAATGGACGAGCGCGCTGG |
| | 1401-159111474 (−) | ACTTCTAATCCAGAGGTTCCGGGTTCGAGTC |
| 48 | Arg_TCT_chr6:27529 | GGCTCTGTGGCGCAATGGATAGCGCATTGGA |
| | 963-27530049 (+) | CTTCTAGCCTAAATCAAGAGATTCAAAGGTT |
| 49 | Asn_GTT_chr1:16151 | GTCTCTGTGGCGCAATCGGTTAGCGCGTTCG |
| | 0031-161510104 (+) | GCTGTTAACCGAAAGGTTGGTGGTTCGATCC |
| 50 | Asn_GTT_chr1:14387 | GTCTCTGTGGCGCAATCGGCTAGCGCGTTTG |
| | 9832-143879905 (−) | GCTGTTAACTAAAAGGTTGGCGGTTCGAACC |
| 51 | Asn_GTT_chr1:14430 | GTCTCTGTGGTGCAATCGGTTAGCGCGTTCC |
| | 1611-144301684 (+) | GCTGTTAACCGAAAGCTTGGTGGTTCGAGCC |
| | | C |
| 52 | Asn_GTT_chr1:14932 | GTCTCTGTGGCGCAATCGGCTAGCGCGTTTG |
| | 6272-149326345 (−) | GCTGTTAACTAAAAAGTTGGTGGTTCGAACA |
| 53 | Asn_GTT_chr1:14824 | GTCTCTGTGGCGCAATCGGTTAGCGCGTTCG |
| | 8115-148248188 (+) | GCTGTTAACCGAAAGGTTGGTGGTTCGAGCC |
| 54 | Asn_GTT_chr1:14859 | GTCTCTGTGGCGCAATCGGTTAGCGCATTCG |
| | 8314-148598387 (−) | GCTGTTAACCGAAAGGTTGGTGGTTCGAGCC |
| 55 | Asn_GTT_chr1:17216 | GTCTCTGTGGCGCAATCGGTTAGCGCGTTCG |
| | 172-17216245 (+) | GCTGTTAACCGAAAGATTGGTGGTTCGAGCC |
| 56 | Asn_GTT_chr1:16847 | GTCTCTGTGGCGCAATCGGTTAGCGCGTTCG |
| | 080-16847153 (−) | GCTGTTAACTGAAAGGTTGGTGGTTCGAGCC |
| 57 | Asn_GTT_chr1:14923 | GTCTCTGTGGCGCAATGGGTTAGCGCGTTCG |
| | 0570-149230643 (−) | GCTGTTAACCGAAAGGTTGGTGGTTCGAGCC |
| 58 | Asn_GTT_chr1:14800 | GTCTCTGTGGCGTAGTCGGTTAGCGCGTTCG |
| | 0805-148000878 (+) | GCTGTTAACCGAAAAGTTGGTGGTTCGAGCC |
| 59 | Asn_GTT_chr1:14971 | GTCTCTGTGGCGCAATCGGCTAGCGCGTTTG |
| | 1798-149711871 (−) | GCTGTTAACTAAAAGGTTGGTGGTTCGAACC |
| 60 | Asn_GTT_chr1:14597 | GTCTCTGTGGCGCAATCGGTTAGCGCGTTCG |
| | 9034-145979107 (−) | GCTGTTAACTGAAAGGTTAGTGGTTCGAGCC |
| 61 | Asp_GTC_chr12:9889 | TCCTCGTTAGTATAGTGGTTAGTATCCCCGC |
| | 7281-98897352 (+) | CTGTCACGCGGGAGACCGGGGTTCAATTCCC |
| | | C |
| 62 | Asp_GTC_chr1:16141 | TCCTCGTTAGTATAGTGGTGAGTATCCCCGC |
| | 0615-161410686 (−) | CTGTCACGCGGGAGACCGGGGTTCGATTCCC |
| | | C |
| 63 | Asp_GTC_chr6:27551 | TCCTCGTTAGTATAGTGGTGAGTGTCCCCGT |
| | 236-27551307 (−) | CTGTCACGCGGGAGACCGGGGTTCGATTCCC |
| | | C |
| 64 | Cys_GCA_chr7:14900 | GGGGGCATAGCTCAGTGGTAGAGCATTTGAC |
| | 7281-149007352 (+) | TGCAGATCAAGAGGTCCCTGGTTCAAATCCA |
| 65 | Cys_GCA_chr7:14907 | GGGGGTATAGCTCAGGGGTAGAGCATTTGAC |
| | 4601-149074672 (−) | TGCAGATCAAGAGGTCCCTGGTTCAAATCCA |
| 66 | Cys_GCA_chr7:14911 | GGGGGTATAGCTTAGCGGTAGAGCATTTGAC |
| | 2229-149112300 (−) | TGCAGATCAAGAGGTCCCCGGTTCAAATCCG |
| 67 | Cys_GCA_chr7:14934 | GGGGGTATAGCTTAGGGGTAGAGCATTTGAC |
| | 4046-149344117 (−) | TGCAGATCAAAAGGTCCCTGGTTCAAATCCA |
| 68 | Cys_GCA_chr7:14905 | GGGGGTATAGCTCAGGGGTAGAGCATTTGAC |
| | 2766-149052837 (−) | TGCAGATCAAGAGGTCCCCAGTTCAAATCTG |
| 69 | Cys_GCA_chr17:3701 | GGGGGTATAGCTCAGGGGTAGAGCATTTGAC |
| | 7937-37018008 (−) | TGCAGATCAAGAAGTCCCCGGTTCAAATCCG |
| 70 | Cys_GCA_chr7:14928 | GGGGGTATAGCTCAGGGGTAGAGCATTTGAC |
| | 1816-149281887 (+) | TGCAGATCAAGAGGTCTCTGGTTCAAATCCA |
| 71 | Cys_GCA_chr7:14924 | GGGGGTATAGCTCAGGGGTAGAGCACTTGAC |
| | 3631-149243702 (+) | TGCAGATCAAGAAGTCCTTGGTTCAAATCCA |
| 72 | Cys_GCA_chr7:14938 | GGGGATATAGCTCAGGGGTAGAGCATTTGAC |
| | 8272-149388343 (−) | TGCAGATCAAGAGGTCCCCGGTTCAAATCCG |
| 73 | Cys_GCA_chr7: 14907 | GGGGGTATAGTTCAGGGGTAGAGCATTTGAC |
| | 2850-149072921 (−) | TGCAGATCAAGAGGTCCCTGGTTCAAATCCA |
| 74 | Cys_GCA_chr7:14931 | GGGGGTATAGCTCAGGGGTAGAGCATTTGAC |
| | 0156-149310227 (−) | TGCAAATCAAGAGGTCCCTGATTCAAATCCA |
| 75 | Cys_GCA_chr4:12443 | GGGGGTATAGCTCAGTGGTAGAGCATTTGAC |
| | 0005-124430076 (−) | TGCAGATCAAGAGGTCCCCGGTTCAAATCCG |
| 76 | Cys_GCA_chr7:14929 | GGGCGTATAGCTCAGGGGTAGAGCATTTGAC |
| | 5046-149295117 (+) | TGCAGATCAAGAGGTCCCCAGTTCAAATCTG |
| 77 | Cys_GCA_chr7:14936 | GGGGGTATAGCTCACAGGTAGAGCATTTGAC |
| | 1915-149361986 (+) | TGCAGATCAAGAGGTCCCCGGTTCAAATCTG |
| 78 | Cys_GCA_chr7:14925 | GGGCGTATAGCTCAGGGGTAGAGCATTTGAC |
| | 3802-149253871 (+) | TGCAGATCAAGAGGTCCCCAGTTCAAATCTG |
| 79 | Cys_GCA_chr7:14929 | GGGGGTATAGCTCACAGGTAGAGCATTTGAC |
| | 2305-149292376 (−) | TGCAGATCAAGAGGTCCCCGGTTCAAATCCG |
| 80 | Cys_GCA_chr7:14928 | GGGGGTATAGCTCAGGGGTAGAGCACTTGAC |
| | 6164-149286235 (−) | TGCAGATCAAGAGGTCCCTGGTTCAAATCCA |
| 81 | Cys_GCA_chr17:3702 | GGGGGTATAGCTCAGTGGTAGAGCATTTGAC |
| | 5545-37025616 (−) | TGCAGATCAAGAGGTCCCTGGTTCAAATCCG |
| 82 | Cys_GCA_chr15:8003 | GGGGGTATAGCTCAGTGGGTAGAGCATTTGA |
| | 6997-80037069 (+) | CTGCAGATCAAGAGGTCCCCGGTTCAAATCC |
| 83 | Cys_GCA_chr3:13194 | GGGGGTGTAGCTCAGTGGTAGAGCATTTGAC |
| | 7944-131948015 (−) | TGCAGATCAAGAGGTCCCTGGTTCAAATCCA |
| 84 | Cys_GCA_chr1:93981 | GGGGGTATAGCTCAGGTGGTAGAGCATTTGA |
| | 834-93981906 (−) | CTGCAGATCAAGAGGTCCCCGGTTCAAATCC |
| 85 | Cys_GCA_chr14:7342 | GGGGGTATAGCTCAGGGGTAGAGCATTTGAC |
| | 9679-73429750 (+) | TGCAGATCAAGAGGTCCCCGGTTCAAATCCG |
| 86 | Cys_GCA_chr3:13195 | GGGGGTATAGCTCAGGGGTAGAGCATTTGAC |
| | 0642-131950713 (−) | TGCAGATCAAGAGGTCCCTGGTTCAAATCCA |
| 87 | Gln_CTG_chr6:18836 | GGTTCCATGGTGTAATGGTTAGCACTCTGGA |
| | 402-18836473 (+) | CTCTGAATCCAGCGATCCGAGTTCAAATCTC |
| 88 | Gln_CTG_chr6:27515 | GGTTCCATGGTGTAATGGTTAGCACTCTGGA |
| | 531-27515602 (−) | CTCTGAATCCAGCGATCCGAGTTCAAGTCTC |
| 89 | Gln_CTG_chr1:14596 | GGTTCCATGGTGTAATGGTGAGCACTCTGGA |
| | 3304-145963375 (+) | CTCTGAATCCAGCGATCCGAGTTCGAGTCTC |
| 90 | Gln_CTG_chr1:14773 | GGTTCCATGGTGTAATGGTAAGCACTCTGGA |
| | 7382-147737453 (−) | CTCTGAATCCAGCGATCCGAGTTCGAGTCTC |
| 91 | Gln_CTG_chr6:27263 | GGTTCCATGGTGTAATGGTTAGCACTCTGGA |
| | 212-27263283 (+) | CTCTGAATCCGGTAATCCGAGTTCAAATCTC |
| 92 | Gln_CTG_chr6:27759 | GGCCCCATGGTGTAATGGTCAGCACTCTGGA |
| | 135-27759206 (−) | CTCTGAATCCAGCGATCCGAGTTCAAATCTC |
| 93 | Gln_CTG_chr1:14780 | GGTTCCATGGTGTAATGGTAAGCACTCTGGA |
| | 0937-147801008 (+) | CTCTGAATCCAGCCATCTGAGTTCGAGTCTC |
| | | T |
| 94 | Gln_TTG_chr17:4726 | GGTCCCATGGTGTAATGGTTAGCACTCTGGA |
| | 9890-47269961 (+) | CTTTGAATCCAGCGATCCGAGTTCAAATCTC |
| 95 | Gln_TTG_chr6:28557 | GGTCCCATGGTGTAATGGTTAGCACTCTGGA |
| | 156-28557227 (+) | CTTTGAATCCAGCAATCCGAGTTCGAATCTC |
| 96 | Gln_TTG_chr6:26311 | GGCCCCATGGTGTAATGGTTAGCACTCTGGA |
| | 424-26311495 (−) | CTTTGAATCCAGCGATCCGAGTTCAAATCTC |
| 97 | Gln_TTG_chr6:14550 | GGTCCCATGGTGTAATGGTTAGCACTCTGGG |
| | 3859-145503930 (+) | CTTTGAATCCAGCAATCCGAGTTCGAATCTT |
| | | G |
| 98 | Glu_CTC_chr1:14539 | TCCCTGGTGGTCTAGTGGTTAGGATTCGGCG |
| | 9233-145399304 (−) | CTCTCACCGCCGCGGCCCGGGTTCGATTCCC |
| 99 | Glu_CTC_chr1:24916 | TCCCTGGTGGTCTAGTGGTTAGGATTCGGCG |
| | 8447-249168518 (+) | CTCTCACCGCCGCGGCCCGGGTTCGATTCCC |
| 100 | Glu_TTC_chr2:13109 | TCCCATATGGTCTAGCGGTTAGGATTCCTGG |
| | 4701-131094772 (−) | TTTTCACCCAGGTGGCCCGGGTTCGACTCCC |
| | | G |
| 101 | Glu_TTC_chr13:4549 | TCCCACATGGTCTAGCGGTTAGGATTCCTGG |
| | 2062-45492133 (−) | TTTTCACCCAGGCGGCCCGGGTTCGACTCCC |
| | | G |
| 102 | Glu_TTC_chr1:17199 | TCCCTGGTGGTCTAGTGGCTAGGATTCGGCG |
| | 078-17199149 (+) | CTTTCACCGCCGCGGCCCGGGTTCGATTCCC |
| | | G |
| 103 | Glu_TTC_chr1:16861 | TCCCTGGTGGTCTAGTGGCTAGGATTCGGCG |
| | 774-16861845 (−) | CTTTCACCGCCGCGGCCCGGGTTCGATTCCC |
| | | G |
| 104 | Gly_CCC_chr1:16872 | GCATTGGTGGTTCAGTGGTAGAATTCTCGCC |
| | 434-16872504 (−) | TCCCACGCGGGAGACCCGGGTTCAATTCCCG |
| | | G |
| 105 | Gly_CCC_chr2:70476 | GCGCCGCTGGTGTAGTGGTATCATGCAAGAT |
| | 123-70476193 (−) | TCCCATTCTTGCGACCCGGGTTCGATTCCCG |
| | | G |
| 106 | Gly_CCC_chr17:1976 | GCATTGGTGGTTCAATGGTAGAATTCTCGCC |
| | 4175-19764245 (+) | TCCCACGCAGGAGACCCAGGTTCGATTCCTG |
| | | G |
| 107 | Gly_GCC_chr1:16141 | GCATGGGTGGTTCAGTGGTAGAATTCTCGCC |
| | 3094-161413164 (+) | TGCCACGCGGGAGGCCCGGGTTCGATTCCCG |
| 108 | Gly_GCC_chr1:16149 | GCATTGGTGGTTCAGTGGTAGAATTCTCGCC |
| | 3637-161493707 (−) | TGCCACGCGGGAGGCCCGGGTTCGATTCCCG |
| | | G |
| 109 | Gly_GCC_chr16:7081 | GCATTGGTGGTTCAGTGGTAGAATTCTCGCC |
| | 2114-70812184 (−) | TGCCACGCGGGAGGCCCGGGTTTGATTCCCG |
| | | G |
| 110 | Gly_GCC_chr1:16145 | GCATAGGTGGTTCAGTGGTAGAATTCTTGCC |
| | 0356-161450426 (+) | TGCCACGCAGGAGGCCCAGGTTTGATTCCTG |
| 111 | Gly_GCC_chr16:7082 | GCATTGGTGGTTCAGTGGTAGAATTCTCGCC |
| | 2597-70822667 (+) | TGCCATGCGGGCGGCCGGGCTTCGATTCCTG |
| | | G |
| 112 | Gly_TCC_chr19:4724 | GCGTTGGTGGTATAGTGGTTAGCATAGCTGC |
| | 082-4724153 (+) | CTTCCAAGCAGTTGACCCGGGTTCGATTCCC |
| 113 | Gly_TCC_chr1:14539 | GCGTTGGTGGTATAGTGGTGAGCATAGCTGC |
| | 7864-145397935 (−) | CTTCCAAGCAGTTGACCCGGGTTCGATTCCC |
| 114 | Gly_TCC_chr17:8124 | GCGTTGGTGGTATAGTGGTAAGCATAGCTGC |
| | 866-8124937 (+) | CTTCCAAGCAGTTGACCCGGGTTCGATTCCC |
| 115 | Gly_TCC_chr1:16140 | GCGTTGGTGGTATAGTGGTGAGCATAGTTGC |
| | 9961-161410032 (−) | CTTCCAAGCAGTTGACCCGGGCTCGATTCCC |
| 116 | His_GTG_chr1:14539 | GCCGTGATCGTATAGTGGTTAGTACTCTGCG |
| | 6881-145396952 (−) | TTGTGGCCGCAGCAACCTCGGTTCGAATCCG |
| | | A |
| 117 | His_GTG_chr1:14915 | GCCATGATCGTATAGTGGTTAGTACTCTGCG |
| | 5828-149155899 (−) | CTGTGGCCGCAGCAACCTCGGTTCGAATCCG |
| 118 | Ile_AAT_chr6:581492 | GGCCGGTTAGCTCAGTTGGTTAGAGCGTGGC |
| | 54-58149327 (+) | GCTAATAACGCCAAGGTCGCGGGTTCGATCC |
| 119 | Ile_AAT_chr6:276559 | GGCCGGTTAGCTCAGTTGGTTAGAGCGTGGT |
| | 67-27656040 (+) | GCTAATAACGCCAAGGTCGCGGGTTCGATCC |
| 120 | Ile_AAT_chr6:272429 | GGCTGGTTAGCTCAGTTGGTTAGAGCGTGGT |
| | 90-27243063 (−) | GCTAATAACGCCAAGGTCGCGGGTTCGATCC |
| 121 | Ile_AAT_chr17:81303 | GGCCGGTTAGCTCAGTTGGTTAGAGCGTGGT |
| | 09-8130382 (−) | GCTAATAACGCCAAGGTCGCGGGTTCGAACC |
| 122 | Ile_AAT_chr6:265543 | GGCCGGTTAGCTCAGTTGGTTAGAGCGTGGT |
| | 50-26554423 (+) | GCTAATAACGCCAAGGTCGCGGGTTCGATCC |
| 123 | Ile_AAT_chr6:267452 | GGCCGGTTAGCTCAGTTGGTTAGAGCGTGGT |
| | 55-26745328 (−) | GCTAATAACGCTAAGGTCGCGGGTTCGATCC |
| 124 | Ile_AAT_chr6:267212 | GGCCGGTTAGCTCAGTTGGTCAGAGCGTGGT |
| | 21-26721294 (−) | GCTAATAACGCCAAGGTCGCGGGTTCGATCC |
| 125 | Ile_AAT_chr6:276363 | GGCCGGTTAGCTCAGTCGGCTAGAGCGTGGT |
| | 62-27636435 (+) | GCTAATAACGCCAAGGTCGCGGGTTCGATCC |
| 126 | Ile_AAT_chr6:272417 | GGCTGGTTAGTTCAGTTGGTTAGAGCGTGGT |
| | 39-27241812 (+) | GCTAATAACGCCAAGGTCGTGGGTTCGATCC |
| 127 | Ile_GAT_chrX:37564 | GGCCGGTTAGCTCAGTTGGTAAGAGCGTGGT |
| | 18-3756491 (−) | GCTGATAACACCAAGGTCGCGGGCTCGACTC |
| 128 | Ile_TAT_chr19:39902 | GCTCCAGTGGCGCAATCGGTTAGCGCGCGGT |
| | 808-39902900 (−) | ACTTATATGACAGTGCGAGCGGAGCAATGCC |
| 129 | Ile_TAT_chr2:430376 | GCTCCAGTGGCGCAATCGGTTAGCGCGCGGT |
| | 76-43037768 (+) | ACTTATACAGCAGTACATGCAGAGCAATGCC |
| 130 | Ile_TAT_chr6:269881 | GCTCCAGTGGCGCAATCGGTTAGCGCGCGGT |
| | 25-26988218 (+) | ACTTATATGGCAGTATGTGTGCGAGTGATGC |
| 131 | Ile_TAT_chr6:275992 | GCTCCAGTGGCGCAATCGGTTAGCGCGCGGT |
| | 00-27599293 (+) | ACTTATACAACAGTATATGTGCGGGTGATGC |
| 132 | Ile_TAT_chr6:285053 | GCTCCAGTGGCGCAATCGGTTAGCGCGCGGT |
| | 67-28505460 (+) | ACTTATAAGACAGTGCACCTGTGAGCAATGC |
| 133 | Leu_AAG_chr5:1805 | GGTAGCGTGGCCGAGCGGTCTAAGGCGCTGG |
| | 24474-180524555 (−) | ATTAAGGCTCCAGTCTCTTCGGAGGCGTGGG |
| 134 | Leu_AAG_chr5:1806 | GGTAGCGTGGCCGAGCGGTCTAAGGCGCTGG |
| | 14701-180614782 (+) | ATTAAGGCTCCAGTCTCTTCGGGGGCGTGGG |
| 135 | Leu_AAG_chr6:2895 | GGTAGCGTGGCCGAGCGGTCTAAGGCGCTGG |
| | 6779-28956860 (+) | ATTAAGGCTCCAGTCTCTTCGGGGGCGTGGG |
| 136 | Leu_AAG_chr6:2844 | GGTAGCGTGGCCGAGTGGTCTAAGACGCTGG |
| | 6400-28446481 (−) | ATTAAGGCTCCAGTCTCTTCGGGGGCGTGGG |
| 137 | Leu_CAA_chr6:28864 | GTCAGGATGGCCGAGTGGTCTAAGGCGCCAG |
| | 000-28864105 (−) | ACTCAAGCTAAGCTTCCTCCGCGGTGGGGAT |
| 138 | Leu_CAA_chr6:28908 | GTCAGGATGGCCGAGTGGTCTAAGGCGCCAG |
| | 830-28908934 (+) | ACTCAAGCTTGGCTTCCTCGTGTTGAGGATT |
| | | C |
| 139 | Leu_CAA_chr6:27573 | GTCAGGATGGCCGAGTGGTCTAAGGCGCCAG |
| | 417-27573524 (−) | ACTCAAGCTTACTGCTTCCTGTGTTCGGGTC |
| | | T |
| 140 | Leu_CAA_chr6:27570 | GTCAGGATGGCCGAGTGGTCTAAGGCGCCAG |
| | 348-27570454 (−) | ACTCAAGTTGCTACTTCCCAGGTTTGGGGCT |
| | | T |
| 141 | Leu_CAA_chr1:24916 | GTCAGGATGGCCGAGTGGTCTAAGGCGCCAG |
| | 8054-249168159 (+) | ACTCAAGGTAAGCACCTTGCCTGCGGGCTTT |
| 142 | Leu_CAA_chr11:9296 | GCCTCCTTAGTGCAGTAGGTAGCGCATCAGT |
| | 790-9296863 (+) | CTCAAAATCTGAATGGTCCTGAGTTCAAGCC |
| 143 | Leu_CAA_chr1:16158 | GTCAGGATGGCCGAGCAGTCTTAAGGCGCTG |
| | 1736-161581819 (−) | CGTTCAAATCGCACCCTCCGCTGGAGGCGTG |
| 144 | Leu_CAG_chr1:16141 | GTCAGGATGGCCGAGCGGTCTAAGGCGCTGC |
| | 1323-161411405 (+) | GTTCAGGTCGCAGTCTCCCCTGGAGGCGTGG |
| 145 | Leu_CAG_chr16:5733 | GTCAGGATGGCCGAGCGGTCTAAGGCGCTGC |
| | 3863-57333945 (+) | GTTCAGGTCGCAGTCTCCCCTGGAGGCGTGG |
| 146 | Leu_TAA_chr6:14453 | ACCAGGATGGCCGAGTGGTTAAGGCGTTGGA |
| | 7684-144537766 (+) | CTTAAGATCCAATGGACATATGTCCGCGTGG |
| 147 | Leu_TAA_chr6:27688 | ACCGGGATGGCCGAGTGGTTAAGGCGTTGGA |
| | 898-27688980 (−) | CTTAAGATCCAATGGGCTGGTGCCCGCGTGG |
| 148 | Leu_TAA_chr11:5931 | ACCAGAATGGCCGAGTGGTTAAGGCGTTGGA |
| | 9228-59319310 (+) | CTTAAGATCCAATGGATTCATATCCGCGTGG |
| 149 | Leu_TAA_chr6:27198 | ACCGGGATGGCTGAGTGGTTAAGGCGTTGGA |
| | 334-27198416 (−) | CTTAAGATCCAATGGACAGGTGTCCGCGTGG |
| 150 | Leu_TAG_chr17:8023 | GGTAGCGTGGCCGAGCGGTCTAAGGCGCTGG |
| | 632-8023713 (−) | ATTTAGGCTCCAGTCTCTTCGGAGGCGTGGG |
| 151 | Leu_TAG_chr14:2109 | GGTAGTGTGGCCGAGCGGTCTAAGGCGCTGG |
| | 3529-21093610 (+) | ATTTAGGCTCCAGTCTCTTCGGGGGCGTGGG |
| 152 | Leu_TAG_chr16:2220 | GGTAGCGTGGCCGAGTGGTCTAAGGCGCTGG |
| | 7032-22207113 (−) | ATTTAGGCTCCAGTCATTTCGATGGCGTGGG |
| | | T |
| 153 | Lys_CTT_chr14:5870 | GCCCGGCTAGCTCAGTCGGTAGAGCATGGGA |
| | 6613-58706685 (−) | CTCTTAATCCCAGGGTCGTGGGTTCGAGCCC |
| 154 | Lys_CTT_chr19:3606 | GCCCAGCTAGCTCAGTCGGTAGAGCATAAGA |
| | 6750-36066822 (+) | CTCTTAATCTCAGGGTTGTGGATTCGTGCCC |
| | | C |
| 155 | Lys_CTT_chr19:5242 | GCAGCTAGCTCAGTCGGTAGAGCATGAGACT |
| | 5393-52425466 (−) | CTTAATCTCAGGGTCATGGGTTCGTGCCCCA |
| | | T |
| 156 | Lys_CTT_chr1:14539 | GCCCGGCTAGCTCAGTCGGTAGAGCATGAGA |
| | 5522-145395594 (−) | CTCTTAATCTCAGGGTCGTGGGTTCGAGCCC |
| | | C |
| 157 | Lys_CTT_chr16:3207 | GCCCGGCTAGCTCAGTCGGTAGAGCATGAGA |
| | 406-3207478 (−) | CCCTTAATCTCAGGGTCGTGGGTTCGAGCCC |
| 158 | Lys_CTT_chr16:3241 | GCCCGGCTAGCTCAGTCGGTAGAGCATGGGA |
| | 501-3241573 (+) | CTCTTAATCTCAGGGTCGTGGGTTCGAGCCC |
| | | C |
| 159 | Lys_CTT_chr16:3230 | GCCCGGCTAGCTCAGTCGATAGAGCATGAGA |
| | 555-3230627 (−) | CTCTTAATCTCAGGGTCGTGGGTTCGAGCCG |
| 160 | Lys_CTT_chr1:55423 | GCCCAGCTAGCTCAGTCGGTAGAGCATGAGA |
| | 542-55423614 (−) | CTCTTAATCTCAGGGTCATGGGTTTGAGCCC |
| | | C |
| 161 | Lys_CTT_chr16:3214 | GCCTGGCTAGCTCAGTCGGCAAAGCATGAGA |
| | 939-3215011 (+) | CTCTTAATCTCAGGGTCGTGGGCTCGAGCTC |
| | | C |
| 162 | Lys_CTT_chr5:26198 | GCCCGACTACCTCAGTCGGTGGAGCATGGGA |
| | 539-26198611 (−) | CTCTTCATCCCAGGGTTGTGGGTTCGAGCCC |
| | | C |
| 163 | Lys_TTT_chr16:7351 | GCCTGGATAGCTCAGTTGGTAGAGCATCAGA |
| | 2216-73512288 (−) | CTTTTAATCTGAGGGTCCAGGGTTCAAGTCC |
| | | C |
| 164 | Lys_TTT_chr12:2784 | ACCCAGATAGCTCAGTCAGTAGAGCATCAGA |
| | 3306-27843378 (+) | CTTTTAATCTGAGGGTCCAAGGTTCATGTCC |
| | | C |
| 165 | Lys_TTT_chr11:1224 | GCCTGGATAGCTCAGTTGGTAGAGCATCAGA |
| | 30655-122430727 (+) | CTTTTAATCTGAGGGTCCAGGGTTCAAGTCC |
| | | C |
| 166 | Lys_TTT_chr1:20447 | GCCCGGATAGCTCAGTCGGTAGAGCATCAGA |
| | 5655-204475727 (+) | CTTTTAATCTGAGGGTCCAGGGTTCAAGTCC |
| | | C |
| 167 | Lys_TTT_chr6:27559 | GCCTGGATAGCTCAGTCGGTAGAGCATCAGA |
| | 593-27559665 (−) | CTTTTAATCTGAGGGTCCAGGGTTCAAGTCC |
| | | C |
| 168 | Lys_TTT_chr11:5932 | GCCCGGATAGCTCAGTCGGTAGAGCATCAGA |
| | 3902-59323974 (+) | CTTTTAATCTGAGGGTCCGGGGTTCAAGTCC |
| | | C |
| 169 | Lys_TTT_chr6:27302 | GCCTGGGTAGCTCAGTCGGTAGAGCATCAGA |
| | 769-27302841 (−) | CTTTTAATCTGAGGGTCCAGGGTTCAAGTCC |
| | | C |
| 170 | Lys_TTT_chr6:28715 | GCCTGGATAGCTCAGTTGGTAGAACATCAGA |
| | 521-28715593 (+) | CTTTTAATCTGACGGTGCAGGGTTCAAGTCC |
| | | C |
| 171 | Met_CAT_chr8:12416 | GCCTCGTTAGCGCAGTAGGTAGCGCGTCAGT |
| | 9470-124169542 (−) | CTCATAATCTGAAGGTCGTGAGTTCGATCCT |
| | | C |
| 172 | Met_CAT_chr16:7146 | GCCCTCTTAGCGCAGTGGGCAGCGCGTCAGT |
| | 0396-71460468 (+) | CTCATAATCTGAAGGTCCTGAGTTCGAGCCT |
| 173 | Met_CAT_chr6:28912 | GCCTCCTTAGCGCAGTAGGCAGCGCGTCAGT |
| | 352-28912424 (+) | CTCATAATCTGAAGGTCCTGAGTTCGAACCT |
| 174 | Met_CAT_chr6:26735 | GCCCTCTTAGCGCAGCGGGCAGCGCGTCAGT |
| | 574-26735646 (−) | CTCATAATCTGAAGGTCCTGAGTTCGAGCCT |
| 175 | Met_CAT_chr6:26701 | GCCCTCTTAGCGCAGCTGGCAGCGCGTCAGT |
| | 712-26701784 (+) | CTCATAATCTGAAGGTCCTGAGTTCAAGCCT |
| 176 | Met_CAT_chr16:8741 | GCCTCGTTAGCGCAGTAGGCAGCGCGTCAGT |
| | 7628-87417700 (−) | CTCATAATCTGAAGGTCGTGAGTTCGAGCCT |
| 177 | Met_CAT_chr6:58168 | GCCCTCTTAGTGCAGCTGGCAGCGCGTCAGT |
| | 492-58168564 (−) | TTCATAATCTGAAAGTCCTGAGTTCAAGCCT |
| | | C |
| 178 | Phe_GAA_chr6:28758 | GCCGAAATAGCTCAGTTGGGAGAGCGTTAGA |
| | 499-28758571 (−) | CTGAAGATCTAAAGGTCCCTGGTTCGATCCC |
| 179 | Phe_GAA_chr11:5933 | GCCGAAATAGCTCAGTTGGGAGAGCGTTAGA |
| | 3853-59333925 (−) | CTGAAGATCTAAAGGTCCCTGGTTCAATCCC |
| 180 | Phe_GAA_chr6:28775 | GCCGAGATAGCTCAGTTGGGAGAGCGTTAGA |
| | 610-28775682 (−) | CTGAAGATCTAAAGGTCCCTGGTTCAATCCC |
| 181 | Phe_GAA_chr6:28791 | GCCGAAATAGCTCAGTTGGGAGAGCGTTAGA |
| | 093-28791166 (−) | CCGAAGATCTTAAAGGTCCCTGGTTCAATCC |
| 182 | Phe_GAA_chr6:28731 | GCTGAAATAGCTCAGTTGGGAGAGCGTTAGA |
| | 374-28731447 (−) | CTGAAGATCTTAAAGTTCCCTGGTTCAACCC |
| | | T |
| 183 | Pro_AGG_chr16:3241 | GGCTCGTTGGTCTAGGGGTATGATTCTCGCT |
| | 989-3242060 (+) | TAGGATGCGAGAGGTCCCGGGTTCAAATCCC |
| | | G |
| 184 | Pro_AGG_chr1:16768 | GGCTCGTTGGTCTAGGGGTATGATTCTCGCT |
| | 4725-167684796 (−) | TAGGGTGCGAGAGGTCCCGGGTTCAAATCCC |
| | | G |
| 185 | Pro_CGG_chr1:16768 | GGCTCGTTGGTCTAGGGGTATGATTCTCGCT |
| | 3962-167684033 (+) | TCGGGTGCGAGAGGTCCCGGGTTCAAATCCC |
| | | G |
| 186 | Pro_CGG_chr6:27059 | GGCTCGTTGGTCTAGGGGTATGATTCTCGCT |
| | 521-27059592 (+) | TCGGGTGTGAGAGGTCCCGGGTTCAAATCCC |
| | | G |
| 187 | Pro_TGG_chr14:2110 | GGCTCGTTGGTCTAGTGGTATGATTCTCGCT |
| | 1165-21101236 (+) | TTGGGTGCGAGAGGTCCCGGGTTCAAATCCC |
| | | G |
| 188 | Pro_TGG_chr11:7594 | GGCTCGTTGGTCTAGGGGTATGATTCTCGGT |
| | 6869-75946940 (−) | TTGGGTCCGAGAGGTCCCGGGTTCAAATCCC |
| | | G |
| 189 | Pro_TGG_chr5:18061 | GGCTCGTTGGTCTAGGGGTATGATTCTCGCT |
| | 5854-180615925 (−) | TTGGGTGCGAGAGGTCCCGGGTTCAAATCCC |
| | | G |
| 190 | SeC_TCA_chr19:4598 | GCCCGGATGATCCTCAGTGGTCTGGGGTGCA |
| | 1859-45981945 (−) | GGCTTCAAACCTGTAGCTGTCTAGCGACAGA |
| 191 | SeC_TCA_chr22:4454 | GCTCGGATGATCCTCAGTGGTCTGGGGTGCA |
| | 6537-44546620 (+) | GGCTTCAAACCTGTAGCTGTCTAGTGACAGA |
| 192 | Ser_AGA_chr6:27509 | GTAGTCGTGGCCGAGTGGTTAAGGCGATGGA |
| | 554-27509635 (−) | CTAGAAATCCATTGGGGTTTCCCCGCGCAGG |
| 193 | Ser_AGA_chr6:26327 | GTAGTCGTGGCCGAGTGGTTAAGGCGATGGA |
| | 817-26327898 (+) | CTAGAAATCCATTGGGGTCTCCCCGCGCAGG |
| 194 | Ser_AGA_chr6:27499 | GTAGTCGTGGCCGAGTGGTTAAGGCGATGGA |
| | 987-27500068 (+) | CTAGAAATCCATTGGGGTTTCCCCACGCAGG |
| 195 | Ser_AGA_chr6:27521 | GTAGTCGTGGCCGAGTGGTTAAGGTGATGGA |
| | 192-27521273 (−) | CTAGAAACCCATTGGGGTCTCCCCGCGCAGG |
| 196 | Ser_CGA_chr17:8042 | GCTGTGATGGCCGAGTGGTTAAGGCGTTGGA |
| | 199-8042280 (−) | CTCGAAATCCAATGGGGTCTCCCCGCGCAGG |
| 197 | Ser_CGA_chr6:27177 | GCTGTGATGGCCGAGTGGTTAAGGCGTTGGA |
| | 628-27177709 (+) | CTCGAAATCCAATGGGGTCTCCCCGCGCAGG |
| 198 | Ser_CGA_chr6:27640 | GCTGTGATGGCCGAGTGGTTAAGGTGTTGGA |
| | 229-27640310 (−) | CTCGAAATCCAATGGGGGTTCCCCGCGCAGG |
| 199 | Ser_CGA_chr12:5658 | GTCACGGTGGCCGAGTGGTTAAGGCGTTGGA |
| | 4148-56584229 (+) | CTCGAAATCCAATGGGGTTTCCCCGCACAGG |
| 200 | Ser_GCT_chr6:27065 | GACGAGGTGGCCGAGTGGTTAAGGCGATGGA |
| | 085-27065166 (+) | CTGCTAATCCATTGTGCTCTGCACGCGTGG |
| 201 | Ser_GCT_chr6:27265 | GACGAGGTGGCCGAGTGGTTAAGGCGATGGA |
| | 775-27265856 (+) | CTGCTAATCCATTGTGCTCTGCACGCGTGG |
| 202 | Ser_GCT_chr11:6611 | GACGAGGTGGCCGAGTGGTTAAGGCGATGGA |
| | 5591-66115672 (+) | CTGCTAATCCATTGTGCTTTGCACGCGTGGG |
| 203 | Ser_GCT_chr6:28565 | GACGAGGTGGCCGAGTGGTTAAGGCGATGGA |
| | 117-28565198 (−) | CTGCTAATCCATTGTGCTCTGCACGCGTGG |
| 204 | Ser_GCT_chr6:28180 | GACGAGGTGGCCGAGTGGTTAAGGCGATGGA |
| | 815-28180896 (+) | CTGCTAATCCATTGTGCTCTGCACACGTGG |
| 205 | Ser_GCT_chr6:26305 | GGAGAGGCCTGGCCGAGTGGTTAAGGCGATG |
| | 718-26305801 (−) | GACTGCTAATCCATTGTGCTCTGCACGCGTG |
| 206 | Ser_TGA_chr10:6952 | GCAGCGATGGCCGAGTGGTTAAGGCGTTGGA |
| | 4261-69524342 (+) | CTTGAAATCCAATGGGGTCTCCCCGCGCAGG |
| 207 | Ser_TGA_chr6:27513 | GTAGTCGTGGCCGAGTGGTTAAGGCGATGGA |
| | 468-27513549 (+) | CTTGAAATCCATTGGGGTTTCCCCGCGCAGG |
| 208 | Ser_TGA_chr6:26312 | GTAGTCGTGGCCGAGTGGTTAAGGCGATGGA |
| | 824-26312905 (−) | CTTGAAATCCATTGGGGTCTCCCCGCGCAGG |
| 209 | Ser_TGA_chr6:27473 | GTAGTCGTGGCCGAGTGGTTAAGGCGATGGA |
| | 607-27473688 (−) | CTTGAAATCCATTGGGGTTTCCCCGCGCAGG |
| 210 | Thr_AGT_chr17:8090 | GGCGCCGTGGCTTAGTTGGTTAAAGCGCCTG |
| | 478-8090551 (+) | TCTAGTAAACAGGAGATCCTGGGTTCGAATC |
| 211 | Thr_AGT_chr6:26533 | GGCTCCGTGGCTTAGCTGGTTAAAGCGCCTG |
| | 145-26533218 (−) | TCTAGTAAACAGGAGATCCTGGGTTCGAATC |
| 212 | Thr_AGT_chr6:28693 | GGCTCCGTAGCTTAGTTGGTTAAAGCGCCTG |
| | 795-28693868 (+) | TCTAGTAAACAGGAGATCCTGGGTTCGACTC |
| 213 | Thr_AGT_chr6:27694 | GGCTTCGTGGCTTAGCTGGTTAAAGCGCCTG |
| | 473-27694546 (+) | TCTAGTAAACAGGAGATCCTGGGTTCGAATC |
| 214 | Thr_AGT_chr17:8042 | GGCGCCGTGGCTTAGCTGGTTAAAGCGCCTG |
| | 770-8042843 (−) | TCTAGTAAACAGGAGATCCTGGGTTCGAATC |
| 215 | Thr_AGT_chr6:27130 | GGCCCTGTGGCTTAGCTGGTCAAAGCGCCTG |
| | 050-27130123 (+) | TCTAGTAAACAGGAGATCCTGGGTTCGAATC |
| 216 | Thr_CGT_chr6:28456 | GGCTCTATGGCTTAGTTGGTTAAAGCGCCTG |
| | 770-28456843 (−) | TCTCGTAAACAGGAGATCCTGGGTTCGACTC |
| | | C |
| 217 | Thr_CGT_chr16:1437 | GGCGCGGTGGCCAAGTGGTAAGGCGTCGGTC |
| | 9750-14379821 (+) | TCGTAAACCGAAGATCACGGGTTCGAACCCC |
| 218 | Thr_CGT_chr6:28615 | GGCTCTGTGGCTTAGTTGGCTAAAGCGCCTG |
| | 984-28616057 (−) | TCTCGTAAACAGGAGATCCTGGGTTCGAATC |
| 219 | Thr_CGT_chr17:2987 | GGCGCGGTGGCCAAGTGGTAAGGCGTCGGTC |
| | 7093-29877164 (+) | TCGTAAACCGAAGATCGCGGGTTCGAACCCC |
| 220 | Thr_CGT_chr6:27586 | GGCCCTGTAGCTCAGCGGTTGGAGCGCTGGT |
| | 135-27586208 (+) | CTCGTAAACCTAGGGGTCGTGAGTTCAAATC |
| 221 | Thr_TGT_chr6:28442 | GGCTCTATGGCTTAGTTGGTTAAAGCGCCTG |
| | 329-28442402 (−) | TCTTGTAAACAGGAGATCCTGGGTTCGAATC |
| | | C |
| 222 | Thr_TGT_chr1:22263 | GGCTCCATAGCTCAGTGGTTAGAGCACTGGT |
| | 8347-222638419 (+) | CTTGTAAACCAGGGGTCGCGAGTTCGATCCT |
| 223 | Thr_TGT_chr14:2108 | GGCTCCATAGCTCAGGGGTTAGAGCGCTGGT |
| | 1949-21082021 (−) | CTTGTAAACCAGGGGTCGCGAGTTCAATTCT |
| 224 | Thr_TGT_chr14:2109 | GGCTCCATAGCTCAGGGGTTAGAGCACTGGT |
| | 9319-21099391 (−) | CTTGTAAACCAGGGGTCGCGAGTTCAAATCT |
| 225 | Thr_TGT_chr14:2114 | GGCCCTATAGCTCAGGGGTTAGAGCACTGGT |
| | 9849-21149921 (+) | CTTGTAAACCAGGGGTCGCGAGTTCAAATCT |
| 226 | Thr_TGT_chr5:18061 | GGCTCCATAGCTCAGGGGTTAGAGCACTGGT |
| | 8687-180618758 (−) | CTTGTAAACCAGGGTCGCGAGTTCAAATCTC |
| 227 | Trp_CCA_chr17:8124 | GGCCTCGTGGCGCAACGGTAGCGCGTCTGAC |
| | 187-8124258 (−) | TCCAGATCAGAAGGTTGCGTGTTCAAATCAC |
| 228 | Trp_CCA_chr17:1941 | GACCTCGTGGCGCAATGGTAGCGCGTCTGAC |
| | 1494-19411565 (+) | TCCAGATCAGAAGGTTGCGTGTTCAAGTCAC |
| 229 | Trp_CCA_chr6:26319 | GACCTCGTGGCGCAACGGTAGCGCGTCTGAC |
| | 330-26319401 (−) | TCCAGATCAGAAGGTTGCGTGTTCAAATCAC |
| 230 | Trp_CCA_chr12:9889 | GACCTCGTGGCGCAACGGTAGCGCGTCTGAC |
| | 8030-98898101 (+) | TCCAGATCAGAAGGCTGCGTGTTCGAATCAC |
| 231 | Trp_CCA_chr7:99067 | GACCTCGTGGCGCAACGGCAGCGCGTCTGAC |
| | 307-99067378 (+) | TCCAGATCAGAAGGTTGCGTGTTCAAATCAC |
| 232 | Tyr_ATA_chr2:21911 | CCTTCAATAGTTCAGCTGGTAGAGCAGAGGA |
| | 0549-219110641 (+) | CTATAGCTACTTCCTCAGTAGGAGACGTCCT |
| | | T |
| 233 | Tyr_GTA_chr6:26569 | CCTTCGATAGCTCAGTTGGTAGAGCGGAGGA |
| | 086-26569176 (+) | CTGTAGTTGGCTGTGTCCTTAGACATCCTTA |
| | | G |
| 234 | Tyr_GTA_chr2:27273 | CCTTCGATAGCTCAGTTGGTAGAGCGGAGGA |
| | 650-27273738 (+) | CTGTAGTGGATAGGGCGTGGCAATCCTTAGG |
| 235 | Tyr_GTA_chr6:26577 | CCTTCGATAGCTCAGTTGGTAGAGCGGAGGA |
| | 332-26577420 (+) | CTGTAGGCTCATTAAGCAAGGTATCCTTAGG |
| 236 | Tyr_GTA_chr14:2112 | CCTTCGATAGCTCAGCTGGTAGAGCGGAGGA |
| | 5623-21125716 (−) | CTGTAGATTGTATAGACATTTGCGGACATCC |
| | | T |
| 237 | Tyr_GTA_chr8:67025 | CCTTCGATAGCTCAGCTGGTAGAGCGGAGGA |
| | 602-67025694 (+) | CTGTAGCTACTTCCTCAGCAGGAGACATCCT |
| | | T |
| 238 | Tyr_GTA_chr8:67026 | CCTTCGATAGCTCAGCTGGTAGAGCGGAGGA |
| | 223-67026311 (+) | CTGTAGGCGCGCGCCCGTGGCCATCCTTAGG |
| 239 | Tyr_GTA_chr14:2112 | CCTTCGATAGCTCAGCTGGTAGAGCGGAGGA |
| | 1258-21121351 (−) | CTGTAGCCTGTAGAAACATTTGTGGACATCC |
| 240 | Tyr_GTA_chr14:2113 | CCTTCGATAGCTCAGCTGGTAGAGCGGAGGA |
| | 1351-21131444 (−) | CTGTAGATTGTACAGACATTTGCGGACATCC |
| 241 | Tyr_GTA_chr14:2115 | CCTTCGATAGCTCAGCTGGTAGAGCGGAGGA |
| | 1432-21151520 (+) | CTGTAGTACTTAATGTGTGGTCATCCTTAGG |
| | | T |
| 242 | Tyr_GTA_chr6:26595 | CCTTCGATAGCTCAGCTGGTAGAGCGGAGGA |
| | 102-26595190 (+) | CTGTAGGGGTTTGAATGTGGTCATCCTTAGG |
| | | T |
| 243 | Tyr_GTA_chr14:2112 | CCTTCGATAGCTCAGCTGGTAGAGCGGAGGA |
| | 8117-21128210 (−) | CTGTAGACTGCGGAAACGTTTGTGGACATCC |
| 244 | Tyr_GTA_chr6:26575 | CTTTCGATAGCTCAGTTGGTAGAGCGGAGGA |
| | 798-26575887 (+) | CTGTAGGTTCATTAAACTAAGGCATCCTTAG |
| 245 | Tyr_GTA_chr8:66609 | TCTTCAATAGCTCAGCTGGTAGAGCGGAGGA |
| | 532-66609619 (−) | CTGTAGGTGCACGCCCGTGGCCATTCTTAGG |
| 246 | Val_AAC_chr3:16949 | GTTTCCGTAGTGTAGTGGTTATCACGTTCGC |
| | 0018-169490090 (+) | CTAACACGCGAAAGGTCCCCGGTTCGAAACC |
| | | G |
| 247 | Val_AAC_chr5:18061 | GTTTCCGTAGTGTAGTGGTCATCACGTTCGC |
| | 5416-180615488 (−) | CTAACACGCGAAAGGTCCCCGGTTCGAAACC |
| | | G |
| 248 | Val_AAC_chr6:27618 | GTTTCCGTAGTGTAGTGGTTATCACGTTCGC |
| | 707-27618779 (−) | CTAACACGCGAAAGGTCCCTGGATCAAAACC |
| | | A |
| 249 | Val_AAC_chr6:27648 | GTTTCCGTAGTGTAGTGGTTATCACGTTCGC |
| | 885-27648957 (−) | CTAACACGCGAAAGGTCCGCGGTTCGAAACC |
| | | G |
| 250 | Val_AAC_chr6:27203 | GTTTCCGTAGTGTAGTGGTTATCACGTTTGC |
| | 288-27203360 (+) | CTAACACGCGAAAGGTCCCCGGTTCGAAACC |
| | | G |
| 251 | Val_AAC_chr6:28703 | GGGGGTGTAGCTCAGTGGTAGAGCGTATGCT |
| | 206-28703277 (−) | TAACATTCATGAGGCTCTGGGTTCGATCCCC |
| 252 | Val_CAC_chr1:16136 | GTTTCCGTAGTGTAGTGGTTATCACGTTCGC |
| | 9490-161369562 (−) | CTCACACGCGAAAGGTCCCCGGTTCGAAACC |
| | | G |
| 253 | Val_CAC_chr6:27248 | GCTTCTGTAGTGTAGTGGTTATCACGTTCGC |
| | 049-27248121 (−) | CTCACACGCGAAAGGTCCCCGGTTCGAAACC |
| | | G |
| 254 | Val_CAC_chr19:4724 | GTTTCCGTAGTGTAGCGGTTATCACATTCGC |
| | 647-4724719 (−) | CTCACACGCGAAAGGTCCCCGGTTCGATCCC |
| | | G |
| 255 | Val_CAC_chr1:14929 | GTTTCCGTAGTGTAGTGGTTATCACGTTCGC |
| | 8555-149298627 (−) | CTCACACGCGAAAGGTCCCCGGTTCGAAACT |
| | | G |
| 256 | Val_CAC_chr1:14968 | GTTTCCGTAGTGTAGTGGTTATCACGTTCGC |
| | 4088-149684161 (−) | CTCACACGCGTAAAGGTCCCCGGTTCGAAAC |
| | | C |
| 257 | Val_CAC_chr6:27173 | GTTTCCGTAGTGGAGTGGTTATCACGTTCGC |
| | 867-27173939 (−) | CTCACACGCGAAAGGTCCCCGGTTTGAAACC |
| | | A |
| 258 | Val_TAC_chr11:5931 | GGTTCCATAGTGTAGTGGTTATCACGTCTGC |
| | 8102-59318174 (−) | TTTACACGCAGAAGGTCCTGGGTTCGAGCCC |
| | | C |
| 259 | Val_TAC_chr11:5931 | GGTTCCATAGTGTAGCGGTTATCACGTCTGC |
| | 8460-59318532 (−) | TTTACACGCAGAAGGTCCTGGGTTCGAGCCC |
| | | C |
| 260 | Val_TAC_chr10:5895 | GGTTCCATAGTGTAGTGGTTATCACATCTGC |
| | 674-5895746 (−) | TTTACACGCAGAAGGTCCTGGGTTCAAGCCC |
| | | C |
| 261 | Val_TAC_chr6:27258 | GTTTCCGTGGTGTAGTGGTTATCACATTCGC |
| | 405-27258477 (+) | CTTACACGCGAAAGGTCCTCGGGTCGAAACC |
| | | G |
| 262 | iMet_CAT_chr1:1536 | AGCAGAGTGGCGCAGCGGAAGCGTGCTGGGC |
| | 43726-153643797 (+) | CCATAACCCAGAGGTCGATGGATCGAAACC |
| 263 | iMet_CAT_chr6:2774 | AGCAGAGTGGCGCAGCGGAAGCGTGCTGGGC |
| | 5664-27745735 (+) | CCATAACCCAGAGGTCGATGGATCTAAACC |
| 264 | Glu_TTC_chr1:16861 | TCCCTGGTGGTCTAGTGGCTAGGATTCGGCG |
| | 773-16861845 (−) | CTTTCACCGCCGCGGCCCGGGTTCGATTCCC |
| | | G |
| 265 | Gly_CCC_chr1:17004 | GCGTTGGTGGTTTAGTGGTAGAATTCTCGCC |
| | 765-17004836 (−) | TCCCATGCGGGAGACCCGGGTTCAATTCCCG |
| | | G |
| 266 | Gly_CCC_chr1:17053 | GGCCTTGGTGGTGCAGTGGTAGAATTCTCGC |
| | 779-17053850 (+) | CTCCCACGTGGGAGACCCGGGTTCAATTCCC |
| 267 | Glu_TTC_chr1:17199 | GTCCCTGGTGGTCTAGTGGCTAGGATTCGGC |
| | 077-17199149 (+) | GCTTTCACCGCCGCGGCCCGGGTTCGATTCC |
| | | C |
| 268 | Asn_GTT_chr1:17216 | TGTCTCTGTGGCGCAATCGGTTAGCGCGTTC |
| | 171-17216245 (+) | GGCTGTTAACCGAAAGATTGGTGGTTCGAGC |
| | | C |
| 269 | Arg_TCT_chr1:94313 | TGGCTCCGTGGCGCAATGGATAGCGCATTGG |
| | 128-94313213 (+) | ACTTCTAGAGGCTGAAGGCATTCAAAGGTTC |
| 270 | Lys_CTT_chr1:14539 | GCCCGGCTAGCTCAGTCGGTAGAGCATGAGA |
| | 5521-145395594 (−) | CTCTTAATCTCAGGGTCGTGGGTTCGAGCCC |
| | | C |
| 271 | His_GTG_chr1:14539 | GCCGTGATCGTATAGTGGTTAGTACTCTGCG |
| | 6880-145396952 (−) | TTGTGGCCGCAGCAACCTCGGTTCGAATCCG |
| | | A |
| 272 | Gly_TCC_chr1:14539 | GCGTTGGTGGTATAGTGGTGAGCATAGCTGC |
| | 7863-145397935 (−) | CTTCCAAGCAGTTGACCCGGGTTCGATTCCC |
| 273 | Glu_CTC_chr1:14539 | TCCCTGGTGGTCTAGTGGTTAGGATTCGGCG |
| | 9232-145399304 (−) | CTCTCACCGCCGCGGCCCGGGTTCGATTCCC |
| 274 | Gln_CTG_chr1:14596 | AGGTTCCATGGTGTAATGGTGAGCACTCTGG |
| | 3303-145963375 (+) | ACTCTGAATCCAGCGATCCGAGTTCGAGTCT |
| 275 | Asn_GTT_chr1:14800 | TGTCTCTGTGGCGTAGTCGGTTAGCGCGTTC |
| | 0804-148000878 (+) | GGCTGTTAACCGAAAAGTTGGTGGTTCGAGC |
| | | C |
| 276 | Asn_GTT_chr1:14824 | TGTCTCTGTGGCGCAATCGGTTAGCGCGTTC |
| | 8114-148248188 (+) | GGCTGTTAACCGAAAGGTTGGTGGTTCGAGC |
| | | C |
| 277 | Asn_GTT_chr1:14859 | GTCTCTGTGGCGCAATCGGTTAGCGCATTCG |
| | 8313-148598387 (−) | GCTGTTAACCGAAAGGTTGGTGGTTCGAGCC |
| 278 | Asn_GTT_chr1:14923 | GTCTCTGTGGCGCAATGGGTTAGCGCGTTCG |
| | 0569-149230643 (−) | GCTGTTAACCGAAAGGTTGGTGGTTCGAGCC |
| 279 | Val_CAC_chr1:14929 | GCACTGGTGGTTCAGTGGTAGAATTCTCGCC |
| | 4665-149294736 (−) | TCACACGCGGGACACCCGGGTTCAATTCCCG |
| 280 | Val_CAC_chr1:14929 | GTTTCCGTAGTGTAGTGGTTATCACGTTCGC |
| | 8554-149298627 (−) | CTCACACGCGAAAGGTCCCCGGTTCGAAACT |
| | | G |
| 281 | Gly_CCC_chr1:14968 | GCACTGGTGGTTCAGTGGTAGAATTCTCGCC |
| | 0209-149680280 (−) | TCCCACGCGGGAGACCCGGGTTTAATTCCCG |
| 282 | Val_CAC_chr1:14968 | GTTTCCGTAGTGTAGTGGTTATCACGTTCGC |
| | 4087-149684161 (−) | CTCACACGCGTAAAGGTCCCCGGTTCGAAAC |
| | | C |
| 283 | Met_CAT_chr1:15364 | TAGCAGAGTGGCGCAGCGGAAGCGTGCTGGG |
| | 3725-153643797 (+) | CCCATAACCCAGAGGTCGATGGATCGAAAC |
| 284 | Val_CAC_chr1:16136 | GTTTCCGTAGTGTAGTGGTTATCACGTTCGC |
| | 9489-161369562 (−) | CTCACACGCGAAAGGTCCCCGGTTCGAAACC |
| | | G |
| 285 | Asp_GTC_chr1:16141 | TCCTCGTTAGTATAGTGGTGAGTATCCCCGC |
| | 0614-161410686 (−) | CTGTCACGCGGGAGACCGGGGTTCGATTCCC |
| | | C |
| 286 | Gly_GCC_chr1:16141 | TGCATGGGTGGTTCAGTGGTAGAATTCTCGC |
| | 3093-161413164 (+) | CTGCCACGCGGGAGGCCCGGGTTCGATTCCC |
| 287 | Glu_CTC_chr1:16141 | TCCCTGGTGGTCTAGTGGTTAGGATTCGGCG |
| | 7017-161417089 (−) | CTCTCACCGCCGCGGCCCGGGTTCGATTCCC |
| 288 | Asp_GTC_chr1:16149 | ATCCTTGTTACTATAGTGGTGAGTATCTCTG |
| | 2934-161493006 (+) | CCTGTCATGCGTGAGAGAGGGGGTCGATTCC |
| | | C |
| 289 | Gly_GCC_chr1:16149 | GCATTGGTGGTTCAGTGGTAGAATTCTCGCC |
| | 3636-161493707 (−) | TGCCACGCGGGAGGCCCGGGTTCGATTCCCG |
| | | G |
| 290 | Leu_CAG_chr1:16150 | GTCAGGATGGCCGAGCGGTCTAAGGCGCTGC |
| | 0131-161500214 (−) | GTTCAGGTCGCAGTCTCCCCTGGAGGCGTGG |
| 291 | Gly_TCC_chr1:16150 | CGCGTTGGTGGTATAGTGGTGAGCATAGCTG |
| | 0902-161500974 (+) | CCTTCCAAGCAGTTGACCCGGGTTCGATTCC |
| | | C |
| 292 | Asn_GTT_chr1:16151 | CGTCTCTGTGGCGCAATCGGTTAGCGCGTTC |
| | 0030-161510104 (+) | GGCTGTTAACCGAAAGGTTGGTGGTTCGATC |
| 293 | Glu_TTC_chr1:16158 | CGCGTTGGTGGTGTAGTGGTGAGCACAGCTG |
| | 2507-161582579 (+) | CCTTTCAAGCAGTTAACGCGGGTTCGATTCC |
| | | C |
| 294 | Pro_CGG_chr1:16768 | CGGCTCGTTGGTCTAGGGGTATGATTCTCGC |
| | 3961-167684033 (+) | TTCGGGTGCGAGAGGTCCCGGGTTCAAATCC |
| | | C |
| 295 | Pro_AGG_chr1:16768 | GGCTCGTTGGTCTAGGGGTATGATTCTCGCT |
| | 4724-167684796 (−) | TAGGGTGCGAGAGGTCCCGGGTTCAAATCCC |
| | | G |
| 296 | Lys_TTT_chr1:20447 | CGCCCGGATAGCTCAGTCGGTAGAGCATCAG |
| | 5654-204475727 (+) | ACTTTTAATCTGAGGGTCCAGGGTTCAAGTC |
| 297 | Lys_TTT_chr1:20447 | GCCCGGATAGCTCAGTCGGTAGAGCATCAGA |
| | 6157-204476230 (−) | CTTTTAATCTGAGGGTCCAGGGTTCAAGTCC |
| | | C |
| 298 | Leu_CAA_chr1:24916 | TGTCAGGATGGCCGAGTGGTCTAAGGCGCCA |
| | 8053-249168159 (+) | GACTCAAGGTAAGCACCTTGCCTGCGGGCTT |
| 299 | Glu_CTC_chr1:24916 | TTCCCTGGTGGTCTAGTGGTTAGGATTCGGC |
| | 8446-249168518 (+) | GCTCTCACCGCCGCGGCCCGGGTTCGATTCC |
| | | C |
| 300 | Tyr_GTA_chr2:27273 | GCCTTCGATAGCTCAGTTGGTAGAGCGGAGG |
| | 649-27273738 (+) | ACTGTAGTGGATAGGGCGTGGCAATCCTTAG |
| 301 | Ala_AGC_chr2:27274 | CGGGGGATTAGCTCAAATGGTAGAGCGCTCG |
| | 081-27274154 (+) | CTTAGCATGCGAGAGGTAGCGGGATCGATGC |
| 302 | Ile_TAT_chr2:430376 | AGCTCCAGTGGCGCAATCGGTTAGCGCGCGG |
| | 75-43037768 (+) | TACTTATACAGCAGTACATGCAGAGCAATGC |
| 303 | Gly_CCC_chr2:70476 | GCGCCGCTGGTGTAGTGGTATCATGCAAGAT |
| | 122-70476193 (−) | TCCCATTCTTGCGACCCGGGTTCGATTCCCG |
| | | G |
| 304 | Glu_TTC_chr2:13109 | TCCCATATGGTCTAGCGGTTAGGATTCCTGG |
| | 4700-131094772 (−) | TTTTCACCCAGGTGGCCCGGGTTCGACTCCC |
| | | G |
| 305 | Ala_CGC_chr2:15725 | GGGGGATGTAGCTCAGTGGTAGAGCGCGCGC |
| | 7280-157257352 (+) | TTCGCATGTGTGAGGTCCCGGGTTCAATCCC |
| | | C |
| 306 | Gly_GCC_chr2:15725 | GCATTGGTGGTTCAGTGGTAGAATTCTCGCC |
| | 7658-157257729 (−) | TGCCACGCGGGAGGCCCGGGTTCGATTCCCG |
| | | G |
| 307 | Arg_ACG_chr3:45730 | GGGCCAGTGGCGCAATGGATAACGCGTCTGA |
| | 490-45730563 (−) | CTACGGATCAGAAGATTCTAGGTTCGACTCC |
| 308 | Val_AAC_chr3:16949 | GGTTTCCGTAGTGTAGTGGTTATCACGTTCG |
| | 0017-169490090 (+) | CCTAACACGCGAAAGGTCCCCGGTTCGAAAC |
| | | C |
| 309 | Val_AAC_chr5:18059 | AGTTTCCGTAGTGTAGTGGTTATCACGTTCG |
| | 6609-180596682 (+) | CCTAACACGCGAAAGGTCCCCGGTTCGAAAC |
| | | C |
| 310 | Leu_AAG_chr5:1806 | AGGTAGCGTGGCCGAGCGGTCTAAGGCGCTG |
| | 14700-180614782 (+) | GATTAAGGCTCCAGTCTCTTCGGGGGCGTGG |
| 311 | Val_AAC_chr5:18061 | GTTTCCGTAGTGTAGTGGTCATCACGTTCGC |
| | 5415-180615488 (−) | CTAACACGCGAAAGGTCCCCGGTTCGAAACC |
| | | G |
| 312 | Pro_TGG_chr5:18061 | GGCTCGTTGGTCTAGGGGTATGATTCTCGCT |
| | 5853-180615925 (−) | TTGGGTGCGAGAGGTCCCGGGTTCAAATCCC |
| | | G |
| 313 | Thr_TGT_chr5:18061 | GGCTCCATAGCTCAGGGGTTAGAGCACTGGT |
| | 8686-180618758 (−) | CTTGTAAACCAGGGTCGCGAGTTCAAATCTC |
| 314 | Ala_TGC_chr5:18063 | TGGGGATGTAGCTCAGTGGTAGAGCGCATGC |
| | 3867-180633939 (+) | TTTGCATGTATGAGGCCCCGGGTTCGATCCC |
| | | C |
| 315 | Lys_CTT_chr5:18063 | CGCCCGGCTAGCTCAGTCGGTAGAGCATGAG |
| | 4754-180634827 (+) | ACTCTTAATCTCAGGGTCGTGGGTTCGAGCC |
| 316 | Val_AAC_chr5:18064 | GTTTCCGTAGTGTAGTGGTTATCACGTTCGC |
| | 5269-180645342 (−) | CTAACACGCGAAAGGTCCCCGGTTCGAAACC |
| | | G |
| 317 | Lys_CTT_chr5:18064 | GCCCGGCTAGCTCAGTCGGTAGAGCATGAGA |
| | 8978-180649051 (−) | CTCTTAATCTCAGGGTCGTGGGTTCGAGCCC |
| | | C |
| 318 | Val_CAC_chr5:18064 | GTTTCCGTAGTGTAGTGGTTATCACGTTCGC |
| | 9394-180649467 (−) | CTCACACGCGAAAGGTCCCCGGTTCGAAACC |
| | | G |
| 319 | Met_CAT_chr6:26286 | CAGCAGAGTGGCGCAGCGGAAGCGTGCTGGG |
| | 753-26286825 (+) | CCCATAACCCAGAGGTCGATGGATCGAAAC |
| 320 | Ser_GCT_chr6:26305 | GGAGAGGCCTGGCCGAGTGGTTAAGGCGATG |
| | 717-26305801 (−) | GACTGCTAATCCATTGTGCTCTGCACGCGTG |
| 321 | Gln_TTG_chr6:26311 | GGCCCCATGGTGTAATGGTTAGCACTCTGGA |
| | 423-26311495 (−) | CTTTGAATCCAGCGATCCGAGTTCAAATCTC |
| 322 | Gln_TTG_chr6:26311 | GGCCCCATGGTGTAATGGTTAGCACTCTGGA |
| | 974-26312046 (−) | CTTTGAATCCAGCGATCCGAGTTCAAATCTC |
| 323 | Ser_TGA_chr6:26312 | GTAGTCGTGGCCGAGTGGTTAAGGCGATGGA |
| | 823-26312905 (−) | CTTGAAATCCATTGGGGTCTCCCCGCGCAGG |
| 324 | Met_CAT_chr6:26313 | AGCAGAGTGGCGCAGCGGAAGCGTGCTGGGC |
| | 351-26313423 (−) | CCATAACCCAGAGGTCGATGGATCGAAACC |
| 325 | Arg_TCG_chr6:26323 | GGACCACGTGGCCTAATGGATAAGGCGTCTG |
| | 045-26323118 (+) | ACTTCGGATCAGAAGATTGAGGGTTCGAATC |
| 326 | Ser_AGA_chr6:26327 | TGTAGTCGTGGCCGAGTGGTTAAGGCGATGG |
| | 816-26327898 (+) | ACTAGAAATCCATTGGGGTCTCCCCGCGCAG |
| 327 | Met_CAT_chr6:26330 | AGCAGAGTGGCGCAGCGGAAGCGTGCTGGGC |
| | 528-26330600 (−) | CCATAACCCAGAGGTCGATGGATCGAAACC |
| 328 | Leu_CAG_chr6:26521 | CGTCAGGATGGCCGAGCGGTCTAAGGCGCTG |
| | 435-26521518 (+) | CGTTCAGGTCGCAGTCTCCCCTGGAGGCGTG |
| 329 | Thr_AGT_chr6:26533 | GGCTCCGTGGCTTAGCTGGTTAAAGCGCCTG |
| | 144-26533218 (−) | TCTAGTAAACAGGAGATCCTGGGTTCGAATC |
| 330 | Arg_ACG_chr6:26537 | AGGGCCAGTGGCGCAATGGATAACGCGTCTG |
| | 725-26537798 (+) | ACTACGGATCAGAAGATTCCAGGTTCGACTC |
| 331 | Val_CAC_chr6:26538 | GGTTTCCGTAGTGTAGTGGTTATCACGTTCG |
| | 281-26538354 (+) | CCTCACACGCGAAAGGTCCCCGGTTCGAAAC |
| | | C |
| 332 | Ala_CGC_chr6:26553 | AGGGGATGTAGCTCAGTGGTAGAGCGCATGC |
| | 730-26553802 (+) | TTCGCATGTATGAGGTCCCGGGTTCGATCCC |
| | | C |
| 333 | Ile_AAT_chr6:265543 | TGGCCGGTTAGCTCAGTTGGTTAGAGCGTGG |
| | 49-26554423 (+) | TGCTAATAACGCCAAGGTCGCGGGTTCGATC |
| 334 | Pro_AGG_chr6:26555 | CGGCTCGTTGGTCTAGGGGTATGATTCTCGC |
| | 497-26555569 (+) | TTAGGGTGCGAGAGGTCCCGGGTTCAAATCC |
| | | C |
| 335 | Lys_CTT_chr6:26556 | AGCCCGGCTAGCTCAGTCGGTAGAGCATGAG |
| | 773-26556846 (+) | ACTCTTAATCTCAGGGTCGTGGGTTCGAGCC |
| 336 | Tyr_GTA_chr6:26569 | TCCTTCGATAGCTCAGTTGGTAGAGCGGAGG |
| | 085-26569176 (+) | ACTGTAGTTGGCTGTGTCCTTAGACATCCTT |
| | | A |
| 337 | Ala_AGC_chr6:26572 | GGGGAATTAGCTCAAATGGTAGAGCGCTCGC |
| | 091-26572164 (−) | TTAGCATGCGAGAGGTAGCGGGATCGATGCC |
| 338 | Met_CAT_chr6:26766 | CGCCCTCTTAGCGCAGCGGGCAGCGCGTCAG |
| | 443-26766516 (+) | TCTCATAATCTGAAGGTCCTGAGTTCGAGCC |
| | | T |
| 339 | Ile_TAT_chr6:269881 | TGCTCCAGTGGCGCAATCGGTTAGCGCGCGG |
| | 24-26988218 (+) | TACTTATATGGCAGTATGTGTGCGAGTGATG |
| 340 | His_GTG_chr6:27125 | TGCCGTGATCGTATAGTGGTTAGTACTCTGC |
| | 905-27125977 (+) | GTTGTGGCCGCAGCAACCTCGGTTCGAATCC |
| | | G |
| 341 | Ile_AAT_chr6:271449 | GGCCGGTTAGCTCAGTTGGTTAGAGCGTGGT |
| | 93-27145067 (−) | GCTAATAACGCCAAGGTCGCGGGTTCGATCC |
| 342 | Val_AAC_chr6:27203 | AGTTTCCGTAGTGTAGTGGTTATCACGTTTG |
| | 287-27203360 (+) | CCTAACACGCGAAAGGTCCCCGGTTCGAAAC |
| | | C |
| 343 | Val_CAC_chr6:27248 | GCTTCTGTAGTGTAGTGGTTATCACGTTCGC |
| | 048-27248121 (−) | CTCACACGCGAAAGGTCCCCGGTTCGAAACC |
| | | G |
| 344 | Asp_GTC_chr6:27447 | TTCCTCGTTAGTATAGTGGTGAGTATCCCCG |
| | 452-27447524 (+) | CCTGTCACGCGGGAGACCGGGGTTCGATTCC |
| | | C |
| 345 | Ser_TGA_chr6:27473 | GTAGTCGTGGCCGAGTGGTTAAGGCGATGGA |
| | 606-27473688 (−) | CTTGAAATCCATTGGGGTTTCCCCGCGCAGG |
| 346 | Gln_CTG_chr6:27487 | AGGTTCCATGGTGTAATGGTTAGCACTCTGG |
| | 307-27487379 (+) | ACTCTGAATCCAGCGATCCGAGTTCAAATCT |
| 347 | Asp_GTC_chr6:27551 | TCCTCGTTAGTATAGTGGTGAGTGTCCCCGT |
| | 235-27551307 (−) | CTGTCACGCGGGAGACCGGGGTTCGATTCCC |
| | | C |
| 348 | Val_AAC_chr6:27618 | GTTTCCGTAGTGTAGTGGTTATCACGTTCGC |
| | 706-27618779 (−) | CTAACACGCGAAAGGTCCCTGGATCAAAACC |
| | | A |
| 349 | Ile_AAT_chr6:276559 | CGGCCGGTTAGCTCAGTTGGTTAGAGCGTGG |
| | 66-27656040 (+) | TGCTAATAACGCCAAGGTCGCGGGTTCGATC |
| 350 | Gln_CTG_chr6:27759 | GGCCCCATGGTGTAATGGTCAGCACTCTGGA |
| | 134-27759206 (−) | CTCTGAATCCAGCGATCCGAGTTCAAATCTC |
| 351 | Gln_TTG_chr6:27763 | GGCCCCATGGTGTAATGGTTAGCACTCTGGA |
| | 639-27763711 (−) | CTTTGAATCCAGCGATCCGAGTTCAAATCTC |
| 352 | Ala_AGC_chr6:28574 | TGGGGGTGTAGCTCAGTGGTAGAGCGCGTGC |
| | 932-28575004 (+) | TTAGCATGTACGAGGTCCCGGGTTCAATCCC |
| 353 | Ala_AGC_chr6:28626 | GGGGATGTAGCTCAGTGGTAGAGCGCATGCT |
| | 013-28626085 (−) | TAGCATGCATGAGGTCCCGGGTTCGATCCCC |
| 354 | Ala_CGC_chr6:28697 | AGGGGGTGTAGCTCAGTGGTAGAGCGCGTGC |
| | 091-28697163 (+) | TTCGCATGTACGAGGCCCCGGGTTCGACCCC |
| 355 | Ala_AGC_chr6:28806 | GGGGGTGTAGCTCAGTGGTAGAGCGCGTGCT |
| | 220-28806292 (−) | TAGCATGCACGAGGCCCCGGGTTCAATCCCC |
| 356 | Ala_AGC_chr6:28831 | GGGGGTGTAGCTCAGTGGTAGAGCGCGTGCT |
| | 461-28831533 (−) | TAGCATGCACGAGGCCCCGGGTTCAATCCCC |
| 357 | Leu_CAA_chr6:28863 | GTCAGGATGGCCGAGTGGTCTAAGGCGCCAG |
| | 999-28864105 (−) | ACTCAAGCTAAGCTTCCTCCGCGGTGGGGAT |
| 358 | Leu_CAA_chr6:28908 | TGTCAGGATGGCCGAGTGGTCTAAGGCGCCA |
| | 829-28908934 (+) | GACTCAAGCTTGGCTTCCTCGTGTTGAGGAT |
| | | T |
| 359 | Gln_CTG_chr6:28909 | GGTTCCATGGTGTAATGGTTAGCACTCTGGA |
| | 377-28909449 (−) | CTCTGAATCCAGCGATCCGAGTTCAAATCTC |
| 360 | Leu_AAG_chr6:2891 | GGTAGCGTGGCCGAGCGGTCTAAGGCGCTGG |
| | 1398-28911480 (−) | ATTAAGGCTCCAGTCTCTTCGGGGGCGTGGG |
| 361 | Met_CAT_chr6:28912 | TGCCTCCTTAGCGCAGTAGGCAGCGCGTCAG |
| | 351-28912424 (+) | TCTCATAATCTGAAGGTCCTGAGTTCGAACC |
| | | T |
| 362 | Lys_TTT_chr6:28918 | AGCCCGGATAGCTCAGTCGGTAGAGCATCAG |
| | 805-28918878 (+) | ACTTTTAATCTGAGGGTCCAGGGTTCAAGTC |
| 363 | Met_CAT_chr6:28921 | GCCTCCTTAGCGCAGTAGGCAGCGCGTCAGT |
| | 041-28921114 (−) | CTCATAATCTGAAGGTCCTGAGTTCGAACCT |
| 364 | Glu_CTC_chr6:28949 | TTCCCTGGTGGTCTAGTGGTTAGGATTCGGC |
| | 975-28950047 (+) | GCTCTCACCGCCGCGGCCCGGGTTCGATTCC |
| | | C |
| 365 | Leu_TAA_chr6:14453 | CACCAGGATGGCCGAGTGGTTAAGGCGTTGG |
| | 7683-144537766 (+) | ACTTAAGATCCAATGGACATATGTCCGCGTG |
| 366 | Pro_AGG_chr7:12842 | TGGCTCGTTGGTCTAGGGGTATGATTCTCGC |
| | 3503-128423575 (+) | TTAGGGTGCGAGAGGTCCCGGGTTCAAATCC |
| | | C |
| 367 | Arg_CCT_chr7:13902 | AGCCCCAGTGGCCTAATGGATAAGGCATTGG |
| | 5445-139025518 (+) | CCTCCTAAGCCAGGGATTGTGGGTTCGAGTC |
| 368 | Cys_GCA_chr7:14938 | GGGGATATAGCTCAGGGGTAGAGCATTTGAC |
| | 8271-149388343 (−) | TGCAGATCAAGAGGTCCCCGGTTCAAATCCG |
| 369 | Tyr_GTA_chr8:67025 | CCCTTCGATAGCTCAGCTGGTAGAGCGGAGG |
| | 601-67025694 (+) | ACTGTAGCTACTTCCTCAGCAGGAGACATCC |
| 370 | Tyr_GTA_chr8:67026 | CCCTTCGATAGCTCAGCTGGTAGAGCGGAGG |
| | 222-67026311 (+) | ACTGTAGGCGCGCGCCCGTGGCCATCCTTAG |
| 371 | Ala_AGC_chr8:67026 | TGGGGGATTAGCTCAAATGGTAGAGCGCTCG |
| | 423-67026496 (+) | CTTAGCATGCGAGAGGTAGCGGGATCGATGC |
| 372 | Ser_AGA_chr8:96281 | GTAGTCGTGGCCGAGTGGTTAAGGCGATGGA |
| | 884-96281966 (−) | CTAGAAATCCATTGGGGTCTCCCCGCGCAGG |
| 373 | Met_CAT_chr8:12416 | GCCTCGTTAGCGCAGTAGGTAGCGCGTCAGT |
| | 9469-124169542 (−) | CTCATAATCTGAAGGTCGTGAGTTCGATCCT |
| | | C |
| 374 | Arg_TCT_chr9:13110 | GGCTCTGTGGCGCAATGGATAGCGCATTGGA |
| | 2354-131102445 (−) | CTTCTAGCTGAGCCTAGTGTGGTCATTCAAA |
| 375 | Asn_GTT_chr10:2251 | GTCTCTGTGGCGCAATCGGTTAGCGCGTTCG |
| | 8437-22518511 (−) | GCTGTTAACCGAAAGGTTGGTGGTTCGAGCC |
| 376 | Ser_TGA_chr10:6952 | GGCAGCGATGGCCGAGTGGTTAAGGCGTTGG |
| | 4260-69524342 (+) | ACTTGAAATCCAATGGGGTCTCCCCGCGCAG |
| 377 | Val_TAC_chr11:5931 | GGTTCCATAGTGTAGTGGTTATCACGTCTGC |
| | 8101-59318174 (−) | TTTACACGCAGAAGGTCCTGGGTTCGAGCCC |
| | | C |
| 378 | Val_TAC_chr11:5931 | GGTTCCATAGTGTAGCGGTTATCACGTCTGC |
| | 8459-59318532 (−) | TTTACACGCAGAAGGTCCTGGGTTCGAGCCC |
| | | C |
| 379 | Arg_TCT_chr11:5931 | TGGCTCTGTGGCGCAATGGATAGCGCATTGG |
| | 8766-59318852 (+) | ACTTCTAGATAGTTAGAGAAATTCAAAGGTT |
| 380 | Leu_TAA_chr11:5931 | TACCAGAATGGCCGAGTGGTTAAGGCGTTGG |
| | 9227-59319310 (+) | ACTTAAGATCCAATGGATTCATATCCGCGTG |
| 381 | Lys_TTT_chr11:5932 | GGCCCGGATAGCTCAGTCGGTAGAGCATCAG |
| | 3901-59323974 (+) | ACTTTTAATCTGAGGGTCCGGGGTTCAAGTC |
| 382 | Phe_GAA_chr11:5932 | GCCGAAATAGCTCAGTTGGGAGAGCGTTAGA |
| | 4969-59325042 (−) | CTGAAGATCTAAAGGTCCCTGGTTCGATCCC |
| 383 | Lys_TTT_chr11:5932 | GCCCGGATAGCTCAGTCGGTAGAGCATCAGA |
| | 7807-59327880 (−) | CTTTTAATCTGAGGGTCCAGGGTTCAAGTCC |
| | | C |
| 384 | Phe_GAA_chr11:5933 | GCCGAAATAGCTCAGTTGGGAGAGCGTTAGA |
| | 3852-59333925 (−) | CTGAAGATCTAAAGGTCCCTGGTTCAATCCC |
| 385 | Ser_GCT_chr11:6611 | GGACGAGGTGGCCGAGTGGTTAAGGCGATGG |
| | 5590-66115672 (+) | ACTGCTAATCCATTGTGCTTTGCACGCGTGG |
| 386 | Pro_TGG_chr11:7594 | GGCTCGTTGGTCTAGGGGTATGATTCTCGGT |
| | 6868-75946940 (−) | TTGGGTCCGAGAGGTCCCGGGTTCAAATCCC |
| | | G |
| 387 | Ser_CGA_chr12:5658 | AGTCACGGTGGCCGAGTGGTTAAGGCGTTGG |
| | 4147-56584229 (+) | ACTCGAAATCCAATGGGGTTTCCCCGCACAG |
| 388 | Asp_GTC_chr12:9889 | CTCCTCGTTAGTATAGTGGTTAGTATCCCCG |
| | 7280-98897352 (+) | CCTGTCACGCGGGAGACCGGGGTTCAATTCC |
| | | C |
| 389 | Trp_CCA_chr12:9889 | GGACCTCGTGGCGCAACGGTAGCGCGTCTGA |
| | 8029-98898101 (+) | CTCCAGATCAGAAGGCTGCGTGTTCGAATCA |
| 390 | Ala_TGC_chr12:1254 | GGGGATGTAGCTCAGTGGTAGAGCGCATGCT |
| | 06300-125406372 (−) | TTGCATGTATGAGGCCCCGGGTTCGATCCCC |
| 391 | Phe_GAA_chr12:1254 | GCCGAAATAGCTCAGTTGGGAGAGCGTTAGA |
| | 12388-125412461 (−) | CTGAAGATCTAAAGGTCCCTGGTTCGATCCC |
| 392 | Ala_TGC_chr12:1254 | AGGGGATGTAGCTCAGTGGTAGAGCGCATGC |
| | 24511-125424583 (+) | TTTGCACGTATGAGGCCCCGGGTTCAATCCC |
| 393 | Asn_GTT_chr13:3124 | GTCTCTGTGGCGCAATCGGTTAGCGCGTTCG |
| | 8100-31248174 (−) | GCTGTTAACCGAAAGGTTGGTGGTTCGAGCC |
| 394 | Glu_TTC_chr13:4549 | TCCCACATGGTCTAGCGGTTAGGATTCCTGG |
| | 2061-45492133 (−) | TTTTCACCCAGGCGGCCCGGGTTCGACTCCC |
| | | G |
| 395 | Thr_TGT_chr14:2108 | GGCTCCATAGCTCAGGGGTTAGAGCGCTGGT |
| | 1948-21082021 (−) | CTTGTAAACCAGGGGTCGCGAGTTCAATTCT |
| 396 | Leu_TAG_chr14:2109 | TGGTAGTGTGGCCGAGCGGTCTAAGGCGCTG |
| | 3528-21093610 (+) | GATTTAGGCTCCAGTCTCTTCGGGGGCGTGG |
| 397 | Thr_TGT_chr14:2109 | GGCTCCATAGCTCAGGGGTTAGAGCACTGGT |
| | 9318-21099391 (−) | CTTGTAAACCAGGGGTCGCGAGTTCAAATCT |
| 398 | Pro_TGG_chr14:2110 | TGGCTCGTTGGTCTAGTGGTATGATTCTCGC |
| | 1164-21101236 (+) | TTTGGGTGCGAGAGGTCCCGGGTTCAAATCC |
| | | C |
| 399 | Tyr_GTA_chr14:2113 | CCTTCGATAGCTCAGCTGGTAGAGCGGAGGA |
| | 1350-21131444 (−) | CTGTAGATTGTACAGACATTTGCGGACATCC |
| 400 | Thr_TGT_chr14:2114 | AGGCCCTATAGCTCAGGGGTTAGAGCACTGG |
| | 9848-21149921 (+) | TCTTGTAAACCAGGGGTCGCGAGTTCAAATC |
| 401 | Tyr_GTA_chr14:2115 | TCCTTCGATAGCTCAGCTGGTAGAGCGGAGG |
| | 1431-21151520 (+) | ACTGTAGTACTTAATGTGTGGTCATCCTTAG |
| | | G |
| 402 | Pro_TGG_chr14:2115 | TGGCTCGTTGGTCTAGGGGTATGATTCTCGC |
| | 2174-21152246 (+) | TTTGGGTGCGAGAGGTCCCGGGTTCAAATCC |
| | | C |
| 403 | Lys_CTT_chr14:5870 | GCCCGGCTAGCTCAGTCGGTAGAGCATGGGA |
| | 6612-58706685 (−) | CTCTTAATCCCAGGGTCGTGGGTTCGAGCCC |
| 404 | Ile_AAT_chr14:10278 | CGGCCGGTTAGCTCAGTTGGTTAGAGCGTGG |
| | 3428-102783502 (+) | TGCTAATAACGCCAAGGTCGCGGGTTCGATC |
| 405 | Glu_TTC_chr15:2632 | TCCCACATGGTCTAGCGGTTAGGATTCCTGG |
| | 7380-26327452 (−) | TTTTCACCCAGGCGGCCCGGGTTCGACTCCC |
| | | G |
| 406 | Ser_GCT_chr15:4088 | GACGAGGTGGCCGAGTGGTTAAGGCGATGGA |
| | 6022-40886104 (−) | CTGCTAATCCATTGTGCTCTGCACGCGTGG |
| 407 | His_GTG_chr15:4549 | GCCGTGATCGTATAGTGGTTAGTACTCTGCG |
| | 0803-45490875 (−) | TTGTGGCCGCAGCAACCTCGGTTCGAATCCG |
| | | A |
| 408 | His_GTG_chr15:4549 | CGCCGTGATCGTATAGTGGTTAGTACTCTGC |
| | 3348-45493420 (+) | GTTGTGGCCGCAGCAACCTCGGTTCGAATCC |
| 409 | Gln_CTG_chr15:6616 | GGTTCCATGGTGTAATGGTTAGCACTCTGGA |
| | 1399-66161471 (−) | CTCTGAATCCAGCGATCCGAGTTCAAATCTC |
| 410 | Lys_CTT_chr15:7915 | TGCCCGGCTAGCTCAGTCGGTAGAGCATGGG |
| | 2903-79152976 (+) | ACTCTTAATCCCAGGGTCGTGGGTTCGAGCC |
| 411 | Arg_TCG_chr15:8987 | GGGCCGCGTGGCCTAATGGATAAGGCGTCTG |
| | 8303-89878376 (+) | ACTTCGGATCAGAAGATTGCAGGTTCGAGTC |
| 412 | Gly_CCC_chr16:6867 | GCGCCGCTGGTGTAGTGGTATCATGCAAGAT |
| | 35-686806 (−) | TCCCATTCTTGCGACCCGGGTTCGATTCCCG |
| | | G |
| 413 | Arg_CCG_chr16:3200 | GGGCCGCGTGGCCTAATGGATAAGGCGTCTG |
| | 674-3200747 (+) | ATTCCGGATCAGAAGATTGAGGGTTCGAGTC |
| 414 | Arg_CCT_chr16:3202 | CGCCCCGGTGGCCTAATGGATAAGGCATTGG |
| | 900-3202973 (+) | CCTCCTAAGCCAGGGATTGTGGGTTCGAGTC |
| 415 | Lys_CTT_chr16:3207 | GCCCGGCTAGCTCAGTCGGTAGAGCATGAGA |
| | 405-3207478 (−) | CCCTTAATCTCAGGGTCGTGGGTTCGAGCCC |
| 416 | Thr_CGT_chr16:1437 | AGGCGCGGTGGCCAAGTGGTAAGGCGTCGGT |
| | 9749-14379821 (+) | CTCGTAAACCGAAGATCACGGGTTCGAACCC |
| 417 | Leu_TAG_chr16:2220 | GGTAGCGTGGCCGAGTGGTCTAAGGCGCTGG |
| | 7031-22207113 (−) | ATTTAGGCTCCAGTCATTTCGATGGCGTGGG |
| | | T |
| 418 | Leu_AAG_chr16:223 | GGGTAGCGTGGCCGAGCGGTCTAAGGCGCTG |
| | 08460-22308542 (+) | GATTAAGGCTCCAGTCTCTTCGGGGGCGTGG |
| 419 | Leu_CAG_chr16:5733 | AGTCAGGATGGCCGAGCGGTCTAAGGCGCTG |
| | 3862-57333945 (+) | CGTTCAGGTCGCAGTCTCCCCTGGAGGCGTG |
| 420 | Leu_CAG_chr16:5733 | GTCAGGATGGCCGAGCGGTCTAAGGCGCTGC |
| | 4391-57334474 (−) | GTTCAGGTCGCAGTCTCCCCTGGAGGCGTGG |
| 421 | Met_CAT_chr16:8741 | GCCTCGTTAGCGCAGTAGGCAGCGCGTCAGT |
| | 7627-87417700 (−) | CTCATAATCTGAAGGTCGTGAGTTCGAGCCT |
| 422 | Leu_TAG_chr17:8023 | GGTAGCGTGGCCGAGCGGTCTAAGGCGCTGG |
| | 631-8023713 (−) | ATTTAGGCTCCAGTCTCTTCGGAGGCGTGGG |
| 423 | Arg_TCT_chr17:8024 | TGGCTCTGTGGCGCAATGGATAGCGCATTGG |
| | 242-8024330 (+) | ACTTCTAGTGACGAATAGAGCAATTCAAAGG |
| 424 | Gly_GCC_chr17:8029 | CGCATTGGTGGTTCAGTGGTAGAATTCTCGC |
| | 063-8029134 (+) | CTGCCACGCGGGAGGCCCGGGTTCGATTCCC |
| 425 | Ser_CGA_chr17:8042 | GCTGTGATGGCCGAGTGGTTAAGGCGTTGGA |
| | 198-8042280 (−) | CTCGAAATCCAATGGGGTCTCCCCGCGCAGG |
| 426 | Thr_AGT_chr17:8042 | GGCGCCGTGGCTTAGCTGGTTAAAGCGCCTG |
| | 769-8042843 (−) | TCTAGTAAACAGGAGATCCTGGGTTCGAATC |
| 427 | Trp_CCA_chr17:8089 | CGACCTCGTGGCGCAACGGTAGCGCGTCTGA |
| | 675-8089747 (+) | CTCCAGATCAGAAGGTTGCGTGTTCAAATCA |
| 428 | Ser_GCT_chr17:8090 | AGACGAGGTGGCCGAGTGGTTAAGGCGATGG |
| | 183-8090265 (+) | ACTGCTAATCCATTGTGCTCTGCACGCGTG |
| 429 | Thr_AGT_chr17:8090 | CGGCGCCGTGGCTTAGTTGGTTAAAGCGCCT |
| | 477-8090551 (+) | GTCTAGTAAACAGGAGATCCTGGGTTCGAAT |
| 430 | Trp_CCA_chr17:8124 | GGCCTCGTGGCGCAACGGTAGCGCGTCTGAC |
| | 186-8124258 (−) | TCCAGATCAGAAGGTTGCGTGTTCAAATCAC |
| 431 | Gly_TCC_chr17:8124 | AGCGTTGGTGGTATAGTGGTAAGCATAGCTG |
| | 865-8124937 (+) | CCTTCCAAGCAGTTGACCCGGGTTCGATTCC |
| | | C |
| 432 | Asp_GTC_chr17:8125 | TCCTCGTTAGTATAGTGGTGAGTATCCCCGC |
| | 555-8125627 (−) | CTGTCACGCGGGAGACCGGGGTTCGATTCCC |
| | | C |
| 433 | Pro_CGG_chr17:8126 | GGCTCGTTGGTCTAGGGGTATGATTCTCGCT |
| | 150-8126222 (−) | TCGGGTGCGAGAGGTCCCGGGTTCAAATCCC |
| | | G |
| 434 | Thr_AGT_chr17:8129 | GGCGCCGTGGCTTAGTTGGTTAAAGCGCCTG |
| | 552-8129626 (−) | TCTAGTAAACAGGAGATCCTGGGTTCGAATC |
| 435 | Ser_AGA_chr17:8129 | GTAGTCGTGGCCGAGTGGTTAAGGCGATGGA |
| | 927-8130009 (−) | CTAGAAATCCATTGGGGTCTCCCCGCGCAGG |
| 436 | Trp_CCA_chr17:1941 | TGACCTCGTGGCGCAATGGTAGCGCGTCTGA |
| | 1493-19411565 (+) | CTCCAGATCAGAAGGTTGCGTGTTCAAGTCA |
| 437 | Thr_CGT_chr17:2987 | AGGCGCGGTGGCCAAGTGGTAAGGCGTCGGT |
| | 7092-29877164 (+) | CTCGTAAACCGAAGATCGCGGGTTCGAACCC |
| 438 | Cys_GCA_chr17:3702 | AGGGGGTATAGCTCAGTGGTAGAGCATTTGA |
| | 3897-37023969 (+) | CTGCAGATCAAGAGGTCCCCGGTTCAAATCC |
| 439 | Cys_GCA_chr17:3702 | GGGGGTATAGCTCAGTGGTAGAGCATTTGAC |
| | 5544-37025616 (−) | TGCAGATCAAGAGGTCCCTGGTTCAAATCCG |
| 440 | Cys_GCA_chr17:3730 | GGGGGTATAGCTCAGTGGTAGAGCATTTGAC |
| | 9986-37310058 (−) | TGCAGATCAAGAGGTCCCCGGTTCAAATCCG |
| 441 | Gln_TTG_chr17:4726 | AGGTCCCATGGTGTAATGGTTAGCACTCTGG |
| | 9889-47269961 (+) | ACTTTGAATCCAGCGATCCGAGTTCAAATCT |
| 442 | Arg_CCG_chr17:6601 | GACCCAGTGGCCTAATGGATAAGGCATCAGC |
| | 6012-66016085 (−) | CTCCGGAGCTGGGGATTGTGGGTTCGAGTCC |
| 443 | Arg_CCT_chr17:7303 | AGCCCCAGTGGCCTAATGGATAAGGCACTGG |
| | 0000-73030073 (+) | CCTCCTAAGCCAGGGATTGTGGGTTCGAGTC |
| 444 | Arg_CCT_chr17:7303 | GCCCCAGTGGCCTAATGGATAAGGCACTGGC |
| | 0525-73030598 (−) | CTCCTAAGCCAGGGATTGTGGGTTCGAGTCC |
| 445 | Arg_TCG_chr17:7303 | AGACCGCGTGGCCTAATGGATAAGGCGTCTG |
| | 1207-73031280 (+) | ACTTCGGATCAGAAGATTGAGGGTTCGAGTC |
| 446 | Asn_GTT_chr19:1383 | CGTCTCTGTGGCGCAATCGGTTAGCGCGTTC |
| | 561-1383635 (+) | GGCTGTTAACCGAAAGGTTGGTGGTTCGAGC |
| 447 | Gly_TCC_chr19:4724 | GGCGTTGGTGGTATAGTGGTTAGCATAGCTG |
| | 081-4724153 (+) | CCTTCCAAGCAGTTGACCCGGGTTCGATTCC |
| | | C |
| 448 | Val_CAC_chr19:4724 | GTTTCCGTAGTGTAGCGGTTATCACATTCGC |
| | 646-4724719 (−) | CTCACACGCGAAAGGTCCCCGGTTCGATCCC |
| | | G |
| 449 | Thr_AGT_chr19:3366 | TGGCGCCGTGGCTTAGTTGGTTAAAGCGCCT |
| | 7962-33668036 (+) | GTCTAGTAAACAGGAGATCCTGGGTTCGAAT |
| 450 | Ile_TAT_chr19:39902 | GCTCCAGTGGCGCAATCGGTTAGCGCGCGGT |
| | 807-39902900 (−) | ACTTATATGACAGTGCGAGCGGAGCAATGCC |
| 451 | Gly_GCC_chr21:1882 | GCATGGGTGGTTCAGTGGTAGAATTCTCGCC |
| | 7106-18827177 (−) | TGCCACGCGGGAGGCCCGGGTTCGATTCCCG |

### Non-Naturally Occurring Modification

A TREM, a TREM core fragment or a TREM fragment described herein may or may not comprise a non-naturally occurring modification, e.g., a modification described in any one of Table 4. A non-naturally occurring modification can be made according to methods known in the art. Exemplary methods of making non-naturally occurring modifications are provided in Examples 1-3.

In an embodiment, a non-naturally occurring modification is a modification that a cell, e.g., a human cell, does not make on an endogenous tRNA.

In an embodiment, a non-naturally occurring modification is a modification that a cell, e.g., a human cell, can make on an endogenous tRNA, but wherein such modification is in a location in which it does not occur on a native tRNA. In an embodiment, the non-naturally occurring modification is in a domain, linker or arm which does not have such modification in nature. In an embodiment, the non-naturally occurring modification is at a position within a domain, linker or arm, which does not have such modification in nature. In an embodiment, the non-naturally occurring modification is on a nucleotide which does not have such modification in nature. In an embodiment, the non-naturally occurring modification is on a nucleotide at a position within a domain, linker or arm, which does not have such modification in nature. In an embodiment, a TREM, a TREM core fragment or a TREM fragment described herein comprises a non-naturally occurring modification provided in Table 4, or a combination thereof.

TABLE 4
Exemplary non-naturally occurring modifications
Chemical Modification

| | |
|---|---|
| (S)-constrained ethyl (cEt) | 5-(methoxycarbonyl-methyl)uracil |
| (±)1-(2-Hydroxypropyl)pseudouridine | 5-(methyl) 2(thio)uracil |
| (2R)-1-(2-Hydroxypropyl)pseudouridine | 5-(methyl) 2,4 (dithio)uracil |
| (2S)-1-(2-Hydroxypropyl)pseudouridine | 5-(methyl) 4 (thio)uracil |
| (3-(3-amino-3-carboxypropyl)uridine | 5-(methyl)-2-(thio)pseudouracil |
| (E)-5-(2-Bromo-vinyl)ara-uridine | 5-(methyl)-2-(thio)uracil |
| (E)-5-(2-Bromo-vinyl)cytidine | 5-(methyl)-2,4 (dithio)pseudouracil |
| (E)-5-(2-Bromo-vinyl)uridine | 5-(methyl)-2,4-(dithio)uracil |
| (E)-vinylphosphonate | 5-(methyl)-4 (thio)pseudouracil |
| (R) 5′-C-methyl | 5-(methyl)isocarbostyrilyl |
| (R) 5′-C-methyl with phosphate | 5-(methyl)pseudouracil |
| (S) 5′-C-methyl | 5-(methylaminomethyl)-2 (thio)uracil |
| (S) 5′-C-methyl with phosphate | 5-(methylaminomethyl)-2,4(dithio)uracil |
| (Z)-5-(2-Bromo-vinyl)ara-uridine | 5-(methylaminomethyl)-4-(thio)uracil |
| (Z)-5-(2-Bromo-vinyl)uridine | 5-(propynyl)uracil |
| 1(4-Nitro-phenyl)pseudouridine | 5-(propynyl)cytosine |
| 1-(aminocarbonylethylenyl)-2(thio)- | 5-(trifluoromethyl)cytosine |
| pseudouracil | |
| 1-(aminocarbonylethylenyl)-2,4- | 5-(trifluoromethyl)uracil |
| (dithio)pseudouracil | |
| 1-(2,2,2-Trifluoroethyl)-pseudouridine | 5,2′-O-dimethylcytidine |
| 1-(2,2,3,3,3-Pentafluoropropyl)pseudouridine | 5,2′-O-dimethyluridine |
| 1-(2,2-Diethoxyethyl)pseudouridine | 5,6-dihydro-uridine |
| 1-(2,4,6-Trimethylbenzyl)pseudouridine | 5-Aminoallyl-cytosine |
| 1-(2,4,6-Trimethyl-benzyl)pseudo-uridine | 5-aminoallyl-uridine |
| 1-(2,4,6-Trimethyl-phenyl)pseudo-uridine | 5-aminomethy1-2-thiouridine |
| 1-(2-Amino-2-carboxyethyl)pseudo-uridine | 5-aza-2-thio-zebularine |
| 1-(2-Amino-ethyl)pseudouridine | 5-aza-cytidine |
| 1-(2-Hydroxyethyl)pseudouridine | 5-aza-uridine |
| 1-(2-Methoxyethyl)pseudouridine | 5-aza-zebularine |
| 1-(3,4-Bis- | 5-bromo-cytidine |
| trifluoromethoxybenzyl)pseudouridine | |
| 1-(3,4-Dimethoxybenzyl)pseudouridine | 5-bromo-uridine |
| 1-(3-Amino-3-carboxypropyl)pseudo-uridine | 5-carbamoylmethyl-2′-O-methyluridine |
| 1-(3-Amino-propyl)pseudouridine | 5-carbamoylmethyluridine |
| 1-(3-Cyclopropyl-prop-2-ynyl)pseudouridine | 5-carboxyhydroxymethyluridine |
| TP | |
| 1-(4-Amino-4-carboxybutyl)pseudouridine | 5-carboxyhydroxymethyluridine methyl ester |
| 1-(4-Amino-benzyl)pseudouridine | 5-carboxymethylaminomethyl-2-thiouridine |
| 1-(4-Amino-butyl)pseudouridine | 5-carboxymethylaminomethyl-2′-O- |
| | methyluridine |
| 1-(4-Amino-phenyl)pseudouridine | 5-carboxymethylaminomethyl-2-thiouridine |
| 1-(4-Azidobenzyl)pseudouridine | 5-carboxymethylaminomethyluridine |
| 1-(4-Bromobenzyl)pseudouridine | 5-carboxymethyluridine |
| 1-(4-Chlorobenzyl)pseudouridine | 5-Cyanocytidine |
| 1-(4-Fluorobenzyl)pseudouridine | 5-Cyanouridine |
| 1-(4-Iodobenzyl)pseudouridine | 5-Dimethylaminouridine |
| 1-(4-Methanesulfonylbenzyl)pseudouridine | 5-Ethynylara-cytidine |
| 1-(4-Methoxybenzyl)pseudouridine | 5-Ethynylcytidine |
| 1-(4-Methoxy-phenyl)pseudouridine | 5-formyl-2′-O-methylcytidine |
| 1-(4-Methylbenzyl)pseudouridine | 5-formylcytidine |
| 1-(4-Nitrobenzyl)pseudouridine | 5′-Homo-adenosine |
| 1-(4-Thiomethoxybenzyl)pseudouridine | 5′-Homo-cytidine |
| 1-(4-Trifluoromethoxybenzyl)pseudouridine | 5′-Homo-guanosine |
| 1-(4-Trifluoromethylbenzyl)pseudouridine | 5′-Homo-uridine |
| 1-(5-Amino-pentyl)pseudouridine | 5-hydroxymethylcytidine |
| 1-(6-Amino-hexyl)pseudouridine | 5-hydroxyuridine |
| 1-(aminoalkylamino-carbonylethylenyl)- | 5-iodo-2′-fluoro-deoxyuridine |
| 2(thio)-pseudouracil | |
| 1-(aminoalkylaminocarbonylethylenyl)-2,4- | 5-iodo-cytidine |
| (dithio)pseudouracil | |
| 1-(aminoalkylaminocarbonylethylenyl)- | 5-iodo-uridine |
| pseudouracil | |
| 1-(aminoalkylaminocarbonylethylenyl)-4- | 5-methoxycarbonylmethy1-2-thiouridine |
| (thio)pseudouracil | |
| 1-(aminocarbonylethylenyl)-4- | 5-methoxycarbonylmethyl-2′-O- |
| (thio)pseudouracil | methyluridine |
| 1-(aminocarbonylethylenyl)-pseudouracil | 5-methoxycarbonylmethyluridine |
| 1-(aza)-2-(thio)-3-(aza)-phenoxazin-1-yl | 5-Methoxycytidine |
| 1,2′-O-dimethyladenosine | 5-methoxyuridine |
| 1,2′-O-dimethylguanosine | 5-methyl-2-thiouridine |
| 1,2′-O-dimethylinosine | 5-methylaminomethyl-2-selenouridine |
| 1,3-(diaza)-2-(oxo)-phenthiazin-1-yl | 5-methylaminomethyl-2-thiouridine |
| 1,3-(diaza)-2-(oxo)-phenoxazin-1-yl | 5-methylaminomethyluridine |
| 1,3,5-(triaza)-2,6-(dioxa)-naphthalene | 5-methylcytidine |
| 1,6-Dimethyl-pseudouridine | 5-Methyldihydrouridine |
| 1-{3-[2-(2-Aminoethoxy)-ethoxy]- | 5-methyluridine |
| propionvl}pseudouridine | |
| 1-Acetylpseudouridine | 5-methyl-zebularine |
| 1-Allylpseudouridine | 5-nitroindole |
| 1-Aminomethyl-pseudo-uridine | 5-Oxyacetic acid- Uridine |
| 1-Benzoylpseudouridine | 5-Oxyacetic acid-methyl ester-Uridin Nl- |
| | methyl-pseudouridine |
| 1-Benzyloxymethylpseudouridine | 5-Phenylethynyluridine |
| 1-Benzyl-pseudo-uridine | 5′-phosphorothioate |
| 1-Biotinylpseudouridine | 5-propynyl cytosine |
| 1-Butyl-pseudo-uridine | 5-propynyl uracil |
| 1-carboxymethyl-pseudouridine | 5-taurinomethyl-2-thiouridine |
| 1-Cyanomethylpseudouridine | 5-taurinomethyluridine |
| 1-Cyclobutylmethyl-pseudo-uridine | 5-Trideuteromethyl-6-deuterouridine |
| 1-Cyclobutyl-pseudo-uridine | 5-Trifluoromethyl-Cytidine |
| 1-Cycloheptylmethyl-pseudo-uridine | 5-Trifluoromethyl-Uridine |
| 1-Cycloheptyl-pseudo-uridine | 5-uracil |
| 1-Cyclohexylmethyl-pseudo-uridine | 5-Vinylarauridine |
| 1-Cyclohexyl-pseudo-uridine | 6 (azo)uracil |
| 1-Cyclooctylmethyl-pseudo-uridine | 6-(2,2,2-Trifluoroethyl)-pseudo-uridine |
| 1-Cyclooctyl-pseudo-uridine | 6-(4-Morpholino)-pseudo-uridine |
| 1-Cyclopentylmethyl-pseudo-uridine | 6-(4-Thiomorpholino)-pseudo-uridine |
| 1-Cyclopentyl-pseudo-uridine | 6-(alkyl)guanine |
| 1-Cyclopropylmethyl-pseudo-uridine | 6-(alkyl)adenine |
| 1-Cyclopropyl-pseudo-uridine | 6-(aza)pyrimidine |
| 1-deazaadenosine | 6-(azo)cytosine |
| 1-Ethyl-pseudo-uridine | 6-(azo)thymine |
| 1-Hexyl-pseudo-uridine | 6-(azo)uracil |
| 1-Homoallylpseudouridine | 6-(methyl)-7-(aza)indolyl |
| 1-Hydroxymethylpseudouridine | 6-(methyl)adenine |
| 1-iso-propyl-pseudo-uridine | 6-(methyl)guanine |
| 1-Me-2-thio-pseudo-uridine | 6-(Substituted-Phenyl)-pseudo-uridine |
| 1-Me-4-thio-pseudo-uridine | 6-Amino-pseudo-uridine |
| 1-Me-alpha-thio-pseudo-uridine | 6-aza-cytidine |
| 1-Me-guanosine | 6-aza-uridine |
| 1-Methanesulfonylmethylpseudouridine | 6-Azido-pseudo-uridine |
| 1-Methoxymethylpseudouridine | 6-Bromo-pseudo-uridine |
| 1-Methyl-6-amino-pseudo-uridine | 6-Butyl-pseudo-uridine |
| 1-Methyl-6-bromo-pseudo-uridine | 6-Chloro-pseudo-uridine |
| 1-Methyl-6-cyano-pseudo-uridine | 6-chloro-purine |
| 1-Methyl-6-hydroxyamino-pseudo-uridine | 6-Cyano-pseudo-uridine |
| 1-Methyl-6-trifluoromethoxy-pseudo-uridine | 6-Dimethylamino-pseudo-uridine |
| 1-methyladenosine | 6-Ethoxy-pseudo-uridine |
| 1-methylguanosine | 6-Ethylcarboxylate-pseudo-uridine |
| 1-methylinosine | 6-Ethyl-pseudo-uridine |
| 1-methylpseduouridine | 6-Fluoro-pseudo-uridine |
| 1-methyl-pseudoisocytidine | 6-Formyl-pseudo-uridine |
| 1-methyl-pseudouridine | 6-Hydroxyamino-pseudo-uridine |
| 1-Methyl-pseudo-UTP | 6-Hydroxy-pseudo-uridine |
| 1-Morpholinomethylpseudouridine | 6-Iodo-pseudo-uridine |
| 1-Pentyl-pseudo-uridine | 6-iso-Propyl-pseudo-uridine |
| 1-Phenyl-pseudo-uridine | 6-methoxy-guanosine |
| 1-Pivaloylpseudouridine | 6-Methoxy-pseudo-uridine |
| 1-Propargylpseudouridine | 6-Methylamino-pseudo-uridine |
| 1-Propyl-pseudo-uridine | 6-methyl-guanosine |
| 1-propynyl-pseudouridine | 6-Methyl-pseudo-uridine |
| 1-propynyl-uridine | 6-Phenyl-pseudo-uridine |
| 1-p-tolyl-pseudo-uridine | 6-phenyl-pyrrolo-pyrimidin-2-on-3-yl |
| 1-substituted 2-(thio)-pseudouracil | 6-Propyl-pseudo-uridine |
| 1-substituted 2,4-(dithio)pseudouracil | 6-tert-Butyl-pseudo- uridine |
| 1-substituted 4-(thio)pseudouracil | 6-thio-7-deaza-8-aza-guanosine |
| 1-substituted pseudouracil | 6-thio-7-deaza-guanosine |
| 1-taurinomethyl-pseudouridine | 6-thio-7-methyl-guanosine |
| 1-tert-Butyl-pseudo-uridine | 6-thio-guanosine |
| 1-Thiomethoxymethylpseudouridine | 6-Trifluoromethoxy-pseudo-uridine |
| 1-Thiomorpholinomethylpseudouridine | 6-Trifluoromethyl-pseudo-uridine |
| 1-Trifluoroacetylpseudouridine | 7-(alkyl)guanine |
| 1-Trifluoromethylpseudouridine | 7-(aminoalkylhydroxy)-1-(aza)-2-(thio)-3- |
| | (aza)-phenthiazin-1-yl |
| 1-Vinylpseudouridine | 7-(aminoalkylhydroxy)-1-(aza)-2-(thio)-3- |
| | (aza)-phenoxazin-1-yl |
| 2-(amino)purine | 7-(aminoalkylhydroxy)-1,3-(diaza)-2-(oxo)- |
| | phenthiazin-1-yl |
| 2-(thio)pseudouracil | 7-(aminoalkylhydroxy)-1,3-(diaza)-2-(oxo)- |
| | phenoxazin-1-yl |
| 2′-alpha-Ethynylcytidine | 7-(aza)indolyl |
| 2′-alpha-Ethynylguanosine | 7-(deaza)adenine |
| 2′-alpha-Ethynyluridine | 7-(deaza)guanine |
| 2′-alpha-Trifluoromethyladenosine | 7-(guanidiniumalkylhydroxy)-1-(aza)-2- |
| | (thio)-3-(aza)-phenoxazinl-yl |
| 2′-alpha-Trifluoromethylguanosine | 7-(guanidiniumalkylhydroxy)-1-(aza)-2- |
| | (thio)-3-(aza)-phenthiazin-1-yl |
| 2′-alpha-Trifluoromethyluridine | 7-(guanidiniumalkylhydroxy)-1-(aza)-2- |
| | (thio)-3-(aza)-phenoxazin-1-yl |
| 2′-Amino-2′-deoxycytosine | 7-(guanidiniumalkyl-hydroxy)-1,3-(diaza)-2- |
| | (oxo)-phenthiazin-1-yl |
| 2′-amino-2′-deoxyribose | 7-(guanidiniumalkylhydroxy)-1,3-(diaza)-2- |
| | (oxo)- phenoxazin-1-yl |
| 2′-alpha-Trifluoromethylcytidine | 7-(methyl)guanine |
| 2′-Azido-2′-deoxycytosine | 7-(propynyl)isocarbostyrilyl |
| 2′-azido-2′-deoxyribose | 7-(propynyl)isocarbostyrilyl |
| 2′-Azido-deoxyuridine | 7-propynyl(aza)indolyl |
| 2′-beta-Ethynyladenosine | 7-aminomethyl-7-deazaguanosine |
| 2′-beta-Ethynylguanosine | 7-cyano-7-deazaguanosine |
| 2′-beta-Ethynyluridine | 7-deaza-2- aminopurine |
| 2′-beta-Trifluoromethyluridine | 7-deaza-2,6-diaminopurine |
| 2′-beta-Ethynylcytidine | 7-deaza-2-amino-purine |
| 2′-bromo-deoxyuridine | 7-deaza-8-aza-2,6-diaminopurine |
| 2′-deoxyuridine | 7-deaza-8-aza-2-aminopurine |
| 2′-Deoxy-2′,2′-difluoroadenosine | 7-deaza-8-aza-adenine |
| 2′-Deoxy-2′,2′-difluorocytidine | 7-deaza-8-aza-adenosine |
| 2′-Deoxy-2′,2′-difluoroguanosine | 7-deaza-8-aza-guanosine |
| 2′-Deoxy-2′,2′-difluorouridine | 7-deaza-adenosine |
| 2′-Deoxy-2′-alpha-aminocytidine | 7-deaza-guanosine |
| 2′-Deoxy-2′-alpha-aminouridine TP | 7-deaza-inosinyl |
| 2′-Deoxy-2′-alpha-azidocytidine | 7-methyl-8-oxo-guanosine |
| 2′-Deoxy-2′-alpha-azidouridine TP | 7-methyladenine |
| 2′-Deoxy-2′-alpha-mercaptoadenosine | 7-methylguanosine |
| 2′-Deoxy-2′-alpha-mercaptocytidine | 7-methylinosine |
| 2′-Deoxy-2′-alpha-mercaptoguanosine | 7-substituted 1-(aza)-2-(thio)-3-(aza)- |
| | phenoxazin-1-yl |
| 2′-Deoxy-2′-alpha-thiomethoxyadenosine | 7-substituted 1,3-(diaza)-2-(oxo)-phenoxazin- |
| | 1-yl |
| 2′-Deoxy-2′-alpha-thiomethoxycytidine | 8-(alkenyl)adenine |
| 2′-Deoxy-2′-alpha-thiomethoxyguanosine | 8-(alkenyl)guanine |
| 2′-Deoxy-2′-alpha-thiomethoxyuridine | 8-(alkyl)adenine |
| 2′-Deoxy-2′-alpha-mercaptouridine | 8-(alkyl)guanine |
| 2′-Deoxy-2′-beta-aminoadenosine | 8-(alkynyl)adenine |
| 2′-Deoxy-2′-beta-aminoguanosine | 8-(alkynyl)guanine |
| 2′-Deoxy-2′-beta-aminouridine | 8-(amino)adenine |
| 2′-Deoxy-2′-beta-azidoadenosine | 8-(amino)guanine |
| 2′-Deoxy-2′-beta-azidocytidine | 8-(halo)adenine |
| 2′-Deoxy-2′-beta-azidoguanosine | 8-(halo)guanine |
| 2′-Deoxy-2′-beta-azidouridine | 8-(hydroxyl)adenine |
| 2′-Deoxy-2′-beta-aminocytidine | 8-(hydroxyl)guanine |
| 2′-Deoxy-2′-beta-bromoadenosine | 8-(thioalkyl)adenine |
| 2′-Deoxy-2′-beta-bromocytidine | 8-(thioalkyl)guanine |
| 2′-Deoxy-2′-beta-bromoguanosine | 8-(thiol)adenine |
| 2′-Deoxy-2′-beta-bromouridine | 8-(thiol)guanine |
| 2′-Deoxy-2′-beta-chloroadenosine | 8-Aza-adenosine |
| 2′-Deoxy-2′-beta-chlorocytidine | 8-azido-adenosine |
| 2′-Deoxy-2′-beta-chloroguanosine | 8-bromo-adenosine |
| 2′-Deoxy-2′-beta-chlorouridine | 8-bromo-guanosine |
| 2′-Deoxy-2′-beta-fluoroadenosine | 8-oxo-guanosine |
| 2′-Deoxy-2′-beta-fluorocytidine | 8-Trifluoromethyladenosine |
| 2′-Deoxy-2′-beta-fluoroguanosine | 9-(methyl)-imidizopyridinyl |
| 2′-Deoxy-2′-beta-fluorouridine | 9-Deazaadenosine |
| 2′-Deoxy-2′-beta-iodoadenosine | 9-Deazaguanosine |
| 2′-Deoxy-2′-beta-iodocytidine | alkene containing backbones |
| 2′-Deoxy-2′-beta-iodoguanosine | alkyl phosphonates |
| 2′-Deoxy-2′-beta-iodouridine | allyamino-thymidine |
| 2′-Deoxy-2′-beta-mercaptoadenosine | allyamino-uracil |
| 2′-Deoxy-2′-beta-mercaptocytidine | alpha-thio-cytidine |
| 2′-Deoxy-2′-beta-mercaptoguanosine | alpha-thio-guanosine |
| 2′-Deoxy-2′-beta-mercaptouridine | alpha-thio-pseudo-uridine |
| 2′-Deoxy-2′-beta-thiomethoxyadenosine | alpha-thio-uridine |
| 2′-Deoxy-2′-beta-thiomethoxycytidine TP | altriol |
| 2′-Deoxy-2′-beta-thiomethoxyuridine | aminoalkylphosphoramidates |
| 2′-deoxyuridine | aminoalkylphosphotriesters |
| 2′-F-5-Methyl-2′-deoxyuridine | aminoindolyl |
| 2′-Fluoro | anthracenyl |
| 2′-fluoro-modified bases | archaeosine |
| 2′-fluorouridine | aza cytosine |
| 2′-methyl, 2′-amino, 2′-azido, 2′-fluoro- | aza thymidine |
| adenine | |
| 2′-methyl, 2′-amino, 2′-azido, 2′-fluroo- | aza uracil |
| cytidine | |
| 2′-OH-ara-adenosine | aza adenine |
| 2′-OH-ara-cytidine | azaguanine |
| 2′-OH-ara-guanosine | bis-ortho-(aminoalkylhydroxy)-6-phenyl- |
| | pyrrolo-nvrimidin-2-on-3-yl |
| 2′-OH-ara-uridine | bis-ortho-substituted-6-phenyl-pyrrolo- |
| | pyrimidin-2-on-3-yl |
| 2′-OMe-2-Aminoadenosine | boranophosphates |
| 2′-OMe-5-Me-uridine | —CH2—O—N(CH3)—CH2— |
| 2′-OMe-pseudouridine | —CH2—N(CH3)—N(CH3)—CH2— |
| 2′-O-Methyl-5-(1-propynyl)cytidine | —CH2—NH—CH2— |
| 2′-O-Methyl-5-(1-propynyl)uridine | chiral phosphonates |
| 2′-O-methyladenosine | chiral phosphorothioates |
| 2′-O-methylation | Constrained nucleic acid (CNA) |
| 2′-O-methylcytidine | deaza cytosine |
| 2′-O-methylguanosine | deaza guanine |
| 2′-O-methylinosine | deaza thymidine |
| 2′-O-methyl-ribose | deaza uracil |
| 2′-O-methyluridine | deazaadenine |
| 2′-O-ribosyladenosine (phosphate) | deoxy-thymidine |
| 2-(alkyl)guanine | difluorotolyl |
| 2-(alkyl)adenine | dihydropseudouridine |
| 2-(amino)adenine | dihydrouridine |
| 2-(aminoalkyl)adenine | DNA |
| 2-(aminopropyl)adenine | epoxyqueuosine |
| 2-(halo)adenine | Fluoro hexitol nucleic acid (FHNA) |
| 2-(methylthio) N6 (isopentenyl)adenine | formacetyl and thioformacetyl backbones |
| 2-(propyl)adenine | Formycin A |
| 2-(propyl)guanine | Formycin B |
| 2-(thio)cytosine | galactosyl-queuosine |
| 2-(thio)uracil | GNA (glycol nucleic acid) |
| 2,2′-anhydro-cytidine | hydroxywybutosine |
| 2,2′-anhydro-uridine | hypoxanthine |
| 2,4-(dithio)pseudouracil | imidizopyridinyl |
| 2,4,5-(trimethyl)phenyl | inosinyl |
| 2,6-(diamino)purine | isocarbostyrilyl |
| 2,6-diaminopurine | isoguanosine |
| 2′-alpha-ethynyladenosine | isopentenyladenosine |
| 2′-Amino-2′-deoxy-guanosine | isowyosme |
| 2′-Amino-2′-deoxy-uridine | 1-Alkyl-6-homoallyl-pseudo-uridine |
| 2-amino-6-Chloro-purine | 1-Methyl-6-(2,2,2-Trifluoroethyl)pseudo- |
| | uridine |
| 2-aminoadenine | 1-Methyl-6-(4-thiomorpholino)-pseudo- |
| | uridine |
| 2-Aminoadenosine | 1-Methyl-6-azido-pseudo-uridine |
| 2-aminopurine | 1-Methyl-6-chloro-pseudo-uridine |
| 2-Amino-riboside | 1-Methyl-6-dimethylamino-pseudo-uridine |
| 2-aza-inosinyl | 1-Methyl-6-ethoxy-pseudo-uridine |
| 2′-azido-2′-deoxyadenosine | 1-Methyl-6-ethylcarboxylate-pseudo-uridine |
| 2′-Azido-2′-deoxy-guanosine | 1-Methyl-6-fluoro-pseudo-uridine |
| 2′-Azido-2′-deoxy-uridine | 1-Methyl-6-hydroxy-pseudo-uridine |
| 2-Azidoadenosine | 1-Methyl-6-iodo-pseudo-uridine |
| 2′-beta-Trifluoromethyladenosine | 1-Methyl-6-methylamino-pseudo-uridine |
| 2′-beta-Trifluoromethylguanosine | 1-[3-(2-{2-[2-(2-Aminoethoxy)-ethoxy]- |
| | ethoxy}-ethoxy)-propionyl]pseudouridine |
| 2-Bromoadenosine | 1-Alkyl-6-(1-propynyl)-pseudo-uridine |
| 2′-beta-Trifluoromethylcytidine | 1-Alkyl-6-(2-propynyl)-pseudo-uridine |
| 2-Chloroadenosine | 1-Alkyl-6-allyl-pseudo-uridine |
| 2′-Deoxy-2′-alpha-aminoadenosine | 1-Alkyl-6-ethynyl-pseudo-uridine |
| 2′-Deoxy-2′-alpha-aminoguanosine | 1-Alkyl-6-vinyl-pseudo-uridine |
| 2′-Deoxy-2′-alpha-azidoadenosine | 1-Biotinyl-PEG2-pseudouridine |
| 2′-Deoxy-2′-alpha-azidoguanosine | 1-methyl-1-deaza-pseudoisocytidine |
| 2′-Deoxy-2′-beta-thiomethoxyguanosine | 1-methyl-1-deaza-pseudouridine |
| 2′-Fluor-N4-Bz-cytidine | 1-Methyl-3-(3-amino-3- |
| | carboxyproovl)pseudo-Uridine |
| 2′-fluoro-2′-deoxyribose | 1-Methyl-3-(3-amino-3-carboxypropyl) |
| | pseudouridine |
| 2-Fluoroadenosine | 1-methyl-3-(3-amino-5- |
| | carboxypropyl)pseudouridine |
| 2′-Fluoro-N2-isobutyl-guanosine | 1-Methyl-6-(4-morpholino)-pseudo-uridine |
| 2′-Fluoro-N4-Acetyl-cytidine | 1-Methyl-6-(substituted phenyl)pseudo- |
| | uridine |
| 2′-Fluoro-N6-Bz-deoxyadenosine | 1-Methyl-6-butyl-pseudo-uridine |
| 2-Iodoadenosine | 1-Methyl-6-ethyl-pseudo-uridine |
| 2-Mercaptoadenosine | 1-Methyl-6-formyl-pseudo-uridine |
| 2-methoxy-4-thio-pseudouridine | 1-Methyl-6-iso-propyl-pseudo-uridine |
| 2-methoxy-4-thio-uridine | 1-Methyl-6-methoxy-pseudo-uridine |
| 2-methoxy-5-methyl-cytidine | 1-Methyl-6-phenyl-pseudo-uridine |
| 2-methoxy-adenine | 1-Methyl-6-propyl-pseudo-uridine |
| 2-methoxy-cytidine | 1-Methyl-6-tert-butyl-pseudo-uridine |
| 2-methoxyuridine | 1-methyl-6-thio-guanosine |
| 2′-methyl, 2′-amino, 2′-azido, 2′-fluro- | 1-Methyl-6-trifluoromethyl-pseudo-uridine |
| guanosine | |
| 2′-methyl, 2′-amino, 2′-azido, 2′fluro-uridine | Locked nucleic acid (LNA) |
| 2-methyladenosine | 1-taurinomethyl-1-methyl-uridine |
| 2-methylpseudouridine | 1-taurinomethyl-4-thio-uridine |
| 2-methylthioadenine | lysidine |
| 2-methylthio-N6 isopentenyladenosine | mannosyl-queuosine |
| 2-methylthio-N6-(cis- | Methyl phosphonate |
| hydroxyisopentenyl)adenosine | |
| 2-methylthio-N6-hydroxynorvalyl | methylene (methylimino) |
| carbamoyladenosine | |
| 2-methylthio-N6-isopentenyladenosine | methylene formacetyl and thioformacetyl |
| | backbones |
| 2-methylthio-N6-methyladenosine | methyleneimino and methylenehydrazino |
| | backbones |
| 2-methylthio-N6-threonyl | methylphosphonates |
| carbamoyladenosine | |
| 2′-O-methoxyethyl (MOE) | methylwyosine |
| 2′-O-methoxyethylribose (MOE) | morpholino linkages |
| 2′-O-methyl | mosme |
| 2′-O-methyladenosine | N (methyl)guanine |
| 2′-O-methylcytidine | —N(CH3)—CH2—CH2— |
| 2′-O-methylguanosine | N-(methyl)guanine |
| 2′-O-methylinosine | N2,7,2′-O-trimethylguanosine |
| 2′O-methyl-N2-isobutyl-guanosine | N2,2′-O-dimethylguanosine |
| 2′-O-Methyl-N4-Acetyl-cytidine | N2,7-dimethylguanosine |
| 2′-O-methyl-N4-Bz-cytidine | N2,N2,2′-O-trimethylguanosine |
| 2′-O-methyl-N6-Bz-deoxyadenosine | N2,N2,7-trimethylguanosine |
| 2′-O-methylpseudouridine | N2,N2-dimethyl-6-thio-guanosine |
| 2′-O-methyluridine | N2,N2-dimethylguanosine |
| 2′-O-ribosyladenosine (phosphate) | N2-isobutyl-guanosine |
| 2′-O-ribosylguanosine (phosphate) | N2-methyl-6-thio-guanosine |
| 2-oxo-7-aminopyridopyrimidin-3-yl | N2-methylguanosine |
| 2-oxo-pyridopyrimidine-3-yl | N2-substituted purines |
| 2-pyridinone | N3 (methyl)uracil |
| 2-thio-1-methyl-1-deaza-pseudouridine | N4 (acetyl)cytosine |
| 2-thio-1-methyl-pseudouridine | N4,2′-O-dimethylcytidine |
| 2-thio-2′-O-methyluridine | N4,N4-Dimethyl-2′-OMe-Cytidine |
| 2-thio-5-aza-uridine | N4-acetyl-2′-O-methylcytidine |
| 2-thio-5-methyl-cytidine | N4-acetylcytidine |
| 2-thiocytidine | N4-Amino-cytidine |
| 2-thio-dihydropseudouridine | N4-Benzoyl-cytidine |
| 2-thio-dihydrouridine | N4-methylcytidine |
| 2-thio-pseudouridine | N6-(19-Amino-pentaoxanonadecyl)adenosine |
| 2-thiouridine | N6-(cis-hydroxyisopentenyl)adenosine |
| 2-thio-zebularine | N6-(isopentyl)adenine |
| 2-Trifluoromethyladenosine | N6-(methyl)adenine |
| 3-(deaza)-5-(aza)cytosine | N6,N6 (dimethyl)adenine |
| 3-(methyl)cytosine | N6,2′-O-dimethyladenosine |
| 3-nitropyrrole | N6,N6,2′-O-trimethyladenosine |
| 3-(3-amino-3-carboxypropyl)uracil | N6,N6-dimethyladenosine |
| 3-(3-amino-3-carboxypropyl)uridine | N6-acetyladenosine |
| 3-(alkyl)cytosine | N6-cis-hydroxy-isopentenyl-adenosine |
| 3-(methyl)-7-(propynyl)isocarbostyrilyl | N6-glycinylcarbamoyladenosine |
| 3-(methyl)cytidine | N6-hydroxynorvalylcarbamoyladenosine |
| 3-(methyl)isocarbostyrilyl | N6-isopentenyladenosine |
| 3,2′-0-dimethyluridine | N6-methyl-2-amino-purine |
| 3′-alkylene phosphonates | N6-methyladenosine |
| 3-alkyl-pseudouridine | N6-methyl-N6-threonylcarbamoyladenosine |
| 3′-aminophosphoramidate | N6-substituted purines |
| 3-deaza-3-bromoadenosine | N6-threonylcarbamoyladenosine |
| 3-deaza-3-chloroadenosine | N-alkylated derivative |
| 3-deaza-3-fluoroadenosine | napthalenyl |
| 3-deaza-3-iodoadenosine | nitrobenzimidazolyl |
| 3-deazaadenosine | nitroimidazolyl |
| 3′-ethynylcytidine | nitroindazolyl |
| 3-methylcytidine | nitropyrazolyl |
| 3-methyl-pseudouridine | N1-methyl-adenosine |
| 3-methyluridine | N1-methyl-guanosine |
| 4′-azidoadenosine | nubularine |
| 4′-azidouridine | O6-substituted purines |
| 4′-ethynyladenosine | O-alkylated derivative |
| 4′-ethynylcytidine | oligonucleosides with heteroatom |
| | internucleoside linkage |
| 4′-ethynylguanosine | ortho-(aminoalkylhydroxy)-6-phenyl-pyrrolo- |
| | pyrimidin-2-on-3-yl |
| 4′-ethynyluridine | ortho-substituted-6-phenyl-pyrrolo-pyrimidin- |
| | 2-on-3-yl |
| 4-(fluoro)-6-(methyl)benzimidazole | Oxoformycin TP |
| 4-(methyl)benzimidazole | para-(aminoalkylhydroxy)-6-phenyl-pyrrolo- |
| | pyrimidin-2-on-3-yl |
| 4-(methyl)indolyl | para-substituted-6-phenyl-pyrrolo-pyrimidin- |
| | 2-on-3-yl |
| 4-(thio)pseudouracil | pentacenyl |
| 4-(thio)uracil | peroxywybutosine |
| 4,2′-O-dimethylcytidine | phenanthracenyl |
| 4,6-(dimethyl)indolyl | phenyl |
| 4′-azidocytidine | phosphinates |
| 4′-azidoguanosine | phosphonoacetates |
| 4′-carbocyclic adenosine | phosphoramidates |
| 4′-carbocyclic cytidine | Phosphorodiamidate Morpholino Oligomer |
| | (PMO) |
| 4′-carbocyclic guanosine | phosphorodithioates |
| 4′-carbocyclic uridine | Phosphorothioate |
| 4-demethylwyosine | phosphorothioate internucleoside linkages |
| 4-methoxy-1-methyl-pseudoisocytidine | phosphorothioates |
| 4-methoxy-2-thio-pseudouridine | phosphotriesters |
| 4-methoxy-pseudoisocytidine | PNA |
| 4-methoxy-pseudouridine | propynyl-7-(aza)indolyl |
| 4-methylcytidine | pseudoisocytidine |
| 4-thio-1-methyl-1-deaza-pseudoisocytidine | Pseudo-iso-cytidine |
| 4-thio-1-methyl-pseudoisocytidine | pseudouracil |
| 4-thio-1-methyl-pseudouridine | pseudouridine |
| 4-thio-pseudoisocytidine | Pseudouridine 1-(4-methylbenzenesulfonic |
| | acid) |
| 4-thio-pscudouridinc | Pseudouridinc 1-(4-methylbenzoic acid) TP |
| 4-thiouracil | Pseudouridine 1-methylphosphonic acid |
| 4-thiouridine | Pseudouridine 1-[3-(2-ethoxy)]propionic acid |
| 5 (halo)cytosine | Pseudouridine 1-[3-{2-(2-[2-(2-ethoxy)- |
| | ethoxy]-ethoxy)-ethoxy}]propionic acid |
| 5 (methyl) 4 (thio)uracil | Pseudouridine 1-[3-{2-(2-[2-{2(2-ethoxy)- |
| | ethoxy }-ethoxy]-ethoxy)-ethoxy}propionic |
| | acid |
| 5 (methyl)cytosine | Pseudouridine 1-[3-{2-(2-[2-ethoxy]-ethoxy)- |
| | ethoxv}]propionic acid |
| 5 (methylaminomethyl)-2 (thio)uracil | Pseudouridine 1-[3-{2-(2-ethoxy)-ethoxv}] |
| | propionic acid |
| 5 (methylaminomethyl)-2,4 (dithio)uracil | Pseudouridine TP 1-methylphosphonic acid |
| | diethyl ester |
| 5 (methylaminomethyl)-4 (thio)uracil | Pseudo-uridine-1-2-ethanoic acid |
| 5 (propynyl)cytosine | Pseudo-uridine-N1-5-pentanoic acid |
| 5 (propynyl)uracil | Pseudo-uridine-N1-3-propionic acid |
| 5 (trifluoromethyl)cytosine | Pseudo-uridine-N1-4-butanoic acid |
| 5 (trifluoromethyl)uracil | Pseudo-uridine-N1-6-hexanoic acid |
| 5 nitroindole | Pseudo-uridine-N1-methy 1-p-benzoic acid |
| 5 substituted pyrimidines | Pseudo-uridine-N1-p-benzoic acid |
| 5-(1,3-diazole-1-alkyl)uracil | Pseudo-uridine-N1-7-heptanoic acid |
| 5-(1-Propynyl)ara-cytidine | pyrenyl |
| 5-(1-Propynyl)ara-uridine | pyridin-4-one ribonucleoside |
| 5-(2-aminopropyl)uracil | pyridopyrimidin-3-yl |
| 5-(2-carbomethoxyvinyl)uridine | pyridopyrimidin-3-y1, 2-oxo-7-amino- |
| | pyridopyrimidin-3-yl |
| 5-(2-Chloro-phenyl)-2-thiocytidine | pyrrolo-cytidine |
| 5-(2-Furanyl)uridine | pyrrolo-pseudoisocytidine |
| 5-(4-Amino-phenyl)-2-thiocytidine | pyrrolo-pyrimidin-2-on-3-yl |
| 5-(alky1)-2-(thio)pseudouracil | pyrrolopyrimidinyl |
| 5-(alky1)-4 (thio)pseudouracil | pyrrolopyrizinyl |
| 5-(alkyl)-2,4 (dithio)pseudouracil | Pyrrolosine |
| 5-(alkyl)cytosine | siloxane backbones |
| 5-(alkyl)pseudouracil | stilbenzyl |
| 5-(alkyl)uracil | substituted 1,2,4-triazoles |
| 5-(alkynyl)cytosine | sulfamate backbones |
| 5-(alkynyl)uracil | sulfide sulfoxide and sulfone backbones |
| 5-(allylamino)uracil | sulfonate and sulfonamide backbones |
| 5-(aminoalkyl)uracil | tetracenyl |
| 5-(carboxyhydroxymethyl)uridine | thio-adenosine |
| 5-(carboxyhydroxymethyl)uridine methyl | thionoalkylphosphonates |
| ester | |
| 5-(cyanoalkyl)uracil | thionoalkylphosphotriesters |
| 5-(dialkylaminoalkyl)uracil | thionophosphoramidates |
| 5-(dimethylaminoalkyl)uracil | Tricyclo-DNA (tcDNA) |
| 5-(guanidiniumalkyl)uracil | tubercidine |
| 5-(halo)cytosine | undermodified hydroxywybutosine |
| 5-(halo)uracil | uridine 5-oxyacetic acid |
| 5-(iso-Pentenylaminomethyl)-2-thiouridine | uridine 5-oxyacetic acid methyl ester |
| 5-(iso-Pentenylaminomethyl)-2′-O- | wybutosine |
| methyluridine | |
| 5-(iso-Pentenylaminomethyl)uridine | wyosme |
| 5-(1,3-diazole-1-alkyl)uracil | xanthine |
| 5-(methoxy)uracil | Xanthosine |
| 5-(methoxycarbonylmethyl)-2-(thio)uracil | zebularine |

### TREM, TREM Core Fragment and TREM Fragment Fusions

In an embodiment, a TREM, a TREM core fragment or a TREM fragment disclosed herein comprises an additional moiety, e.g., a fusion moiety. In an embodiment, the fusion moiety can be used for purification, to alter folding of the TREM, TREM core fragment or TREM fragment, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM, TREM core fragment or TREM fragment. In an embodiment, the fusion moiety can be encoded by the same or different nucleic acid molecule that encodes the TREM, TREM core fragment or TREM fragment.

### TREM Consensus Sequence

In an embodiment, a TREM disclosed herein comprises a consensus sequence provided herein.

In an embodiment, a TREM disclosed herein comprises a consensus sequence of Formula I_{ZZZ}, wherein ZZZ indicates any of the twenty amino acids and Formula I corresponds to all species.

In an embodiment, a TREM disclosed herein comprises a consensus sequence of Formula II_{ZZZ}, wherein ZZZ indicates any of the twenty amino acids and Formula II corresponds to mammals.

In an embodiment, a TREM disclosed herein comprises a consensus sequence of Formula III_{ZZZ}, wherein ZZZ indicates any of the twenty amino acids and Formula III corresponds to humans.

In an embodiment, ZZZ indicates any of the twenty amino acids: alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In an embodiment, a TREM disclosed herein comprises a property selected from the following:
a) under physiological conditions residue R₀ forms a linker region, e.g., a Linker 1 region;
b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;
c) under physiological conditions residues R₈-R₉ forms a linker region, e.g., a Linker 2 region;
d) under physiological conditions residues -R₁₀-R₁₁-R₁₂-R₁₃-R₁₄, R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region;
e) under physiological conditions residue -R₂₉ forms a linker region, e.g., a Linker 3 Region;
f) under physiological conditions residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;
g) under physiological conditions residue -[R₄₇]ₓ comprises a variable region, e.g., as described herein;
h) under physiological conditions residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or
i) under physiological conditions residue R₇₂ forms a linker region, e.g., a Linker 4 region.

### Alanine TREM Consensus Sequence

In an embodiment, a TREM disclosed herein comprises the sequence of Formula I_{ALA} (SEQ ID NO: 562),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂, wherein R is a ribonucleotide residue and the consensus for Ala is: R₀=absent; R₁₄, R₅₇=are independently A or absent; R₂₆=A, C, G or absent; R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₈, R₅₉, R₆₃, R₆₄, R₆₆, R₆₇=are independently N or absent; R₁₁, R₃₅, R₆₅=are independently A, C, U or absent; R₁, R₉, R₂₀, R₃₈, R₄₀, R₅₁, R₅₂, R₅₆=are independently A, G or absent; R₇, R₂₂, R₂₅, R₂₇, R₂₉, R₄₆, R₅₃, R₇₂=are independently A, G, U or absent; R₂₄, R₆₉=are independently A, U or absent; R₇₀, R₇₁=are independently C or absent; R₃, R₄=are independently C, G or absent; R₁₂, R₃₃, R₃₆, R₆₂, R₆₈=are independently C, G, U or absent; R₁₃, R₁₇, R₂₈, R₃₉, R₅₅, R₆₀, R₆₁=are independently C, U or absent; R₁₀, R₁₉, R₂₃=are independently G or absent; R₂=G, U or absent; R₈, R₁₈, R₅₄=are independently U or absent; [R₄₇]ₓ=N or absent; wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271),
provided that the TREM has one or both of the following properties: no more than 15% of the residues are N; or no more than 20 residues are absent.

In an embodiment, a TREM disclosed herein comprises the sequence of Formula II_{ALA} (SEQ ID NO: 563),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Ala is:
R₀, R₁₈=are absent;
R₁₄, R₂₄, R₅₇=are independently A or absent;
R₁₅, R₂₆, R₆₄=are independently A, C, G or absent;
R₁₆, R₃₁, R₅₀, R₅₉=are independently N or absent;
R₁₁, R₃₂, R₃₇, R₄₁, R₄₃, R₄₅, R₄₉, R₆₅, R₆₆=are independently A, C, U or absent;
R₁, R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃, R₇₂=are independently A, G, U or absent;
R₆, R₃₅, R₆₉=are independently A, U or absent;
R₅₅, R₆₀, R₇₀, R₇₁=are independently C or absent;
R₃=C, G or absent;
R₁₂, R₃₆, R₄₈=are independently C, G, U or absent;
R₁₃, R₁₇, R₂₅, R₃₀, R₃₄, R₃₉, R₅₈, R₆₁, R₆₂, R₆₇, R₆₈=are independently C, U or absent;
R₄, R₁₀, R₁₉, R₂₀, R₂₃, R₅₂=are independently G or absent;
R₂, R₈, R₃₃=are independently G, U or absent;
R₂₁, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ALA}(SEQ ID NO: 564),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Ala is:
R₀, R₁₈=are absent;
R₁₄, R₂₄, R₅₇, R₇₂=are independently A or absent;
R₁₅, R₂₆, R₆₄=are independently A, C, G or absent;
R₁₆, R₃₁, R₅₀=are independently N or absent;
R₁₁, R₃₂, R₃₇, R₄₁, R₄₃, R₄₅, R₄₉, R₆₅, R₆₆=are independently A, C, U or absent;
R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃=are independently A, G, U or absent;
R₆, R₃₅=are independently A, U or absent;
R₅₅, R₆₀, R₆₁, R₇₀, R₇₁=are independently C or absent;
R₁₂, R₄₈, R₅₉=are independently C, G, U or absent;
R₁₃, R₁₇, R₂₅, R₃₀, R₃₄, R₃₉, R₅₈, R₆₂, R₆₇, R₆₈=are independently C, U or absent;
R₁, R₂, R₃, R₄, R₁₀, R₁₉, R₂₀, R₂₃, R₅₂=are independently G or absent;
R₃₃, R₃₆=are independently G, U or absent;
R₈, R₂₁, R₅₄, R₆₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Arginine TREM Consensus Sequence

In an embodiment, a TREM disclosed herein comprises the sequence of Formula I_{ARG} (SEQ ID NO: 565),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Arg is:
R₅₇=A or absent;
R₉, R₂₇=are independently A, C, G or absent;
R₁, R₂, R₃, R₄, R₅, R₆, R₇, R₁₁, R₁₂, R₁₆, R₂₁, R₂₂, R₂₃, R₂₅, R₂₆, R₂₉, R₃₀, R₃₁, R₃₂, R₃₃, R₃₄, R₃₇, R₄₂, R₄₄, R₄₅,
R₄₆, R₄₈, R₄₉, R₅₀, R₅₁, R₅₈, R₆₂, R₆₃, R₆₄, R₆₅, R₆₆, R₆₇, R₆₈, R₆₉, R₇₀, R₇₁=are independently N or
absent;
R₁₃, R₁₇, R₄₁=are independently A, C, U or absent;
R₁₉, R₂₀, R₂₄, R₄₀, R₅₆=are independently A, G or absent;
R₁₄, R₁₈, R₇₂=are independently A, G, U or absent;
R₁₈=A, U or absent;
R₃₈=C or absent;
R₃₅, R₄₃, R₆₁=are independently C, G, U or absent;
R₂₈, R₅₅, R₅₉, R₆₀=are independently C, U or absent;
R₀, R₁₀, R₅₂=are independently G or absent;
R₈, R₃₉=are independently G, U or absent;
R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ARG} (SEQ ID NO: 566),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Arg is:
R₁₈=absent;
R₂₄, R₅₇=are independently A or absent;
R₄₁=A, C or absent;
R₃, R₇, R₃₄, R₅₀=are independently A, C, G or absent;
R₂, R₅, R₆, R₁₂, R₂₆, R₃₂, R₃₇, R₄₄, R₅₈, R₆₆, R₆₇, R₆₈, R₇₀=are independently N or absent;
R₄₉, R₇₁=are independently A, C, U or absent;
R₁, R₁₅, R₁₉, R₂₅, R₂₇, R₄₀, R₄₅, R₄₆, R₅₆, R₇₂=are independently A, G or absent;
R₁₄, R₂₉, R₆₃=are independently A, G, U or absent;
R₁₆, R₂₁=are independently A, U or absent;
R₃₈, R₆₁=are independently C or absent;
R₃₃, R₄₈=are independently C, G or absent;
R₄, R₉, R₁₁, R₄₃, R₆₂, R₆₄, R₆₉=are independently C, G, U or absent;
R₁₃, R₂₂, R₂₈, R₃₀, R₃₁, R₃₅, R₅₅, R₆₀, R₆₅=are independently C, U or absent;
R₀, R₁₀, R₂₀, R₂₃, R₅₁, R₅₂=are independently G or absent;
R₈, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ARG (SEQ ID NO: 567),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Arg is:
R₁₈=is absent;
R₁₅, R₂₁, R₂₄, R₄₁, R₅₇=are independently A or absent;
R₃₄, R₄₄=are independently A, C or absent;
R₃, R₅, R₅₈=are independently A, C, G or absent;
R₂, R₆, R₆₆, R₇₀=are independently N or absent;
R₃₇, R₄₉=are independently A, C, U or absent;
R₁, R₂₅, R₂₉, R₄₀, R₄₅, R₄₆, R₅₀=are independently A, G or absent;
R₁₄, R₆₃, R₆₈=are independently A, G, U or absent;
R₁₆=A, U or absent;
R₃₈, R₆₁=are independently C or absent;
R₇, R₁₁, R₁₂, R₂₆, R₄₈=are independently C, G or absent;
R₆₄, R₆₇, R₆₉=are independently C, G, U or absent;
R₄, R₁₃, R₂₂, R₂₈, R₃₀, R₃₁, R₃₅, R₄₃, R₅₅, R₆₀, R₆₂, R₆₅, R₇₁=are independently C, U or absent;
R₀, R₁₀, R₁₉, R₂₀, R₂₃, R₂₇, R₃₃, R₅₁, R₅₂, R₅₆, R₇₂=are independently G or absent;
R₈, R₉, R₃₂, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Asparagine TREM Consensus Sequence

In an embodiment, a TREM disclosed herein comprises the sequence of Formula I_{ASN} (SEQ ID NO: 568),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Asn is:
R₀, R₁₈=are absent;
R₄₁=A or absent;
R₁₄, R₄₈, R₅₆=are independently A, C, G or absent;
R₂, R₄, R₅, R₆, R₁₂, R₁₇, R₂₆, R₂₉, R₃₀, R₃₁, R₄₄, R₄₅, R₄₆, R₄₉, R₅₀, R₅₈, R₆₂, R₆₃, R₆₅, R₆₆, R₆₇, R₆₈, R₇₀, R₇₁=are independently N or absent;
R₁₁, R₁₃, R₂₂, R₄₂, R₅₅, R₅₉=are independently A, C, U or absent;
R₉, R₁₅, R₂₄, R₂₇, R₃₄, R₃₇, R₅₁, R₇₂=are independently A, G or absent;
R₁, R₇, R₂₅, R₆₉=are independently A, G, U or absent;
R₄₀, R₅₇=are independently A, U or absent;
R₆₀=C or absent;
R₃₃=C, G or absent;
R₂₁, R₃₂, R₄₃, R₆₄=are independently C, G, U or absent;
R₃, R₁₆, R₂₈, R₃₅, R₃₆, R₆₁=are independently C, U or absent;
R₁₀, R₁₉, R₂₀, R₅₂=are independently G or absent;
R₅₄=G, U or absent;
R₈, R₂₃, R₃₈, R₃₉, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ASN}(SEQ ID NO: 569),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Asn is:
R₀, R₁₈=are absent
R₂₄, R₄₁, R₄₆, R₆₂=are independently A or absent;
R₅₉=A, C or absent;
R₁₄, R₅₆, R₆₆=are independently A, C, G or absent;
R₁₇, R₂₉=are independently N or absent;
R₁₁, R₂₆, R₄₂, R₅₅=are independently A, C, U or absent;
R₁, R₉, R₁₂, R₁₅, R₂₅, R₃₄, R₃₇, R₄₈, R₅₁, R₆₇, R₆₈, R₆₉, R₇₀, R₇₂=are independently A, G or absent;
R₄₄, R₄₅, R₅₈=are independently A, G, U or absent;
R₄₀, R₅₇=are independently A, U or absent;
R₅, R₂₈, R₆₀=are independently C or absent;
R₃₃, R₆₅=are independently C, G or absent;
R₂₁, R₄₃, R₇₁=are independently C, G, U or absent;
R₃, R₆, R₁₃, R₂₂, R₃₂, R₃₅, R₃₆, R₆₁, R₆₃, R₆₄=are independently C, U or absent;
R₇, R₁₀, R₁₉, R₂₀, R₂₇, R₄₉, R₅₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ASN (SEQ ID NO: 570),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₅-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Asn is:
R₀, R₁₈=are absent
R₂₄, R₄₀, R₄₁, R₄₆, R₆₂=are independently A or absent;
R₅₉=A, C or absent;
R₁₄, R₅₆, R₆₆=are independently A, C, G or absent;
R₁₁, R₂₆, R₄₂, R₅₅=are independently A, C, U or absent;
R₁, R₉, R₁₂, R₁₅, R₃₄, R₃₇, R₄₈, R₅₁, R₆₇, R₆₈, R₆₉, R₇₀=are independently A, G or absent;
R₄₄, R₄₅, R₅₈=are independently A, G, U or absent;
R₅₇=A, U or absent;
R₅, R₂₈, R₆₀=are independently C or absent;
R₃₃, R₆₅=are independently C, G or absent;
R₁₇, R₂₁, R₂₉=are independently C, G, U or absent;
R₃, R₆, R₁₃, R₂₂, R₃₂, R₃₅, R₃₆, R₄₃, R₆₁, R₆₃, R₆₄, R₇₁=are independently C, U or absent;
R₇, R₁₀, R₁₉, R₂₀, R₂₅, R₂₇, R₄₉, R₅₂, R₇₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Aspartate TREM Consensus Sequence

In an embodiment, a TREM disclosed herein comprises the sequence of Formula I_{ASP} (SEQ ID NO: 571),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Asp is:
R₀=absent
R₂₄, R₇₁=are independently A, C or absent;
R₃₃, R₄₆=are independently A, C, G or absent;
R₂, R₃, R₄, R₅, R₆, R₁₂, R₁₆, R₂₂, R₂₆, R₂₉, R₃₁, R₃₂, R₄₄, R₄₅, R₄₉, R₅₈, R₆₃, R₆₄, R₆₆, R₆₇, R₆₈, R₆₉=are independently N or absent;
R₁₃, R₂₁, R₃₄, R₄₁, R₅₇, R₆₅=are independently A, C, U or absent;
R₉, R₁₀, R₁₄, R₁₅, R₂₀, R₂₇, R₃₇, R₄₀, R₅₁, R₅₆, R₇₂=are independently A, G or absent;
R₇, R₂₅, R₄₂=are independently A, G, U or absent;
R₃₉=C or absent;
R₅₀, R₆₂=are independently C, G or absent;
R₃₀, R₄₃, R₄₅, R₅₅, R₇₀=are independently C, G, U or absent;
R₈, R₁₁, R₁₇, R₁₈, R₂₈, R₃₅, R₅₃, R₅₉, R₆₀, R₆₁=are independently C, U or absent;
R₁₉, R₅₂=are independently G or absent;
R₁=G, U or absent;
R₂₃, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ASP} (SEQ ID NO: 572),
R₀-R₁-R₂-R₃-R₄-R₅-R₆-R₇-R₈-R₉-R₁₀-R₁₁-R₁₂-R₁₃-R₁₄-R₁₅-R₁₀-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Asp is:
R₀, R₁₇, R₁₈, R₂₃=are independently absent;
R₉, R₄₀=are independently A or absent;
R₂₄, R₇₁=are independently A, C or absent;
R₆₇, R₆₈=are independently A, C, G or absent;
R₂, R₆, R₆₆=are independently N or absent;
R₅₇, R₆₃=are independently A, C, U or absent;
R₁₀, R₁₄, R₂₇, R₃₃, R₃₇, R₄₄, R₄₆, R₅₁, R₅₆, R₆₄, R₇₂=are independently A, G or absent;
R₇, R₁₂, R₂₆, R₆₅=are independently A, U or absent;
R₃₉, R₆₁, R₆₂=are independently C or absent;
R₃, R₃₁, R₄₅, R₇₀=are independently C, G or absent;
R₄, R₅, R₂₉, R₄₃, R₅₅=are independently C, G, U or absent;
R₈, R₁₁, R₁₃, R₃₀, R₃₂, R₃₄, R₃₅, R₄₁, R₄₈, R₅₃, R₅₉, R₆₀=are independently C, U or absent;
R₁₅, R₁₉, R₂₀, R₂₅, R₄₂, R₅₀, R₅₂=are independently G or absent;
R₁, R₂₂, R₄₉, R₅₈, R₆₉=are independently G, U or absent;
R₁₆, R₂₁, R₂₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ASP} (SEQ ID NO: 573),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Asp is:
R₀, R₁₇, R₁₈, R₂₃=are absent
R₉, R₁₂, R₄₀, R₆₅, R₇₁=are independently A or absent;
R₂, R₂₄, R₅₇=are independently A, C or absent;
R₆, R₁₄, R₂₇, R₄₆, R₅₁, R₅₆, R₆₄, R₆₇, R₆₈=are independently A, G or absent;
R₃, R₃₁, R₃₅, R₃₉, R₆₁, R₆₂=are independently C or absent;
R₆₆=C, G or absent;
R₅, R₈, R₂₉, R₃₀, R₃₂, R₃₄, R₄₁, R₄₃, R₄₈, R₅₅, R₅₉, R₆₀, R₆₃=are independently C, U or absent;
R₁₀, R₁₅, R₁₉, R₂₀, R₂₅, R₃₃, R₃₇, R₄₂, R₄₄, R₄₅, R₄₉, R₅₀, R₅₂, R₆₉, R₇₀, R₇₂=are independently G or absent;
R₂₂, R₅₈=are independently G, U or absent;
R₁, R₄, R₇, R₁₁, R₁₃, R₁₆, R₂₁, R₂₆, R₂₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Cysteine TREM Consensus Sequence

In an embodiment, a TREM disclosed herein comprises the sequence of Formula I_{CYS} (SEQ ID NO: 574),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Cys is:
R₀=absent
R₁₄, R₃₉, R₅₇=are independently A or absent;
R₄₁=A, C or absent;
R₁₀, R₁₅, R₂₇, R₃₃, R₆₂=are independently A, C, G or absent;
R₃, R₄, R₅, R₆, R₁₂, R₁₃, R₁₆, R₂₄, R₂₆, R₂₉, R₃₀, R₃₁, R₃₂, R₃₄, R₄₂, R₄₄, R₄₅, R₄₆, R₄₃, R₄₉, R₅₈, R₆₃, R₆₄, R₆₆, R₆₇, R₆₈, R₆₉, R₇₀=are independently N or absent;
R₆₅=A, C, U or absent;
R₉, R₂₅, R₃₇, R₄₀, R₅₂, R₅₆=are independently A, G or absent;
R₇, R₂₀, R₅₁=are independently A, G, U or absent;
R₁₈, R₃₈, R₅₅=are independently C or absent;
R₂=C, G or absent;
R₂₁, R₂₈, R₄₃, R₅₀=are independently C, G, U or absent;
R₁₁, R₂₂, R₂₃, R₃₅, R₃₆, R₅₉, R₆₀, R₆₁, R₇₁, R₇₂=are independently C, U or absent;
R₁, R₁₉=are independently G or absent;
R₁₇=G, U or absent;
R₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{CYS} (SEQ ID NO: 575),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Cys is:
R₀, R₁₈, R₂₃=are absent;
R₁₄, R₂₄, R₂₆, R₂₉, R₃₉, R₄₁, R₄₅, R₅₇=are independently A or absent;
R₄₄=A, C or absent;
R₂₇, R₆₂=are independently A, C, G or absent;
R₁₆=A, C, G, U or absent;
R₃₀, R₇₀=are independently A, C, U or absent;
R₅, R₇, R₉, R₂₅, R₃₄, R₃₇, R₄₀, R₄₆, R₅₂, R₅₆, R₅₈, R₆₆=are independently A, G or absent;
R₂₀, R₅₁=are independently A, G, U or absent;
R₃₅, R₃₈, R₄₃, R₅₅, R₆₉=are independently C or absent;
R₂, R₄, R₁₅=are independently C, G or absent;
R₁₃=C, G, U or absent;
R₆, R₁₁, R₂₈, R₃₆, R₄₈, R₄₉, R₅₀, R₆₀, R₆₁, R₆₇, R₆₈, R₇₁, R₇₂=are independently C, U or absent;
R₁, R₃, R₁₀, R₁₉, R₃₃, R₆₃=are independently G or absent; R₈, R₁₇, R₂₁, R₆₄=are independently G, U or absent;
R₁₂, R₂₂, R₃₁, R₃₂, R₄₂, R₅₃, R₅₄, R₆₅=are independently U or absent;
R₅₉=U, or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{CYS} (SEQ ID NO: 576),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Cys is:
R₀, R₁₈, R₂₃=are absent
R₁₄, R₂₄, R₂₆, R₂₉, R₃₄, R₃₉, R₄₁, R₄₅, R₅₇, R₅₈=are independently A or absent;
R₄₄, R₇₀=are independently A, C or absent;
R₆₂=A, C, G or absent;
R₁₆=N or absent;
R₅, R₇, R₉, R₂₀, R₄₀, R₄₆, R₅₁, R₅₂, R₅₆, R₆₆=are independently A, G or absent;
R₂₈, R₃₅, R₃₈, R₄₃, R₅₅, R₆₇, R₆₉=are independently C or absent;
R₄, R₁₅=are independently C, G or absent;
R₆, R₁₁, R₁₃, R₃₀, R₄₈, R₄₉, R₅₀, R₆₀, R₆₁, R₆₈, R₇₁, R₇₂=are independently C, U or absent;
R₁, R₂, R₃, R₁₀, R₁₉, R₂₅, R₂₇, R₃₃, R₃₇, R₆₃=are independently G or absent;
R₈, R₂₁, R₆₄=are independently G, U or absent;
R₁₂, R₁₇, R₂₂, R₃₁, R₃₂, R₃₆, R₄₂, R₅₃, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamine TREM Consensus Sequence

In an embodiment, a TREM disclosed herein comprises the sequence of Formula I_{GLN} (SEQ ID NO: 577),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Gln is:
R₀, R₁₈=are absent;
R₁₄, R₂₄, R₅₇=are independently A or absent;
R₉, R₂₆, R₂₇, R₃₃, R₅₆=are independently A, C, G or absent;
R₂, R₄, R₅, R₆, R₁₂, R₁₃, R₁₆, R₂₁, R₂₂, R₂₅, R₂₉, R₃₀, R₃₁, R₃₂, R₃₄, R₄₁, R₄₂, R₄₄, R₄₅, R₄₆, R₄₈, R₄₉, R₅₀, R₅₈, R₆₂, R₆₃, R₆₆, R₆₇, R₆₈, R₆₉, R₇₀=are independently N or absent;
R₁₇, R₂₃, R₄₃, R₆₈, R₇₁=are independently A, C, U or absent;
R₁₅, R₄₀, R₅₁, R₅₂=are independently A, G or absent;
R₁, R₇, R₇₂=are independently A, G, U or absent;
R₃, R₁₁, R₃₇, R₆₀, R₆₄=are independently C, G, U or absent;
R₂₉, R₃₅, R₅₅, R₅₉, R₆₁=are independently C, U or absent;
R₁₀, R₁₉, R₂₀=are independently G or absent;
R₃₉=G, U or absent;
R₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLN} (SEQ ID NO: 578),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Gln is:
R₀, R₁₈, R₂₃=are absent
R₁₄, R₂₄, R₅₇=are independently A or absent;
R₁₇, R₇₁=are independently A, C or absent;
R₂₅, R₂₆, R₃₃, R₄₄, R₄₆, R₅₆, R₆₉=are independently A, C, G or absent;
R₄, R₅, R₁₂, R₂₂, R₂₉, R₃₀, R₄₈, R₄₉, R₆₃, R₆₇, R₆₈=are independently N or absent;
R₃₁, R₄₃, R₆₂, R₆₅, R₇₀=are independently A, C, U or absent;
R₁₅, R₂₇, R₃₄, R₄₀, R₄₁, R₅₁, R₅₂=are independently A, G or absent;
R₂, R₇, R₂₁, R₄₅, R₅₀, R₅₈, R₆₆, R₇₂=are independently A, G, U or absent;
R₃, R₁₃, R₃₂, R₃₇, R₄₂, R₆₀, R₆₄=are independently C, G, U or absent;
R₆, R₁₁, R₂₈, R₃₅, R₅₈, R₅₉, R₆₁=are independently C, U or absent;
R₉, R₁₀, R₁₉, R₂₀=are independently G or absent;
R₁, R₁₆, R₃₉=are independently G, U or absent;
R₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLN} (SEQ ID NO: 579),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Gln is:
R₀, R₁, R₂₃=are absent
R₁₄, R₂₄, R₄₁, R₅₇=are independently A or absent;
R₁₇, R₇₁=are independently A, C or absent;
R₅, R₂₅, R₂₆, R₄₆, R₅₆, R₆₉=are independently A, C, G or absent;
R₄, R₂₂, R₂₉, R₃₀, R₄₈, R₄₉, R₆₃, R₆₈=are independently N or absent;
R₄₃, R₆₂, R₆₈, R₇₀=are independently A, C, U or absent;
R₁₅, R₂₇, R₃₃, R₃₄, R₄₀, R₅₁, R₅₂=are independently A, G or absent;
R₂, R₇, R₁₂, R₄₅, R₅₀, R₅₈, R₆₆=are independently A, G, U or absent;
R₃₁=A, U or absent;
R₃₂, R₄₄, R₆₀=are independently C, G or absent;
R₃, R₁₃, R₃₇, R₄₂, R₆₄, R₆₇=are independently C, G, U or absent;
R₆, R₁₁, R₂₈, R₃₅, R₅₅, R₅₉, R₆₁=are independently C, U or absent;
R₉, R₁₀, R₁₉, R₂₀=are independently G or absent;
R₁, R₂₁, R₃₉, R₇₂=are independently G, U or absent;
R₈, R₁₆, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamate TREM Consensus Sequence

In an embodiment, a TREM disclosed herein comprises the sequence of Formula I_{GLU} (SEQ ID NO: 580),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Glu is:
R₀=absent;
R₃₄, R₄₃, R₆₈, R₆₉=are independently A, C, G or absent;
R₁, R₂, R₅, R₆, R₉, R₁₂, R₁₆, R₂₀, R₂₁, R₂₆, R₂₇, R₂₉, R₃₀, R₃₁, R₃₂, R₃₃, R₄₁, R₄₄, R₄₅, R₄₆, R₄₈, R₅₀, R₅₁, R₅₈, R₆₃, R₆₄, R₆₅, R₆₆, R₇₀, R₇₁=are independently N or absent;
R₁₃, R₁₇, R₂₃, R₆₁=are independently A, C, U or absent;
R₁₀, R₁₄, R₂₄, R₄₀, R₅₂, R₅₆=are independently A, G or absent;
R₇, R₁₅, R₂₅, R₆₇, R₇₂=are independently A, G, U or absent;
R₁₁, R₅₇=are independently A, U or absent;
R₃₉=C, G or absent;
R₃, R₄, R₂₂, R₄₂, R₄₉, R₅₅, R₆₂=are independently C, G, U or absent;
R₁₈, R₂₈, R₃₅, R₃₇, R₅₃, R₅₉, R₆₀=are independently C, U or absent;
R₁₉=G or absent;
R₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II GLU (SEQ ID NO: 581),
R₀-R₁-R₂-R₃-R₄-R₅-R₆-R₇-R₈-R₉-R₁₀-R₁₁-R₁₂-R₁₃-R₁₄-R₁₈-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R&4-R₆₅-R₆₆-R₆₇-R₆₈-R₆₉-R₇₀-R₇₁-R₇₂
wherein R is a ribonucleotide residue and the consensus for Glu is:
R₀, R₁₈, R₂₃=are absent
R₁₇, R₄₀=are independently A or absent;
R₂₆, R₂₇, R₃₄, R₄₃, R₆₈, R₆₉, R₇₁=are independently A, C, G or absent;
R₁, R₂, R₅, R₁₂, R₂₁, R₃₁, R₃₃, R₄₁, R₄₅, R₄₈, R₅₁, R₅₈, R₆₆, R₇₀=are independently N or absent;
R₄₄, R₆₁=are independently A, C, U or absent;
R₉, R₁₄, R₂₄, R₂₅, R₅₂, R₅₆, R₆₃=are independently A, G or absent;
R₇, R₁₅, R₄₆, R₅₀, R₆₇, R₇₂=are independently A, G, U or absent;
R₂₉, R₅₇=are independently A, U or absent;
R₆₀=C or absent;
R₃₉=C, G or absent;
R₃, R₆, R₂₀, R₃₀, R₃₂, R₄₂, R₅₅, R₆₂, R₆₅=are independently C, G, U or absent;
R₄, R₈, R₁₆, R₂₈, R₃₅, R₃₇, R₄₉, R₅₃, R₅₉=are independently C, U or absent;
R₁₀, R₁₉=are independently G or absent;
R₂₂, R₆₄=are independently G, U or absent;
R₁₁, R₁₃, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLU} (SEQ ID NO: 582),
R₀-R₁-R₂-R₃-R₄-R₅-R₆-R₇-R₈-R₉-R₁₀-R₁₁-R₁₂-R₁₃-R₁₄-R₁₅-R₁₀-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Glu is:
R₀, R₁₇, R₁₈, R₂₃=are absent
R₁₄, R₂₇, R₄₀, R₇₁=are independently A or absent;
R₄₄=A, C or absent;
R₄₃=A, C, G or absent;
R₁, R₃₁, R₃₃, R₄₅, R₅₁, R₆₆=are independently N or absent;
R₂₁, R₄₁=are independently A, C, U or absent;
R₇, R₂₄, R₂₅, R₅₀, R₅₂, R₅₆, R₆₃, R₆₈, R₇₀=are independently A, G or absent;
R₅, R₄₆=are independently A, G, U or absent;
R₂₉, R₅₇, R₆₇, R₇₂=are independently A, U or absent;
R₂, R₃₉, R₆₀=are independently C or absent;
R₃, R₁₂, R₂₀, R₂₆, R₃₄, R₆₉=are independently C, G or absent;
R₆, R₃₀, R₄₂, R₄₈, R₆₅=are independently C, G, U or absent;
R₄, R₁₆, R₂₈, R₃₅, R₃₇, R₄₉, R₅₃, R₅₅, R₅₈, R₆₁, R₆₂=are independently C, U or absent;
R₉, R₁₀, R₁₉, R₆₄=are independently G or absent;
R₁₅, R₂₂, R₃₂=are independently G, U or absent;
R₈, R₁₁, R₁₃, R₃₆, R₃₈, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glycine TREM Consensus Sequence

In an embodiment, a TREM disclosed herein comprises the sequence of Formula I_{GLY} (SEQ ID NO: 583),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Gly is:
R₀=absent;
R₂₄=A or absent;
R₃, R₉, R₄₀, R₅₀, R₅₁=are independently A, C, G or absent;
R₄, R₅, R₆, R₇, R₁₂, R₁₆, R₂₁, R₂₂, R₂₆, R₂₉, R₃₀, R₃₁, R₃₂, R₃₃, R₃₄, R₄₁, R₄₂, R₄₃, R₄₄, R₄₅, R₄₆, R₄₈, R₄₉, R₅₈, R₆₃, R₆₄, R₆₅, R₆₆, R₆₇, R₆₈=are independently N or absent;
R₅₉=A, C, U or absent;
R₁, R₁₀, R₁₄, R₁₈, R₂₇, R₅₆=are independently A, G or absent;
R₂₀, R₂₅=are independently A, G, U or absent;
R₅₇, R₇₂=are independently A, U or absent;
R₃₈, R₃₉, R₆₀=are independently C or absent;
R₅₂=C, G or absent;
R₂, R₁₉, R₃₇, R₅₄, R₅₅, R₆₁, R₆₂, R₆₉, R₇₀=are independently C, G, U or absent;
R₁₁, R₁₃, R₁₇, R₂₈, R₃₅, R₃₆, R₇₁=are independently C, U or absent;
R₈, R₁₈, R₂₃, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLY} (SEQ ID NO: 584),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₈-R₅₇-R₅₈-R₅₉-R₆₀-R₆₁-R₂-R₆₃-R₆₄-R₆₅-R₆₆-R₆₇-R₆₈-R₆₉-R₇₀-R₇₁-R₇₂
wherein R is a ribonucleotide residue and the consensus for Gly is:
R₀, R₁₈, R₂₃=are absent
R₂₄, R₂₇, R₄₀, R₇₂=are independently A or absent;
R₂₆=A, C or absent;
R₃, R₇, R₆₈=are independently A, C, G or absent;
R₅, R₃₀, R₄₁, R₄₂, R₄₄, R₄₉, R₆₇=are independently A, C, G, U or absent;
R₃₁, R₃₂, R₃₄=are independently A, C, U or absent;
R₉, R₁₀, R₁₄, R₁₅, R₃₃, R₅₀, R₅₆=are independently A, G or absent;
R₁₂, R₁₆, R₂₂, R₂₅, R₂₉, R₄₆=are independently A, G, U or absent;
R₅₇=A, U or absent;
R₁₇, R₃₈, R₃₉, R₆₀, R₆₁, R₇₁=are independently C or absent;
R₆, R₅₂, R₆₄, R₆₈=are independently C, G or absent;
R₂, R₄, R₃₇, R₄₈, R₅₅, R₆₅=are independently C, G, U or absent;
R₁₃, R₃₅, R₄₃, R₆₂, R₆₉=are independently C, U or absent;
R₁, R₁₉, R₂₀, R₅₁, R₇₀=are independently G or absent;
R₂₁, R₄₅, R₆₃=are independently G, U or absent;
R₈, R₁₁, R₂₈, R₃₆, R₅₃, R₅₄, R₅₈, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLY} (SEQ ID NO: 585),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Gly is:
R₀, R₁₈, R₂₃=are absent
R₂₄, R₂₇, R₄₀, R₇₂=are independently A or absent;
R₂₆=A, C or absent;
R₃, R₇, R₄₉, R₆₈=are independently A, C, G or absent;
R₅, R₃₀, R₄₁, R₄₄, R₆₇=are independently N or absent;
R₃₁, R₃₂, R₃₄=are independently A, C, U or absent;
R₉, R₁₀, R₁₄, R₁₅, R₃₃, R₅₀, R₅₆=are independently A, G or absent;
R₁₂, R₂₅, R₂₉, R₄₂, R₄₆=are independently A, G, U or absent;
R₁₆, R₅₇=are independently A, U or absent;
R₁₇, R₃₈, R₃₉, R₆₀, R₆₁, R₇₁=are independently C or absent;
R₆, R₅₂, R₆₄, R₆₆=are independently C, G or absent;
R₃₇, R₄₈, R₆₅=are independently C, G, U or absent;
R₂, R₄, R₁₃, R₃₅, R₄₃, R₅₅, R₆₂, R₆₉=are independently C, U or absent;
R₁, R₁₉, R₂₀, R₅₁, R₇₀=are independently G or absent;
R₂₁, R₂₂, R₄₅, R₆₃=are independently G, U or absent;
R₈, R₁₁, R₂₈, R₃₆, R₅₃, R₅₄, R₅₈, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Histidine TREM Consensus Sequence

In an embodiment, a TREM disclosed herein comprises the sequence of Formula I_{HIS} (SEQ ID NO: 586),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for His is:
R₂₃=absent;
R₁₄, R₂₄, R₅₇=are independently A or absent;
R₇₂=A, C or absent;
R₉, R₂₇, R₄₃, R₄₈, R₆₉=are independently A, C, G or absent;
R₃, R₄, R₅, R₆, R₁₂, R₂₅, R₂₆, R₂₉, R₃₀, R₃₁, R₃₄, R₄₂, R₄₅, R₄₆, R₄₉, R₅₀, R₅₈, R₆₂, R₆₃, R₆₆, R₆₇, R₆₈=are independently N or absent;
R₁₃, R₂₁, R₄₁, R₄₄, R₆₅=are independently A, C, U or absent;
R₄₀, R₅₁, R₅₆, R₇₀=are independently A, G or absent;
R₇, R₃₂=are independently A, G, U or absent;
R₅₅, R₆₀=are independently C or absent;
R₁₁, R₁₆, R₃₃, R₆₄=are independently C, G, U or absent;
R₂, R₁₇, R₂₂, R₂₈, R₃₅, R₅₃, R₅₉, R₆₁, R₇₁=are independently C, U or absent;
R₁, R₁₀, R₁₅, R₁₉, R₂₀, R₃₇, R₃₉, R₅₂=are independently G or absent;
R₀=G, U or absent;
R₈, R₁₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II HIS (SEQ ID NO: 587),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for His is:
R₀, R₁₇, R₁₈, R₂₃=are absent;
R₇, R₁₂, R₁₄, R₂₄, R₂₇, R₄₅, R₅₇, R₅₈, R₆₃, R₆₇, R₇₂=are independently A or absent;
R₃=A, C, U or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₂₅=C, G or absent;
R₉=C, G, U or absent;
R₈, R₁₃, R₂₆, R₃₃, R₃₅, R₅₀, R₅₃, R₆₁, R₆₈=are independently C, U or absent;
R₁, R₆, R₁₀, R₁₅, R₁₉, R₂₀, R₃₂, R₃₄, R₃₇, R₃₉, R₄₀, R₄₆, R₅₁, R₅₂, R₆₂, R₆₄, R₆₉=are independently G or absent;
R₁₆=G, U or absent;
R₅, R₁₁, R₂₁, R₂₂, R₂₉, R₃₁, R₃₆, R₃₈, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{His} (SEQ ID NO: 588),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for His is:
R₀, R₁₇, R₁₈, R₂₃=are absent
R₇, R₁₂, R₁₄, R₂₄, R₂₇, R₄₅, R₅₇, R₅₈, R₆₃, R₆₇, R₇₂=are independently A or absent;
R₃=A, C or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₈, R₉, R₂₆, R₃₃, R₃₅, R₅₀, R₆₁, R₆₈=are independently C, U or absent;
R₁, R₆, R₁₀, R₁₅, R₁₉, R₂₀, R₂₅, R₃₂, R₃₄, R₃₇, R₃₉, R₄₀, R₄₆, R₅₁, R₅₂, R₆₂, R₆₄, R₆₉=are independently G or absent;
R₅, R₁₁, R₁₃, R₁₆, R₂₁, R₂₂, R₂₉, R₃₁, R₃₆, R₃₈, R₅₃, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Isoleucine TREM Consensus Sequence

In an embodiment, a TREM disclosed herein comprises the sequence of Formula I_{ILE} (SEQ ID NO: 589),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Ile is:
R₂₃=absent;
R₃₈, R₄₁, R₅₇, R₇₂=are independently A or absent;
R₁, R₂₆=are independently A, C, G or absent;
R₀, R₃, R₄, R₆, R₁₆, R₃₁, R₃₂, R₃₄, R₃₇, R₄₂, R₄₃, R₄₄, R₄₅, R₄₆, R₄₈, R₄₉, R₅₀, R₅₈, R₅₉, R₆₂, R₆₃, R₆₄, R₆₆, R₆₇, R₆₈, R₆₉=are independently N or absent;
R₂₂, R₆₁, R₆₅=are independently A, C, U or absent;
R₉, R₁₄, R₁₅, R₂₄, R₂₇, R₄₀=are independently A, G or absent;
R₇, R₂₅, R₂₉, R₅₁, R₅₆=are independently A, G, U or absent;
R₁₈, R₅₄=are independently A, U or absent;
R₆₀=C or absent;
R₂, R₅₂, R₇₀=are independently C, G or absent;
R₅, R₁₂, R₂₁, R₃₀, R₃₃, R₇₁=are independently C, G, U or absent;
R₁₁, R₁₃, R₁₇, R₂₈, R₃₅, R₅₃, R₅₅=are independently C, U or absent;
R₁₀, R₁₉, R₂₀=are independently G or absent;
R₈, R₃₆, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ILE} (SEQ ID NO: 590),
R₀-R₁-R₂-R₃-R₄-R₅-R₆-R₇-R₈-R₉-R₁₀-R₁₁-R₁₂-R₁₃-R₁₄-R₁₈-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Ile is:
R₀, R₁₈, R₂₃=are absent
R₂₄, R₃₈, R₄₀, R₄₁, R₅₇, R₇₂=are independently A or absent;
R₂₆, R₆₅=are independently A, C or absent;
R₅₈, R₅₉, R₆₇=are independently N or absent;
R₂₂=A, C, U or absent;
R₆, R₉, R₁₄, R₁₅, R₂₉, R₃₄, R₄₃, R₄₆, R₄₈, R₅₀, R₅₁, R₆₃, R₆₉=are independently A, G or absent;
R₃₇, R₅₆=are independently A, G, U or absent;
R₅₄=A, U or absent;
R₂₈, R₃₅, R₆₀, R₆₂, R₇₁=are independently C or absent;
R₂, R₅₂, R₇₀=are independently C, G or absent;
R₅=C, G, U or absent;
R₃, R₄, R₁₁, R₁₃, R₁₇, R₂₁, R₃₀, R₄₂, R₄₄, R₄₅, R₄₉, R₅₃, R₅₅, R₆₁, R₆₄, R₆₆=are independently C, U or absent;
R₁, R₁₀, R₁₉, R₂₀, R₂₅, R₂₇, R₃₁, R₆₈=are independently G or absent;
R₇, R₁₂, R₃₂=are independently G, U or absent;
R₈, R₁₆, R₃₃, R₃₆, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ILE} (SEQ ID NO: 591),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Ile is:
R₀, R₁₈, R₂₃=are absent
R₁₄, R₂₄, R₃₈, R₄₀, R₄₁, R₅₇, R₇₂=are independently A or absent;
R₂₆, R₆₅=are independently A, C or absent;
R₂₂, R₅₉=are independently A, C, U or absent;
R₆, R₉, R₁₅, R₃₄, R₄₃, R₄₆, R₅₁, R₅₆, R₆₃, R₆₉=are independently A, G or absent;
R₃₇=A, G, U or absent;
R₁₃, R₂₈, R₃₅, R₄₄, R₅₅, R₆₀, R₆₂, R₇₁=are independently C or absent;
R₂, R₅, R₇₀=are independently C, G or absent;
R₅₈, R₆₇=are independently C, G, U or absent;
R₃, R₄, R₁₁, R₁₇, R₂₁, R₃₀, R₄₂, R₄₅, R₄₉, R₅₃, R₆₁, R₆₄, R₆₆=are independently C, U or absent;
R₁, R₁₀, R₁₉, R₂₀, R₂₅, R₂₇, R₂₉, R₃₁, R₃₂, R₄₈, R₅₀, R₅₂, R₆₈=are independently G or absent;
R₇, R₁₂=are independently G, U or absent;
R₈, R₁₆, R₃₃, R₃₆, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Methionine TREM Consensus Sequence

In an embodiment, a TREM disclosed herein comprises the sequence of Formula I_{MET} (SEQ ID NO: 592),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Met is:
R₀, R₂₃=are absent;
R₁₄, R₃₈, R₄₀, R₅₇=are independently A or absent;
R₆₀=A, C or absent;
R₃₃, R₄₈, R₇₀=are independently A, C, G or absent;
R₁, R₃, R₄, R₅, R₆, R₁₁, R₁₂, R₁₆, R₁₇, R₂₁, R₂₂, R₂₆, R₂₇, R₂₉, R₃₀, R₃₁, R₃₂, R₄₂, R₄₄, R₄₅, R₄₆, R₄₉, R₅₀, R₅₈, R₆₂, R₆₃, R₆₆, R₆₇, R₆₈, R₆₉, R₇₁=are independently N or absent;
R₁₈, R₃₅, R₄₁, R₅₉, R₆₅=are independently A, C, U or absent;
R₉, R₁₅, R₅₁=are independently A, G or absent;
R₇, R₂₄, R₂₅, R₃₄, R₅₃, R₅₆=are independently A, G, U or absent;
R₇₂=A, U or absent;
R₃₇=C or absent;
R₁₀, R₅₅=are independently C, G or absent;
R₂, R₁₃, R₂₈, R₄₃, R₆₄=are independently C, G, U or absent;
R₃₆, R₆₁=are independently C, U or absent;
R₁₉, R₂₀, R₅₂=are independently G or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{MET} (SEQ ID NO: 593),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Met is:
R₀, R₁₈, R₂₂, R₂₃=are absent
R₁₄, R₂₄, R₃₈, R₄₀, R₄₁, R₅₇, R₇₂=are independently A or absent;
R₅₉, R₆₀, R₆₂, R₆₅=are independently A, C or absent;
R₆, R₄₅, R₆₇=are independently A, C, G or absent;
R₄=N or absent;
R₂₁, R₄₂=are independently A, C, U or absent;
R₁, R₉, R₂₇, R₂₉, R₃₂, R₄₆, R₅₁=are independently A, G or absent;
R₁₇, R₄₉, R₅₃, R₅₆, R₅₈=are independently A, G, U or absent;
R₆₃=A, U or absent;
R₃, R₁₃, R₃₇=are independently C or absent;
R₄₈, R₅₅, R₆₄, R₇₀=are independently C, G or absent;
R₂, R₅, R₆₆, R₆₈=are independently C, G, U or absent;
R₁₁, R₁₆, R₂₆, R₂₈, R₃₀, R₃₁, R₃₅, R₃₆, R₄₃, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀=are independently G, U or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{MET} (SEQ ID NO: 594),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₈-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Met is:
R₀, R₁₈, R₂₂, R₂₃=are absent
R₁₄, R₂₄, R₃₈, R₄₀, R₄₁, R₅₇, R₇₂=are independently A or absent;
R₅₉, R₆₂, R₆₅=are independently A, C or absent;
R₆, R₆₇=are independently A, C, G or absent;
R₄, R₂₁=are independently A, C, U or absent;
R₁, R₉, R₂₇, R₂₉, R₃₂, R₄₅, R₄₆, R₅₁=are independently A, G or absent;
R₁₇, R₅₆, R₅₈=are independently A, G, U or absent;
R₄₉, R₅₃, R₆₃=are independently A, U or absent;
R₃, R₁₃, R₂₆, R₃₇, R₄₃, R₆₀=are independently C or absent;
R₂, R₄₈, R₅₅, R₆₄, R₇₀=are independently C, G or absent;
R₅, R₆₆=are independently C, G, U or absent;
R₁₁, R₁₆, R₂₈, R₃₀, R₃₁, R₃₅, R₃₆, R₄₂, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀, R₆₈=are independently G, U or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Leucine TREM Consensus Sequence

In an embodiment, a TREM disclosed herein comprises the sequence of Formula I_{LEU} (SEQ ID NO: 595),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Leu is:
R₀=absent;
R₃₈, R₅₇=are independently A or absent;
R₆₀=A, C or absent;
R₁, R₁₃, R₂₇, R₄₈, R₅₁, R₅₆=are independently A, C, G or absent;
R₂, R₃, R₄, R₅, R₆, R₇, R₉, R₁₀, R₁₁, R₁₂, R₁₆, R₂₃, R₂₆, R₂₈, R₂₉, R₃₀, R₃₁, R₃₂, R₃₃, R₃₄, R₃₇, R₄₁, R₄₂, R₄₃, R₄₄, R₄₅, R₄₆, R₄₉, R₅₀, R₅₈, R₆₂, R₆₃, R₆₅, R₆₆, R₆₇, R₆₈, R₆₉, R₇₀=are independently N or absent;
R₁₇, R₁₈, R₂₁, R₂₂, R₂₅, R₃₅, R₅₅=are independently A, C, U or absent;
R₁₄, R₁₅, R₃₉, R₇₂=are independently A, G or absent;
R₂₄, R₄₀=are independently A, G, U or absent;
R₅₂, R₆₁, R₆₄, R₇₁=are independently C, G, U or absent;
R₃₆, R₅₃, R₅₉=are independently C, U or absent;
R₁₉=G or absent;
R₂₀=G, U or absent;
R₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{LEU} (SEQ ID NO: 596),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R₅₀, R₅₆, R₆₉=are independently A, C, G or absent;
R₆, R₃₃, R₄₁, R₄₃, R₄₆, R₄₉, R₅₈, R₆₃, R₆₆, R₇₀=are independently N or absent;
R₁₁, R₁₂, R₁₇, R₂₁, R₂₂, R₂₈, R₃₁, R₃₇, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₂₆, R₃₀, R₅₂, R₆₂, R₆₄, R₆₅, R₆₇, R₆₈=are independently C, G, U or absent;
R₁₈, R₃₅, R₄₂, R₅₃, R₅₉, R₆₁, R₇₁=are independently C, U or absent;
R₁₉, R₅₁=are independently G or absent;
R₁₀, R₂₀=are independently G, U or absent;
R₈, R₂₃, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{LEU} (SEQ ID NO: 597),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R₅₀, R₅₆, R₅₈, R₆₉=are independently A, C, G or absent;
R₆, R₃₃, R₄₃, R₄₆, R₄₉, R₆₃, R₆₆, R₇₀=are independently N or absent;
R₁₁, R₁₂, R₁₇, R₂₁, R₂₂, R₂₈, R₃₁, R₃₇, R₄₁, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₃₀, R₅₂, R₆₂, R₆₄, R₆₇, R₆₈=are independently C, G, U or absent;
R₁₈, R₃₅, R₄₂, R₅₃, R₅₉, R₆₁, R₆₅, R₇₁=are independently C, U or absent;
R₁₉, R₅₁=are independently G or absent;
R₁₀, R₂₀, R₂₆=are independently G, U or absent;
R₈, R₂₃, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Lysine TREM Consensus Sequence

In an embodiment, a TREM disclosed herein comprises the sequence of Formula I_{LYS} (SEQ ID NO: 598),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Lys is:
R₀=absent
R₁₄=A or absent;
R₄₀, R₄₁=are independently A, C or absent;
R₃₄, R₄₃, R₅₁=are independently A, C, G or absent;
R₁, R₂, R₃, R₄, R₅, R₆, R₇, R₁₁, R₁₂, R₁₆, R₂₁, R₂₆, R₃₀, R₃₁, R₃₂, R₄₄, R₄₅, R₄₆, R₄₈, R₄₉, R₅₀, R₅₈, R₆₂, R₆₃, R₆₅, R₆₆, R₆₇, R₆₈, R₆₉, R₇₀=are independently N or absent;
R₁₃, R₁₇, R₅₉, R₇₁=are independently A, C, U or absent;
R₉, R₁₅, R₁₉, R₂₀, R₂₅, R₂₇, R₅₂, R₅₆=are independently A, G or absent;
R₂₄, R₂₉, R₇₂=are independently A, G, U or absent;
R₁₈, R₅₇=are independently A, U or absent;
R₁₀, R₃₃=are independently C, G or absent;
R₄₂, R₆₁, R₆₄=are independently C, G, U or absent;
R₂₈, R₃₅, R₃₆, R₃₇, R₅₃, R₅₅, R₆₀=are independently C, U or absent;
R₈, R₂₂, R₂₃, R₃₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{LYS} (SEQ ID NO: 599),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Lys is:
R₀, R₁₈, R₂₃=are absent
R₁₄=A or absent;
R₄₀, R₄₁, R₄₃=are independently A, C or absent;
R₃, R₇=are independently A, C, G or absent;
R₁, R₆, R₁₁, R₃₁, R₄₅, R₄₈, R₄₉, R₆₃, R₆₅, R₆₆, R₆₈=are independently N or absent;
R₂, R₁₂, R₁₃, R₁₇, R₄₄, R₆₇, R₇₁=are independently A, C, U or absent;
R₉, R₁₅, R₁₉, R₂₀, R₂₅, R₂₇, R₃₄, R₅₀, R₅₂, R₅₆, R₇₀, R₇₂=are independently A, G or absent;
R₅, R₂₄, R₂₆, R₂₉, R₃₂, R₄₆, R₆₉=are independently A, G, U or absent;
R₅₇=A, U or absent;
R₁₀, R₆₁=are independently C, G or absent;
R₄, R₁₆, R₂₁, R₃₀, R₅₈, R₆₄=are independently C, G, U or absent;
R₂₈, R₃₅, R₃₆, R₃₇, R₄₂, R₅₃, R₅₅, R₅₉, R₆₀, R₆₂=are independently C, U or absent;
R₃₃, R₅₁=are independently G or absent;
R₈=G, U or absent;
R₂₂, R₃₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{LYS} (SEQ ID NO: 600),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Lys is:
R₀, R₁₈, R₂₃=absent
R₉, R₁₄, R₃₄, R₄₁=are independently A or absent;
R₄₀=A, C or absent;
R₁, R₃, R₇, R₃₁=are independently A, C, G or absent;
R₄₈, R₆₅, R₆₈=are independently N or absent;
R₂, R₁₃, R₁₇, R₄₄, R₆₃, R₆₆=are independently A, C, U or absent;
R₅, R₁₅, R₁₉, R₂₀, R₂₅, R₂₇, R₂₉, R₅₀, R₅₂, R₅₆, R₇₀, R₇₂=are independently A, G or absent;
R₆, R₂₄, R₃₂, R₄₉=are independently A, G, U or absent;
R₁₂, R₂₆, R₄₆, R₅₇=are independently A, U or absent;
R₁₁, R₂₈, R₃₅, R₄₃=are independently C or absent;
R₁₀, R₄₅, R₆₁=are independently C, G or absent;
R₄, R₂₁, R₆₄=are independently C, G, U or absent;
R₃₇, R₅₃, R₅₅, R₅₉, R₆₀, R₆₂, R₆₇, R₇₁=are independently C, U or absent;
R₃₃, R₅₁=are independently G or absent;
R₈, R₃₀, R₅₈, R₆₉=are independently G, U or absent;
R₁₆, R₂₂, R₃₆, R₃₈, R₃₉, R₄₂, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Phenylalanine TREM Consensus Sequence

In an embodiment, a TREM disclosed herein comprises the sequence of Formula I_{PHE} (SEQ ID NO: 601),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Phe is:
R₀, R₂₃=are absent
R₉, R₁₄, R₃₈, R₃₉, R₅₇, R₇₂=are independently A or absent;
R₇₁=A, C or absent;
R₄₁, R₇₀=are independently A, C, G or absent;
R₄, R₅, R₆, R₃₀, R₃₁, R₃₂, R₃₄, R₄₂, R₄₄, R₄₅, R₄₆, R₄₈, R₄₉, R₅₈, R₆₂, R₆₃, R₆₆, R₆₇, R₆₈, R₆₉=are independently N or absent;
R₁₆, R₆₁, R₆₅=are independently A, C, U or absent; R₁₅, R₂₆, R₂₇, R₂₉, R₄₀, R₅₆=are independently A, G or absent;
R₇, R₅₁=are independently A, G, U or absent;
R₂₂, R₂₄=are independently A, U or absent;
R₅₅, R₆₀=are independently C or absent;
R₂, R₃, R₂₁, R₃₃, R₄₃, R₅₀, R₆₄=are independently C, G, U or absent;
R₁₁, R₁₂, R₁₃, R₁₇, R₂₈, R₃₅, R₃₆, R₅₉=are independently C, U or absent;
R₁₀, R₁₉, R₂₀, R₂₅, R₃₇, R₅₂=are independently G or absent;
R₁=G, U or absent;
R₈, R₁₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{PHE} (SEQ ID NO: 602),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₈-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Phe is:
R₀, R₁₈, R₂₃=absent
R₁₄, R₂₄, R₃₈, R₃₉, R₅₇, R₇₂=are independently A or absent;
R₄₆, R₇₁=are independently A, C or absent;
R₄, R₇₀=are independently A, C, G or absent;
R₄₅=A, C, U or absent;
R₆, R₇, R₁₅, R₂₆, R₂₇, R₃₂, R₃₄, R₄₀, R₄₁, R₅₆, R₆₉=are independently A, G or absent;
R₂₉=A, G, U or absent;
R₅, R₉, R₆₇=are independently A, U or absent;
R₃₅, R₄₉, R₅₈, R₆₀=are independently C or absent;
R₂₁, R₄₃, R₆₂=are independently C, G or absent;
R₂, R₃₃, R₆₈=are independently C, G, U or absent;
R₃, R₁₁, R₁₂, R₁₃, R₂₈, R₃₀, R₃₆, R₄₂, R₄₄, R₄₈, R₅₈, R₅₉, R₆₁, R₆₆=are independently C, U or absent;
R₁₀, R₁₉, R₂₀, R₂₅, R₃₇, R₅₁, R₅₂, R₆₃, R₆₄=are independently G or absent;
R₁, R₃₁, R₅₀=are independently G, U or absent;
R₈, R₁₆, R₁₇, R₂₂, R₅₃, R₅₄, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III PHE (SEQ ID NO: 603),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Phe is:
R₀, R₁₈, R₂₂, R₂₃=absent
R₅, R₇, R₁₄, R₂₄, R₂₆, R₃₂, R₃₄, R₃₈, R₃₉, R₄₁, R₅₇, R₇₂=are independently A or absent;
R₄₆=A, C or absent;
R₇₀=A, C, G or absent;
R₄, R₆, R₁₈, R₅₆, R₆₉=are independently A, G or absent;
R₉, R₄₅=are independently A, U or absent;
R₂, R₁₁, R₁₃, R₃₅, R₄₃, R₄₉, R₅₈, R₆₀, R₆₈, R₇₁=are independently C or absent;
R₃₃=C, G or absent;
R₃, R₂₈, R₃₆, R₄₈, R₅₈, R₅₉, R₆₁=are independently C, U or absent;
R₁, R₁₀, R₁₉, R₂₀, R₂₁, R₂₅, R₂₇, R₂₉, R₃₇, R₄₀, R₅₁, R₅₂, R₆₂, R₆₃, R₆₄=are independently G or absent;
R₈, R₁₂, R₁₆, R₁₇, R₃₀, R₃₁, R₄₂, R₄₄, R₅₀, R₅₃, R₅₄, R₆₅, R₆₆, R₆₇=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Proline TREM Consensus Sequence

In an embodiment, a TREM disclosed herein comprises the sequence of Formula I_{PRO} (SEQ ID NO: 604),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Pro is:
R₀=absent
R₁₄, R₅₇=are independently A or absent;
R₇₀, R₇₂=are independently A, C or absent;
R₉, R₂₆, R₂₇=are independently A, C, G or absent;
R₄, R₅, R₆, R₁₆, R₂₁, R₂₉, R₃₀, R₃₁, R₃₂, R₃₃, R₃₄, R₃₇, R₄₁, R₄₂, R₄₃, R₄₄, R₄₅, R₄₆, R₄₈, R₄₉, R₅₀, R₅₈, R₆₁, R₆₂, R₆₃, R₆₄, R₆₆, R₆₇, R₆₈=are independently N or absent;
R₃₅, R₆₅=are independently A, C, U or absent;
R₂₄, R₄₀, R₅₆=are independently A, G or absent;
R₇, R₂₅, R₅₁=are independently A, G, U or absent;
R₅₈, R₆₀=are independently C or absent;
R₁, R₃, R₇₁=are independently C, G or absent;
R₁₁, R₁₂, R₂₀, R₆₉=are independently C, G, U or absent;
R₁₃, R₁₇, R₁₈, R₂₂, R₂₃, R₂₈, R₅₉=are independently C, U or absent;
R₁₀, R₁₅, R₁₉, R₃₈, R₃₉, R₅₂=are independently G or absent;
R₂=are independently G, U or absent;
R₈, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{PRO} (SEQ ID NO: 605),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₂-R₆₃-R₆₄-R₆₅-R₆₆-R₆₇-R₆₈-R₆₉-R₇₀-R₇₁-R₇₂
wherein R is a ribonucleotide residue and the consensus for Pro is:
R₀, R₁₇, R₁₈, R₂₂, R₂₃=absent;
R₁₄, R₄₅, R₅₆, R₅₇, R₅₈, R₆₅, R₆₈=are independently A or absent;
R₆₁=A, C, G or absent;
R₄₃=N or absent;
R₃₇=A, C, U or absent;
R₂₄, R₂₇, R₃₃, R₄₀, R₄₄, R₆₃=are independently A, G or absent;
R₃, R₁₂, R₃₀, R₃₂, R₄₈, R₅₅, R₆₀, R₇₀, R₇₁, R₇₂=are independently C or absent;
R₅, R₃₄, R₄₂, R₆₆=are independently C, G or absent;
R₂₀=C, G, U or absent;
R₃₅, R₄₁, R₄₉, R₆₂=are independently C, U or absent;
R₁, R₂, R₆, R₉, R₁₀, R₁₅, R₁₉, R₂₆, R₃₈, R₃₉, R₄₆, R₅₀, R₅₁, R₅₂, R₆₄, R₆₇, R₆₉=are independently G or absent;
R₁₁, R₁₆=are independently G, U or absent;
R₄, R₇, R₈, R₁₃, R₂₁, R₂₅, R₂₈, R₂₉, R₃₁, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{PRO} (SEQ ID NO: 606),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Pro is:
R₀, R₁₇, R₁₈, R₂₂, R₂₃=absent
R₁₄, R₄₅, R₅₆, R₅₇, R₅₈, R₆₅, R₆₈=are independently A or absent;
R₃₇=A, C, U or absent;
R₂₄, R₂₇, R₄₀=are independently A, G or absent;
R₃, R₅, R₁₂, R₃₀, R₃₂, R₄₈, R₄₉, R₅₅, R₆₀, R₆₁, R₆₂, R₆₆, R₇₀, R₇₁, R₇₂=are independently C or absent;
R₃₄, R₄₂=are independently C, G or absent;
R₄₃=C, G, U or absent;
R₄₁=C, U or absent;
R₁, R₂, R₆, R₉, R₁₀, R₁₅, R₁₉, R₂₀, R₂₆, R₃₃, R₃₈, R₃₉, R₄₄, R₄₆, R₅₀, R₅₁, R₅₂, R₆₃, R₆₄, R₆₇, R₆₉=are independently G or absent;
R₁₆=G, U or absent;
R₄, R₇, R₈, R₁₁, R₁₃, R₂₁, R₂₅, R₂₈, R₂₉, R₃₁, R₃₅, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Serine TREM Consensus Sequence

In an embodiment, a TREM disclosed herein comprises the sequence of Formula I_{SER} (SEQ ID NO: 607),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Ser is:
R₀=absent;
R₁₄, R₂₄, R₅₇=are independently A or absent;
R₄₁=A, C or absent;
R₂, R₃, R₄, R₅, R₆, R₇, R₉, R₁₀, R₁₁, R₁₂, R₁₃, R₁₆, R₂₁, R₂₅, R₂₆, R₂₇, R₂₈, R₃₀, R₃₁, R₃₂, R₃₃, R₃₄, R₃₇, R₄₂, R₄₃, R₄₄, R₄₅, R₄₆, R₄₈, R₄₉, R₅₀, R₆₂, R₆₃, R₆₄, R₆₈, R₆₆, R₆₇, R₆₈, R₆₉, R₇₀=are independently N or absent;
R₁₈=A, C, U or absent;
R₁₅, R₄₀, R₅₁, R₅₆=are independently A, G or absent;
R₁, R₂₉, R₅₈, R₇₂=are independently A, G, U or absent;
R₃₉=A, U or absent;
R₆₀=C or absent;
R₃₈=C, G or absent;
R₁₇, R₂₂, R₂₃, R₇₁=are independently C, G, U or absent;
R₈, R₃₅, R₃₆, R₅₅, R₅₉, R₆₁=are independently C, U or absent;
R₁₉, R₂₀=are independently G or absent;
R₅₂=G, U or absent;
R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{SER} (SEQ ID NO: 608),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Ser is:
R₀, R₂₃=absent
R₁₄, R₂₄, R₄₁, R₅₇=are independently A or absent;
R₄₄=A, C or absent;
R₂₅, R₄₅, R₄₈=are independently A, C, G or absent;
R₂, R₃, R₄, R₅, R₃₇, R₅₀, R₆₂, R₆₆, R₆₇, R₆₉, R₇₀=are independently N or absent;
R₁₂, R₂₈, R₆₅=are independently A, C, U or absent;
R₉, R₁₅, R₂₉, R₃₄, R₄₀, R₅₆, R₆₃=are independently A, G or absent;
R₇, R₂₆, R₃₀, R₃₃, R₄₆, R₅₈, R₇₂=are independently A, G, U or absent;
R₃₉=A, U or absent;
R₁₁, R₃₅, R₆₀, R₆₁=are independently C or absent;
R₁₃, R₃₈=are independently C, G or absent;
R₆, R₁₇, R₃₁, R₄₃, R₆₄, R₆₈=are independently C, G, U or absent;
R₃₆, R₄₂, R₄₉, R₅₅, R₅₉, R₇₁=are independently C, U or absent;
R₁₀, R₁₉, R₂₀, R₂₇, R₅₁=are independently G or absent;
R₁, R₁₆, R₃₂, R₅₂=are independently G, U or absent;
R₈, R₁₈, R₂₁, R₂₂, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{SER} (SEQ ID NO: 609),
R₀-R₁-R₂-R₃-R₄-R₅-R₆-R₇-R₈-R₉-R₁₀-R₁₁-R₁₂-R₁₃-R₁₄-R₁₈-R₁₀-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Ser is:
R₀, R₂₃=absent
R₁₄, R₂₄, R₄₁, R₅₇, R₅₈=are independently A or absent;
R₄₄=A, C or absent;
R₂₅, R₄₈=are independently A, C, G or absent;
R₂, R₃, R₅, R₃₇, R₆₆, R₆₇, R₆₉, R₇₀=are independently N or absent;
R₁₂, R₂₈, R₆₂=are independently A, C, U or absent;
R₇, R₉, R₁₅, R₂₉, R₃₃, R₃₄, R₄₀, R₄₅, R₅₆, R₆₃=are independently A, G or absent;
R₄, R₂₆, R₄₆, R₅₀=are independently A, G, U or absent;
R₃₀, R₃₉=are independently A, U or absent;
R₁₁, R₁₇, R₃₅, R₆₀, R₆₁=are independently C or absent;
R₁₃, R₃₈=are independently C, G or absent;
R₆, R₆₄=are independently C, G, U or absent;
R₃₁, R₄₂, R₄₃, R₄₉, R₅₅, R₅₉, R₆₅, R₆₈, R₇₁=are independently C, U or absent;
R₁₀, R₁₉, R₂₀, R₂₇, R₅₁, R₅₂=are independently G or absent;
R₁, R₁₆, R₃₂, R₇₂=are independently G, U or absent;
R₈, R₁₈, R₂₁, R₂₂, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Threonine TREM Consensus Sequence

In an embodiment, a TREM disclosed herein comprises the sequence of Formula I_{THR} (SEQ ID NO: 610),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Thr is:
R₀, R₂₃=absent
R₁₄, R₄₁, R₅₇=are independently A or absent;
R₅₆, R₇₀=are independently A, C, G or absent;
R₄, R₅, R₆, R₇, R₁₂, R₁₆, R₂₆, R₃₀, R₃₁, R₃₂, R₃₄, R₃₇, R₄₂, R₄₄, R₄₅, R₄₆, R₄₈, R₄₉, R₅₀, R₅₈, R₆₂, R₆₃, R₆₄, R₆₅, R₆₆, R₆₇, R₆₈, R₇₂=are independently N or absent;
R₁₃, R₁₇, R₂₁, R₃₅, R₆₁=are independently A, C, U or absent;
R₁, R₉, R₂₄, R₂₇, R₂₉, R₆₉=are independently A, G or absent;
R₁₈, R₂₅, R₅₁=are independently A, G, U or absent;
R₄₀, R₅₃=are independently A, U or absent;
R₃₃, R₄₃=are independently C, G or absent;
R₂, R₃, R₅₉=are independently C, G, U or absent;
R₁₁, R₁₈, R₂₂, R₂₈, R₃₆, R₅₄, R₅₅, R₆₀, R₇₁=are independently C, U or absent;
R₁₀, R₂₀, R₃₈, R₅₂=are independently G or absent;
R₁₉=G, U or absent;
R₈, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{THR} (SEQ ID NO: 611),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Thr is:
R₀, R₁₈, R₂₃=absent
R₁₄, R₄₁, R₅₇=are independently A or absent;
R₉, R₄₂, R₄₄, R₄₈, R₅₆, R₇₀=are independently A, C, G or absent;
R₄, R₆, R₁₂, R₂₆, R₄₉, R₅₈, R₆₃, R₆₄, R₆₆, R₆₈=are independently N or absent;
R₁₃, R₂₁, R₃₁, R₃₇, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₇=are independently A, G, U or absent;
R₄₀, R₅₃=are independently A, U or absent;
R₃₅=C or absent;
R₃₃, R₄₃=are independently C, G or absent;
R₂, R₃, R₅, R₁₆, R₃₂, R₃₄, R₅₉, R₆₅, R₇₂=are independently C, G, U or absent;
R₁₁, R₁₇, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{THR} (SEQ ID NO: 612),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₈-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Thr is:
R₀, R₁₈, R₂₃=absent
R₁₄, R₄₀, R₄₁, R₅₇=are independently A or absent;
R₄₄=A, C or absent;
R₉, R₄₂, R₄₈, R₅₆=are independently A, C, G or absent;
R₄, R₆, R₁₂, R₂₆, R₅₈, R₆₄, R₆₆, R₆₈=are independently N or absent;
R₁₃, R₂₁, R₃₁, R₃₇, R₄₉, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₃, R₆₇=are independently A, G, U or absent;
R₅₃=A, U or absent;
R₃₅=C or absent;
R₂, R₃₃, R₄₃, R₇₀=are independently C, G or absent;
R₅, R₁₀, R₃₄, R₅₉, R₆₅=are independently C, G, U or absent;
R₃, R₁₁, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₃₂=G, U or absent;
R₈, R₁₇, R₃₉, R₅₄, R₇₂=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tryptophan TREM Consensus Sequence

In an embodiment, a TREM disclosed herein comprises the sequence of Formula I_{TRP} (SEQ ID NO: 613),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Trp is:
R₀=absent;
R₂₄, R₃₉, R₄₁, R₅₇=are independently A or absent;
R₂, R₃, R₂₆, R₂₇, R₄₀, R₄₈=are independently A, C, G or absent;
R₄, R₅, R₆, R₂₉, R₃₀, R₃₁, R₃₂, R₃₄, R₄₂, R₄₄, R₄₅, R₄₆, R₄₉, R₅₁, R₅₈, R₆₃, R₆₆, R₆₇, R₆₈=are independently N or absent;
R₁₃, R₁₄, R₁₆, R₁₈, R₂₁, R₆₁, R₆₅, R₇₁=are independently A, C, U or absent;
R₁, R₉, R₁₀, R₁₅, R₃₃, R₅₀, R₅₆=are independently A, G or absent;
R₇, R₂₅, R₇₂=are independently A, G, U or absent;
R₃₇, R₃₈, R₅₅, R₆₀=are independently C or absent;
R₁₂, R₃₅, R₄₃, R₆₄, R₆₉, R₇₀=are independently C, G, U or absent;
R₁₁, R₁₇, R₂₂, R₂₈, R₅₉, R₆₂=are independently C, U or absent;
R₁₉, R₂₀, R₅₂=are independently G or absent;
R₈, R₂₃, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{TRP} (SEQ ID NO: 614),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{TRP} (SEQ ID NO: 615),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tyrosine TREM Consensus Sequence

In an embodiment, a TREM disclosed herein comprises the sequence of Formula I_{TYR} (SEQ ID NO: 616),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Tyr is:
R₀=absent
R₁₄, R₃₉, R₅₇=are independently A or absent;
R₄₁, R₄₈, R₅₁, R₇₁=are independently A, C, G or absent;
R₃, R₄, R₅, R₆, R₉, R₁₀, R₁₂, R₁₃, R₁₆, R₂₅, R₂₆, R₃₀, R₃₁, R₃₂, R₄₂, R₄₄, R₄₅, R₄₆, R₄₉, R₅₀, R₅₈, R₆₂, R₆₃, R₆₆, R₆₇, R₆₈, R₆₉, R₇₀=are independently N or absent;
R₂₂, R₆₅=are independently A, C, U or absent; R₁₅, R₂₄, R₂₇, R₃₃, R₃₇, R₄₀, R₅₆=are independently A, G or absent;
R₇, R₂₉, R₃₄, R₇₂=are independently A, G, U or absent;
R₂₃, R₅₃=are independently A, U or absent;
R₃₅, R₆₀=are independently C or absent;
R₂₀=C, G or absent;
R₁, R₂, R₂₈, R₆₁, R₆₄=are independently C, G, U or absent;
R₁₁, R₁₇, R₂₁, R₄₃, R₅₅=are independently C, U or absent;
R₁₉, R₅₂=are independently G or absent;
R₈, R₁₈, R₃₆, R₃₈, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{TYR} (SEQ ID NO: 617),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇=are independently A or absent;
R₄₄, R₆₉=are independently A, C or absent;
R₇₁=A, C, G or absent;
R₆₈=N or absent;
R₅₈=A, C, U or absent;
R₃₃, R₃₇, R₄₁, R₅₆, R₆₂, R₆₃=are independently A, G or absent;
R₆, R₂₉, R₇₂=are independently A, G, U or absent;
R₃₁, R₄₅, R₅₃=are independently A, U or absent;
R₁₃, R₃₅, R₄₉, R₆₀=are independently C or absent;
R₂₀, R₄₈, R₆₄, R₆₇, R₇₀=are independently C, G or absent;
R₁, R₂, R₅, R₁₆, R₆₆=are independently C, G, U or absent;
R₁₁, R₂₁, R₂₈, R₄₃, R₅₅, R₆₁=are independently C, U or absent;
R₁₀, R₁₅, R₁₉, R₂₅, R₂₇, R₄₀, R₅₁, R₅₂=are independently G or absent;
R₃, R₄, R₃₀, R₃₂, R₄₂, R₄₆=are independently G, U or absent;
R₈, R₁₂, R₁₇, R₂₂, R₃₆, R₃₈, R₅₀, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{TYR} (SEQ ID NO: 618),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇, R₇₂=are independently A or absent;
R₄₄, R₆₉=are independently A, C or absent;
R₇₁=A, C, G or absent;
R₃₇, R₄₁, R₅₆, R₆₂, R₆₃=are independently A, G or absent;
R₆, R₂₉, R₆₈=are independently A, G, U or absent;
R₃₁, R₄₅, R₅₈=are independently A, U or absent;
R₁₃, R₂₈, R₃₅, R₄₉, R₆₀, R₆₁=are independently C or absent;
R₅, R₄₈, R₆₄, R₆₇, R₇₀=are independently C, G or absent;
R₁, R₂=are independently C, G, U or absent;
R₁₁, R₁₆, R₂₁, R₄₃, R₅₅, R₆₆=are independently C, U or absent;
R₁₀, R₁₅, R₁₉, R₂₀, R₂₅, R₂₇, R₃₃, R₄₀, R₅₁, R₅₂=are independently G or absent;
R₃, R₄, R₃₀, R₃₂, R₄₂, R₄₆=are independently G, U or absent; RN, R₁₂, R₁₇, R₂₂, R₃₆, R₃₈, R₅₀, R₅₃, R₅₄, R₅₉, R₆₈=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Valine TREM Consensus Sequence

In an embodiment, a TREM disclosed herein comprises the sequence of Formula I_{VAL} (SEQ ID NO: 619),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Val is:
R₀, R₂₃=absent;
R₂₄, R₃₈, R₅₇=are independently A or absent;
R₉, R₇₂=are independently A, C, G or absent;
R₂, R₄, R₅, R₆, R₇, R₁₂, R₁₅, R₁₆, R₂₁, R₂₅, R₂₆, R₂₉, R₃₁, R₃₂, R₃₃, R₃₄, R₃₇, R₄₁, R₄₂, R₄₃, R₄₄, R₄₅, R₄₆, R₄₈, R₄₉, R₅₀, R₅₈, R₆₁, R₆₂, R₆₃, R₆₄, R₆₈, R₆₆, R₆₇, R₆₈, R₆₉, R₇₀=are independently N or absent;
R₁₇, R₃₅, R₅₉=are independently A, C, U or absent;
R₁₀, R₁₄, R₂₇, R₄₀, R₅₂, R₅₆=are independently A, G or absent;
R₁, R₃, R₅₁, R₅₃=are independently A, G, U or absent;
R₃₉=C or absent;
R₁₃, R₃₀, R₅₅=are independently C, G, U or absent;
R₁₁, R₂₂, R₂₈, R₆₀, R₇₁=are independently C, U or absent;
R₁₉=G or absent;
R₂₀=G, U or absent;
R₈, R₁₈, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{VAL} (SEQ ID NO: 620),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Val is:
R₀, R₁₈, R₂₃=absent;
R₂₄, R₃₈, R₅₇=are independently A or absent;
R₆₄, R₇₀, R₇₂=are independently A, C, G or absent;
R₁₅, R₁₆, R₂₆, R₂₉, R₃₁, R₃₂, R₄₃, R₄₄, R₄₅, R₄₉, R₅₀, R₅₈, R₆₂, R₆₅=are independently N or absent;
R₆, R₁₇, R₃₄, R₃₇, R₄₁, R₅₉=are independently A, C, U or absent;
R₉, R₁₀, R₁₄, R₂₇, R₄₀, R₄₆, R₅₁, R₅₂, R₅₆=are independently A, G or absent;
R₇, R₁₂, R₂₅, R₃₃, R₅₃, R₆₃, R₆₆, R₆₈=are independently A, G, U or absent;
R₆₉=A, U or absent;
R₃₉=C or absent;
R₅, R₆₇=are independently C, G or absent;
R₂, R₄, R₁₃, R₄₈, R₅₅, R₆₁=are independently C, G, U or absent;
R₁₁, R₂₂, R₂₈, R₃₀, R₃₅, R₆₀, R₇₁=are independently C, U or absent;
R₁₉=G or absent;
R₁, R₃, R₂₀, R₄₂=are independently G, U or absent;
R₈, R₂₁, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{VAL} (SEQ ID NO: 621),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Val is:
R₀, R₁₈, R₂₃=absent
R₂₄, R₃₈, R₄₀, R₅₇, R₇₂=are independently A or absent;
R₂₉, R₆₄, R₇₀=are independently A, C, G or absent;
R₄₉, R₅₀, R₆₂=are independently N or absent;
R₁₆, R₂₆, R₃₁, R₃₂, R₃₇, R₄₁, R₄₃, R₅₉, R₆₅=are independently A, C, U or absent;
R₉, R₁₄, R₂₇, R₄₆, R₅₂, R₅₆, R₆₆=are independently A, G or absent;
R₇, R₁₂, R₂₅, R₃₃, R₄₄, R₄₅, R₅₃, R₅₈, R₆₃, R₆₈=are independently A, G, U or absent;
R₆₉=A, U or absent;
R₃₉=C or absent;
R₅, R₆₇=are independently C, G or absent;
R₂, R₄, R₁₃, R₁₅, R₄₈, R₅₅=are independently C, G, U or absent;
R₆, R₁₁, R₂₂, R₂₈, R₃₀, R₃₄, R₃₅, R₆₀, R₆₁, R₇₁=are independently C, U or absent;
R₁₀, R₁₉, R₅₁=are independently G or absent;
R₁, R₃, R₂₀, R₄₂=are independently G, U or absent;
R₈, R₁₇, R₂₁, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Variable Region Consensus Sequence

In an embodiment, a TREM disclosed herein comprises a variable region at position R₄₇. In an embodiment, the variable region is 1-271 ribonucleotides in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 ribonucleotides). In an embodiment, the variable region comprises any one, all or a combination of Adenine, Cytosine, Guanine or Uracil. Bethany Beach, Delaware.

In an embodiment, the variable region comprises a ribonucleic acid (RNA) sequence encoded by a deoxyribonucleic acid (DNA) sequence disclosed in Table 9, e.g., any one of SEQ ID NOs: 452-561 disclosed in Table 9.

TABLE 9
Exemplary variable region sequences.

| | SEQ ID NO | SEQUENCE |
|---|---|---|
| 1 | 452 | AAAATATAAATATATTTC |
| 2 | 453 | AAGCT |
| 3 | 454 | AAGTT |
| 4 | 455 | AATTCTTCGGAATGT |
| 5 | 456 | AGA |
| 6 | 457 | AGTCC |
| 7 | 458 | CAACC |
| 8 | 459 | CAATC |
| 9 | 460 | CAGC |
| 10 | 461 | CAGGCGGGTTCTGCCCGCGC |
| 11 | 462 | CATACCTGCAAGGGTATC |
| 12 | 463 | CGACCGCAAGGTTGT |
| 13 | 464 | CGACCTTGCGGTCAT |
| 14 | 465 | CGATGCTAATCACATCGT |
| 15 | 466 | CGATGGTGACATCAT |
| 16 | 467 | CGATGGTTTACATCGT |
| 17 | 468 | CGCCGTAAGGTGT |
| 18 | 469 | CGCCTTAGGTGT |
| 19 | 470 | CGCCTTTCGACGCGT |
| 20 | 471 | CGCTTCACGGCGT |
| 21 | 472 | CGGCAGCAATGCTGT |
| 22 | 473 | CGGCTCCGCCTTC |
| 23 | 474 | CGGGTATCACAGGGTC |
| 24 | 475 | CGGTGCGCAAGCGCTGT |
| 25 | 476 | CGTACGGGTGACCGTACC |
| 26 | 477 | CGTCAAAGACTTC |
| 27 | 478 | CGTCGTAAGACTT |
| 28 | 479 | CGTTGAATAAACGT |
| 29 | 480 | CTGTC |
| 30 | 481 | GGCC |
| 31 | 482 | GGGGATT |
| 32 | 483 | GGTC |
| 33 | 484 | GGTTT |
| 34 | 485 | GTAG |
| 35 | 486 | TAACTAGATACTTTCAGAT |
| 36 | 487 | TACTCGTATGGGTGC |
| 37 | 488 | TACTTTGCGGTGT |
| 38 | 489 | TAGGCGAGTAACATCGTGC |
| 39 | 490 | TAGGCGTGAATAGCGCCTC |
| 40 | 491 | TAGGTCGCGAGAGCGGCGC |
| 41 | 492 | TAGGTCGCGTAAGCGGCGC |
| 42 | 493 | TAGGTGGTTATCCACGC |
| 43 | 494 | TAGTC |
| 44 | 495 | TAGTT |
| 45 | 496 | TATACGTGAAAGCGTATC |
| 46 | 497 | TATAGGGTCAAAAACTCTATC |
| 47 | 498 | TATGCAGAAATACCTGCATC |
| 48 | 499 | TCCCCATACGGGGGC |
| 49 | 500 | TCCCGAAGGGGTTC |
| 50 | 501 | TCTACGTATGTGGGC |
| 51 | 502 | TCTCATAGGAGTTC |
| 52 | 503 | TCTCCTCTGGAGGC |
| 53 | 504 | TCTTAGCAATAAGGT |
| 54 | 505 | TCTTGTAGGAGTTC |
| 55 | 506 | TGAACGTAAGTTCGC |
| 56 | 507 | TGAACTGCGAGGTTCC |
| 57 | 508 | TGAC |
| 58 | 509 | TGACCGAAAGGTCGT |
| 59 | 510 | TGACCGCAAGGTCGT |
| 60 | 511 | TGAGCTCTGCTCTC |
| 61 | 512 | TGAGGCCTCACGGCCTAC |
| 62 | 513 | TGAGGGCAACTTCGT |
| 63 | 514 | TGAGGGTCATACCTCC |
| 64 | 515 | TGAGGGTGCAAATCCTCC |
| 65 | 516 | TGCCGAAAGGCGT |
| 66 | 517 | TGCCGTAAGGCGT |
| 67 | 518 | TGCGGTCTCCGCGC |
| 68 | 519 | TGCTAGAGCAT |
| 69 | 520 | TGCTCGTATAGAGCTC |
| 70 | 521 | TGGACAATTGTCTGC |
| 71 | 522 | TGGACAGATGTCCGT |
| 72 | 523 | TGGACAGGTGTCCGC |
| 73 | 524 | TGGACGGTTGTCCGC |
| 74 | 525 | TGGACTTGTGGTC |
| 75 | 526 | TGGAGATTCTCTCCGC |
| 76 | 527 | TGGCATAGGCCTGC |
| 77 | 528 | TGGCTTATGTCTAC |
| 78 | 529 | TGGGAGTTAATCCCGT |
| 79 | 530 | TGGGATCTTCCCGC |
| 80 | 531 | TGGGCAGAAATGTCTC |
| 81 | 532 | TGGGCGTTCGCCCGC |
| 82 | 533 | TGGGCTTCGCCCGC |
| 83 | 534 | TGGGGGATAACCCCGT |
| 84 | 535 | TGGGGGTTTCCCCGT |
| 85 | 536 | TGGT |
| 86 | 537 | TGGTGGCAACACCGT |
| 87 | 538 | TGGTTTATAGCCGT |
| 88 | 539 | TGTACGGTAATACCGTACC |
| 89 | 540 | TGTCCGCAAGGACGT |
| 90 | 541 | TGTCCTAACGGACGT |
| 91 | 542 | TGTCCTATTAACGGACGT |
| 92 | 543 | TGTCCTTCACGGGCGT |
| 93 | 544 | TGTCTTAGGACGT |
| 94 | 545 | TGTGCGTTAACGCGTACC |
| 95 | 546 | TGTGTCGCAAGGCACC |
| 96 | 547 | TGTTCGTAAGGACTT |
| 97 | 548 | TTCACAGAAATGTGTC |
| 98 | 549 | TTCCCTCGTGGAGT |
| 99 | 550 | TTCCCTCTGGGAGC |
| 100 | 551 | TTCCCTTGTGGATC |
| 101 | 552 | TTCCTTCGGGAGC |
| 102 | 553 | TTCTAGCAATAGAGT |
| 103 | 554 | TTCTCCACTGGGGAGC |
| 104 | 555 | TTCTCGAGAGGGAGC |
| 105 | 556 | TTCTCGTATGAGAGC |
| 106 | 557 | TTTAAGGTTTTCCCTTAAC |
| 107 | 558 | TTTCATTGTGGAGT |
| 108 | 559 | TTTCGAAGGAATCC |
| 109 | 560 | TTTCTTCGGAAGC |
| 110 | 561 | TTTGGGGCAACTCAAC |

### Corresponding Nucleotide Positions

To determine if a selected nucleotide position in a candidate sequence corresponds to a selected position in a reference sequence (e.g., SEQ ID NO: 622, SEQ ID NO: 623, SEQ ID NO: 624), one or more of the following Evaluations is performed.

### Evaluation A:

1. The candidate sequence is aligned with each of the consensus sequences in Tables 10A and 10B. The consensus sequence(s) having the most positions aligned (and which has at least 60% of the positions of the candidate sequence aligned) is selected.

The alignment is performed as is follows. The candidate sequence and an isodecoder consensus sequence from Tables 10A-10B are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and the consensus sequence by counting the number of matched positions in the alignment, dividing it by the larger of the number of non-N bases in the candidate sequence or the consensus sequence, and multiplying the result by 100. In cases where multiple alignments (of the candidate and a single consensus sequence) tie for the same score, the percent similarity is the largest percent similarity calculated from the tied alignments. This process is repeated for the candidate sequence with each of the remaining isodecoder consensus sequences in Tables 10A-10B, and the alignment resulting in the greatest percent similarity is selected. If this alignment has a percent similarity equal to or greater than 60%, it is considered a valid alignment and used to relate positions in the candidate sequence to those in the consensus sequence, otherwise the candidate sequence is considered to have not aligned to any of the isodecoder consensus sequences. If there is a tie at this point, all tied consensus sequences are taken forward to step 2 in the analysis.
2. Using the selected consensus sequence(s) from step 1, one determines the consensus sequence position number that aligns with the selected position (e.g., a modified position) in the candidate sequence. One then assigns the position number of the aligned position in the consensus sequence to the selected position in the candidate sequence, in other words, the selected position in the candidate sequence is numbered according to the numbering of the consensus sequence. If there were tied consensus sequences from step one, and they give different position numbers in this step 2, then all such position numbers are taken forward to step 5.
3. The reference sequence is aligned with the consensus sequence chosen in step 1. The alignment is performed as described in step 1.
4. From the alignment in step 3, one determines the consensus sequence position number that aligns with the selected position (e.g., a modified position) in the reference sequence. One then assigns the position number of the aligned position in the consensus sequence to the selected position in the reference sequence, in other words, the selected position in the reference sequence is numbered according to the numbering of the consensus sequence. If there is a tie at this point, all tied consensus sequences are taken forward to step 5 in the analysis.
5. If a value for a position number determined for the reference sequence in step 2 is the same as the value for the position number determined for the candidate sequence in step 4, the positions are defined as corresponding.

### Evaluation B:

The reference sequence (e.g., a TREM sequence described herein) and the candidate sequence are aligned with one another. The alignment is performed as follows.

The reference sequence and the candidate sequence are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and reference sequence by counting the number of matched based in the alignment, dividing it by the larger of the number of non-N bases in the candidate or reference sequence, and multiplying the result by 100. In cases where multiple alignments tie for the same score, the percent similarity is the largest percent similarity calculated from the tied alignments. If this alignment has a percent similarity equal to or greater than 60%, it is considered a valid alignment and used to relate positions in the candidate sequence to those in the reference sequence, otherwise the candidate sequence is considered to have not aligned to the reference sequence.

If the selected nucleotide position in the reference sequence (e.g., a modified position) is paired with a selected nucleotide position (e.g., a modified position) in the candidate sequence, the positions are defined as corresponding.

If the selected position in the reference sequence and the candidate sequence are found to be corresponding in at least one of Evaluations A and B, the positions correspond. Thus, e.g., if two positions are found to be corresponding under Evaluation A, but do not correspond under Evaluation B, the positions are defined as corresponding.

The numbering given above is used for ease of presentation and does not imply a required sequence. If more than one Evaluation is performed, they can be performed in any order.

TABLE 10A
Consensus sequence computationally generated for each isodecoder by aligning
members of the isodecoder family

| SEQ ID | Amino | | |
|---|---|---|---|
| NO. | Acid | Anticodon | Consensus sequence |
| 1200 | Ala | AGC | GGGGAATTAGCTCAAGTGGTAGAGCGCTTG |
| | | | CTTAGCATGCAAGAGGTAGTGGGATCGATG |
| | | | CCCACATTCTCCA |
| 1201 | Ala | CGC | GGGGATGTAGCTCAGTGGTAGAGCGCATGC |
| | | | TTCGCATGTATGAGGTCCCGGGTTCGATCCC |
| | | | CGGCATCTCCA |
| 1202 | Ala | TGC | GGGGGTGTAGCTCAGTGGTAGAGCGCATGC |
| | | | TTTGCATGTATGAGGCCCCGGGTTCGATCCC |
| | | | CGGCACCTCCA |
| 1203 | Arg | ACG | GGGCCAGTGGCGCAATGGATAACGCGTCTG |
| | | | ACTACGGATCAGAAGATTCCAGGTTCGACTC |
| | | | CTGGCTGGCTCG |
| 1204 | Arg | CCG | GGCCGCGTGGCCTAATGGATAAGGCGTCTG |
| | | | ATTCCGGATCAGAAGATTGAGGGTTCGAGTC |
| | | | CCTTCGTGGTCG |
| 1205 | Arg | CCT | GCCCCAGTGGCCTAATGGATAAGGCACTGG |
| | | | CCTCCTAAGCCAGGGATTGTGGGTTCGAGTC |
| | | | CCACCTGGGGTA |
| 1206 | Arg | TCG | GACCGCGTGGCCTAATGGATAAGGCGTCTG |
| | | | ACTTCGGATCAGAAGATTGAGGGTTCGAGTC |
| | | | CCTCCGTGGTCG |
| 1207 | Arg | TCT | GGCTCTGTGGCGCAATGGATNAGCGCATTG |
| | | | GACTTCTAATTCAAAGGTTGCGGGTTCGAGT |
| | | | CCCNCCAGAGTCG |
| 1208 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACG |
| 1209 | Asp | GTC | TCCTCGTTAGTATAGTGGTGAGTATCCCCGC |
| | | | CTGTCACGCGGGAGACCGGGGTTCGATTCCC |
| | | | CGACGGGGAG |
| 1210 | Cys | GCA | GGGGGTATAGCTCAGNGGGTAGAGCATTTG |
| | | | ACTGCAGATCAAGAGGTCCCCGGTTCAAATC |
| | | | CGGGTGCCCCCT |
| 1211 | Gln | CTG | GGTTCCATGGTGTAATGGTNAGCACTCTGGA |
| | | | CTCTGAATCCAGCGATCCGAGTTCAAGTCTC |
| | | | GGTGGAACCT |
| 1212 | Gln | TTG | GGTCCCATGGTGTAATGGTTAGCACTCTGGA |
| | | | CTTTGAATCCAGCGATCCGAGTTCAAATCTC |
| | | | GGTGGGACCT |
| 1213 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAA |
| 1214 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAA |
| 1215 | Gly | CCC | GCATTGGTGGTTCAGTGGTAGAATTCTCGCC |
| | | | TCCCACGCNGGAGACCCGGGTTCGATTCCCG |
| | | | GCCAATGCA |
| 1216 | Gly | GCC | GCATTGGTGGTTCAGTGGTAGAATTCTCGCC |
| | | | TGCCACGCGGGAGGCCCGGGTTCGATTCCCG |
| | | | GCCAATGCA |
| 1217 | Gly | TCC | GCGTTGGTGGTATAGTGGTGAGCATAGCTGC |
| | | | CTTCCAAGCAGTTGACCCGGGTTCGATTCCC |
| | | | GGCCAACGCA |
| 1218 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCA |
| 1219 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCA |
| 1220 | Leu | AAG | GGTAGCGTGGCCGAGCGGTCTAAGGCGCTG |
| | | | GATTAAGGCTCCAGTCTCTTCGGGGGCGTGG |
| | | | GTTCGAATCCCACCGCTGCCA |
| 1221 | Leu | CAA | GTCAGGATGGCCGAGTGGTCNTAAGGCGCC |
| | | | AGACTCAAGTTCTGGTCTCCGNATGGAGGCG |
| | | | TGGGTTCGAATCCCACTTCTGACA |
| 1222 | Leu | CAG | GTCAGGATGGCCGAGCGGTCTAAGGCGCTG |
| | | | CGTTCAGGTCGCAGTCTCCCCTGGAGGCGTG |
| | | | GGTTCGAATCCCACTCCTGACA |
| 1223 | Lcu | TAA | ACCAGGATGGCCGAGTGGTTAAGGCGTTGG |
| | | | ACTTAAGATCCAATGGACAGATGTCCGCGTG |
| | | | GGTTCGAACCCCACTCCTGGTA |
| 1224 | Leu | TAG | GGTAGCGTGGCCGAGCGGTCTAAGGCGCTG |
| | | | GATTTAGGCTCCAGTCTCTTCGGNGGCGTGG |
| | | | GTTCGAATCCCACCGCTGCCA |
| 1225 | Lys | CTT | GCCCGGCTAGCTCAGTCGGTAGAGCATGAG |
| | | | ACTCTTAATCTCAGGGTCGTGGGTTCGAGCC |
| | | | CCACGTTGGGCGNNN |
| 1226 | Lys | TTT | GCCTGGATAGCTCAGTCGGTAGAGCATCAG |
| | | | ACTTTTAATCTGAGGGTCCAGGGTTCAAGTC |
| | | | CCTGTTCAGGCG |
| 1227 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCA |
| 1228 | Phe | GAA | GCCGAAATAGCTCAGTTGGGAGAGCGTTAG |
| | | | ACTGAAGATCNTAAAGGTCCCTGGTTCAATC |
| | | | CCGGGTTTCGGCA |
| 1229 | Pro | AGG | GGCTCGTTGGTCTAGGGGTATGATTCTCGCT |
| | | | TAGGATGCGAGAGGTCCCGGGTTCAAATCC |
| | | | CGGACGAGCCC |
| 1230 | Pro | CGG | GGCTCGTTGGTCTAGGGGTATGATTCTCGCT |
| | | | TCGGGTGCGAGAGGTCCCGGGTTCAAATCCC |
| | | | GGACGAGCCC |
| 1231 | Pro | TGG | GGCTCGTTGGTCTAGGGGTATGATTCTCGCT |
| | | | TTGGGTGCGAGAGGTCCCGGGTTCAAATCCC |
| | | | GGACGAGCCC |
| 1232 | Ser | AGA | GTAGTCGTGGCCGAGTGGTTAAGGCGATGG |
| | | | ACTAGAAATCCATTGGGGTTTCCCCGCGCAG |
| | | | GTTCGAATCCTGCCGACTACG |
| 1233 | Ser | CGA | GCTGTGATGGCCGAGTGGTTAAGGCGTTGG |
| | | | ACTCGAAATCCAATGGGGTCTCCCCGCGCAG |
| | | | GTTCGAATCCTGCTCACAGCG |
| 1234 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCG |
| 1235 | Ser | TGA | GTAGTCGTGGCCGAGTGGTTAAGGCGATGG |
| | | | ACTTGAAATCCATTGGGGTCTCCCCGCGCAG |
| | | | GTTCGAATCCTGCCGGCTACG |
| 1236 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCT |
| 1237 | Thr | CGT | GGCNCTGTGGCTNAGTNGGNTAAAGCGCCG |
| | | | GTCTCGTAAACCNGGAGATCNTGGGTTCGA |
| | | | ATCCCANCNGGGCCT |
| 1238 | Thr | TGT | GGCTCCATAGCTCAGNGGGTTAGAGCACTG |
| | | | GTCTTGTAAACCAGGGGTCGCGAGTTCAAAT |
| | | | CTCGCTGGGGCCT |
| 1239 | Trp | CCA | GACCTCGTGGCGCAACGGTAGCGCGTCTGA |
| | | | CTCCAGATCAGAAGGTTGCGTGTTCAAATCA |
| | | | CGTCGGGGTCA |
| 1240 | Tyr | GTA | CCTTCGATAGCTCAGCTGGTAGAGCGGAGG |
| | | | ACTGTAGATCCTTAGGTCGCTGGTTCGATTC |
| | | | CGGCTCGAAGGA |
| 1241 | Val | AAC | GTTTCCGTAGTGTAGTGGTTATCACGTTCGC |
| | | | CTAACACGCGAAAGGTCCCCGGTTCGAAAC |
| | | | CGGGCGGAAACA |
| 1242 | Val | CAC | GTTTCCGTAGTGTAGTGGTTATCACGTTCGC |
| | | | CTCACACGCGAAAGGTCCCCGGTTCGAAAC |
| | | | CGGGCGGAAACA |
| 1243 | Val | TAC | GGTTCCATAGTGTAGTGGTTATCACGTCTGC |
| | | | TTTACACGCAGAAGGTCCTGGGTTCGAGCCC |
| | | | CAGTGGAACCA |
| 1244 | iMet | CAT | AGCAGAGTGGCGCAGCGGAAGCGTGCTGGG |
| | | | CCCATAACCCAGAGGTCGATGGATCGAAAC |
| | | | CATCCTCTGCTA |

TABLE 10B
Consensus sequence computationally generated for each isodecoder by aligning
members of the isodecoder family

| SEQ ID | Amino | | |
|---|---|---|---|
| NO | Acid | Anticodon | Consensus sequence |
| 1245 | Ala | AGC | GGGGAATTAGCTCAAGTGGTAGAGCGCTTGC |
| | | | TTAGCATGCAAGAGGTAGTGGGATCGATGCC |
| | | | CACATTCTCCANNN |
| 1246 | Ala | CGC | GGGGATGTAGCTCAGTGGTAGAGCGCATGCT |
| | | | TCGCATGTATGAGGTCCCGGGTTCGATCCCC |
| | | | GGCATCTCCANNN |
| 1247 | Ala | TGC | GGGGGTGTAGCTCAGTGGTAGAGCGCATGCT |
| | | | TTGCATGTATGAGGCCCCGGGTTCGATCCCC |
| | | | GGCACCTCCANNN |
| 1248 | Arg | ACG | GGGCCAGTGGCGCAATGGATAACGCGTCTGA |
| | | | CTACGGATCAGAAGATTCCAGGTTCGACTCC |
| | | | TGGCTGGCTCGNNN |
| 1249 | Arg | CCG | GGCCGCGTGGCCTAATGGATAAGGCGTCTGA |
| | | | TTCCGGATCAGAAGATTGAGGGTTCGAGTCC |
| | | | CTTCGTGGTCGNNN |
| 1250 | Arg | CCT | GCCCCAGTGGCCTAATGGATAAGGCACTGGC |
| | | | CTCCTAAGCCAGGGATTGTGGGTTCGAGTCC |
| | | | CACCTGGGGTANNN |
| 1251 | Arg | TCG | GACCGCGTGGCCTAATGGATAAGGCGTCTGA |
| | | | CTTCGGATCAGAAGATTGAGGGTTCGAGTCC |
| | | | CTCCGTGGTCGNNN |
| 1252 | Arg | TCT | GGCTCTGTGGCGCAATGGATNAGCGCATTGG |
| | | | ACTTCTAATTCAAAGGTTGCGGGTTCGAGTC |
| | | | CCNCCAGAGTCGNNN |
| 1253 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACGNNN |
| 1254 | Asp | GTC | TCCTCGTTAGTATAGTGGTGAGTATCCCCGCC |
| | | | TGTCACGCGGGAGACCGGGGTTCGATTCCCC |
| | | | GACGGGGAGNNN |
| 1255 | Cys | GCA | GGGGGTATAGCTCAGNGGGTAGAGCATTTGA |
| | | | CTGCAGATCAAGAGGTCCCCGGTTCAAATCC |
| | | | GGGTGCCCCCTNNN |
| 1256 | Gln | CTG | GGTTCCATGGTGTAATGGTNAGCACTCTGGA |
| | | | CTCTGAATCCAGCGATCCGAGTTCAAGTCTC |
| | | | GGTGGAACCTNNN |
| 1257 | Gln | TTG | GGTCCCATGGTGTAATGGTTAGCACTCTGGA |
| | | | CTTTGAATCCAGCGATCCGAGTTCAAATCTC |
| | | | GGTGGGACCTNNN |
| 1258 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAANNN |
| 1259 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAANNN |
| 1260 | Gly | CCC | GCATTGGTGGTTCAGTGGTAGAATTCTCGCCT |
| | | | CCCACGCNGGAGACCCGGGTTCGATTCCCGG |
| | | | CCAATGCANNN |
| 1261 | Gly | GCC | GCATTGGTGGTTCAGTGGTAGAATTCTCGCCT |
| | | | GCCACGCGGGAGGCCCGGGTTCGATTCCCGG |
| | | | CCAATGCANNN |
| 1262 | Gly | TCC | GCGTTGGTGGTATAGTGGTGAGCATAGCTGC |
| | | | CTTCCAAGCAGTTGACCCGGGTTCGATTCCC |
| | | | GGCCAACGCANNN |
| 1263 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCANNN |
| 1264 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCANNN |
| 1265 | Leu | AAG | GGTAGCGTGGCCGAGCGGTCTAAGGCGCTGG |
| | | | ATTAAGGCTCCAGTCTCTTCGGGGGCGTGGG |
| | | | TTCGAATCCCACCGCTGCCANNN |
| 1266 | Lcu | CAA | GTCAGGATGGCCGAGTGGTCNTAAGGCGCCA |
| | | | GACTCAAGTTCTGGTCTCCGNATGGAGGCGT |
| | | | GGGTTCGAATCCCACTTCTGACANNN |
| 1267 | Leu | CAG | GTCAGGATGGCCGAGCGGTCTAAGGCGCTGC |
| | | | GTTCAGGTCGCAGTCTCCCCTGGAGGCGTGG |
| | | | GTTCGAATCCCACTCCTGACANNN |
| 1268 | Leu | TAA | ACCAGGATGGCCGAGTGGTTAAGGCGTTGGA |
| | | | CTTAAGATCCAATGGACAGATGTCCGCGTGG |
| | | | GTTCGAACCCCACTCCTGGTANNN |
| 1269 | Leu | TAG | GGTAGCGTGGCCGAGCGGTCTAAGGCGCTGG |
| | | | ATTTAGGCTCCAGTCTCTTCGGNGGCGTGGG |
| | | | TTCGAATCCCACCGCTGCCANNN |
| 1270 | Lys | CTT | GCCCGGCTAGCTCAGTCGGTAGAGCATGAGA |
| | | | CTCTTAATCTCAGGGTCGTGGGTTCGAGCCCC |
| | | | ACGTTGGGCGNNNNNN |
| 1271 | Lys | TTT | GCCTGGATAGCTCAGTCGGTAGAGCATCAGA |
| | | | CTTTTAATCTGAGGGTCCAGGGTTCAAGTCCC |
| | | | TGTTCAGGCGNNN |
| 1272 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCANNN |
| 1273 | Phe | GAA | GCCGAAATAGCTCAGTTGGGAGAGCGTTAGA |
| | | | CTGAAGATCNTAAAGGTCCCTGGTTCAATCC |
| | | | CGGGTTTCGGCANNN |
| 1274 | Pro | AGG | GGCTCGTTGGTCTAGGGGTATGATTCTCGCTT |
| | | | AGGATGCGAGAGGTCCCGGGTTCAAATCCCG |
| | | | GACGAGCCCNNN |
| 1275 | Pro | CGG | GGCTCGTTGGTCTAGGGGTATGATTCTCGCTT |
| | | | CGGGTGCGAGAGGTCCCGGGTTCAAATCCCG |
| | | | GACGAGCCCNNN |
| 1276 | Pro | TGG | GGCTCGTTGGTCTAGGGGTATGATTCTCGCTT |
| | | | TGGGTGCGAGAGGTCCCGGGTTCAAATCCCG |
| | | | GACGAGCCCNNN |
| 1277 | Ser | AGA | GTAGTCGTGGCCGAGTGGTTAAGGCGATGGA |
| | | | CTAGAAATCCATTGGGGTTTCCCCGCGCAGG |
| | | | TTCGAATCCTGCCGACTACGNNN |
| 1278 | Ser | CGA | GCTGTGATGGCCGAGTGGTTAAGGCGTTGGA |
| | | | CTCGAAATCCAATGGGGTCTCCCCGCGCAGG |
| | | | TTCGAATCCTGCTCACAGCGNNN |
| 1279 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCGNNN |
| 1280 | Ser | TGA | GTAGTCGTGGCCGAGTGGTTAAGGCGATGGA |
| | | | CTTGAAATCCATTGGGGTCTCCCCGCGCAGG |
| | | | TTCGAATCCTGCCGGCTACGNNN |
| 1281 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCTNNN |
| 1282 | Thr | CGT | GGCNCTGTGGCTNAGTNGGNTAAAGCGCCGG |
| | | | TCTCGTAAACCNGGAGATCNTGGGTTCGAAT |
| | | | CCCANCNGGGCCTNNN |
| 1283 | Thr | TGT | GGCTCCATAGCTCAGNGGGTTAGAGCACTGG |
| | | | TCTTGTAAACCAGGGGTCGCGAGTTCAAATC |
| | | | TCGCTGGGGCCTNNN |
| 1284 | Trp | CCA | GACCTCGTGGCGCAACGGTAGCGCGTCTGAC |
| | | | TCCAGATCAGAAGGTTGCGTGTTCAAATCAC |
| | | | GTCGGGGTCANNN |
| 1285 | Tyr | GTA | CCTTCGATAGCTCAGCTGGTAGAGCGGAGGA |
| | | | CTGTAGATCCTTAGGTCGCTGGTTCGATTCCG |
| | | | GCTCGAAGGANNN |
| 1286 | Val | AAC | GTTTCCGTAGTGTAGTGGTTATCACGTTCGCC |
| | | | TAACACGCGAAAGGTCCCCGGTTCGAAACCG |
| | | | GGCGGAAACANNN |
| 1287 | Val | CAC | GTTTCCGTAGTGTAGTGGTTATCACGTTCGCC |
| | | | TCACACGCGAAAGGTCCCCGGTTCGAAACCG |
| | | | GGCGGAAACANNN |
| 1288 | Val | TAC | GGTTCCATAGTGTAGTGGTTATCACGTCTGCT |
| | | | TTACACGCAGAAGGTCCTGGGTTCGAGCCCC |
| | | | AGTGGAACCANNN |
| 1289 | iMet | CAT | AGCAGAGTGGCGCAGCGGAAGCGTGCTGGG |
| | | | CCCATAACCCAGAGGTCGATGGATCGAAACC |
| | | | ATCCTCTGCTANNN |

TABLE 11
Score values alignment

| | Candidate | Reference | Match |
|---|---|---|---|
| Row | nucleotide | nucleotide | score |
| 1 | A | A | 1 |
| 2 | T | T | 1 |
| 3 | U | T | 1 |
| 4 | C | C | 1 |
| 5 | G | G | 1 |
| 6 | A | N | 0 |
| 7 | T | N | 0 |
| 8 | C | N | 0 |
| 9 | G | N | 0 |
| 10 | N | A | 0 |
| 11 | N | T | 0 |
| 12 | N | C | 0 |
| 13 | N | G | 0 |
| 14 | N | N | 0 |

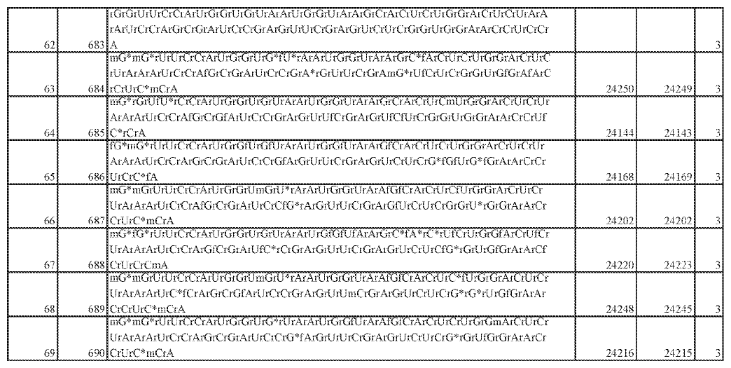

A TREM may comprise any of the nucleotide sequences of the tRNA consensus sequences described herein. For example, the TREM may comprise the nucleotide sequence of an arginine tRNA consensus sequence, e.g., a nucleotide sequence of Formula I_{ARG} (SEQ ID NO: 565), Formula II_{ARG} (SEQ ID NO: 566), or Formula III ARG (SEQ ID NO: 567). In an embodiment, a TREM comprising the nucleotide sequence of an arginine tRNA consensus sequence has an anticodon that is complimentary to a stop codon, e.g., TGA, TAG, or TAA. In an embodiment, a TREM comprising the nucleotide sequence of an arginine tRNA consensus sequence has an anticodon that is complimentary to the TGA stop codon. In an embodiment, a TREM comprising the nucleotide sequence of an arginine tRNA consensus sequence has an anticodon that is complimentary to the TAG stop codon. In an embodiment, a TREM comprising the nucleotide sequence of an arginine tRNA consensus sequence has an anticodon that is complimentary to the TAA stop codon. In an embodiment, a TREM comprises a nucleotide sequence listed in FIG. 6. In an embodiment, a TREM comprises a nucleotide sequence having about 60%, 65%, 70%, 75%, 80%, 85%, 90%, 95%, 99%, or 99.9% sequence identity, e.g., relative to a nucleotide sequence listed in FIG. 6. In an embodiment, a TREM comprises a nucleotide sequence that comprises a nucleotide mutation, e.g., relative to a nucleotide sequence listed in FIG. 6. In an embodiment, a TREM comprises a nucleotide sequence that comprises a nucleotide substitution, e.g., relative to a nucleotide sequence listed in FIG. 6. In an embodiment, a TREM comprises a nucleotide sequence that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 nucleotide mutations, e.g., relative to a nucleotide sequence in FIG. 6. In an embodiment, a TREM comprises a nucleotide sequence that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 nucleotide substitutions, relative toa nucleotide sequence in FIG. 6. In an embodiment, a TREM comprises the nucleotide sequence of any one of SEQ ID NOs: 622 and 626-675, e.g., listed in FIG. 6.

A TREM described herein may comprise the nucleotide sequence of a glutamine tRNA consensus sequence, e.g., a nucleotide sequence of Formula I GLN (SEQ ID NO: 577), Formula II GLN (SEQ ID NO: 578), or Formula III_{GLN} (SEQ ID NO: 579). In an embodiment, a TREM comprising the nucleotide sequence of a glutamine tRNA consensus sequence has an anticodon that is complimentary to a stop codon, e.g., TGA, TAG, or TAA. In an embodiment, a TREM comprising the nucleotide sequence of a glutamine tRNA consensus sequence has an anticodon that is complimentary to the TGA stop codon. In an embodiment, a TREM comprising the nucleotide sequence of a glutamine tRNA consensus sequence has an anticodon that is complimentary to the TAG stop codon. In an embodiment, a TREM comprising the nucleotide sequence of a glutamine tRNA consensus sequence has an anticodon that is complimentary to the TAA stop codon. In an embodiment, a TREM comprises a nucleotide sequence listed in FIG. 6. In an embodiment, a TREM comprises a nucleotide sequence having about 60%, 65%, 70%, 75%, 80%, 85%, 90%, 95%, 99%, or 99.9% sequence identity, e.g., relative to a nucleotide sequence listed in FIG. 6. In an embodiment, a TREM comprises a nucleotide sequence that comprises a nucleotide mutation, e.g., relative to a nucleotide sequence listed in FIG. 6. In an embodiment, a TREM comprises a nucleotide sequence that comprises a nucleotide substitution, e.g., relative to a nucleotide sequence listed in FIG. 6. In an embodiment, a TREM comprises a nucleotide sequence that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 nucleotide mutations, e.g., relative to a nucleotide sequence in FIG. 6. In an embodiment, a TREM comprises a nucleotide sequence that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 nucleotide substitutions, relative toa nucleotide sequence in FIG. 6. In an embodiment, a TREM comprises the nucleotide sequence of any one of SEQ ID NOs: 624 and 676-690, e.g., listed in FIG. 6.

A TREM described herein may comprise the nucleotide sequence of a serine tRNA consensus sequence, e.g., a nucleotide sequence of Formula I_{SER} (SEQ ID NO: 607), Formula II SER (SEQ ID NO: 608), or Formula III SER (SEQ ID NO: 609). In an embodiment, a TREM comprising the nucleotide sequence of a serine tRNA consensus sequence has an anticodon that is complimentary to any of the stop codons, e.g., TGA, TAG, or TAA. In an embodiment, a TREM comprising the nucleotide sequence of a serine tRNA consensus sequence has an anticodon that is complimentary to the TGA stop codon. In an embodiment, a TREM comprising the nucleotide sequence of a serine tRNA consensus sequence has an anticodon that is complimentary to the TAG stop codon. In an embodiment, a TREM comprising the nucleotide sequence of a serine tRNA consensus sequence has an anticodon that is complimentary to the TAA stop codon. In an embodiment, a TREM comprises a nucleotide sequence listed in FIG. 6. In an embodiment, a TREM comprises a nucleotide sequence having about 60%, 65%, 70%, 75%, 80%, 85%, 90%, 95%, 99%, or 99.9% sequence identity, e.g., relative to a nucleotide sequence listed in FIG. 6. In an embodiment, a TREM comprises a nucleotide sequence that comprises a nucleotide mutation, e.g., relative to a nucleotide sequence listed in FIG. 6. In an embodiment, a TREM comprises a nucleotide sequence that comprises a nucleotide substitution, e.g., relative to a nucleotide sequence listed in FIG. 6. In an embodiment, a TREM comprises a nucleotide sequence that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 nucleotide mutations, e.g., relative to a nucleotide sequence in FIG. 6. In an embodiment, a TREM comprises a nucleotide sequence that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 nucleotide substitutions, relative to a nucleotide sequence in FIG. 6. In an embodiment, a TREM comprises the nucleotide sequence of any one of SEQ ID NOs: 623 or 625, e.g., listed in FIG. 6.

### Proliferative Diseases

A TREM composition disclosed herein can be used to treat a proliferative disease, such as a cancer. In an embodiment, the cancer is characterized by a PTC signature. In some embodiments, the PTC signature comprises a nonsense mutation. Exemplary proliferative diseases (e.g., cancers) are listed in Tables 12-14.

In an embodiment, the subject has a disease or disorder provided in any one of Tables 12-14. In an embodiment, the cell is associated with, e.g., is obtained from a subject who has, a disorder or a disease listed in any of Tables 12-14.

For example, the disorder or disease can be chosen from the left column of Table 12. As another example, the disorder or disease is chosen from the left column of Table 12, and in embodiments the PTC is in a gene chosen from the right column of Table 12, e.g., any one of the genes provided in the right column of Table 12. In some embodiments, the PTC is in a gene corresponding the disorder or disease provided in the left column of Table 12. As a further non-limiting example, the PTC can be at a position provided in Table 12.

As another example, the disorder or symptom is chosen from a disorder or disease provided in Table 13.

As yet another example, the disorder or symptom is chosen from a disorder or disease provided in Table 14. In an embodiment, the disorder or symptom is chosen from a disorder or disease provided in Table 14, and in embodiments, the PTC is in any gene provided in Table 14. In an embodiment, the disorder or symptom is chosen from a disorder or disease provided in Table 14, and the PTC is in a corresponding gene provided in Table 14, e.g., a gene corresponding to the disease or disorder. In an embodiment, the disorder or symptom is chosen from a disorder or disease provided in Table 14 and the PTC is not in a gene provided in Table 14.

In an embodiment of any of the methods disclosed herein, the PTC is at any position within the ORF of the gene, e.g., upstream of the naturally occurring stop codon.

Include a section on characterizing the tumor.

In some examples, the tumor comprises a discrete tumor with defined boundaries. In various embodiments, the tumor is a solid tumor or localized tumor mass. For example, the biomaterial-containing device is placed directly onto the tumor mass, into the tumor mass, or adjacent to the tumor mass (i.e., physically in contact with or in close proximity to) the tumor mass itself rather than at a site remote (e.g., more than 10 mm from) from the tumor mass, e.g., placed under the skin at a site remote from the tumor. Using the system described above, there is no need for patient-derived material, e.g., a patient-derived or biopsied tumor lysate or processed antigen, as a component of the device that serves as a tumor antigen, because dying tumor cells themselves provide any antigen required for generation of an adaptive immune cell response. In some embodiments, the scaffold or device does not comprise a tumor antigen prior to being administered to the subject.

In some embodiments, the tumor is a solid tumor. In some embodiments, the tumor is about 0.1 mm to about 20 cm in diameter, e.g., about 0.1 mm to about 0.5 mm, about 0.5 mm to about 1.0 mm, about 1.0 mm to about 5.0 mm, about 5.0 mm to about 1 cm, about 1 cm to about 5 cm, about 5 cm to about 10 cm, about 10 cm to about 15 cm, about 15 cm to about 20 cm.

In some examples, the tumor comprises a diffuse tumor (e.g., a solid tumor without defined borders or boundaries). In some embodiments, the diffuse tumor is a solid tumor (e.g., brain tumor, e.g., diffuse midline gliomas, glioblastomas). In some embodiments, the diffuse tumor is a hematological tumor. In some embodiments, the hematological tumor is a malignancy of the bone marrow, of the blood, and/or the lymph nodes. In some embodiments, the hematological tumor is a leukemia or lymphoma. For example, the hematological tumor is acute lymphoblastic leukemia (ALL), acute myelogenous leukemia (AML), chronic myelogenous leukemia (CML), chronic lymphoblatic leukemia (CLL), hairy cell leukemia, Hodgkin's disease, Non-Hodgkin's lymphoma, multiple myeloma, myelodysplastic syndrome, myelofibrosis, or myeloproliferative disease.

In some embodiments, the tumor comprises necrotic tissue. In some embodiments, the TREM is administered via injection into the center of the tumor. In some embodiments, the TREM is administered via injection adjacent to the tumor. In some embodiments, the TREM is administered to non-cancerous tissue adjacent to the tumor.

Aspects of the present subject matter relate to the treatment of solid tumors. For example, the tumor is of a cancer that is other than a cancer of blood cells, such as leukemia. In certain embodiments, the cancer is metastatic. In various embodiments, the tumor is a skin cancer, such as melanoma. Implementations of the present subject matter relate to the treatment of cancer for which tumors may be biopsied (while avoiding the need for a biopsy to, e.g., produce a tumor antigen such as tumor cell lysate). In some embodiments, the tumor is a sarcoma or carcinoma tumor. Non-limiting tumors which may be targeted in embodiments of the present subject matter include breast cancer, testicular cancer, prostate cancer, ovarian cancer, pancreatic cancer, lung cancer, thyroid cancer, liver cancer (e.g., non-small cell lung cancer), colon, esophagus cancer, stomach cancer, cervical, brain cancer, renal cancer, retinolastoma, osteosarcoma, osteosarcoma, chondroblastoma, chondrosarcoma, Ewing sarcoma, Wilms tumor, malignant rhabdoid, hepatoblastoma, hepatocellular carcinoma, neuroblastoma, medulloblastoma, glioblastoma, adrenocortical carcinoma, nasopharyngeal carcinoma, rhabdomyosarcoma, desmoid, fibrosarcoma, or liposarcoma tumor. In embodiments relating to the injection of a device of scaffold of the invention, the needle may be guided visually and/or with the assistance of an imaging device such as an X-ray (e.g., using a computerized tomography (CT) scan), ultrasound, endoscope, or laparoscope device.

In certain embodiments, the tumor is a cancerous tumor. In some embodiments, the cancerous tumor is metastatic. In certain embodiments, the tumor is a precancerous tumor. In certain embodiments, the tumor is a benign tumor. In some embodiments, the subject has a disease associated with tumor growth. For example, the subject has a PTC disease associated with tumor growth. In certain embodiments, the PTC disease is any one of those listed in Tables 12, 13, or 14.

TABLE 12
Exemplary diseases or disorders

| | |
|---|---|
| Disease/disorder or protein | Exemplary Point Mutation |

G to A point mutations

| | |
|---|---|
| Lynch syndrome | NM 000251.2(MSH2): c.212-1G > A |
| Breast-ovarian cancer, familial 1 | NM 007294.3(BRCA1): c.963G > A |
| | (p.Trp321Ter) |
| BRCA2 | NM 000059.3(BRCA2): c.582G > A |
| Familial cancer of breast Breast- | (p.Trpl94Ter) |
| ovarian cancer, familial 2 | |
| MENl | NM 130799.2(MEN1): c.1269G > A |
| Hereditary cancer-predisposing | (p.Trp423Ter) |
| syndrome | |
| MLHl | NM 000249.3(MLH1): c.1998G > A |
| Lynch syndrome | (p.Trp666Ter) |
| TSC2 | NM 000548.4(TSC2): c.2108G > A |
| Tuberous sclerosis 21Tuberous | (p.Trp703Ter) |
| sclerosis syndrome 46 | |
| NFl | NM 000267.3(NF1): c.7044G > A |
| Neurofibromatosis, type 1 | (p.Trp2348Ter) |
| MSH6 | NM 000179.2(MSH6): c.3020G > A |
| Lynch syndrome | (p.Trpl007Ter) |
| BRCAl | NM 007294.3(BRCA1): c.5511G > |
| Familial cancer ofbreastlBreast- | A (p.Trpl837Ter) |
| ovarian cancer, familial 1 | |
| MYBPC3 | NM 000256.3(MYBPC3): c.3293G > |
| Primary familial hypertrophic | A (p.Trpl098Ter) |
| cardiomyopathy | |
| APC | NM 000038.5(APC): c.1262G > A |
| Familial adenomatous polyposis 1 | (p.Trp421Ter) |

T to C point mutations

| | |
|---|---|
| Familial cancer of breast, Breast- | NM_000059.3(BRCA2): c.316 + |
| ovarian cancer, familial 2, | 2T > C |
| Hereditary cancer predisposing | |
| syndrome | |

TABLE 13
Additional exemplary disorders

| | Autoimmune lymphoproliferative |
|---|---|
| 5q-syndrome | syndrome type IA |
| Autoimmune lymphoproliferative | Carney complex type 1 |
| syndrome type V | |

TABLE 14
Exemplary genes with ORFs comprising a PTC and exemplary disorders

| Gene | Disease/Disorder |
|---|---|
| ABRAXAS1 | Hereditary breast and ovarian cancer syndrome |
| APC | Adenomatous colonic polyposis; Adenomatous polyposis coli with |
| | congenital cholesteatoma; Brain tumor-polyposis syndrome-2; Carcinoma |
| | of colon; Colon adenocarcinoma; Colorectal cancer; Craniopharyngioma; |
| | Desmoid disease; Desmoid tumors; Duodenal polyposis; Familial |
| | adenomatous polyposis; Familial adenomatous polyposis-1; Familial |
| | multiple polyposis syndrome; Gardner syndrome; Gastric polyposis; |
| | Hepatocellular carcinoma; Hereditary cancer-predisposing syndrome; |
| | Hyperplastic colonic polyposis; Intestinal polyp; Malignant colorectal |
| | neoplasm; Neoplasm of stomach; Neoplasm of the large intestine; |
| | Periampullary adenoma |
| ATM | Ataxia-telangiectasia syndrome; Familial cancer of breast; Hereditary breast |
| | and ovarian cancer syndrome; Hereditary cancer-predisposing syndrome; |
| | Ovarian neoplasms |
| ATM, C11orf65, | Ataxia-telangiectasia syndrome; Ataxia-telangiectasia without |
| ATP13A2 | immunodeficiency; Breast cancer; Familial cancer of breast; Hereditary |
| | breast and ovarian cancer syndrome; Hereditary cancer-predisposing |
| | syndrome; Neoplasm of the breast; Susceptibility to Kufor-Rakeb syndrome |
| AXIN2 | Oligodontia-colorectal cancer syndrome |
| BAP1 | Hereditary cancer-predisposing syndrome; Tumor susceptibility linked to |
| | germline BAP1 mutations |
| BARD1 | Breast cancer; Familial cancer of breast; Hereditary breast and ovarian |
| | cancer syndrome; Hereditary cancer-predisposing syndrome; Triple- |
| | negative breast cancer |
| BLM | Bloom syndrome; Hereditary breast and ovarian cancer syndrome; |
| | Hereditary cancer-predisposing syndrome |
| BMPR1A | Hereditary cancer-predisposing syndrome; Juvenile polyposis syndrome |
| BRCA1 | Breast and/or ovarian cancer; Breast carcinoma; Familial breast-ovarian |
| | cancer-1; Dysgerminoma; Complementation group S Fanconi anemia; |
| | Familial cancer of breast; Hereditary breast and ovarian cancer syndrome; |
| | Hereditary cancer-predisposing syndrome; Infiltrating duct carcinoma of |
| | breast; Neoplasm of ovary; Neoplasm of the breast; Ovarian neoplasms; |
| | Ovarian serous surface papillary adenocarcinoma; Ovarian cancer; |
| | Pancreatic cancer; Pancreatic cancer-4; Porokeratosis punctata palmaris et |
| | plantaris; Rhabdomyosarcoma; Bilateral breast cancer; Breast cancer |
| BRCA2 | Asthma; BRCA2-related disorders; Breast and/or ovarian cancer; Breast |
| | carcinoma; Breast-ovarian cancer; Cancer of the pancreas; Colorectal |
| | cancer; Diffuse intrinsic pontine glioma; Ectopic ossification; Familial |
| | cancer of breast; Complementation group D1 Fanconi anemia; Focal |
| | seizures; Genetic non-acquired premature ovarian failure; Glioma |
| | susceptibility-3; Headache; Hereditary cancer syndrome; Hereditary breast |
| | and ovarian cancer syndrome; Hereditary cancer-predisposing syndrome; |
| | Inborn genetic diseases; Malignant tumor of prostate; Medulloblastoma; |
| | Migraine; Muscle weakness; Neoplasm of the breast; Nephrolithiasis; |
| | Obesity; Ovarian neoplasms; Ovarian cancer; Pancreatic cancer-2; |
| | Polydactyly; Short attention span; Striae distensae; Tracheoesophageal |
| | fistula; Tumor susceptibility linked to germline BAP1 mutations; Wilms |
| | tumor-1 |
| BRIP1 | BRIP1-related disorders; Breast cancer; Carcinoma of colon; Familial |
| | cancer of breast; Complementation group J Fanconi anemia; Hereditary |
| | breast and ovarian cancer syndrome; Hereditary cancer-predisposing |
| | syndrome; Neoplasm of ovary; Neoplasm of the breast; Ovarian Cancers; |
| | Ovarian Neoplasms; Tracheoesophageal fistula |
| C11orf65, | Ataxia-telangiectasia syndrome; Hereditary breast and ovarian cancer |
| ATM | syndrome; Hereditary cancer-predisposing syndrome |
| CBL | Noonan syndrome-like disorder with or without juvenile myelomonocytic |
| | leukemia |
| CDC73 | Parathyroid adenoma; Parathyroid carcinoma |
| CDH1 | Blepharocheilodontic syndrome-1; Lobular breast cancer; Endometrial |
| | carcinoma; Familial cancer of breast; Hereditary cancer-predisposing |
| | syndrome; Hereditary diffuse gastric cancer; Malignant tumor of prostate; |
| | Neoplasm of ovary |
| CDH23 | Pituitary adenoma-5 |
| CDKN2A | Hereditary cancer-predisposing syndrome; Hereditary cutaneous melanoma; |
| | Melanoma-pancreatic cancer syndrome; Neoplasm |
| CHEK2 | Astrocytoma; B Lymphoblastic leukemia/lymphoma; Breast and colorectal |
| | cancer; Breast cancer; CHEK2-related cancer susceptibility; Colitis; |
| | Congenital heart defects; Diffuse intrinsic pontine glioma; Familial cancer |
| | of breast; Hematochezia; Hereditary breast and ovarian cancer syndrome; |
| | Hereditary cancer; Hereditary cancer-predisposing syndrome; Inflammation |
| | of the large intestine; Leiomyosarcoma; Li-Fraumeni syndrome; Li- |
| | Fraumeni syndrome-2; Malignant tumor of prostate; Neoplasm of the |
| | breast; Osteosarcoma; Ovarian neoplasms; Prostate cancer; |
| | Thrombocytopenia |
| CHRNA3 | CHRNA3-related condition |
| CTNNB1 | Hepatocellular carcinoma |
| DDX41 | Acute myeloid leukemia; Susceptibility to familial myeloproliferative, |
| | lymphoproliferative neoplasms |
| DGKE | Nephrotic syndrome type 7 |
| DICER1 | DICER1-related pleuropulmonary blastoma cancer predisposition |
| | syndrome; Hereditary cancer-predisposing syndrome |
| ERCC4 | Pre-B-cell acute lymphoblastic leukemia |
| EXT1 | Sporadic chondrosarcoma; Multiple congenital exostosis; Multiple |
| | exostoses type 1 |
| EXT2 | Multiple exostoses type 2 |
| FANCC | Hereditary cancer-predisposing syndrome |
| FANCC, | Hereditary cancer-predisposing syndrome |
| AOPEP | |
| FANCM | Malignant germ cell tumor of ovary |
| FAS | Autoimmune lymphoproliferative syndrome |
| FH | Hereditary cancer-predisposing syndrome; Hereditary leiomyomatosis and |
| | renal cell cancer |
| FLCN | Hereditary cancer-predisposing syndrome; Multiple fibrofolliculomas |
| GATA1 | Acute megakaryoblastic leukemia |
| GPC3 | Simpson-Golabi-Behmel syndrome, Wilms tumor-1 |
| KHDC3L | Recurrent hydatidiform mole-2 |
| LOC100507346, | Gorlin syndrome; Medulloblastoma |
| PTCH1 | |
| LZTR1 | Noonan syndrome-2; Schwannomatosis-2 |
| MAP2K2 | Rasopathy |
| MAX | Hereditary cancer-predisposing syndrome |
| MEN1 | Hereditary cancer-predisposing syndrome; Somatic lipoma; Multiple |
| | endocrine neoplasia type 1 |
| MLH1 | Carcinoma of colon; Colon cancer; Hereditary cancer-predisposing |
| | syndrome; Hereditary nonpolyposis colon cancer; Lynch syndrome; Lynch |
| | syndrome-I; Lynch syndrome-II; Muir-Torre syndrome; Turcot syndrome |
| MLH3 | Hereditary nonpolyposis colorectal cancer type 7 |
| MRE11 | Hereditary cancer-predisposing syndrome |
| MSH2 | Carcinoma of colon; Colon cancer; Glioblastoma; Hereditary cancer- |
| | predisposing syndrome; Hereditary nonpolyposis colon cancer; Lynch |
| | syndrome; Lynch syndrome-I; Malignant tumor of ascending colon; |
| | Malignant tumor of sigmoid colon; Muir-Torre syndrome; Ovarian |
| | neoplasms; Turcot syndrome |
| MSH6 | Endometrial carcinoma; Hereditary cancer-predisposing syndrome; |
| | Hereditary nonpolyposis colon cancer; Hereditary nonpolyposis colorectal |
| | cancer type 5; Hereditary nonpolyposis colorectal carcinoma; Lynch |
| | syndrome; Lynch syndrome-I; Turcot syndrome |
| MUTYH | Carcinoma of colon; Colon cancer; Familial colorectal cancer; Hereditary |
| | cancer-predisposing syndrome; MUTYH-associated polyposis; MYH- |
| | associated polyposis; Neoplasm of stomach; Pilomatrixoma |
| NBN | Acute lymphoid leukemia; Aplastic anemia; Breast-ovarian cancer; Familial |
| | cancer of breast; Hereditary breast and ovarian cancer syndrome; Hereditary |
| | cancer-predisposing syndrome; Lissencephaly; Microcephaly with normal |
| | intelligence and immunodeficiency; Ovarian neoplasms |
| NCR1, NLRP7 | Recurrent hydatidiform mole-1 |
| NF1 | Axillary freckling; Cafe-au-lait macules with pulmonary stenosis; Focal T2 |
| | hyperintense basal ganglia lesion; Ganglioglioma; Hereditary cancer- |
| | predisposing syndrome; Inborn genetic diseases; Juvenile myelomonocytic |
| | leukemia; Multiple cafe-au-lait spots; Familial spinal neurofibroma; |
| | Neurofibromas; Neurofibromatosis type 1; Neurofibromatosis-Noonan |
| | syndrome; Optic nerve glioma; Pilocytic astrocytoma; Tibial |
| | pseudoarthrosis |
| NF1, | Hereditary cancer-predisposing syndrome; Neurofibromatosis type 1 |
| LOC111811965 | |
| NF2 | Meningioma; Neurofibromatosis type 2 |
| NLRP7 | Recurrent hydatidiform mole-1 |
| NSD1 | Beckwith-Wiedemann syndrome |
| NTHL1 | Familial adenomatous polyposis-3; Hereditary cancer-predisposing |
| | syndrome |
| OSGIN2, | Hereditary cancer-predisposing syndrome; Microcephaly with normal |
| NBN | intelligence and immunodeficiency |
| PALB2 | Basal cell carcinoma; Breast cancer; Cancer of the pancreas; Familial |
| | cancer of breast; Complementation group N Fanconi anemia; Generalized |
| | hypopigmentation; Hereditary breast and ovarian cancer syndrome; |
| | Hereditary cancer; Hereditary cancer-predisposing syndrome; Neoplasm of |
| | the breast; Ovarian neoplasms; PALB2-related disorders; Susceptibility to |
| | pancreatic cancer-3; Pre-B-cell acute lymphoblastic leukemia; |
| | Tracheoesophageal fistula; Tumor susceptibility linked to germline BAP1 |
| | mutations |
| PMS2 | Acute lymphoid leukemia; Burkitt lymphoma; Colorectal cancer; |
| | Glioblastoma; Hereditary cancer; Hereditary cancer-predisposing |
| | syndrome; Hereditary nonpolyposis colon cancer; Hereditary nonpolyposis |
| | colorectal cancer type 4; Lymphoma; Lynch syndrome; Lynch syndrome-I; |
| | Pulmonary arterial hypertension; Pulmonary insufficiency; Respiratory |
| | insufficiency; Tumor susceptibility linked to germline BAP1 mutations; |
| | Turcot syndrome |
| POLE | Colorectal cancer; Hereditary cancer-predisposing syndrome |
| POT1 | Hereditary cancer-predisposing syndrome; Susceptibility to cutaneous |
| | malignant melanoma-10 |
| PRKAR1A | Carney complex type 1 |
| PTCH1 | Gorlin syndrome; Hereditary cancer-predisposing syndrome |
| PTCH2 | Gorlin syndrome; Medulloblastoma |
| PTEN | Cowden syndrome; Cowden syndrome-1; Glioblastoma; Glioma |
| | susceptibility-2; Hemangioma; Hereditary cancer-predisposing syndrome; |
| | Inborn genetic diseases; Macrocephaly/autism syndrome; Malignant tumor |
| | of prostate; Familial meningioma; Neoplasm of brain; Neoplasm of the |
| | breast; Neoplasm of the large intestine; Non-small cell lung cancer; Ovarian |
| | neoplasms; PTEN hamartoma tumor syndrome; PTEN-related disorder; |
| | Proteus-like syndrome; VACTERL association with hydrocephalus |
| PTPN11 | Metachondromatosis |
| RABL3 | Susceptibility to pancreatic cancer-5 |
| RAD50 | Hereditary cancer-predisposing syndrome |
| RAD51C | Familial breast-ovarian cancer-3; Complementation group O Fanconi |
| | anemia; Hereditary breast and ovarian cancer syndrome; Hereditary cancer- |
| | predisposing syndrome; Ovarian neoplasms; RAD51C-related disorders |
| RAD51D, | Familial breast-ovarian cancer-4; Hereditary breast and ovarian cancer |
| RAD51L3-RFFL | syndrome; Hereditary cancer-predisposing syndrome; Ovarian neoplasms |
| RAD51L3- | Familial breast-ovarian cancer-4; Hereditary cancer-predisposing syndrome |
| RFFL, RAD51D | |
| RB1 | Hereditary cancer-predisposing syndrome; Neoplasm; Osteosarcoma; |
| | Trilateral retinoblastoma; Small cell lung cancer; Urinary bladder cancer |
| RECQL | Hereditary cancer-predisposing syndrome |
| RECQL, | Hereditary cancer-predisposing syndrome |
| PYROXD1 | |
| RECQL4 | B lymphoblastic leukemia with t(12; 21)(p13; q22); Baller-Gerold syndrome; |
| | High grade surface osteosarcoma; Rapadilino syndrome; Rothmund- |
| | Thomson syndrome; Rothmund-Thomson syndrome type 2 |
| RUNX1 | Acute myeloid leukemia; Familial platelet disorder with associated myeloid |
| | malignancy |
| SDHA | Carney triad; Dilated cardiomyopathy-1GG; Hereditary cancer-predisposing |
| | syndrome; Leigh syndrome; Mitochondrial complex II deficiency; |
| | Paragangliomas-5; Pilocytic astrocytoma |
| SDHAF2 | Hereditary paraganglioma-pheochromocytoma syndromes |
| SDHB | Carney-Stratakis syndrome; Gastrointestinal stromal tumor; Hereditary |
| | paraganglioma-pheochromocytoma syndromes; Hereditary cancer- |
| | predisposing syndrome; Paragangliomas-4; Pheochromocytoma |
| SDHC | Gastrointestinal stromal tumor; Hereditary paraganglioma- |
| | pheochromocytoma syndromes; Hereditary cancer-predisposing syndrome; |
| | Paragangliomas-3 |
| SDHD | Carney-Stratakis syndrome; Cowden syndrome-3; Hereditary |
| | paraganglioma-pheochromocytoma syndromes; Hereditary cancer- |
| | predisposing syndrome; Paragangliomas-1; Paragangliomas-1 with |
| | sensorineural hearing loss; Pheochromocytoma |
| SH2D1A | X-linked lymphoproliferative syndrome-1; X-Linked Lymphoproliferative |
| | Syndrome |
| SMAD4 | Carcinoma of pancreas; Hereditary cancer-predisposing syndrome; Juvenile |
| | polyposis syndrome; Juvenile polyposis/hereditary hemorrhagic |
| | telangiectasia syndrome; Myhre syndrome |
| SMARCA4 | Neuroblastoma |
| SMARCB1 | Atypical teratoid tumor |
| SMARCE1 | Familial meningioma |
| STK11 | Hereditary cancer-predisposing syndrome; Peutz-Jeghers syndrome |
| SUFU | Gorlin syndrome; Desmoplastic medulloblastoma; Medulloblastoma with |
| | extensive nodularity |
| TMEM127 | Hereditary paraganglioma-pheochromocytoma syndromes; Hereditary |
| | cancer-predisposing syndrome; Pheochromocytoma |
| TNFRSF10B | Squamous cell carcinoma of the head and neck |
| TP53 | Head and neck neoplasms; Hereditary cancer-predisposing syndrome; Li- |
| | Fraumeni syndrome; Li-Fraumeni syndrome-1; Li-Fraumeni-like syndrome; |
| | Multiple myeloma; Neoplasm of the large intestine; Ovarian neoplasms |
| TSC1 | Cortical tubers; Hereditary cancer-predisposing syndrome; |
| | Lymphangiomyomatosis; Tuberous sclerosis-1; Tuberous sclerosis |
| | syndrome; Urinary bladder cancer |
| TSC2 | Lymphangiomyomatosis; Tuberous sclerosis-2; Tuberous sclerosis |
| | syndrome |
| UNC13D | Familial hemophagocytic lymphohistiocytosis-3 |
| VHL | Familial erythrocytosis-2; Hereditary cancer-predisposing syndrome; Von |
| | Hippel-Lindau syndrome |
| VHL, | Familial erythrocytosis-2; Hereditary cancer-predisposing syndrome; |
| LOC107303340 | Papillary renal cell carcinoma-1; Von Hippel-Lindau syndrome |
| WRN | Medulloblastoma; Werner syndrome |
| WT1 | Drash syndrome; Frasier syndrome; Wilms tumor, aniridia, genitourinary |
| | anomalies, and mental retardation syndrome; Wilms tumor-1 |
| WT1, | Drash syndrome; Frasier syndrome; Pre-B-cell acute lymphoblastic |
| LOC107982234 | leukemia; Wilms tumor, aniridia, genitourinary anomalies, and mental |
| | retardation syndrome; Wilms tumor-1 |
| XIAP | X-linked lymphoproliferative syndrome-2 |
| XRCC2 | Complementation group U Fanconi anemia; Hereditary cancer syndrome; |
| | Hereditary breast and ovarian cancer syndrome; Hereditary cancer- |
| | predisposing syndrome; Ovarian neoplasms |
| ZDBF2 | Nasopalpebral lipoma-coloboma syndrome |

In one aspect, the present disclosure features a method of reducing tumor size by administering a TREM or a composition thereof (e.g., a TREM or composition thereof disclosed herein) to a cell or a subject. In an embodiment, a method of reducing tumor size comprises reducing tumor diameter. Tumor size, e.g., tumor diameter, can be measured using typical methods known in the art, for example, imaging (e.g., ultrasound, computerized tomography (CT), positron emission tomography (PET), or magnetic resonance imaging (MRI) scans) or tumor caliper measurements. In an embodiment, reduction of tumor size refers to a reduction in tumor diameter from an initial measurement. In an embodiment, upon administration of a TREM or a composition thereof, the tumor size is reduced by about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, e.g., as compared to the tumor size prior to aministration of a TREM or a composition thereof (e.g., a TREM or composition thereof disclosed herein).

In an embodiment, reduction of tumor size is accomplished by slowing the rate of increased tumor cell growth, e.g., cell cycle arrest. In an embodiment, reduction of tumor size is accomplished by reducing the number of living cancer cells. In some embodiments, reducing the number of living cancer cells is caused by inducing cell death of living cancer cells. Cell death can be caused by a number of cell processes (e.g., apoptosis, autophagy, anoikis, necrosis, entosis). In an embodiment, reduction of tumor size is accomplished by encouraging growth of non-proliferating cells, e.g., increasing the umber of non-proliferating cells.

In another aspect, the present disclosure features a method of reducing tumor mass by administering a TREM or a composition thereof (e.g., a TREM or composition thereof disclosed herein) to a cell or a subject. Tumor mass may be expressed by any known term, including weight/weight (w/w), weight/volume (w/v), or volume/volume (v/v). In an embodiment, reduction of tumor mass refers to a reduction in tumor mass from an initial measurement. In an embodiment, upon administration of a TREM or a composition thereof, the tumor mass is reduced by about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, e.g., as compared to the tumor mass prior to administration of a TREM or a composition thereof (e.g., a TREM or composition thereof disclosed herein).

In another aspect, the present disclosure features a method of reducing tumor proliferation by administering a TREM or a composition thereof (e.g., a TREM or composition thereof disclosed herein) to a cell or a subject. Tumor proliferation may refer to the rate at which the total number of cells within a tumor divide, or to the rate at which the number of total tumors increases in a subject or sample. In an embodiment, reduction of tumor proliferation refers to a reduction in tumor proliferation from an initial measurement. In an embodiment, upon administration of a TREM or a composition thereof, the tumor proliferation is reduced by about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, e.g., as compared to the tumor proliferation prior to administration of a TREM or a composition thereof (e.g., a TREM or composition thereof disclosed herein).

In another aspect, the present disclosure features a method of reducing metastasis of a cancer in a subject by administering a TREM or a composition thereof (e.g., a TREM or composition thereof disclosed herein) to a cell or a subject. Metastasis refers to the development of an additional (e.g., secondary, tertiary) site of cancer growth (e.g., tumor presence), e.g., at a distance from the primary site of a cancer in a subject. Metastasis may comprise a cell or tumor that dissociates from an original cancer site within a subject, travels through the blood or lymph in the subject, arrives at a distant site within the subject compared to the original cancer site, and continues to proliferate. In an embodiment, reduction of metastasis refers to a reduction in metastasis from an initial measurement. In an embodiment, upon administration of a TREM or a composition thereof, the rate of metastasis is reduced by about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, e.g., as compared to the rate of metastasis prior to administration of a TREM or a composition thereof (e.g., a TREM or composition thereof disclosed herein).

In another aspect, the present disclosure features a method of increasing the survival time of a cell or subject by administering a TREM or a composition thereof (e.g., a TREM or composition thereof disclosed herein) to a cell or a subject. Survival time refers to the overall viability a cell or subject, e.g. including overall life span, or time during which the cell or subject is carrying out life functions. In an embodiment, upon administration of a TREM or a composition thereof, the survival time of a cell or subject is increased by about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, e.g., as compared to the survival time in the absence of administration of a TREM or a composition thereof (e.g., a TREM or composition thereof disclosed herein). In an embodiment, the survival time of a cell or subject is increased by about 1 day, 2 days, 3 days, 1 week, 2 weeks, 3 weeks, 1 month, 2 month, 3 months, 4 months, 5 months, 6 months, 1 year, 1.5 years, 2 years, 3 years, 4 years, 5 years, 10 years, or longer.

In another aspect, the present disclosure features a method of reducing a symptom of a cancer in a subject by administering a TREM or a composition thereof (e.g., a TREM or composition thereof disclosed herein) to a cell or a subject. In an embodiment, reducing a symptom of a cancer includes reducing the severity of a symptom and/or reducing the duration of a symptom. Exemplary symptoms of cancer include exhaustion, nausea, decreased appetite, hair loss, reduced immunity, weakness, muscle atrophy, weight loss, weight gain, pain, swelling, sweating, behavioral changes, headaches, constipation, diarrhea, numbness, and coughing. In an embodiment, reduction of a symptom of a cancer refers to a reduction in a symptom of a cancer from an initial measurement. In an embodiment, upon administration of a TREM or a composition thereof, a symptom of a cancer is reduced by about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, e.g., as compared to the symptom of a cancer prior to administration of a TREM or a composition thereof (e.g., a TREM or composition thereof disclosed herein).

### Patient Selection

Methods provided herein involve selecting and treating a subject suitable for treatment. The subject may be a mammal, e.g., a human. In some embodiments, the subject is an adult (e.g., a human over 18 years of age, e.g., over 35 years of age, over 50 years of age). In some embodiments, the subject is a child (e.g., a human under 18 years of age, e.g., under 12 years of age, under 8 years of age, under 5 years of age).

In some embodiments, the subject is naïve to one or more therapies prior to administration of a TREM as described herein. In some embodiments, the subject has received one or more therapies for cancer prior to administration of a TREM as described herein. In some embodiments, the subject is, or is identified as, a complete responder, a partial responder, non-responder, or relapse to one or more therapies for the cancer. In some embodiments, the subject's response to one or more prior treatments is assessed at predetermined time intervals, e.g., before or during treatment with the one or more therapies. If the assessment shows that the patient is a complete responder, the TREM may not administered. If the assessment shows that the subject is a partial responder, or has stable disease in response, the TREM may be administered. If the assessment shows that the subject is a non-responder or relapse, the TREM may be administered, e.g., in combination with an additional therapy.

The subject may have or be diagnosed as having a premature termination codon (PTC)-associated tumor. In some embodiments, the subject does not have, or has not been diagnosed as having, a PTC-associated tumor. Identification of a PTC-associated tumor may be carried out by techniques known in the art. In some embodiments, the diagnosis comprises obtaining a tumor sample from the patient, subjecting the tumor sample to the technique which identifies a PTC mutation, and comparing the tumor sample to a standard sample (e.g., a non-cancerous sample of the same tissue type). Exemplary techniques used to identify a PTC mutation include, but are not limited to, nucleotide sequencing methods, imaging, and affinity labeling, and chromatography (e.g., ELISA, SDS-PAGE, Western blotting).

In some embodiments, the patient has no incidence of cancer prior to receiving a TREM. In some embodiments, the patient has experienced a relapse in cancer. In some embodiments, the patient has refractory cancer. In some embodiments, the patient has been diagnosed with metastatic cancer.

In certain aspects, a TREM and compositions thereof disclosed herein are administered when a PCT mutation in a subject has been identified as a driver mutation. Driver mutations are alterations that give a cancer cell a fundamental growth advantage for neoplastic transformation.

In some embodiments, the PTC driver mutation is found in the adenomatous polyposis coli (APC) tumor suppressor gene. Mutations in the APC gene is the most common mutation in colon cancers, however it can arise in other cancers (e.g., uterine endometrioid carcinoma, ampullary carcinoma, stomach adenocarcinoma, rectal adenocarcinoma, lung adenocarcinoma, pancreatic adenocarcinoma, cervical squamous cell carcinoma, upper tract urothelial carcinoma, goblet cell carcinoid of the appendix, skin cancer, cutaneous melanoma, bladder urothelial carcinoma, glioblastoma multiforme, breast invasive ductal carcinoma, basaloid penile squamous cell carcinoma, adenoid cystic carcinoma, oral cavity squamous cell carcinoma, prostate adenocarcinoma, high-grade spindle cell sarcoma, non-small cell lung cancer). The PTC mutations found in the APC protein once translated, can be at, but are not limited to, amino acid positions 24, 213, 216, 232, 283, 302, 332, 348, 405, 499, 554, 564, 790, 805, 838, 876, 919, 923, 924, 958, 976, 1114, 1158, 1239, 1331, 1386, 1435, 1450, 1463, 1858, 1920, 2166, 2204, 2226, 2237, 2326, 2371, 2560, and 2816.

In some embodiments, the PTC driver mutation is found in the Breast cancer type 1 (BRCA1) tumor suppressor gene. The BRCA1 protein is part of a complex that repairs double-strand breaks in DNA. Mutations in BRCA1 increases the risk for breast cancer as part of a hereditary breast-ovarian cancer syndrome. PTC mutations in BRCA1 have also been found in melanoma, uterine endometrioid carcinoma, and cutaneous melanoma. PTC mutations found in the BRCA1 protein can be at, but are not limited to, amino acid positions 1203, 1443, and 1751.

In some embodiments, the PTC driver mutation is found in the Breast cancer type 2 (BRCA2) tumor suppressor gene. The BRCA2 protein is part of a complex that repairs double-strand breaks in DNA. Mutations in BRCA2 gene increases the risk for breast cancer as part of a hereditary breast-ovarian cancer syndrome. PTC mutations in BRCA1 have also been found in other cancers, e.g., pancreatic adenocarcinoma, head and neck squamous cell carcinoma, gallbladder cancer, and uterine endometrioid carcinoma. PTC mutations found in the BRCA2 protein can be at, but are not limited to, amino acid positions 2318, 250, 2625, 3128, and 3384.

In some embodiments, the PTC driver mutation is found in the SMAD4 gene. SMAD4 serves as a mediator between extracellular growth factors from the TGFβ family and genes inside the cell nucleos. It is also defined as a signal transducer. Mutations in SMAD4 have been found in a number of different cancers, e.g., ampullary carcinoma, cutaneous squamous cell carcinoma, pancreatic adenocarcinoma, bladder urothelial carcinoma, breast invasive lobular carcinoma, intrahepatic cholandiocarcinoma, appendiceal adenocarcinoma, mucinous adenocarcinoma of the appendix, lung adenocarcinoma, colorectal carcinoma, esophageal adenocarcinoma, cervical squamous cell carcinoma, head and neck squamous cell carcinoma, uterine endometrioid carcinoma, and intestinal type stomach adenocarcinoma. PTC mutations found in the SMAD4 protein can be at, but are not limited to, amino acid positions 27, 135, and 445.

In some embodiments, the PTC driver mutation is found in the cyclin-dependent kinase inhibitor 2A (CDKN2A) gene. CDKN2A is ubiquitously expressed in many tissues and cell types. The protein regulates the cell cycle by inhibiting cyclin dependent kinases 4 and 6, thereby activating the retinoblastoma (Rb) family of proteins, which block traversal from G1 to S-phase. Mutations of CDKN2A are common in the majority of human cancers, e.g., ampullary carcinoma, melanoma, thymic carcinoma, esophageal adenocarcinoma, lung adenocarcinoma, oral cavity squamous cell carcinoma, pancreatic adenocarcinoma, renal clear cell carcinoma, uterine endometrioid carcinoma, colon adenocarcinoma, cutaneous squamous cell carcinoma, upper trace urothelial carcinoma, small bowel cancer, cholangiocarcinoma, medullary carcinoma of the colon, glioblastoma multiforme, skin cancer (non-melanoma), esophageal squamous cell carcinoma, prostate adenocarcinoma, bladder urothelial carcinoma, head and neck squamous cell carcinoma, breast invasive ductal carcinoma, papillary renal cell carcinoma, adenoid cystic carcinoma, penile squamous cell carcinoma, myxofibrosarcoma, mucinous ovarian cancer, undifferentiated pleomorphic sarcoma, non-small cell lung cancer, activated B-cell type, diffuse large B-cell lymphoma, and AML with Biallelic mutations. PTC mutations found in the CDKN2A protein can be at, but are not limited to, amino acid positions 58 or 80.

In some embodiments, the PTC driver mutation is found in the SMAD2 gene. SMAD2 mediates the signal of transforming growth factor (TGF)-beta, and thus regulates multiple cellular processes, such as cell proliferation, apoptosis, and differentiation. Mutations in SMAD2 have been found in a number of different cancers, e.g., colorectal adenocarcinoma, skin cancer, esophageal adenocarcinoma, colon adenocarcinoma, mucinous adenocarcinoma of the colon and rectum, uterine serous carcinoma, uterine endometrioid carcinoma, head and neck squamous cell carcinoma, pancreatic adenocarcinoma, and breast invasive ductal carcinoma. PTC mutations found in the SMAD2 protein can be at, but are not limited to, amino acid positions 57, 120, 130, 182, 321, and 427.

In some embodiments, the PTC driver mutation is found in the neurofibromin (NF1) gene. NF1 helps regulate cell growth, and mutations in this gene causes a loss of neurofibromin, which leads to uncontrolled cell growth. Mutations in NF1 have been found in a number of different cancers, e.g., melanoma, breast invasive ductal carcinoma, upper tract urothelial carcinoma, oligodendroglioma, head and neck mucosal melanoma, colon adenocarcinoma, astrocytoma, uterine carcinosarcoma, uterine endometrioid carcinoma, cervical squamous cell carcinoma, renal cell carcinoma, melanoma, breast invasive lobular carcinoma, glioblastoma multiforme, skin cancer (non-melanoma), rectal adenocarcinoma, malignant peripheral nerve sheath tumor, stomach adenocarcinoma, sarcoma, serous ovarian cancer, angiosarcoma, acral melanoma, acute myeloid leukemia, B-lymphoblastic leukemia or lymphoma, anaplastic astrocytoma, rhabdomyosarcoma, and small bowel cancer. PTC mutations found in the NF1 protein can be at, but are not limited to, amino acid positions 103, 192, 304, 366, 416, 440, 461, 681, 816, 1241, 1276, 1306, 1362, 1412, 1534, 1769, 1968, 2258, 2450, 2458, 2517, and 2637.

In some embodiments, the PTC driver mutation is found in the MERLIN (NF2) gene. NF2 is a cytoskeleton protein that is also a tumor suppressor protein. Mutations in NF2 have been found in a number of different cancer, e.g., pleural mesothelioma, renal clear cell carcinoma with sarcomatoid features, atypical meningioma, lung adenocarcinoma, head and neck squamous cell carcinoma, breast invasive ductal carcinoma, melanoma, rectal adenocarcinoma, basal cell carcinoma, intrahepatic cholangiocarcinoma, pleural mesothelioma, cervical squamous cell carcinoma, desmoplastic melanoma, peritoneal mesothelioma, pancreatic adenocarcinoma, mucinous adenocarcinoma of the coon and rectum, high-grade serous ovarian cancer, poorly differentiated carcinoma, uterine serous carcinoma, and upper tract urothelial carcinoma. PTC mutations found in the NF2 protein can be at, but are not limited to, amino acid positions 57, 196, 198, 249, 262, 341, and 466.

In some embodiments, the PTC driver mutation is found in the TP53 gene. The p53 protein is a tumor suppressor gene that has a role in conserving stability by preventing genome mutations. It plays a role in regulation or progression through the cell cycle, apoptosis, and genomic stability. Mutations in T53 have been identified in a number of different cancers, e.g., ampullary carcinoma, stomach adenocarcinoma, pancreatic neuroendocrine tumor, plasma cell myeloma, cutaneous squamous cell carcinoma, rectal adenocarcinoma, anaplastic astrocytoma, breast cancer, gallbladder cancer, small bowel cancer, high-grade serous ovarian cancer, melanoma, glioblastoma multiforme, oral cavity squamous cell carcinoma, pancreatic carcinoma, esophageal adenocarcinoma, adenoid cystic carcinoma, lung cancer, merkel cell carcinoma, mantel cell lymphoma, small cell lung cancer, diffuse large B-cell lymphoma, skin cancer, prostate neuroendocrine carcinoma, oligodendroglioma, cervical squamous cell carcinoma, head and neck squamous cell carcinoma, penile squamous cell carcinoma, and ovarian cancer. PTC mutations found in the NF2 protein can be at, but are not limited to, amino acid positions 65, 196, 209, 213, 280, 306, and 342.

In some embodiments, the PTC driver mutation is found in the phosphatase and tensin homolog (PTEN). The PTEN protein acts as a tumor suppressor gene through the action of its phosphatase protein product. This phosphatase is involved the regulation of the cell cycle, preventing cells from growing and dividing too rapidly. Mutations in PTEN have been identified in a number of different cancers, e.g., Intrahepatic Cholangiocarcinoma, Esophagogastric Adenocarcinoma, breast invasive ductal carcinoma, glioblastoma multiforme, cutaneous squamous cell carcinoma, sinonasal squamous cell carcinoma, ovarian carcinosarcoma, poorly differentiated thyroid cancer, uterine endometrioid carcinoma, prostate carcinoma, gliosarcoma, prostate adenocarcinoma, melanoma, uterine endometrioid carcinoma, colon cancer, head and neck squamous cell carcinoma, lung cancer, adenoid cystic carcinoma, renal non-clear cell carcinoma, germinal center B-cell type, diffuse large B-cell lymphoma, colorectal carcinoma, stomach adenocarcinoma, cervical squamous cell carcinoma prostate cancer, and astrocytoma. PTC mutations found in the PTEN protein can be at, but are not limited to, amino acid positions 15, 84, 130, 189, 233, and 335.

In some embodiments, the PTC driver mutation is found in the retinoblastoma (RB1) gene. Wild-type RB1 prevents excessive cell growth by inhibiting cell cycle progression until a a cell is ready to divide. Mutations in RB1 have been identified in several major cancers, e.g., bladder urothelial carcinoma, lung cancer, diffuse large B-cell lymphoma, leiomyosarcoma, breast cancer, glioblastoma multiforme, retinoblastoma, hepatocellular carcinoma, small cell lung cancer, cutaneous squamous cell carcinoma, esophageal squamous cell carcinoma, head and neck squamous cell carcinoma, stomach adenocarcinoma, serous ovarian cancer, melanoma, merkel cell carcinoma, prostate cancer, bladder/urinary tract cancer, and skin cancer. PTC mutations found in the RB1 protein can be at, but are not limited to, amino acid positions 251, 255, 320, 358, 445, 467, 500, 552, 556, 579, 763, 787, 830, and 908.

In some embodiments, the PTC driver mutation is found in the Von Hippel-Lindau tumor suppressor (VHL) gene. The VHL protein is thought to have E3 ubiquitin ligase activity that results in specific target proteins being marked for degradation. Mutations in VHL have been associated with several cancers, e.g., renal clear cell carcinoma, prostate cancer, and mucinous stomach adenocarcinoma. PTC mutations found in the VHL protein can be at, but are not limited to, amino acid positions 120, 161, and 177.

In some embodiments, the PTC driver mutation is found in the Wilms' tumor (WT1) gene. The WT1 protein is a transcription factor that has an essential role in the normal development of the urogenital system. Mutations in WT1 have been associated with several cancers, e.g., Wilms' tumor, esophageal squamous cell carcinoma, acute myeloid leukemia, prostate adenocarcinoma, colon adenocarcinoma, glioblastoma multiforme, skin cancer, breast cancer, uterine endometrioid carcinoma, and head and neck squamous cell carcinoma. PTC mutations found in the WT1 protein can be at, but are not limited to, amino acid positions 369, 430, and 458.

In some embodiments, the PTC driver mutation is found in the ATM serine/threonine kinase (ATM) gene. The ATM protein is serine/threonine protein kinase that is recruited and activated by DNA double-strand breaks. Mutations in ATM have been associated with several cancers, e.g., lung cancer, colon cancer, bladder urothelial carcinoma, skin cancer, rectal adenocarcinoma, diffuse large B-cell lymphoma, ampullary carcinoma, cutaneous melanoma, uterine carcinosarcoma, mantel cell lymphoma, myeloid neoplasms, pancreatobiliary ampullary carcinoma, chronic lymphocytic leukemia, small bowel cancer, uterine endometrioid carcinoma, angioimmunoblastic T-cell lymphoma, prostate cancer, basal cell carcinoma, leiomyosarcoma, breast cancer. PTC mutations found in the ATM protein can be at, but are not limited to, amino acid positions 23, 35, 62, 248, 250, 447, 568, 805, 1437, 1466, 1618, 1730, 1875, 2034, 2263, 2419, 2443, 2486, 2580, 2598, 2723, 2849, 2993, and 3047.

### Combinations

The present disclosure features methods of administering a TREM or a composition thereof in combination with an additional agent or therapy, for example, a cancer therapy. Exemplary cancer therapies include, for example, surgery, chemotherapy, targeted therapy (e.g., antibody therapy), immunotherapy, and hormonal therapy.

A combination therapy entails the administration of two or more agents. Each agent may be formulated in separate compositions or may be formulated in a single composition. In some embodiments, each agent within the combination therapy is formulated in separate compositions and administered individually (e.g., sequentially or concomitantly). In some embodiments, each agent is formulated and administered as a single formulation. In some embodiments, two agents can be formulated together and administered in combination with another formulation containing a third agent. In some embodiments, the TREM is separately formulated and administered first, e.g., before a second agent. In some embodiments, the TREM is separately formulated and administered after one or more agents. In some embodiments, the TREM is formulated separately and administered concurrently with one or more additional agents.

### Chemotherapy

In some embodiments, a TREM or composition thereof described herein is administered with a chemotherapy. Chemotherapy is the treatment of cancer with drugs that can destroy cancer cells. “Chemotherapy” usually refers to cytotoxic drugs which affect rapidly dividing cells in general, in contrast with targeted therapy. Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include, for example, antimetabolites (e.g., folic acid, purine, and pyrimidine derivatives) and alkylating agents (e.g., nitrogen mustards, nitrosoureas, platinum, alkyl sulfonates, hydrazines, triazenes, aziridines, spindle poison, cytotoxic agents, topoisomerase inhibitors and others). Exemplary agents include Aclarubicin, Actinomycin, Alitretinoin, Altretamine, Aminopterin, Aminolevulinic acid, Amrubicin, Amsacrine, Anagrelide, Arsenic trioxide, Asparaginase, Atrasentan, Belotecan, Bexarotene, endamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein. In an embodiment, the TREM or a composition thereof is administered with a chemotherapeutic agent described herein.

### Targeted Therapy

In some embodiments, a TREM or composition thereof described herein is administered with a targeted therapy. Targeted therapy constitutes the use of agents specific for the cancer cells, e.g., a deregulated proteins associated with a cancer cell. Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, desatinib, erlotinib, imatinib, gefitinib, lapatinib, Lestaurtinib, Nilotinib, Semaxanib, Sorafenib, Sunitinib, and Vandetanib, and also cyclin-dependent kinase inhibitors such as Alvocidib and Seliciclib. Monoclonal antibody therapy is another strategy in which the therapeutic agent is an antibody which specifically binds to a protein on the surface of the cancer cells. Examples include the anti-HER2/neu antibody trastuzumab (Herceptin®) typically used in breast cancer, and the anti-CD20 antibody rituximab and Tositumomab typically used in a variety of B-cell malignancies. Other exemplary antibodies include Cetuximab, Panitumumab, Trastuzumab, Alemtuzumab, Bevacizumab, Edrecolomab, and Gemtuzumab. Exemplary fusion proteins include Aflibercept and Denileukin diftitox. Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell. An example of such therapy includes BEXXAR®. In an embodiment, the TREM or a composition thereof is administered with a targeted therapy described herein.

### Immunotherapy

In some embodiments, a TREM or composition thereof described herein is administered with an immunotherapy. Cancer immunotherapy refers to a diverse set of therapeutic strategies designed to induce the patient's own immune system to fight the tumor. Exemplary immunotherapies include immune checkpoint inhibitors, T-cell therapy, monoclonal antibodies, cancer vaccines, and immune system modulators.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al, 1998); cytokine therapy, e.g., interferons α, β, and γ, IL-1, GM-CSF, and TNF (Bukowski et al, 1998; Davidson et al, 1998; Hellstrand et al, 1998); gene therapy, e.g., TNF, IL-1, IL-2, and p53 (Qin et al, 1998; Austin-Ward and Villaseca, 1998; U.S. Pat. Nos. 5,830,880 and 5,846,945); and monoclonal antibodies, e.g., anti-CD20, anti-ganglioside GM2, and anti-pl85 (Hollander, 2012; Hanibuchi et al, 1998; U.S. Pat. No. 5,824,311). It is contemplated that one or more anti-cancer therapies may be employed with the TREMS described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

Exemplary immune stimulating molecules include cytokines, such as IL-2, IL-4, IL-12, GM-CSF, gamma-IFN, chemokines, such as MIP-1, MCP-1, IL-8, and growth factors, such as FLT3 ligand. Exemplary cancer vaccines include HPV vaccines, and T-VEC.

In some embodiments, a TREM as described herein can be used in combination with an immune effector cell that expresses a chimeric antigen receptor (CAR) or an engineered T cell receptor (TCR). Chimeric antigen receptors are proteins which graft the specificity of a monoclonal antibody (mAb) to the effector function of a T cell. CARs are antigen receptors that are designed to recognize cell surface antigens in a human leukocyte antigen-dependent manner. Their usual form is that of a type I transmembrane domain protein with an antigen recognizing amino terminus, a spacer, a transmembrane domain all connected to a compound endodomain which transmits T-cell survival and activation signals.

The most common forms of these molecules are fusions of single-chain variable fragments (scFv) derived from monoclonal antibodies which recognize a target antigen, fused via a spacer and a trans-membrane domain to a signalling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Current CAR T-cell therapies currently typically consist of a mixture of T-cells comprising of CD4+ T-cells, CD8+ T-cells and T-cells which are naive, stem-cell memory, central memory and effector memory. Other immune effector cells that can also be modified with CARs are natural killer (NK) cells, or B cells. In some embodiments, the immune effector cells can be autologous. In some embodiments, the immune effector cells can be allogeneic.

T cell receptors (TCRs) mediate the recognition of specific major histocompatibility complex (MHC)-restricted peptide antigens by T cells and are essential to the functioning of the cellular arm of the immune system. Most TCRs are composed of two disulfide linked polypeptide chains, the alpha and beta chain. TCRs can be engineered with scFvs to specific peptide antigens to direct immune response.

### Hormonal Therapy

In some embodiments, a TREM or composition thereof described herein is administered with a hormonal therapy. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial. In some embodiments, the hormonal therapy agents can be used in combination with a TREM or a composition described herein.

In some embodiments, the TREM is administered in combination with other oligonucleotides. In some embodiments, the oligonucleotide is an RNA. In some embodiments, the RNA is a mRNA. In some embodiments, the RNA is a miRNA. In some embodiments, the RNA is a snoRNA. In some embodiments, the RNA is a siRNA. In some embodiments, the RNA is a second TREM. In some embodiments, the RNA is a snRNA. In some embodiments, the RNA is a lncRNA. In some embodiments, the RNA is a piRNA.

In some embodiments, the TREM is administered in combination with DNA. In some embodiments, the DNA is an antisense oligonucleotide (ASO). In some embodiments, the DNA is a vector. In some embodiments, the vector is a viral vector. In some embodiments, the viral vector is a lentiviral vector. In some embodiments, the viral vector is an adeno-associated viral vector. In some embodiments, the viral vector is a adenoviral vector.

### Radiation

In some embodiments, the TREM is administered in combination with radiation therapy. In some embodiments, the radiation is administered prior to surgical resection or another type of therapy, e.g., a TREM. In some embodiments, the radiation is administered after surgical resection or another type of therapy, e.g., a TREM. In some embodiments, the radiation therapy is intraoperative radiation therapy. In some embodiments, the radiation therapy is image guided radiation therapy. In some embodiments, the radiation therapy is intensity modulated radiation therapy. In some embodiments, the radiation therapy is volumetric modulated arc therapy. In some embodiments, the radiation is localized to a tumor site or in the area around a tumor, e.g., brachytherapy. In some embodiments, the TREM is administered prior to radiation therapy. In some embodiments, the TREM is administered after radiation therapy.

Tumor resection refers to the physical removal of at least part of a tumor. Tumor resection or treatment of a tumor by surgery can include laser surgery, cryosurgery, electrosurgery, and microscopically controlled surgery. In some embodiments, the TREM is administered before surgical resection of a tumor. In some embodiment, the TREM is administered to reduce the size of the tumor prior to resection. In some embodiments, the TREM is administered after surgical resection of a tumor.

In some embodiments, the additional agent is an agent that targets alternative splicing. (poison exon).

### Method of Making TREMs, TREM Core Fragments, and TREM Fragments

In vitro methods for synthesizing oligonucleotides are known in the art and can be used to make a TREM, a TREM core fragment or a TREM fragment disclosed herein. For example, a TREM, TREM core fragment or TREM fragment can be synthesized using solid state synthesis or liquid phase synthesis.

In an embodiment, a TREM, a TREM core fragment or a TREM fragment made according to an in vitro synthesis method disclosed herein has a different modification profile compared to a TREM expressed and isolated from a cell, or compared to a naturally occurring tRNA.

An exemplary method for making a modified TREM is provided in herein, e.g., Example 1. The method provided in Example 1 can also be used to make a synthetic TREM core fragment or synthetic TREM fragment.

### TREM Composition

In an embodiment, a TREM composition, e.g., a TREM pharmaceutical composition, comprises a pharmaceutically acceptable excipient. Exemplary excipients include those provided in the FDA Inactive Ingredient Database (https://www.accessdata.fda.gov/scripts/cder/iig/index. Cf(m).

In an embodiment, a TREM composition, e.g., a TREM pharmaceutical composition, comprises at least 1, 2, 3, 4, 5, 6, 7, 8, 9, 10, 15, 20, 30, 40, 50, 60, 70, 80, 90, 100 or 150 grams of TREM, TREM core fragment or TREM fragment. In an embodiment, a TREM composition, e.g., a TREM pharmaceutical composition, comprises at least 1, 2, 3, 4, 5, 6, 7, 8, 9, 10, 15, 20, 30, 40, 50 or 100 milligrams of TREM, TREM core fragment or TREM fragment.

In an embodiment, a TREM composition, e.g., a TREM pharmaceutical composition, is at least 10, 20, 30, 40, 50, 60, 70, 80, 90, 95 or 99% dry weight TREMs, TREM core fragments or TREM fragments. In an embodiment, a TREM composition comprises at least 1×10⁶ TREM molecules, at least 1×10⁷ TREM molecules, at least 1×10⁸ TREM molecules or at least 1×10⁹ TREM molecules.

In an embodiment, a TREM composition comprises at least 1×10⁶ TREM core fragment molecules, at least 1×10⁷ TREM core fragment molecules, at least 1×10⁸ TREM core fragment molecules or at least 1×10⁹ TREM core fragment molecules.

In an embodiment, a TREM composition comprises at least 1×10⁶ TREM fragment molecules, at least 1×10⁷ TREM fragment molecules, at least 1×10⁸ TREM fragment molecules or at least 1×10⁹ TREM fragment molecules.

In an embodiment, a TREM composition produced by any of the methods of making disclosed herein can be charged with an amino acid using an in vitro charging reaction as known in the art.

In an embodiment, a TREM composition comprise one or more species of TREMs, TREM core fragments, or TREM fragments. In an embodiment, a TREM composition comprises a single species of TREM, TREM core fragment, or TREM fragment. In an embodiment, a TREM composition comprises a first TREM, TREM core fragment, or TREM fragment species and a second TREM, TREM core fragment, or TREM fragment species. In an embodiment, the TREM composition comprises X TREM, TREM core fragment, or TREM fragment species, wherein X=2, 3, 4, 5, 6, 7, 8, 9, or 10.

In an embodiment, the TREM, TREM core fragment, or TREM fragment has at least 70, 75, 80, 85, 90, or 95, or has 100%, identity with a sequence encoded by a nucleic acid in Table 3.

In an embodiment, the TREM comprises a consensus sequence provided herein.

A TREM composition can be formulated as a liquid composition, as a lyophilized composition or as a frozen composition.

In some embodiments, a TREM composition can be formulated to be suitable for pharmaceutical use, e.g., a pharmaceutical TREM composition. In an embodiment, a pharmaceutical TREM composition is substantially free of materials and/or reagents used to separate and/or purify a TREM, TREM core fragment, or TREM fragment.

In some embodiments, a TREM composition can be formulated with water for injection. In some embodiments, a TREM composition formulated with water for injection is suitable for pharmaceutical use, e.g., comprises a pharmaceutical TREM composition.

### Methods of Delivery

Disclosed herein are methods of providing a tRNA-based effector molecule (TREM) or a composition thereof to a subject having a proliferative disease or disorder, such as a cancer (e.g., to a cancerous cell, tissue, or organ in the subject). Any TREM composition or pharmaceutical composition described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, epidural, intratumoral, rectal, vaginal, oral, sublingual, or buccal. In an embodiment, the TREM or a composition thereof is administered parenterally. In an embodiment, the TREM or a composition thereof is administered by injection. In an embodiment, the TREM or a composition thereof is delivered locally, e.g., to a tumor, e.g., into or adjacent to a tumor. In an embodiment, the TREM or a composition thereof is delivered to a subject intratumorally. As used herein, the term “intratumoral” refers to the area in or around a tumor. In embodiments, the TREM or a composition thereof is delivered to a subject having cancer (e.g., to a cancerous cell, tissue, or organ in a subject), wherein the cancer is caused by or is associated with a premature termination codon (PTC) signature. As used herein, the term “premature termination codon (PTC) signature” refers to detection of the presence of a PTC in the nucleic acid sequence that encodes a protein. The nucleic acid sequence with the PTC signature may be DNA or mRNA. For example, the PTC signature detected in the nucleic acid sequence may be a nonsense mutation.

The delivery of the TREM or a composition thereof (e.g., a pharmaceutical composition) intratumorally disclosed herein can (i) increase the retention of the TREM in the tumor; (ii) increase the levels of the TREM in the tumor compared to the levels of the TREM in peritumoral tissue; (iii) decrease leakage of the TREM to off-target tissue (e.g., peritumoral tissue, or to distant locations, e.g., liver tissue); or (iv) any combination thereof. In an embodiment, the increase or decrease observed for a certain property (e.g., (i)-(iv)) is relative to a corresponding reference composition. In an embodiment, a decrease in leakage can be quantified as increase in the ratio of the TREM in the tumor to TREM in non-tumor tissues, such as peritumoral tissue or to another tissue or organ, e.g., liver tissue.

Disclosed herein are methods for providing a tRNA-based effector molecule (TREM) to a subject having cancer (e.g., to a cancerous cell, tissue, or organ in the subject). For example, the present disclosure provides methods of delivering a TREM to a tumor. The TREM may be administered systemically or locally. In some embodiments, the TREM is delivered locally. Local delivery of a TREM may include delivery to a tumor (i.e., intratumoral delivery) or delivery adjacent to a tumor. In some embodiments, delivery of the TREM comprises delivery into at least one tumor. In some embodiments, delivery of the TREM comprises delivery into more than one tumor. In some embodiments, delivery of the TREM comprises delivery adjacent to the tumor. In some embodiments, delivery of the TREM comprises delivery into non-cancerous tissue adjacent to the tumor.

In some embodiments, a TREM is administered intratumorally by injection. In some embodiments, a TREM is delivered by injection into a tumor. In some embodiments, a TREM is delivered by implant injected into a tumor. In some embodiments, the TREM is released by the implant by controlled release over a period of time. In some embodiments, a TREM is delivered by administration of a patch. In some embodiments, the patch is administered directly to the tumor. In some embodiments, the patch is administered to tissue adjacent to the tumor.

In some embodiments, a TREM is administered locally. For example, a TREM may be administered outside of or adjacent to a tumor, e.g., about 0.01 mm, about 0.05 mm, about 0.075 mm, about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 2.0 mm, about 3.0 mm, about 4.0 mm, about 5.0 mm, about 6.0 mm, about 7.0 mm, about 8.0 mm, about 9.0 mm, about 1.0 cm, about 2.0 cm, about 3.0 cm, about 4.0 cm, about 5.0 cm, about 6.0 cm, about 7.0 cm, about 8.0 cm, about 9.0 cm, about 10 cm, about 20 cm, about 30 cm, about 40 cm, about 50 cm, about 60 cm, about 70 cm, about 80 cm, about 90 cm, or about 100 cm. In some embodiments, a TREM may be administered outside of or adjacent to a tumor, e.g., about 0.01 mm to about 100 cm. In some embodiments, a TREM may be administered outside of or adjacent to a tumor, e.g., about 0.01 mm to about 1 mm, about 1 mm to about 5 mm, about 5 mm to about 1 cm, about 1 cm to about 5 cm, about 5 cm to about 10 cm, about 10 cm to about 20 cm, about 20 cm to about 30 cm, about 30 cm to about 40 cm, about 40 cm to about 50 cm, about 50 cm to about 60 cm, about 60 cm to about 70 cm, about 70 cm to about 80 cm, about 80 cm to about 90 cm, about 90 cm to about 100 cm.

In some embodiments, a TREM is administered systemically. In some embodiments, a TREM is administered by injection. In some embodiments, a TREM is administered by intravenous (IV) injection. In some embodiments, a TREM is administered by intramuscular (IM) injections. In some embodiments, a TREM is administered by subcutaneous (SC) injections. In some embodiments, a TREM is administered orally. In some embodiments, systemic administration comprises routes including ocular, nasal, urogenital, intradermal, dermal, enteral, intravitreal, intracerebral, intrathecal, or epidural.

In some embodiments, a TREM is administered in a pharmaceutical composition. The compositions described herein may be formulated to be compatible with the intended rout of administration. Solutions, suspensions, dispersions, or emulsions may be used for such administrations and may include a sterile diluent, such as water for injection, saline solution, polyethylene glycols, glycerine, propylene glycol or other synthetic solvents; anti-bacterial agents such as benzyl alcohol or methylparabens; antioxidants such as ascorbic acid or sodium bisulfate; chelating agents such as ethylenediaminetetraacetic acid; buffers such as aacetates, citrates or phosphates and agents for the adjustment of tonicity such as sodium chloride or dextrose.

In some embodiments, the TREM composition, e.g., a pharmaceutical composition comprising a TREM, is administered as a function of body weight. In some embodiments, a TREM may be administered as a single dose or in multiple doses. In some embodiments, a TREM composition, e.g., a pharmaceutical composition comprising a TREM, is administered in a dose of about 0.1 mg/kg to 10 mg/kg. In some embodiments, the dose is about 0.1 mg/kg to about 0.5 mg/kg. In some embodiments, the dose is about 0.5 mg/kg to about 1.0 mg/kg. In some embodiments, the dose is about 1.0 mg/kg to about 1.5 mg/kg. In some embodiments, the dose is about 1.5 mg/kg to about 2.0 mg/kg. In some embodiments, the dose is about 2.0 mg/kg to about 2.5 mg/kg. In some embodiments, the dose is about 2.5 mg/kg to about 3.0 mg/kg. In some embodiments, the dose is about 3.0 mg/kg to about 3.5 mg/kg. In some embodiments, the dose is about 3.5 mg/kg to about 4.0 mg/kg. In some embodiments, the dose is about 4.0 mg/kg to about 4.5 mg/kg. In some embodiments, the dose is about 4.5 mg/kg to about 5.0 mg/kg. In some embodiments, the dose is about 5.0 mg/kg to about 5.5 mg/kg. In some embodiments, the dose is about 5.5 mg/kg to about 6.0 mg/kg. In some embodiments, the dose is about 6.0 mg/kg to about 6.5 mg/kg. In some embodiments, the dose is about 6.5 mg/kg to about 7.0 mg/kg. In some embodiments, the dose is about 7.0 mg/kg to about 7.5 mg/kg. In some embodiments, the dose is about 7.5 mg/kg to about 8.0 mg/kg. In some embodiments, the dose is about 8.0 mg/kg to about 8.5 mg/kg. In some embodiments, the dose is about 8.5 mg/kg to about 9.0 mg/kg. In some embodiments, the dose is about 9.0 mg/kg to about 9.5 mg/kg. In some embodiments, the dose is about 9.5 mg/kg to about 10.0 mg/kg.

In some embodiments, the TREM composition, e.g., a pharmaceutical composition comprising a TREM, can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations will be apparent to those skilled in the art.

### Vectors and Carriers

In some embodiments the TREM, TREM core fragment, or TREM fragment or TREM composition described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, or an adenovirus. In some embodiments, the system or components of the system are delivered to cells with a viral-like particle or a virosome. In some embodiments, the delivery uses more than one virus, viral-like particle or virosome.

### Carriers

A TREM, a TREM composition or a pharmaceutical TREM composition described herein may comprise, may be formulated with, or may be delivered in, a carrier.

### Viral Vectors

The carrier may be a viral vector (e.g., a viral vector comprising a sequence encoding a TREM, a TREM core fragment or a TREM fragment). The viral vector may be administered to a cell or to a subject (e.g., a human subject or animal model) to deliver a TREM, a TREM core fragment or a TREM fragment, a TREM composition or a pharmaceutical TREM composition.

A viral vector may be systemically or locally administered (e.g., injected). Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus. cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A TREM, a TREM core fragment or a TREM fragment, a TREM composition or a pharmaceutical TREM composition described herein can be administered to a cell in a vesicle or other membrane-based carrier.

In embodiments, a TREM, a TREM core fragment or a TREM fragment, or TREM composition, or pharmaceutical TREM composition described herein is administered in or via a cell, vesicle or other membrane-based carrier. In one embodiment, the TREM, TREM core fragment, TREM fragment, or TREM composition or pharmaceutical TREM composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the TREM, TREM core fragment, TREM fragment, or TREM composition or pharmaceutical TREM composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exemplary lipid nanoparticles are disclosed in International Application PCT/US2014/053907, the entire contents of which are hereby incorporated by reference. For example, an LNP described in paragraphs [403-406] or [410-413] of PCT/US2014/053907 can be used as a carrier for the TREM, TREM core fragment, TREM fragment, or TREM composition or pharmaceutical TREM composition described herein.

Additional exemplary lipid nanoparticles are disclosed in U.S. Pat. No. 10,562,849 the entire contents of which are hereby incorporated by reference. For example, an LNP of formula (I) as described in columns 1-3 of U.S. Pat. No. 10,562,849 can be used as a carrier for the TREM, TREM core fragment, TREM fragment, or TREM composition or pharmaceutical TREM composition described herein.

Lipids that can be used in nanoparticle formations (e.g., lipid nanoparticles) include, for example those described in Table 4 of WO2019217941, which is incorporated by reference, e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA) described herein includes,

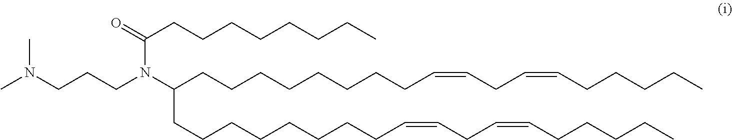 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a TREM composition described herein to the liver and/or hepatocyte cells.

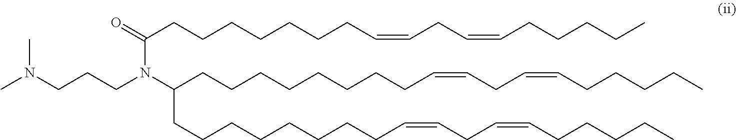 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a TREM composition described herein to the liver and/or hepatocyte cells.

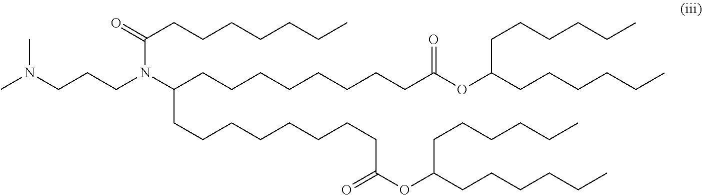 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a TREM composition described herein to the liver and/or hepatocyte cells.

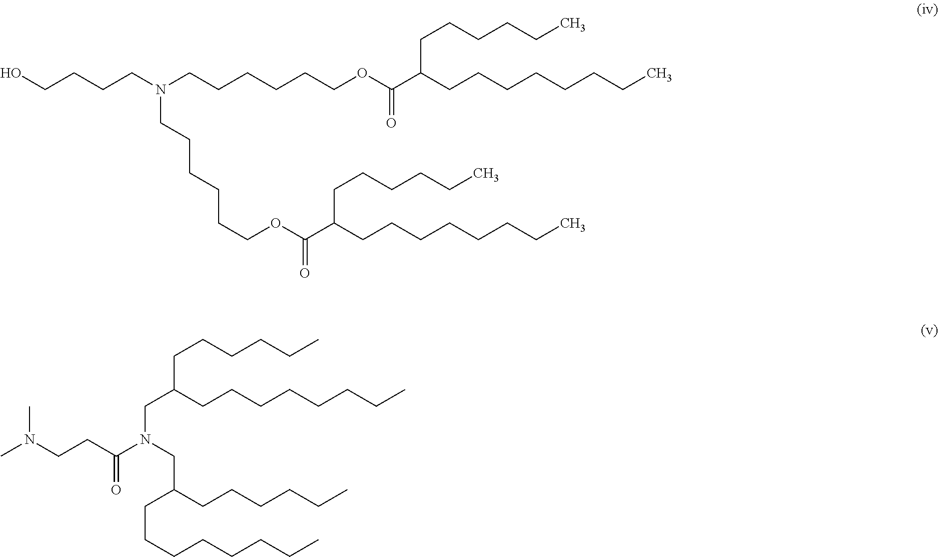

In some embodiments an LNP comprising Formula (v) is used to deliver a TREM composition described herein to the liver and/or hepatocyte cells.

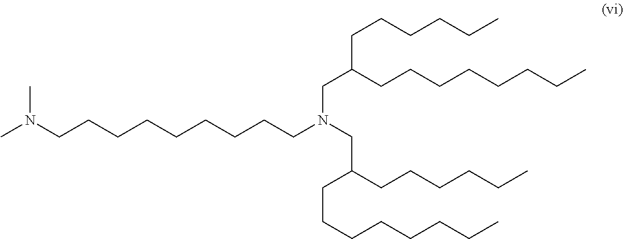 [SMILES: CCCCCCCCC(CCCCCC)CN(CCCCCCCCCN(C)C)CC(CCCCCC)CCCCCCCC]

In some embodiments an LNP comprising Formula (vi) is used to deliver a TREM composition described herein to the liver and/or hepatocyte cells.

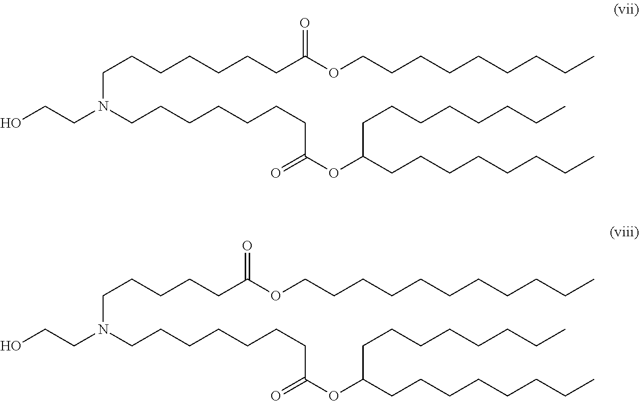 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a TREM composition described herein to the liver and/or hepatocyte cells.

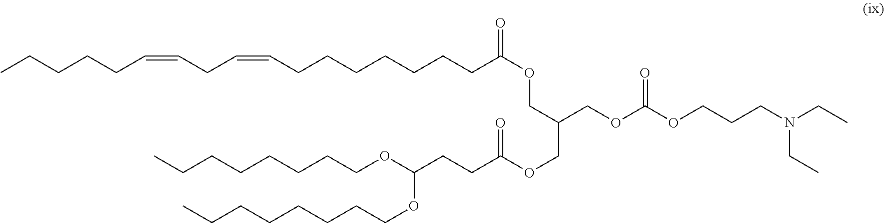 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a TREM composition described herein to the liver and/or hepatocyte cells.

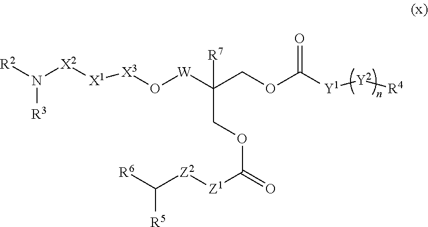

wherein X¹ is O, NR¹ or a direct bond, X² is C2-5 alkylene, X³ is C(═O) or a direct bond, R¹ is H or Me, R³ is Ci-3 alkyl, R² is Ci-3 Alkyl, or R² taken together with the nitrogen atom to which it is attached and 1-3 carbon atoms of X² form a 4-, 5- or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene. Y² is selected from

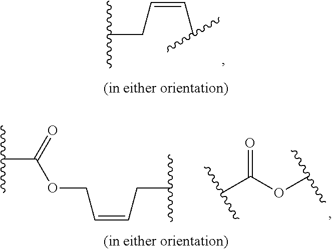 [SMILES: */C=C\C* ; *C/C=C\COC(*)=O ; *OC(*)=O]

(in either orientation).
n is 0 to 3, R⁴ is Ci-15 alkyl, Z¹ is Ci-6 alkylene or a direct bond, Z² is

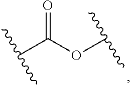 [SMILES: *OC(*)=O]

(in either orientation) or absent, provided that if Z¹ is a direct bond, Z² is absent; R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene. (Y²)n-R⁴ is:

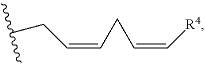

R⁴ is linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a TREM composition described herein to the liver and/or hepatocyte cells.

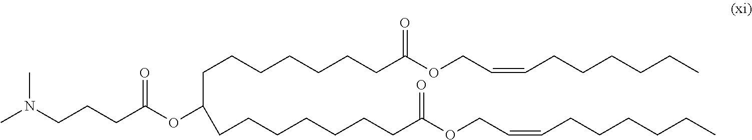 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a TREM composition described herein to the liver and/or hepatocyte cells.

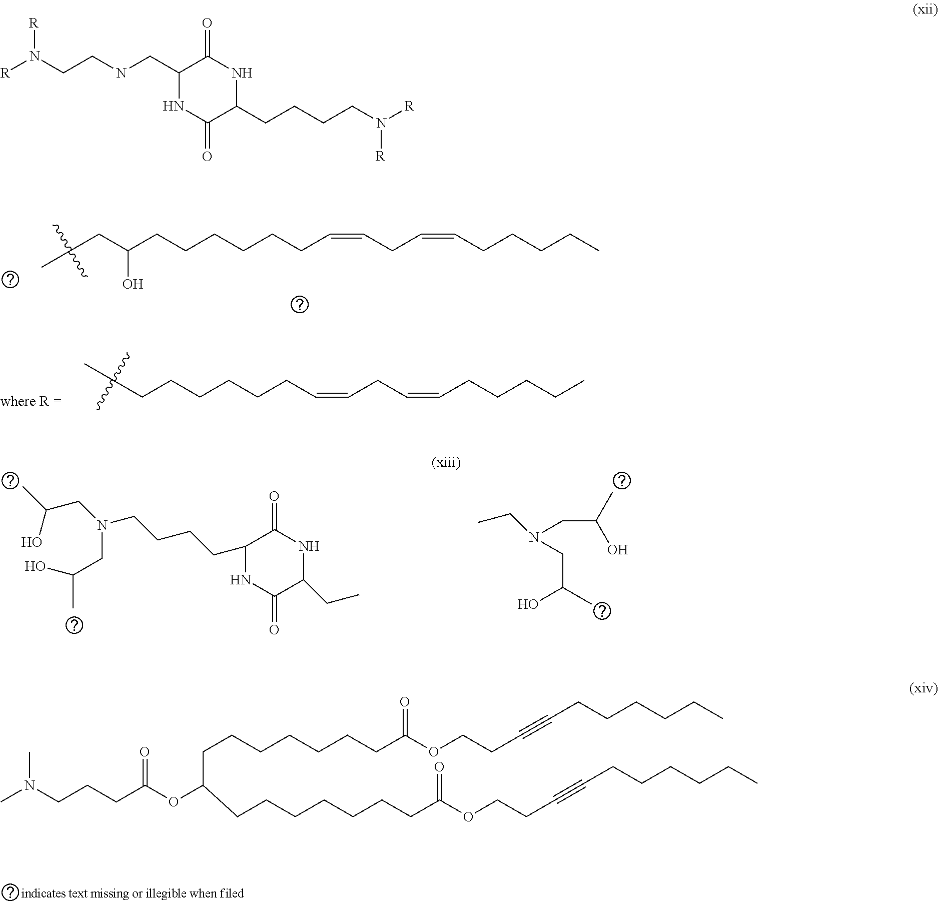

In some embodiments an LNP comprises a compound of Formula (xiii) and a compound of Formula (xiv).

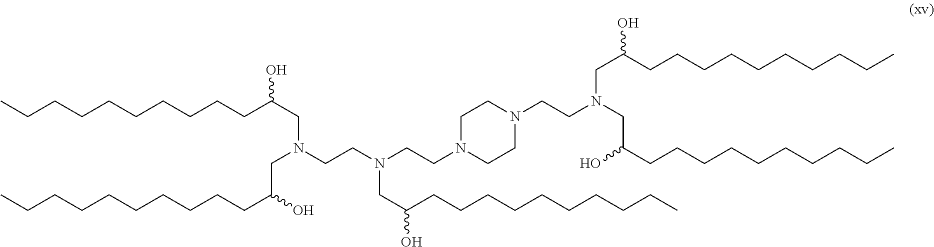 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, an LNP comprising Formula (xv) is used to deliver a TREM composition described herein to the liver and/or hepatocyte cells.

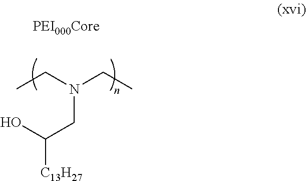

In some embodiments an LNP comprising a formulation of Formula (xvi) is used to deliver a TREM composition described herein to the lung endothelial cells.

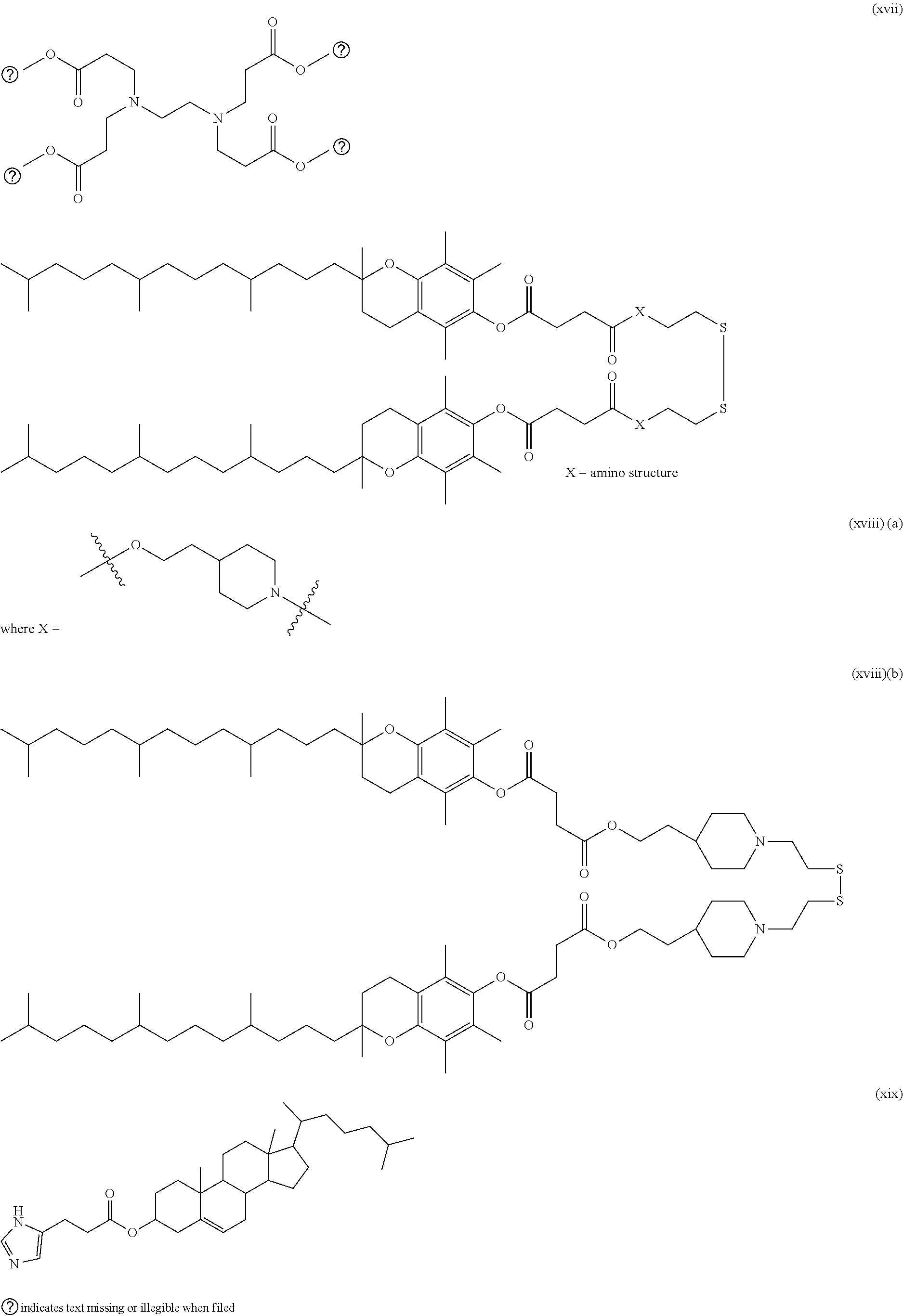

In some embodiments, a lipid compound used to form lipid nanoparticles for the delivery of compositions described herein, e.g., a TREM described herein is made by one of the following reactions:

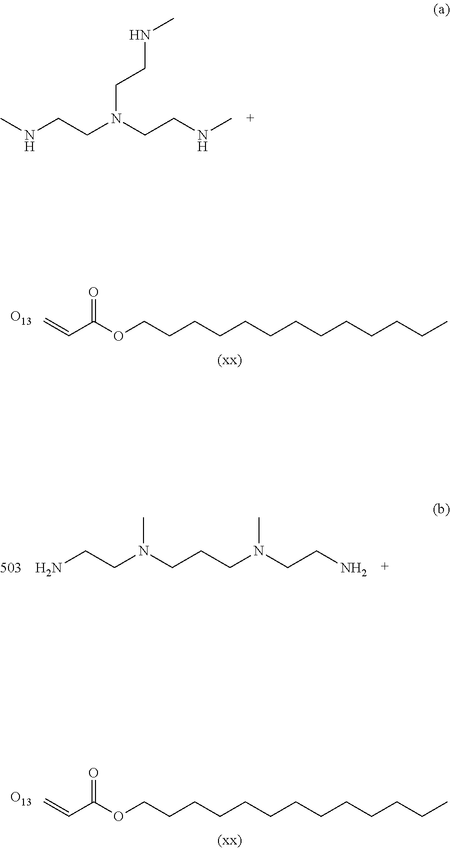

In some embodiments, a composition described herein (e.g., TREM composition) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)-butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H- imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a TREM described herein, encapsulated within or associated with the lipid nanoparticle. In some embodiments, the TREM is co-formulated with the cationic lipid. The TREM may be adsorbed to the surface of an LNP, e.g., an LNP comprising a cationic lipid. In some embodiments, the TREM may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of a TREM.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; 1, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; 111-3 of WO2018/081480; 1-5 or 1-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), diolcoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-buty 1 ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE). PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

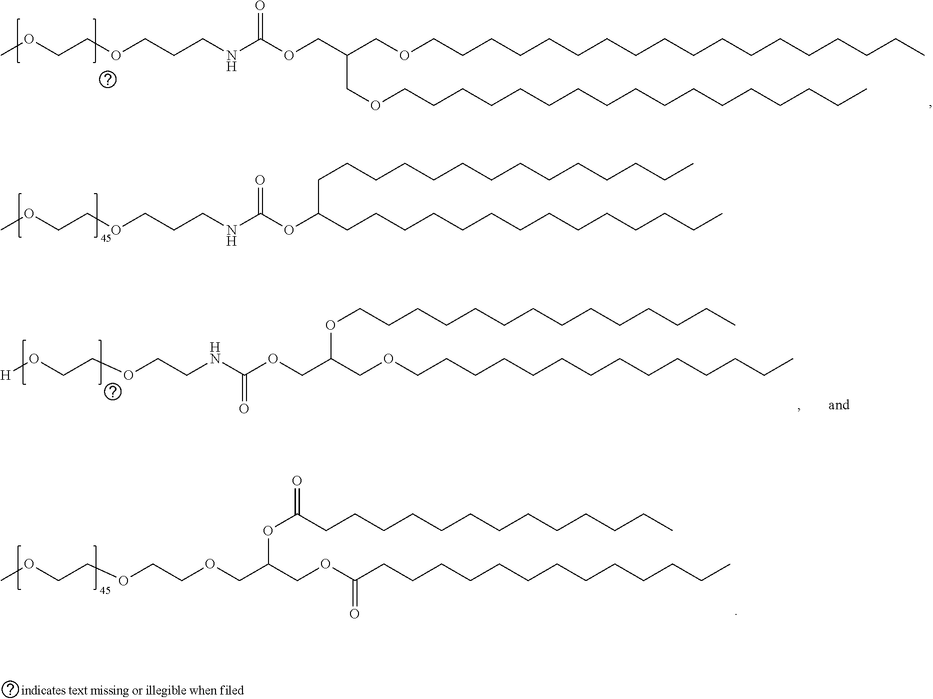

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from I to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

An LNP may, in some instances, be relatively homogenous. A polydispersity index may be used to indicate the homogeneity of an LNP, e.g., the particle size distribution of the lipid nanoparticles. A small (e.g., less than 0.3) polydispersity index generally indicates a narrow particle size distribution. An LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of an LNP may be from about 0.10 to about 0.20.

The zeta potential of an LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of an LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a TREM describes the amount of TREM that is encapsulated or otherwise associated with an LNP after preparation, relative to the initial amount provided. The encapsulation efficiency is desirably high (e.g., close to 100%). The encapsulation efficiency may be measured, for example, by comparing the amount of TREM in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free TREM in a solution. For the lipid nanoparticles described herein, the encapsulation efficiency of a TREM may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

An LNP may optionally comprise one or more coatings. In some embodiments, an LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exosomes can also be used as drug delivery vehicles for the TREM, TREM core fragment, TREM fragment, or TREM compositions or pharmaceutical TREM composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM, TREM core fragment, TREM fragment, or TREM composition, or pharmaceutical TREM composition described herein. See, e.g., WO2015073587; WO2017123646; WO2017123644; WO2018102740; w02016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the TREM, TREM core fragment, TREM fragment, or TREM composition, or pharmaceutical TREM composition described herein.

Virosomes and virus-like particles (VLPs) can also be used as carriers to deliver a TREM, TREM core fragment, TREM fragment, or TREM composition, or pharmaceutical TREM composition described herein to targeted cells.

Plant nanovesicles, e.g., as described in WO2011097480A1, WO2013070324A1, or WO2017004526A1 can also be used as carriers to deliver the TREM, TREM core fragment, TREM fragment, or TREM composition, or pharmaceutical TREM composition described herein.

### Delivery Without a Carrier

A TREM, a TREM core fragment or a TREM fragment, a TREM composition or a pharmaceutical TREM composition described herein can be administered to a cell without a carrier, e.g., via naked delivery of the TREM, a TREM core fragment or a TREM fragment, a TREM composition or a pharmaceutical TREM composition.

In some embodiments, naked delivery as used herein refers to delivery without a carrier. In some embodiments, delivery without a carrier, e.g., naked delivery, comprises delivery with a moiety, e.g., a targeting peptide.

In some embodiments, a TREM, a TREM core fragment or a TREM fragment, or TREM composition, or pharmaceutical TREM composition described herein is delivered to a cell without a carrier, e.g., via naked delivery. In some embodiments, the delivery without a carrier, e.g., naked delivery, comprises delivery with a moiety, e.g., a targeting peptide.

### TREM Characterization

A TREM, TREM core fragment, or TREM fragment, or a TREM composition, e.g., a pharmaceutical TREM composition, produced by any of the methods disclosed herein can be assessed for a characteristic associated with the TREM, TREM core fragment, or TREM fragment or the TREM composition, such as purity, sterility, concentration, structure, or functional activity of the TREM, TREM core fragment, or TREM fragment. Any of the above-mentioned characteristics can be evaluated by providing a value for the characteristic, e.g., by evaluating or testing the TREM, TREM core fragment, or TREM fragment, or the TREM composition, or an intermediate in the production of the TREM composition. The value can also be compared with a standard or a reference value. Responsive to the evaluation, the TREM composition can be classified, e.g., as ready for release, meets production standard for human trials, complies with ISO standards, complies with cGMP standards, or complies with other pharmaceutical standards. Responsive to the evaluation, the TREM composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the TREM composition. For example, the TREM composition can be modulated, processed or re-processed to (i) increase the purity of the TREM composition; (ii) decrease the amount of fragments in the composition; (iii) decrease the amount of endotoxins in the composition; (iv) increase the in vitro translation activity of the composition; (v) increase the TREM concentration of the composition; or (vi) inactivate or remove any viral contaminants present in the composition, e.g., by reducing the pH of the composition or by filtration.

In an embodiment, the TREM, TREM core fragment, or TREM fragment (e.g., TREM composition or an intermediate in the production of the TREM composition) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., TREM composition or an intermediate in the production of the TREM composition) has less than 0.1%, 0.5%, 1%, 2%, 3%, 4%, 5%, 6%, 7%, 8%, 9%, 10%, 15%, 20%, 25% TREM fragments relative to full length TREMs.

In an embodiment, the TREM, TREM core fragment, or TREM fragment (e.g., TREM composition or an intermediate in the production of the TREM composition) has low levels or absence of endotoxins, e.g., a negative result as measured by the Limulus amebocyte lysate (LAL) test.

In an embodiment, the TREM, TREM core fragment, or TREM fragment (e.g., TREM composition or an intermediate in the production of the TREM composition) has in-vitro translation activity, e.g., as measured by an assay described in Examples 12-13.

In an embodiment, the TREM, TREM core fragment, or TREM fragment (e.g., TREM composition or an intermediate in the production of the TREM composition) has a TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/nL, 10,000 ug/mL, or 100,000 ug/mL.

In an embodiment, the TREM, TREM core fragment, or TREM fragment (e.g., TREM composition or an intermediate in the production of the TREM composition) is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

In an embodiment, the TREM, TREM core fragment, or TREM fragment (e.g., TREM composition or an intermediate in the production of the TREM composition) has an undetectable level of viral contaminants, e.g., no viral contaminants. In an embodiment, any viral contaminant, e.g., residual virus, present in the composition is inactivated or removed. In an embodiment, any viral contaminant, e.g., residual virus, is inactivated, e.g., by reducing the pH of the composition. In an embodiment, any viral contaminant, e.g., residual virus, is removed, e.g., by filtration or other methods known in the field.

### Use of TREMs

A TREM composition (e.g., a pharmaceutical TREM composition described herein) can modulate a function in a cell, tissue or subject. In embodiments, a TREM composition (e.g., a pharmaceutical TREM composition) described herein is contacted with a cell or tissue, or administered to a subject in need thereof, in an amount and for a time sufficient to modulate (increase or decrease) one or more of the following parameters: adaptor function (e.g., cognate or non-cognate adaptor function), e.g., the rate, efficiency, robustness, and/or specificity of initiation or elongation of a polypeptide chain; ribosome binding and/or occupancy; regulatory function (e.g., gene silencing or signaling); cell fate; mRNA stability; protein stability; protein transduction; protein compartmentalization. A parameter may be modulated, e.g., by at least 5% (e.g., at least 10%, 15%, 20%, 25%, 30%, 40%, 50%, 60%, 70%, 80%, 90%, 100%, 150%, 200% or more) compared to a reference tissue, cell or subject (e.g., a healthy, wild-type or control cell, tissue or subject).

In some embodiments, a TREM as disclosed herein in the instant application is not a tRNA disclosed in WO2019090169A1. In some embodiments, the TREM as disclosed herein is not delivered intratumorally, e.g., using an injection device, such as an automatic injection apparatus. In some embodiments, the TREM as disclosed herein is not delivered to a cell or subject ex vivo, for example, delivered to the cells of a subject after the cells have been removed from said subject.

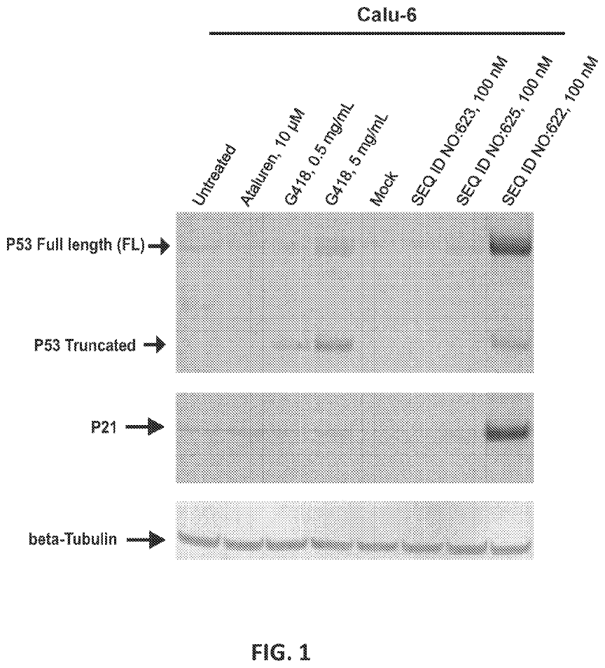

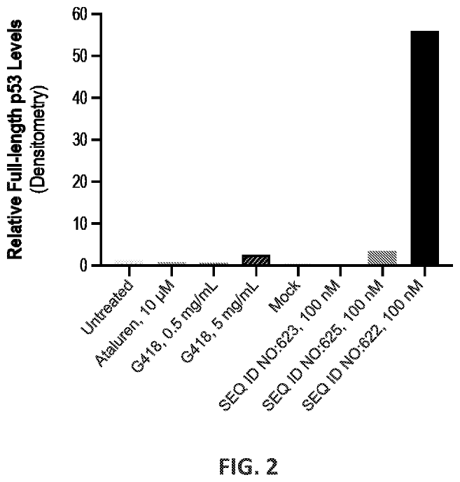

In some embodiments, a TREM described herein may read-through a premature termination codon (PTC) that is associated with a cancer. For example, Calu-6 lung carcinoma cells have a mutation that produces a PTC in the TP53 gene encoding the p53 protein, resulting in truncated p53 and a lung cancer phenotype. In some embodiments, a TREM may read-through the PTC in TP53 in Calu-6 cells and increase the levels of full-length p53, e.g., full-length p53 levels as provided in FIGS. 1-2. In an embodiment, when Calu-6 cells are treated with a TREM, full-length p53 levels increase relative to full-length p53 levels in untreated cells. In an embodiment, when Calu-6 cells are treated with a TREM, full-length p53 levels increase relative to full-length p53 levels in cells treated with a known translational readthrough-inducing drug. In an embodiment, when Calu-6 cells are treated with a TREM, full-length p53 levels increase relative to full-length p53 levels in cells treated with the translational readthrough-inducing drug Ataluren. In an embodiment, when Calu-6 cells are treated with a TREM, full-length p53 levels increase relative to full-length p53 levels in cells treated with the translational readthrough-inducing drug G418. In an embodiment, when Calu-6 cells are treated with a TREM, full-length p53 levels increase relative to full-length p53 levels in mock-transfected cells. In an embodiment, when Calu-6 cells are treated with a TREM, full-length p53 levels increase relative to full-length p53 levels in cells treated with a different TREM. In an embodiment, when Calu-6 cells are treated with SEQ ID NO:622, full-length p53 levels increase relative to full-length p53 levels in untreated cells. In an embodiment, when Calu-6 cells are treated with SEQ ID NO: 622, full-length p53 levels increase relative to full-length p53 levels in cells treated with the translational readthrough-inducing drug Ataluren. In an embodiment, when Calu-6 cells are treated with SEQ ID NO: 622, full-length p53 levels increase relative to full-length p53 levels in cells treated with the translational readthrough-inducing drug G418. In an embodiment, when Calu-6 cells are treated with SEQ ID NO: 622, full-length p53 levels increase relative to full-length p53 levels in mock-transfected cells. In an embodiment, when Calu-6 cells are treated with SEQ ID NO: 622, full-length p53 levels increase relative to full-length p53 levels in cells treated with SEQ ID NO: 623. In an embodiment, when Calu-6 cells are treated with SEQ ID NO: 622, full-length p53 levels increase relative to full-length p53 levels in cells treated with SEQ ID NO:625. In an embodiment, when Calu-6 cells are treated with 100 nM SEQ ID NO: 622, full-length p53 levels increase relative to full-length p53 levels in untreated cells. In an embodiment, when Calu-6 cells are treated with 100 nM SEQ ID NO: 622, full-length p53 levels increase relative to full-length p53 levels in cells treated with 10 μM of the translational readthrough-inducing drug Ataluren. In an embodiment, when Calu-6 cells are treated with 100 nM SEQ ID NO: 622, full-length p53 levels increase relative to full-length p53 levels in cells treated with 0.5 mg/mL of the translational readthrough-inducing drug G418. In an embodiment, when Calu-6 cells are treated with 100 nM SEQ ID NO: 622, full-length p53 levels increase relative to full-length p53 levels in cells treated with 5 mg/mL of the translational readthrough-inducing drug G418. In an embodiment, when Calu-6 cells are treated with 100 nM SEQ ID NO: 622, full-length p53 levels increase relative to full-length p53 levels in mock-transfected cells. In an embodiment, when Calu-6 cells are treated with 100 nM SEQ ID NO: 622, full-length p53 levels increase relative to full-length p53 levels in cells treated with 100 nM SEQ ID NO:623. In an embodiment, when Calu-6 cells are treated with 100 nM SEQ ID NO: 622, full-length p53 levels increase relative to full-length p53 levels in cells treated with 100 nM SEQ ID NO:625. In an embodiment, when Calu-6 cells are treated with 100 nM SEQ ID NO: 622, full-length p53 levels can increase by about 55-fold relative to full-length p53 levels in untreated cells. In an embodiment, when Calu-6 cells are treated with 100 nM SEQ ID NO: 622, full-length p53 levels can increase by about 55-fold relative to full-length p53 levels in cells treated with 10 μM of the translational readthrough-inducing drug Ataluren. In an embodiment, when Calu-6 cells are treated with 100 nM SEQ ID NO: 622, full-length p53 levels can increase by about 55-fold relative to full-length p53 levels in cells treated with 0.5 mg/mL of the translational readthrough-inducing drug G418. In an embodiment, when Calu-6 cells are treated with 100 nM SEQ ID NO: 622, full-length p53 levels can increase by about 14-fold relative to full-length p53 levels in cells treated with 5 mg/mL of the translational readthrough-inducing drug G418. In an embodiment, when Calu-6 cells are treated with 100 nM SEQ ID NO: 622, full-length p53 levels can increase by about 55-fold relative to full-length p53 levels in mock-transfected cells. In an embodiment, when Calu-6 cells are treated with 100 nM SEQ ID NO: 622, full-length p53 levels can increase by about 55-fold relative to full-length p53 levels in cells treated with 100 nM SEQ ID NO:623. In an embodiment, when Calu-6 cells are treated with 100 nM SEQ ID NO: 622, full-length p53 levels can increase by about 11-fold relative to full-length p53 levels in cells treated with 100 nM SEQ ID NO:625.

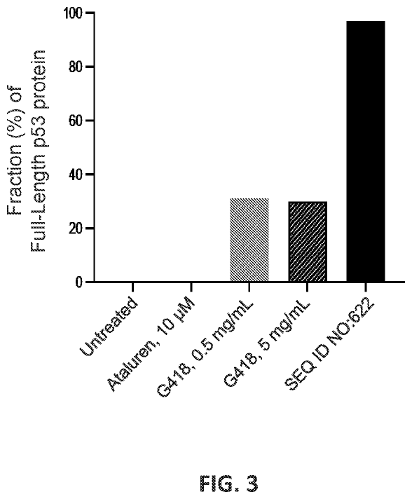

In some embodiments, a TREM may read-through a PTC and increase the fraction of full-length protein relative to truncated protein. For example, readthrough of the PTC in TP53 in Calu-6 lung carcinoma cells may increase the fraction of full-length p53 protein relative to truncated p53. In some embodiments, a TREM can read-through the PTC in TP53 in Calu-6 cells and increase the percent of full-length p53 relative to truncated p53, e.g., the percentage of full-length p53 as provided in FIG. 3. In an embodiment, the percentage of full-length p53 is less than the percentage of truncated p53 in untreated Calu-6 cells. In an embodiment, the percentage of full-length p53 is less than the percentage of truncated p53 in Calu-6 cells treated with a translational readthrough-inducing drug. In an embodiment, the percentage of full-length p53 is less than the percentage of truncated p53 in Calu-6 cells treated with the translational readthrough-inducing drug Ataluren. In an embodiment, the percentage of full-length p53 is less than the percentage of truncated p53 in Calu-6 cells treated with the translational readthrough-inducing drug G418. In an embodiment, the percentage of full-length p53 is greater than the percentage of truncated p53 in Calu-6 cells treated with a TREM. In an embodiment, the percentage of full-length p53 is greater than the percentage of truncated p53 in Calu-6 cells treated with SEQ ID NO: 622. In an embodiment, the percentage of full-length p53 out of the total p53 protein is about 0% in untreated Calu-6 cells. In an embodiment, the percentage of full-length p53 out of the total p53 protein is about 0% in Calu-6 cells treated with 10 μM of the translational readthrough-inducing drug Ataluren. In an embodiment, the percentage of full-length p53 out of the total p53 protein is about 30% in Calu-6 cells treated with 0.5 mg/mL of the translational readthrough-inducing drug G418. In an embodiment, the percentage of full-length p53 out of the total p53 protein is about 30% in Calu-6 cells treated with 5 mg/mL of the translational readthrough-inducing drug G418. In an embodiment, the percentage of full-length p53 of the total p53 protein is about 95% in Calu-6 cells treated with 100 nM SEQ ID NO: 622.

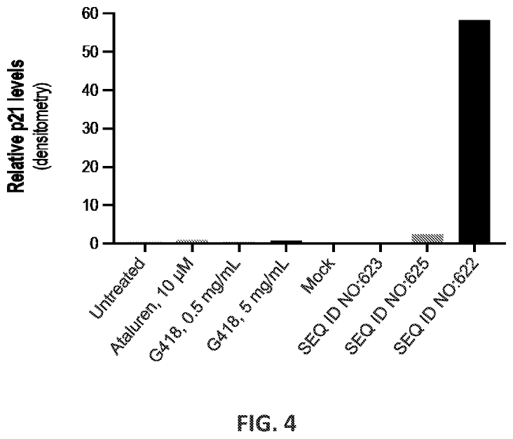

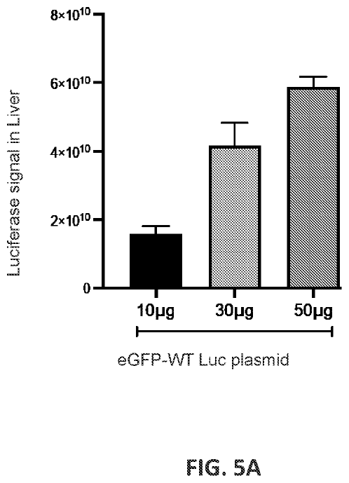

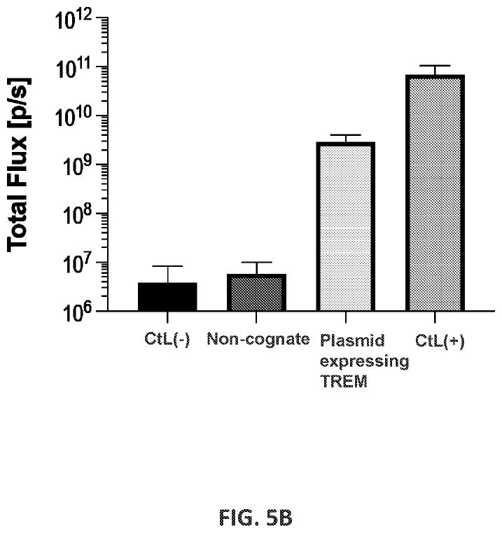

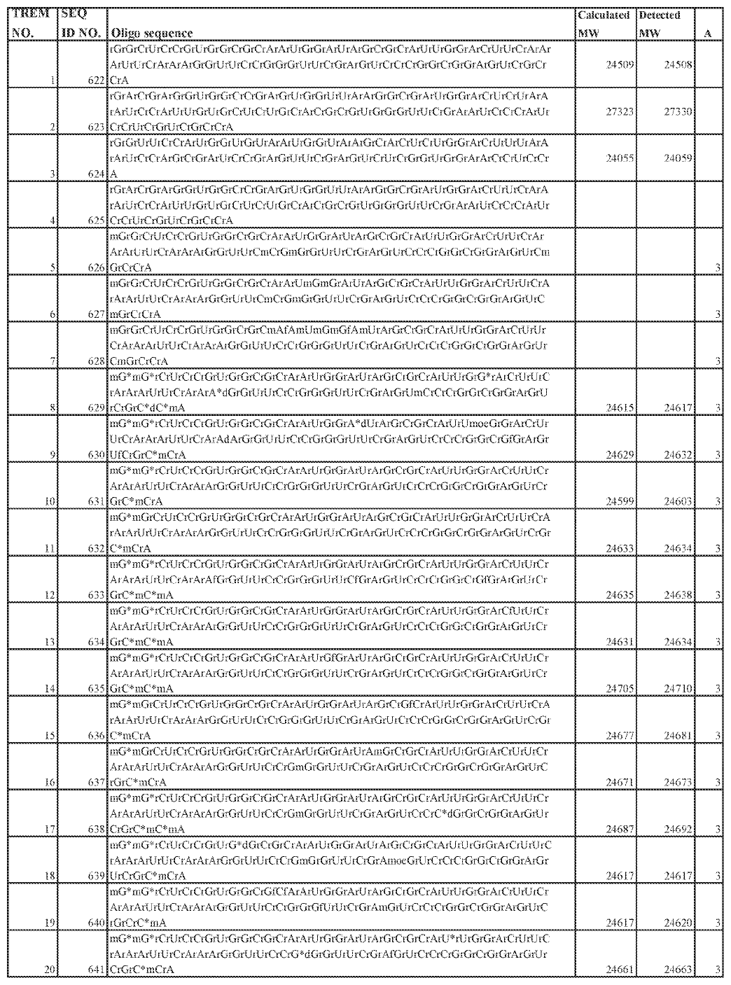

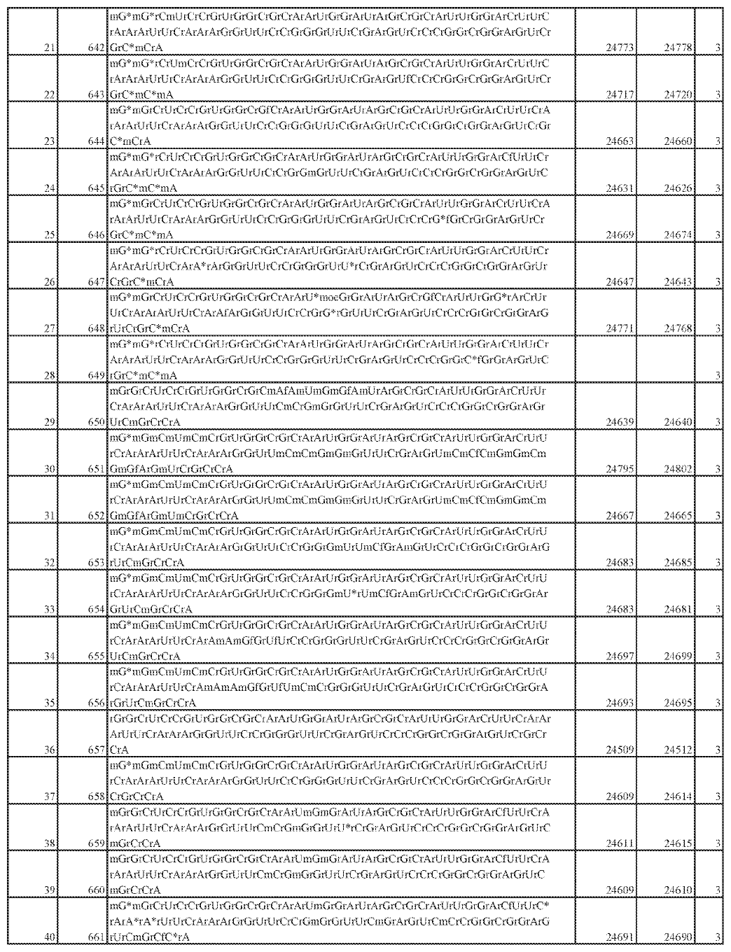

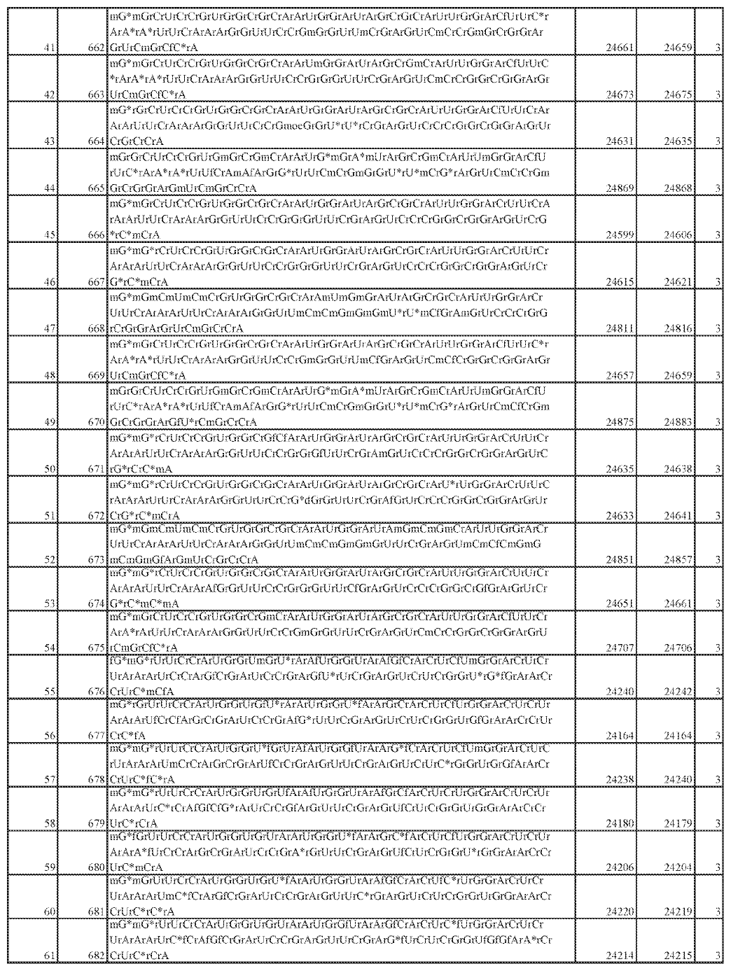

In some embodiments, TREM readthrough of a PTC produces a functional protein capable of acting on its downstream targets. For example, readthrough of the PTC in TP53 in Calu-6 cells may produce functional full-length p53 that increases levels of p21 protein, e.g., p21 levels as provided in FIGS. 1 and 4. In some embodiments, TREM readthrough of the PTC in TP53 in Calu-6 cells increases the levels of p21. In an embodiment, when Calu-6 cells are treated with a TREM, p21 levels increase relative to p21 levels in untreated cells. In an embodiment, when Calu-6 cells are treated with a TREM, p21 levels increase relative to p21 levels in cells treated with a known translational readthrough-inducing drug. In an embodiment, when Calu-6 cells are treated with a TREM, p21 levels increase relative to p21 levels in cells treated with the translational readthrough-inducing drug Ataluren. In an embodiment, when Calu-6 cells are treated with a TREM, p21 levels increase relative to p21 levels in cells treated with the translational readthrough-inducing drug G418. In an embodiment, when Calu-6 cells are treated with a TREM, p21 levels increase relative to p21 levels in mock-transfected cells. In an embodiment, when Calu-6 cells are treated with a TREM, p21 levels increase relative to p21 levels in cells treated with a different TREM. In an embodiment, when Calu-6 cells are treated with SEQ ID NO: 622, p21 levels increase relative to p21 levels in untreated cells. In an embodiment, when Calu-6 cells are treated with SEQ ID NO: 622, p21 levels increase relative to p21 levels in cells treated with a known translational readthrough-inducing drug. In an embodiment, when Calu-6 cells are treated with SEQ ID NO: 622, p21 levels increase relative to p21 levels in cells treated with the translational readthrough-inducing drug Ataluren. In an embodiment, when Calu-6 cells are treated with SEQ ID NO: 622, p21 levels increase relative to p21 levels in cells treated with the translational readthrough-inducing drug G418. In an embodiment, when Calu-6 cells are treated with SEQ ID NO: 622, p21 levels increase relative to p21 levels in mock-transfected cells. In an embodiment, when Calu-6 cells are treated with SEQ ID NO: 622, p21 levels increase relative to p21 levels in cells treated with SEQ ID NO: 623. In an embodiment, when Calu-6 cells are treated with SEQ ID NO: 622, p21 levels increase relative to p21 levels in cells treated with SEQ ID NO:625. In an embodiment, when Calu-6 cells are treated with 100 nM SEQ ID NO: 622, p21 levels increase relative to p21 levels in untreated cells. In an embodiment, when Calu-6 cells are treated with 100 nM SEQ ID NO: 622, p21 levels increase relative to p21 levels in cells treated with 10 μM of the translational readthrough-inducing drug Ataluren. In an embodiment, when Calu-6 cells are treated with 100 nM SEQ ID NO: 622, p21 levels increase relative to p21 levels in cells treated with 0.5 mg/mL of the translational readthrough-inducing drug G418. In an embodiment, when Calu-6 cells are treated with 100 nM SEQ ID NO: 622, p21 levels increase relative to p21 levels in cells treated with 5 mg/mL of the translational readthrough-inducing drug G418. In an embodiment, when Calu-6 cells are treated with 100 nM SEQ ID NO: 622, p21 levels increase relative to p21 levels in mock-transfected cells. In an embodiment, when Calu-6 cells are treated with 100 nM SEQ ID NO: 622, p21 levels increase relative to p21 levels in cells treated with 100 nM SEQ ID NO:623. In an embodiment, when Calu-6 cells are treated with 100 nM SEQ ID NO: 622, p21 levels increase relative to p21 levels in cells treated with 100 nM SEQ ID NO:625. In an embodiment, when Calu-6 cells are treated with 100 nM SEQ ID NO: 622, p21 levels can increase by about 60-fold relative to p21 levels in untreated cells. In an embodiment, when Calu-6 cells are treated with 100 nM SEQ ID NO: 622, p21 levels can increase by about 60-fold relative to p21 levels in cells treated with 10 μM of the translational readthrough-inducing drug Ataluren. In an embodiment, when Calu-6 cells are treated with 100 nM SEQ ID NO: 622, p21 levels can increase by about 60-fold relative to p21 levels in cells treated with 0.5 mg/mL of the translational readthrough-inducing drug G418. In an embodiment, when Calu-6 cells are treated with 100 nM SEQ ID NO: 622, p21 levels can increase by about 60-fold relative to p21 levels in cells treated with 5 mg/mL of the translational readthrough-inducing drug G418. In an embodiment, when Calu-6 cells are treated with 100 nM SEQ ID NO: 622, p21 levels can increase by about 60-fold relative to p21 levels in mock-transfected cells. In an embodiment, when Calu-6 cells are treated with 100 nM SEQ ID NO: 622, p21 levels can increase by about 60-fold relative to p21 levels in cells treated with 100 nM SEQ ID NO:623. In an embodiment, when Calu-6 cells are treated with 100 nM SEQ ID NO: 622, p21 levels can increase by about 30-fold relative to p21 levels in cells treated with 100 nM SEQ ID NO:625. In some embodiments, a premature termination codon (PTC) reporter can be delivered to a host. For example, a luciferase (Luc) reporter may generate a luminescent signal in the liver of a mouse following hydrodynamic delivery, e.g., a luminescent signal as provided in FIG. 5A. In an embodiment, an eGFP-WT Luc plasmid can generate a luminescent signal in the liver after hydrodynamic delivery to a mouse. In an embodiment, an eGFP-WT Luc plasmid can generate a dose-dependent luminescent signal in the liver after hydrodynamic delivery to a mouse. In an embodiment, delivery of 10 μg of an eGFP-WT Luc plasmid results in about 1.8×10¹⁰ relative luminescent units (RLU) in the liver of a mouse after hydrodynamic delivery. In an embodiment, delivery of 30 g of an eGFP-WT Luc plasmid results in about 4×10¹⁰ RLU in the liver of a mouse after hydrodynamic delivery. In an embodiment, delivery of 50 μg of an eGFP-WT Luc plasmid results in about 6×10¹⁰ RLU in the liver of a mouse after hydrodynamic delivery.

In some embodiments, a TREM may read-through a premature termination codon (PTC) to produce a functional, full-length protein in a host. For example, a TREM may read-through a PTC in a nano-luciferase (NanoLuc) reporter to generate a luminescent signal in the liver of a mouse following hydrodynamic delivery of the TREM and NanoLuc reporter, e.g., a luminescent signal as provided in FIG. 5B. In an embodiment, when a negative control plasmid encoding NanoLuc with a TGA PTC (CtL(−)) is administered to a mouse by hydrodynamic delivery, the total flux signal in the liver is about 3×10⁶ p/s. In an embodiment, when a negative control plasmid encoding NanoLuc with a TGA PTC and a non-cognate Ser-TAG TREM (Non-cognate) is administered to a mouse by hydrodynamic delivery, the total flux signal in the liver is about 4×10⁶ p/s. In an embodiment, when a plasmid encoding NanoLuc with a TGA PTC and a cognate Arg-TGA TREM (Plasmid expressing TREM) is administered to a mouse by hydrodynamic delivery, the total flux signal in the liver is about 2×10⁹ p/s. In an embodiment, when a positive control plasmid encoding wildtype NanoLuc (CIL(+))) is administered to a mouse by hydrodynamic delivery, the total flux signal in the liver is about 8×10¹⁰ p/s.

All references and publications cited herein are hereby incorporated by reference.

### ENUMERATED EMBODIMENTS

1. A method of providing a tRNA-based effector molecule (TREM) to a subject having a proliferative disease comprising,
   acquiring a value for the presence of a premature termination codon (PTC) signature in the cancer; and
   responsive to the acquired value, administering a TREM to the subject locally, e.g., intratumorally,
   thereby providing the TREM to the subject.
2. The TREM of embodiment 1, wherein the TREM comprises the sequence of Formula A:
   [L1]-[ASt Domain1]-[L2]-[DH Domain]-[L3]-[ACH Domain]-[VL Domain]-[TH Domain]-[L4]-[ASt Domain2],
   wherein, independently, [L1] and [VL Domain], are optional.
3. The method of any one of embodiments 1-2, wherein the PTC signature comprises a nonsense mutation in a cancer cell (e.g., nonsense mutation in a tumor suppressor gene).
4. The method of any one of embodiments 1-3, wherein the TREM is selected from (i) a TREM that does not comprise a non-naturally occurring modification and (ii) a TREM comprising a non-naturally occurring modification that induces an immune response in a cell or subject.
5. The method of any one of embodiments 1-4, wherein the TREM does not comprise a non-naturally occurring chemical modification.
6. The method of any one of embodiments 1-4, wherein the TREM comprises a non-naturally occurring chemical modification.
7. The method of embodiment 6, wherein the non-naturally occurring modification is present on the nucleobase, sugar, or in the internucleotide linkage of the TREM.
8. The method of any one of embodiments 6-7, wherein the non-naturally occurring modification is present know on the sugar of the TREM.
9. The method of embodiment 8, wherein the non-naturally occurring modification comprises a 2′ modification.
10. The method of embodiment 9, wherein the non-naturally occurring modification comprises a 2′-OMe, 2′-MOE, 2′-halo (e.g., 2′-F), or 2′-deoxy modification.
11. The method of any one of embodiments 6-7, wherein the non-naturally occurring modification comprises an internucleotide modification.
12. The method of embodiment 11, wherein the non-naturally occurring modification comprises a phosphorothioate modification.
13. The method of any one of embodiments 6-12, wherein the non-naturally occurring modification induces an immune response in a cell or subject, e.g., relative to a reference value.
14. The method of embodiment 13, wherein inducing an immune response comprises an increase in the expression or level of a cytokine or in a cytotoxic T cell.
15. The method of any one of embodiments 6-14, wherein the non-naturally occurring modification comprises a sugar modification (e.g., a 2′-OMe, 2′-halo, 2′MOE, or 2′-deoxy) or a modification in the internucleotide region (e.g., phosphorothioate).
16. The method of any one of the preceding embodiments, wherein the TREM comprises a nucleotide sequence provided in FIG. 6.
17. The method of any one of the preceding embodiments, wherein the TREM comprises a nucleotide sequence of an arginine tRNA consensus sequence, e.g., a nucleotide sequence of Formula I_{ARG} (SEQ ID NO: 565), Formula II_{ARG} (SEQ ID NO: 566), or Formula III_{ARG} (SEQ ID NO: 567).
18. The method of any one of the preceding embodiments, wherein the TREM comprises a nucleotide sequence of an arginine tRNA consensus sequence and has an anticodon that is complimentary to a stop codon, e.g., TGA, TAG, or TAA.
19. The method of any one embodiments 1-16, wherein the TREM comprises a nucleotide sequence of a glutamine tRNA consensus sequence, e.g., a nucleotide sequence of Formula I_{GLN} (SEQ ID NO: 577), Formula II_{GLN} (SEQ ID NO: 578), or Formula III_{GLN} (SEQ ID NO: 579).
20. The method of embodiment 19, wherein the TREM comprises a nucleotide sequence of an glutamine tRNA consensus sequence and has an anticodon that is complimentary to a stop codon, e.g., TGA, TAG, or TAA.
21. The method of any one of embodiments 1-16, wherein the TREM comprises a nucleotide sequence of a serine tRNA consensus sequence, e.g., a nucleotide sequence of Formula I_{SER} (SEQ ID NO: 607), Formula II_{SER} (SEQ ID NO: 608), or Formula III_{SER} (SEQ ID NO: 609).
22. The method of embodiment 21, wherein the TREM comprises a nucleotide sequence of an serine tRNA consensus sequence and has an anticodon that is complimentary to a stop codon, e.g., TGA, TAG, or TAA.
23. The method of any one of the preceding embodiments, wherein the TREM comprises a nucleotide sequence having about 60%, 65%, 70%, 75%, 80%, 85%, 90%, 95%, 99%, or 99.9% sequence identity relative to a nucleotide sequence listed in FIG. 6.
24. The method of any one of the preceding embodiments, wherein the TREM comprises a nucleotide sequence that comprises a nucleotide substitution, e.g., relative to a nucleotide sequence listed in FIG. 6.
25. The method of any one of the preceding embodiments, wherein the TREM comprises a nucleotide sequence of any one of SEQ ID NOs: 622 and 626-675, e.g., listed in FIG. 6.
26. The method of any one of the preceding embodiments, wherein the TREM comprises a nucleotide sequence of any one of SEQ ID NOs: 624 and 676-690, e.g., listed in FIG. 6.
27. The method of any one of the preceding embodiments, wherein the TREM comprises a nucleotide sequence of any one of SEQ ID NOs: 623 or 625, e.g., listed in FIG. 6.
28. The method of any one of the preceding embodiments, wherein the TREM has the sequence of any one of SEQ ID NOs: 622-690.
29. The method of any one of the preceding embodiments, wherein the TREM has at least 80%, 85%, 90%, 95%, or 99% sequence identity to a TREM provided in FIG. 6.
30. The method of any one of the preceding embodiments, wherein the TREM comprises SEQ ID NO: 100, or has at least 80%, 85%, 90%, 95%, or 99% sequence identity to SEQ ID NO: 100.
31. The method of any one of embodiments 1-29, wherein the TREM comprises a sequence selected from SEQ ID NO: 622, SEQ ID NO: 623, or SEQ ID NO: 624, or has at least 80%, 85%, 90%, 95%, or 99% sequence identity to a sequence selected from SEQ ID NO: 622, SEQ ID NO: 623, or SEQ ID NO: 624.
32. The method of any one of the preceding embodiments, wherein the premature termination codon (PTC) signature is present in p53.
33. The method of any one of the preceding embodiments, wherein expression or level of full-length p53 protein is increased in a cell, e.g., by about 5%, 10%, 15%, 20%, 25%, 50%, 75%, 90%, relative to a reference value, upon administration of the TREM.
34. The method of any one of embodiments 32-33, wherein the expression or level of the p21 protein is increased in a cell, e.g., by about 5%, 10%, 15%, 20%, 25%, 50%, 75%, 90%, relative to a reference value, upon administration of the TREM.
35. The method of any one of the preceding embodiments, wherein the cancer is selected from a cancer provided in Tables 12-14.
36. The method of any one of the preceding embodiments, further comprising selecting a TREM for administering to the subject, responsive to the acquired value.
37. The method of any one of the preceding embodiments, wherein the TREM is formulated as a pharmaceutical composition.
38. The method of any one of the preceding embodiments, wherein the TREM is formulated for intratumoral injection.
39. The method of any one of the preceding embodiments, wherein the TREM is formulated as a lipid nanoparticle formulation.
40. The method of any one of the preceding embodiments, wherein the TREM is disposed in a syringe, e.g., for intratumoral injection.
41. A method of providing a tRNA-based effector molecule (TREM) to a subject having cancer comprising,
   acquiring a value for the presence of a premature termination codon (PTC) signature in a cancer cell; and
   responsive to the acquired value, administering a TREM to the subject,
   wherein the TREM comprises the sequence of Formula A:
   [L1]-[ASt Domain1]-[L2]-[DH Domain]-[L3]-[ACH Domain]-[VL Domain]-[TH Domain]-[L4]-[ASt Domain2],
   wherein independently, [L1] and [VL Domain], are optional, and
   wherein the TREM does not comprise a non-naturally occurring modification, thereby providing the TREM to the subject.
42. The method of embodiment 41, wherein the PTC signature comprises a nonsense mutation or a missense mutation.
43. The method of embodiment 42, comprising acquiring the value for the presence of a missense mutation or nonsense mutation.
44. The method of embodiment 42, comprising acquiring the value for the presence of a nonsense mutation (e.g., presence of TGA, TAA, or TAG codons).
45. The method of any one of embodiments 41-44, wherein the TREM induces an immune response in a cell or subject, e.g., relative to a reference value.
46. The method of embodiment 37, wherein inducing an immune response comprises an increase in the expression or level of a cytokine or an increase in cytotoxic T cells.
47. The method of any one of embodiment 41-46, wherein the TREM comprises a sequence provided in Table 3.
48. The method of any one of embodiments 41-47, wherein the TREM has the sequence of any one of SEQ ID NOs: 1-451.
49. The method of any one of embodiments 41-48, wherein the TREM has at least 80%, 85%, 90%, 95%, or 99% sequence identity to a TREM provided in Table 3.
50. The method of any one of embodiments 41-49, wherein the TREM comprises SEQ ID NO: 100, or has at least 80%, 85%, 90%, 95%, or 99% sequence identity to SEQ ID NO: 100.
51. The method of any one of embodiments 41-49, wherein the TREM comprises a sequence selected from SEQ ID NO: 622, SEQ ID NO: 623, or SEQ ID NO: 624, or has at least 80%, 85%, 90%, 95%, or 99% sequence identity to a sequence selected from SEQ ID NO: 622, SEQ ID NO: 623, or SEQ ID NO: 624.
52. The method of any one of embodiments 41-51, wherein the premature termination codon (PTC) signature is present in p53.
53. The method of any one of embodiments 41-52, wherein expression or level of full-length p53 protein is increased in a cell, e.g., by about 5%, 10%, 15%, 20%, 25%, 50%, 75%, 90%, relative to a reference value, upon administration of the TREM.
54. The method of any one of embodiments 41-53, wherein the expression or level of the p21 protein is increased in a cell, e.g., by about 5%, 10%, 15%, 20%, 25%, 50%, 75%, 90%, relative to a reference value, upon administration of the TREM.
55. The method of any one of embodiments 41-54, wherein the cancer is selected from a cancer provided in Tables 12-14.
56. The method of any one of embodiments 41-55, further comprising selecting a TREM for administering to the subject, responsive to the acquired value.
57. The method of any one of embodiments 41-56, wherein the TREM is formulated as a pharmaceutical composition.
58. The method of any one of embodiments 41-57, wherein the TREM is formulated for intratumoral injection.
59. The method of any one of embodiments 41-58, wherein the TREM is formulated as a lipid nanoparticle formulation.
60. The method of any one of embodiments 41-59, wherein the TREM is disposed in a syringe, e.g., for intratumoral injection.
61. A method of treating cancer in a subject comprising:
   acquiring a value for the presence of a premature termination codon (PTC) signature in the cancer; and
   responsive to the acquired value, administering a TREM to the subject locally, e.g., intratumorally,
   thereby providing the TREM to the subject.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

Table of Contents for Examples

| | |
|---|---|
| Example 1 | Synthesis of exemplary TREMs |
| Example 2 | HPLC and MS analysis of exemplary TREMs |
| Example 3 | Analysis of exemplary TREMs via anion-exchange HPLC |
| Example 4 | Analysis of exemplary TREMs via PAGE Purification and |
| | Analysis |
| Example 5 | Characterization of exemplary TREMs for readthrough of a |
| | premature termination codon (PTC) in a reporter protein |
| Example 6 | Assessment of an exemplary TREM to rescue expression of |
| | a tumor suppressor gene harboring a PTC mutation |
| Example 7 | In Vivo PTC readthrough and target engagement of TREM by |
| | hydrodynamic gene delivery |
| Example 8 | In vivo xenograft studies to demonstrate endogenous PTC |
| | suppression |

### Example 1: Synthesis of Exemplary TREMs

Generally, TREM molecules (e.g., modified TREMs) are prepared and purified by HPLC according to standard solid phase synthesis methods using phosphoramidite chemistry. (see, e.g., Scaringe S. et al. (2004) Curr Protoc Nucleic Acid Chem, 2.10.1-2.10.16; Usman N. et al. (1987) J. Am. Chem. Soc, 109, 7845-7854). TREMs may be prepared to incorporate the naturally occurring nucleotides, or prepared to include one or more non-naturally occurring modifications. Individually modified TREM molecules containing one or more 2′-methoxy (2′OMe), 2′fluoro (2F), 2′-methoxyethyl (2′-MOE), or phosphorothioate (PS) modifications were prepared according to phosphoramidite technology on solid phase used in oligonucleotide synthesis. For clarity, the arginine non-cognate TREM molecule named Arg-TGA contains the sequence of ARG-UCU-TREM body but with the anticodon sequence corresponding to UCA instead of UCU (i.e., SEQ ID NO: 622). Similarly, a serine non-cognate TREM molecule named Ser-TAG contains the sequence of SER-GCU-TREM body but with the anticodon sequence corresponding to CUA instead of GCU (i.e., SEQ ID NO: 623). A glutamine non-cognate TREM molecule named Gln-TAA contains the sequence of GLN-CUG-TREM body but with the anticodon sequence corresponding to UUA instead of CUG (i.e., SEQ ID NO: 624).

### Example 2: HPLC and MS Analysis of Exemplary TREMs

TREMs prepared as described herein may be analyzed by HPLC, for example, to evaluate the purity and homogeneity of the compositions. A Waters Aquity UPLC system using a Waters BEH C18 column (2.1 mm×50 mm×1.7 μm) may be used for this analysis. Samples may be prepared by dissolving 0.5 nmol of the TREM in 75 μL of water and injecting 2 μL of the solution. The buffers used may be 50 mM dimethylhexylammonium acetate with 10% CH₃CN (acetonitrile) as buffer A and 50 mM dimethylhexylammonium acetate with 75% CH₃CN as buffer B (gradient 25-75% buffer B over 5 mins), with a flow rate of 0.5 mL/min at 60° C. ESI-LCMS data for the chemically modified TREMs may be acquired on a Thermo Ultimate 3000-LTQ-XL mass spectrometer.

### Example 3: Analysis of Exemplary TREMs Via Anion-Exchange HPLC

This example describes the quality control of a synthesized TREM via anion-exchange HPLC. Using the Dionex DNA-Pac-PA-100 column, a gradient is employed using HPLC buffer A and HPLC buffer B. 0.5 ODUs of a sample that has been dissolved in H2O or Tris buffer, pH 7.5 is injected onto the gradient. The gradient employed is based on oligonucleotide length and can be applied according to Table 15. The parameters provided in Table 16 can be used to program a linear gradient on the HPLC analyzer.

TABLE 15
Oligonucleotide length and gradient percentages

| Length | Gradient |
|---|---|
| (bases) | (% B) |
| 0-5 | 0-30 |
| 6-10 | 10-40 |
| 11-16 | 20-50 |
| 17-32 | 30-60 |
| 33-50 | 40-70 |
| >50 | 50-80 |

TABLE 16
Parameters for a linear gradient on HPLC analyzer

| Time | Flow | % Buffer | % Buffer |
|---|---|---|---|
| (min) | (mL/min) | A | B |
| 0 | 1.5 | 100 | 0 |
| 1 | 1.5 | 100 | 0 |
| 3 | 1.5 | 70a | 30a |
| 15 | 1.5 | 40a | 60a |
| 15.5 | 2.5 | 0 | 100 |
| 17 | 2.5 | 0 | 100 |
| 17.25 | 2.5 | 100 | 0 |
| 23 | 2.5 | 100 | 0 |
| s23.1 | 1.5 | 100 | 0 |
| 24 | 1.5 | 100 | 0 |
| 25 | 0.1 | 100 | 0 |

### Example 4: Analysis of Exemplary TREMs Via PAGE Purification and Analysis

This example describes the quality control of an exemplary TREM via PAGE purification and subsequent analysis thereof. Gel purification and analysis of tRNA follows standard protocols for denaturing PAGE (Ellington and Pollard (1998) In Current Protocols in Molecular Biology, Chanda, V). Briefly, the oligo is resuspended in 200 mL of gel loading buffer. Invitrogen™ NuPAGE™ 4-12% Bis-Tris Gels or similar gel is prepared in gel apparatus. Samples are loaded and gel ran at 50-120 W, maintaining the apparatus at 40° C. When complete, the gel is exposed to ultraviolet (UV) light at 254 nm to visualize the purity of the RNA using UV shadowing. If necessary, the desired gel band is excised with a clean razor blade. The gel slice is crushed and 0.3M NaOAc elution buffer is added to the gel particles, and soaked overnight. The mixture is decanted and filtered through a Sephadex column such as Nap-10 or Nap-25.

### Example 5. Characterization of Exemplary TREMs for Readthrough of a Premature Termination Codon (PTC) in a Reporter Protein

This example describes an assay to test the ability of a non-cognate chemically modified TREM to readthrough a PTC in a cell line expressing a reporter protein having a PTC. This Example describes analysis of exemplary TREMs (i.e., Arg-TGA, Ser-TAG, and Gln-TAA), though a non-cognate TREM specifying any one of the other amino acids can also be used.

A cell line engineered to stably express the NanoLuc reporter construct containing a premature termination codon (PTC) may be generated using the FlpIn system according to the manufacturer's instructions. Delivery of the TREMs into the NanoLuc reporter cells is carried out via a reverse transfection reaction using lipofectamine RNAiMAX (ThermoFisher Scientific, USA) according to manufacturer instructions. Briefly, 5 μL of a 2.5 uM solution TREMs sample are diluted in a 20 uL RNAiMAX/OptiMEM mixture. After 30 min gentle mixing at room temperature, the 25 uL TREM/transfection mixture is added to a 96-well plate and kept still for 20-30 min before adding the cells. The NanoLuc reporter cells are harvested and diluted to 4×10⁵ cells/mL in complete growth medium, and 100 μL of the diluted cell suspension is added and mixed to the plate containing the TREM. After 24 h, 100 uL complete growth medium is added to the 96-well plate for cell health.

To monitor the efficacy of the TREMs to read through the PTC in the reporter construct 48 hours after TREM delivery into cells, a NanoGlo bioluminescent assay (Promega, USA) may be performed according to manufacturer instruction. Briefly, cell media is replaced and allowed to equilibrate to room temperature. NanoGlo reagent is prepared by mixing the buffer with substrate in a 50:1 ratio. 50 μL of mixed NanoGlo reagent is added to the 96-well plate and mixed on the shaker at 600 rpm for 10 min. After 2 min, the plate is centrifuged at 1000 g, followed by a 5 min incubation step at room temperature before measuring sample bioluminescence. As a positive control, a host cell expressing the NanoLuc reporter construct without a PTC is used. As a negative control, a host cell expressing the NanoLuc reporter construct with a PTC is used, but no TREM is transfected. The efficacy of the TREMs are measured as a ratio of the NanoLuc luminescence in the experimental sample to the NanoLuc luminescence of the positive control or as a ratio of the NanoLuc luminescence in the experimental sample to the NanoLuc luminescence of the negative control. It is expected that if the sample TREM is functional, it may be able to read-through the stop mutation in the NanoLuc reporter and produce a luminescent reading higher than the luminescent reading measured in the negative control. If the sample TREM is not functional, the stop mutation is not rescued, and luminescence less or equal to the negative control is detected.

The impacts of chemical modifications were evaluated in singly and multiply modified TREM sequences and are summarized in FIG. 6. In this figure, the TREMs are annotated as follows: r: ribonucleotide; m: 2′-OMe; *: PS linkage; f: 2′-fluoro; moe: 2′-moe; d: deoxyribonucleotide; 5MeC: 5-methylcytosine. Thus, for example, mA represents 2′-O-methyl adenosine, moe5MeC represents 2′-MOE nucleotide with 5-methylcytosine nucleobase, and dA represents an adenosine deoxyribonucleotide.

FIG. 6 also summarizes the results of the activity screen in column “A” for measurements made using NanoLuc reporter cells at 48 hours post-transfection, which reported as log 2 fold changes compared with the appropriate unmodified TREM, wherein “1” indicates less than a 1 log 2 fold change; “2” indicates greater than or equal to 1 and less than 3.32 log 2 fold change; and “3” indicates greater than or equal to 3.32 log 2 fold change. The results show that certain modifications were tolerated at many positions, but particular sites were sensitive to modification or exhibited improved activity when modified.

### Example 6: Assessment of an Exemplary TREM to Rescue Expression of a Tumor Suppressor Gene Harboring a PTC Mutation

This example demonstrates the in vitro ability of a TREM as disclosed herein to readthrough an endogenous PTC mutation in Calu-6 lung carcinoma cells.

Calu-6 is a lung cancer cell line known to harbor a nonsense mutation in TP53 (R196X; Arg→TGA) that results in a premature termination codon (PTC). To test PTC readthrough of the TREM, cells were treated with Arg-TGA, Ser-TGA, or Ser-TAG TREMs or with known translational read-through inducing drugs (RIDs). Briefly, 6×10⁵ cells were seeded into a 6-well plate and transfected with 100 nmol of TREM in 15 uL of RNAiMAX+800 uL OptiMEM; 0.5 or 5 mg/mL G418, an aminoglycoside antibiotic; or Ataluren, a TRID that is approved for the treatment of DMD in Europe at the reported maximally effective dose of 10 uM.

To monitor the ability of the TREMs to readthrough the PTC mutation, the expression of TP53 was assessed by Western blotting 48 hours post-TREM delivery into the cells. Cells were washed once with phosphate buffered saline (PBS) and lysed with 100 uL of RIPA buffer (50 mM Tris, pH 8.0, 150 mM NaCl, 1% Nonidet-P40 (NP-40), 0.5% Deoxycholate, 0.1% sodium dodecyl sulfate (SDS)) supplemented with protease inhibitor, for 30 min at 4° C. The protein extracts were then centrifuged at 21,000 g for 15 min and the supernatant was transferred into clean tubes. Protein concentration was determined using a BCA Protein Assay Kit according to the manufacturer's instructions. 20 μg of total protein were loaded into an SDS-PAGE gel and transferred onto nitrocellulose membranes after electrophoresis. The membranes were probed with anti-p53, anti-p21, and anti-β-tubulin antibodies.

The Arg-TGA TREM and, to a lesser extent Ser-TGA TREM, were able to robustly induce PTC suppression and resulted in the production of full-length p53 protein in a manner that was considerably more efficacious relative to either G418 or Ataluren (FIGS. 1-3). Ataluren was ineffective in inducing TP53 stop suppression. Surprisingly, about 90% of the total p53 in the Arg-TGA TREM-treated cells were full length, indicating strong penetrance of PTC suppression (FIG. 3). In contrast, G418 marginally increased the full-length p53 protein levels, but was more effective in rescuing mRNA levels with a subsequent enhancement in the expression of truncated p53 protein. The results indicate that G418 can stabilize TP53 transcripts, but is not as effective as the TREMs in inducing PTC suppression.

To determine whether the rescued p53 protein was functional, cellular p21 protein levels were measured (FIG. 4). p21 is a downstream transcriptional target and well-established biomarker of p53 activity. The Arg-TGA TREM was able to rescue p21 protein levels while the other treatments did not provide significant rescue (FIG. 4).

These results demonstrate the ability of TREMs to functionally rescue an endogenous PTC mutation in cancer cells.

### Example 7: In Vivo PTC Readthrough and Target Engagement of TREM by Hydrodynamic Gene Delivery

Hydrodynamic gene delivery (HGD) is a simple, fast, safe, and effective method for delivering transgenes in rodent models. A set of plasmids expressing both an eGFP-Luc-TGA reporter and a TREM were designed. To evaluate tolerability and determine optimal plasmid concentration for maximum TREM delivery to the liver, the eGFP-WT Luc plasmid was administered to adult CD-1 mice via tail vein hydrodynamic injection at three doses: 10 μg, 30 μg, and 50 μg. As shown in FIG. 5A, plasmids in saline were successfully delivered to liver in a dose-dependent manner as shown by the luciferase readout signal. Next, 50 μg of DNA in saline (100 mg/kg) was administered to mice via tail vein hydrodynamic injection to assess target engagement and PTC readthrough using either 1) eGFP-Nluc TGA reporter plasmid (PL-854), or 2) eGFP-Nluc WT reporter (PL1202), or 3) all-in-one plasmid eGFP-Nluc-TGA reporter with S-TAG (PL-1216), or 4) all-in-one plasmid eGFP-Nluc-TGA reporter with R-TGA (PL-1215). The Arg-TGA selectively rescued the TGA nonsense mutation in the reporter plasmid and showed a ˜1000-fold increase in luciferase signal compared to controls (FIG. 5B).

### Example 8: In Vivo Xenograft Studies to Demonstrate Endogenous PTC Suppression

This example describes administration of a TREM to suppress an endogenous PTC stop in an in vivo xenograft mouse model.

Immunocompromised mice are implanted with a tumor cell line with an endogenous PTC mutation in a gene known to support tumor growth and proliferation. After 14 days of tumor growth and formation, the mice are randomly divided into groups of 6 mice and are administered intratumorally the following: vehicle control, G418 (an aminoglycoside antibiotic), 2,6-diaminopurine (2,6-DAP), and a TREM as described herein. After cancer cell implantation, tumors are allowed to grow for 1-3 days after intratumoral injection, after which the mice are sacrificed and blood and the tumor are collected for analysis.

Successful readthrough of the PTC by a TREM as described herein is measured by TREM quantification, full-length protein expression of the gene of interest (e.g., p53) and proteins important for the function of the gene of interest (e.g., p21), and tumor progression or regression using molecular and biochemical assays known in the art. Biological activity of TREMs in an in vivo mouse model is determined by assessing tumor volume. In vivo studies are used to also determine TREM tolerability and exposure favorable for further studies.

1. A composition for use in treating a proliferative disease in a subject, the composition comprising a tRNA-based effector molecule (TREM), wherein,
prior to administering the TREM to the subject, acquiring a value for the presence of a premature termination codon (PTC) signature in the cancer; and
responsive to the acquired value, administering a TREM to the subject locally, e.g., intratumorally.

2. The composition for use of claim 1, wherein the TREM comprises the sequence of Formula A:
[L1]-[ASt Domain1]-[L2]-[DH Domain]-[L3]-[ACH Domain]-[VL Domain]-[TH Domain]-[L4]-[ASt Domain2],
wherein, independently, [L1] and [VL Domain], are optional.

3. The composition for use of any one of claims 1-2, wherein the PTC signature comprises a nonsense mutation in a cancer cell (e.g., nonsense mutation in a tumor suppressor gene).

4. The composition for use of any one of claims 1-3, wherein the TREM is selected from (i) a TREM that does not comprise a non-naturally occurring modification and (ii) a TREM comprising a non-naturally occurring modification that induces an immune response in a cell or subject.

5. The composition for use of any one of claims 1-4, wherein the TREM does not comprise a non-naturally occurring chemical modification.

6. The composition for use of any one of claims 1-4, wherein the TREM comprises a non-naturally occurring chemical modification.

7. The composition for use of claim 6, wherein the non-naturally occurring modification is present on the nucleobase, sugar, or in the internucleotide linkage of the TREM.

8. The composition for use of any one of claims 6-7, wherein the non-naturally occurring modification is present know on the sugar of the TREM.

9. The composition for use of claim 8, wherein the non-naturally occurring modification comprises a 2′ modification.

10. The composition for use of claim 9, wherein the non-naturally occurring modification comprises a 2′-OMe, 2′-MOE, 2′-halo (e.g., 2′-F), or 2′-deoxy modification.

11. The composition for use of any one of claims 6-7, wherein the non-naturally occurring modification comprises an internucleotide modification.

12. The composition for use of claim 11, wherein the non-naturally occurring modification comprises a phosphorothioate modification.

13. The composition for use of any one of claims 6-12, wherein the non-naturally occurring modification induces an immune response in a cell or subject, e.g., relative to a reference value.

14. The composition for use of claim 13, wherein inducing an immune response comprises an increase in the expression or level of a cytokine or in a cytotoxic T cell.

15. The composition for use of any one of claims 6-14, wherein the non-naturally occurring modification comprises a sugar modification (e.g., a 2′-OMe, 2′-halo, 2′MOE, or 2′-deoxy) or a modification in the internucleotide region (e.g., phosphorothioate).

16. The composition for use of any one of the preceding claims, wherein the TREM comprises a nucleotide sequence provided in FIG. 6.

17. The composition for use of any one of the preceding claims, wherein the TREM comprises a nucleotide sequence of an arginine tRNA consensus sequence, e.g., a nucleotide sequence of Formula I_{ARG} (SEQ ID NO: 565), Formula II_{ARG} (SEQ ID NO: 566), or Formula III_{ARG} (SEQ ID NO: 567).

18. The composition for use of any one of the preceding claims, wherein the TREM comprises a nucleotide sequence of an arginine tRNA consensus sequence and has an anticodon that is complimentary to a stop codon, e.g., TGA, TAG, or TAA.

19. The composition for use of any one claims 1-16, wherein the TREM comprises a nucleotide sequence of a glutamine tRNA consensus sequence, e.g., a nucleotide sequence of Formula I_{GLN} (SEQ ID NO: 577), Formula II_{GLN} (SEQ ID NO: 578), or Formula III_{GLN} (SEQ ID NO: 579).

20. The composition for use of claim 19, wherein the TREM comprises a nucleotide sequence of an glutamine tRNA consensus sequence and has an anticodon that is complimentary to a stop codon, e.g., TGA, TAG, or TAA.

21. The composition for use of any one of claims 1-16, wherein the TREM comprises a nucleotide sequence of a serine tRNA consensus sequence, e.g., a nucleotide sequence of Formula I_{SER} (SEQ ID NO: 607), Formula II_{SER} (SEQ ID NO: 608), or Formula III_{SER} (SEQ ID NO: 609).

22. The composition for use of claim 21, wherein the TREM comprises a nucleotide sequence of an serine tRNA consensus sequence and has an anticodon that is complimentary to a stop codon, e.g., TGA, TAG, or TAA.

23. The composition for use of any one of the preceding claims, wherein the TREM comprises a nucleotide sequence having about 60%, 65%, 70%, 75%, 80%, 85%, 90%, 95%, 99%, or 99.9% sequence identity relative to a nucleotide sequence listed in FIG. 6.

24. The composition for use of any one of the preceding claims, wherein the TREM comprises a nucleotide sequence that comprises a nucleotide substitution, e.g., relative to a nucleotide sequence listed in FIG. 6.

25. The composition for use of any one of the preceding claims, wherein the TREM comprises a nucleotide sequence of any one of SEQ ID NOs: 622 and 626-675, e.g., listed in FIG. 6.

26. The composition for use of any one of the preceding claims, wherein the TREM comprises a nucleotide sequence of any one of SEQ ID NOs: 624 and 676-690, e.g., listed in FIG. 6.

27. The composition for use of any one of the preceding claims, wherein the TREM comprises a nucleotide sequence of any one of SEQ ID NOs: 623 or 625, e.g., listed in FIG. 6.

28. The composition for use of any one of the preceding claims, wherein the TREM has the sequence of any one of SEQ ID NOs: 622-690.

29. The composition for use of any one of the preceding claims, wherein the TREM has at least 80%, 85%, 90%, 95%, or 99% sequence identity to a TREM provided in FIG. 6.

30. The composition for use of any one of the preceding claims, wherein the TREM comprises SEQ ID NO: 100, or has at least 80%, 85%, 90%, 95%, or 99% sequence identity to SEQ ID NO: 100.

31. The composition for use of any one of claims 1-29, wherein the TREM comprises a sequence selected from SEQ ID NO: 622, SEQ ID NO: 623, or SEQ ID NO: 624, or has at least 80%, 85%, 90%, 95%, or 99% sequence identity to a sequence selected from SEQ ID NO: 622, SEQ ID NO: 623, or SEQ ID NO: 624.

32. The composition for use of any one of the preceding claims, wherein the premature termination codon (PTC) signature is present in p53.

33. The composition for use of any one of the preceding claims, wherein expression or level of full-length p53 protein is increased in a cell, e.g., by about 5%, 10%, 15%, 20%, 25%, 50%, 75%, 90%, relative to a reference value, upon administration of the TREM.

34. The composition for use of any one of claims 32-33, wherein the expression or level of the p21 protein is increased in a cell, e.g., by about 5%, 10%, 15%, 20%, 25%, 50%, 75%, 90%, relative to a reference value, upon administration of the TREM.

35. The composition for use of any one of the preceding claims, wherein the cancer is selected from a cancer provided in Tables 12-14.

36. The m composition for use of any one of the preceding claims, further comprising selecting a TREM for administering to the subject, responsive to the acquired value.

37. The composition for use of any one of the preceding claims, wherein the TREM is formulated as a pharmaceutical composition.

38. The composition for use of any one of the preceding claims, wherein the TREM is formulated for intratumoral injection.

39. The composition for use of any one of the preceding claims, wherein the TREM is formulated as a lipid nanoparticle formulation.

40. The composition for use of any one of the preceding claims, wherein the TREM is disposed in a syringe, e.g., for intratumoral injection.

41. A composition for use in treating a cancer in a subject, the composition comprising a tRNA-based effector molecule (TREM), wherein,
prior to administering the TREM to the subject, acquiring a value for the presence of a premature termination codon (PTC) signature in a cancer cell; and
responsive to the acquired value, administering a TREM to the subject,
wherein the TREM comprises the sequence of Formula A:
[L1]-[ASt Domain1]-[L2]-[DH Domain]-[L3]-[ACH Domain]-[VL Domain]-[TH Domain]-[L4]-[ASt Domain2],
wherein independently, [L1] and [VL Domain], are optional, and
wherein the TREM does not comprise a non-naturally occurring modification.

42. The composition for use of claim 41, wherein the PTC signature comprises a nonsense mutation or a missense mutation.

43. The composition for use of claim 42, comprising acquiring the value for the presence of a missense mutation or nonsense mutation.

44. The composition for use of claim 42, comprising acquiring the value for the presence of a nonsense mutation (e.g., presence of TGA, TAA, or TAG codons).

45. The composition for use of any one of claims 41-44, wherein the TREM induces an immune response in a cell or subject, e.g., relative to a reference value.

46. The composition for use of claim 37, wherein inducing an immune response comprises an increase in the expression or level of a cytokine or an increase in cytotoxic T cells.

47. The composition for use of any one of claims 41-46, wherein the TREM comprises a sequence provided in Table 3.

48. The composition for use of any one of claims 41-47, wherein the TREM has the sequence of any one of SEQ ID NOs: 1-451.

49. The composition for use of any one of claims 41-48, wherein the TREM has at least 80%, 85%, 90%, 95%, or 99% sequence identity to a TREM provided in Table 3.

50. The composition for use of any one of claims 41-49, wherein the TREM comprises SEQ ID NO: 100, or has at least 80%, 85%, 90%, 95%, or 99% sequence identity to SEQ ID NO: 100.

51. The composition for use of any one of claims 41-49, wherein the TREM comprises a sequence selected from SEQ ID NO: 622, SEQ ID NO: 623, or SEQ ID NO: 624, or has at least 80%, 85%, 90%, 95%, or 99% sequence identity to a sequence selected from SEQ ID NO: 622, SEQ ID NO: 623, or SEQ ID NO: 624.

52. The composition for use of any one of claims 41-51, wherein the premature termination codon (PTC) signature is present in p53.

53. The composition for use of any one of claims 41-52, wherein expression or level of full-length p53 protein is increased in a cell, e.g., by about 5%, 10%, 15%, 20%, 25%, 50%, 75%, 90%, relative to a reference value, upon administration of the TREM.

54. The composition for use of any one of claims 41-53, wherein the expression or level of the p21 protein is increased in a cell, e.g., by about 5%, 10%, 15%, 20%, 25%, 50%, 75%, 90%, relative to a reference value, upon administration of the TREM.

55. The composition for use of any one of claims 41-54, wherein the cancer is selected from a cancer provided in Tables 12-14.

56. The composition for use of any one of claims 41-55, further comprising selecting a TREM for administering to the subject, responsive to the acquired value.

57. The composition for use of any one of claims 41-56, wherein the TREM is formulated as a pharmaceutical composition.

58. The composition for use of any one of claims 41-57, wherein the TREM is formulated for intratumoral injection.

59. The composition for use of any one of claims 41-58, wherein the TREM is formulated as a lipid nanoparticle formulation.

60. The composition for use of any one of claims 41-59, wherein the TREM is disposed in a syringe, e.g., for intratumoral injection.
